SEQUENCE LISTING (2 sequences)

SEQ ID NO: 1 - protein, 5207 aa, Homo sapiens
MECPSCQHVSKEETPKFCSQCGERLPPAAPIADSENNNSTMASASEGEMECGQELKEEGGPCLFPGSDSWQENPEEPCSKASWTVQESKKKKRKKKKKGNKSASSELASLPLSPASPCHLTLLSNPWPQDTALPHSQAQQSGPTGQPSQPPGTATTPLEGDGLSAPTEVGDSPLQAQALGEAGVATGSEAQSSPQFQDHTEGEDQDASIPSGGRGLSQEGTGPPTSAGEGHSRTEDAAQELLLPESKGGSSEPGTELQTTEQQAGASASMAVDAVAEPANAVKGAGKEMKEKTQRMKQPPATTPPFKTHCQEAETKTKDEMAAAEEKVGKNEQGEPEDLKKPEGKNRSAAAVKNEKEQKNQEADVQEVKASTLSPGGGVTVFFHAIISLHFPFNPDLHKVFIRGGEEFGESKWDSNICELHYTRDLGHDRVLVEGIVCISKKHLDKYIPYKYVIYNGESFEYEFIYKHQQKKGEYVNRCLFIKSSLLGSGDWHQYYDIVYMKPHGRLQKVMNHITDGPRKDLVKGKQIAAALMLDSTFSILQTWDTINLNSFFTQFEQFCFVLQQPMIYEGQAQLWTDLQYREKEVKRYLWQHLKKHVVPLPDGKSTDFLPVDCPVRSKLKTGLIVLFVVEKIELLLEGSLDWLCHLLTSDASSPDEFHRDLSHILGIPQSWRLYLVNLCQRCMDTRTYTWLGALPVLHCCMELAPRHKDAWRQPEDTWAALEGLSFSPFREQMLDTSSLLQFMREKQHLLSIDEPLFRSWFSLLPLSHLVMYMENFIEHLGRFPAHILDCLSGIYYRLPGLEQVLNTQDVQDVQNVQNILEMLLRLLDTYRDKIPEEALSPSYLTVCLKLHEAICSSTKLLKFYELPALSAEIVCRMIRLLSLVDSAGQRDETGNNSVQTVFQGTLAATKRWLREVFTKNMLTSSGASFTYVKEIEVWRRLVEIQFPAEHGWKESLLGDMEWRLTKEEPLSQITAYCNSCWDTKGLEDSVAKTFEKCIIEAVSSACQSQTSILQGFSYSDLRKFGIVLSAVITKSWPRTADNFDDILKHLLTLADVKHVFRLCGTDEKILANVTEDAKRLIAVADSVLTKVVGDLLSGTILVGQLELIIKHKNQFLDIWQLREKSLSPQDEQCAVEEALDWRREELLLLKKEKRCVDSLLKMCGNVKHLIQVDFGVLAVRHSQDLSSKRLNDTVTVRLSTSSNSQRATHYHLSSQVQEMAGKIDLLRDSHIFQLFWREAAEPLSEPKEDQEAAELLSEPEEESERHILELEEVYDYLYQPSYRKFIKLHQDLKSGEVTLAEIDVIFKDFVNKYTDLDSELKIMCTVDHQDQRDWIKDRVEQIKEYHHLHQAVHAAKVILQVKESLGLNGDFSVLNTLLNFTDNFDDFRRETLDQINQELIQAKKLLQDISEARCKGLQALSLRKEFICWVREALGGINELKVFVDLASISAGENDIDVDRVACFHDAVQGYASLLFKLDPSVDFSAFMKHLKKLWKALDKDQYLPRKLCDSARNLEWLKTVNESHGSVERSSLTLATAINQRGIYVIQAPKGGQKISPDTVLHLILPESPGSHEESREYSLEEVKELLNKLMLMSGKKDRNNTEVERFSEVFCSVQRLSQAFIDLHSAGNMLFRTWIAMAYCSPKQGVSLQMDFGLDLVTELKEGGDVTELLAALCRQMEHFLDSWKRFVTQKRMEHFYLNFYTAEQLVYLSTELRKQPPSDAALTMLSFIKSNCTLRDVLRASVGCGSEAARYRMRRVMEELPLMLLSEFSLVDKLRIIMEQSMRCLPAFLPDCLDLETLGHCLAHLAGMGGSPVERCLPRGLQVGQPNLVVCGHSEVLPAALAVYMQTPSQPLPTYDEVLLCTPATTFEEVALLLRRCLTLGSLGHKVYSLLFADQLSYEVARQAEELFHNLCTQQHREDYQLVMVCDGDWEHCYLPSAFSQHKVFVTPQAPLEAIQAYLAGHYRVPKQTLSAAAVFNDRLCVGIVASERAGVGKSLYVKRLHDKMKMQLNVKNVPLKTIRLIDPQVDESRVLGALLPFLDAQYQKVPVLFHLDVTSSVQTGIWVFLFKLLILQYLMDINGKMWLRNPCHLYIVEILERRTSVPSRSSSALRTRVPQFSFLDIFPKVTCRPPKEVIDMELSALRSDTEPGMDLWEFCSETFQRPYQYLRRFNQNQDLDTFQYQEGSVEGTPEECLQHFLFHCGVINPSWSELRNFARFLNYQLRDCEASLFCNPSFIGDTLRGFKKFVVTFMIFMARDFATPSLHTSDQSPGKHMVTMDGVREEDLAPFSLRKRWESEPHPYVFFNDDHTTMTFIGFHLQPNINGSVDAISHLTGKVIKRDVMTRDLYQGLLLQRVPFNVDFDKLPRHKKLERLCLTLGIPQATDPDKTYELTTDNMLKILAIEMRFRCGIPVIIMGETGCGKTRLIKFLSDLRRGGTNADTIKLVKVHGGTTADMIYSRVREAENVAFANKDQHQLDTILFFDEANTTEAISCIKEVLCDHMVDGQPLAEDSGLHIIAACNPYRKHSEEMICRLESAGLGYRVSMEETADRLGSIPLRQLVYRVHALPPSLIPLVWDFGQLSDVAEKLYIQQIVQRLVESISLDENGTRVITEVLCASQGFMRKTEDECSFVSLRDVERCVKVFRWFHEHSAMLLAQLNAFLSKSSVSKNHTERDPVLWSLMLAIGVCYHASLEKKDSYRKAIARFFPKPYDDSRLLLDEITRAQDLFLDGVPLRKTIAKNLALKENVFMMVVCIELKIPLFLVGKPGSSKSLAKTIVADAMQGPAAYSDLFRSLKQVHLVSFQCSPHSTPQGIISTFRQCARFQQGKDLQQYVSVVVLDEVGLAEDSPKMPLKTLHPLLEDGCIEDDPAPHKKVGFVGISNWALDPAKMNRGIFVSRGSPNETELIESAKGICSSDILVQDRVQGYFASFAKAYETVCKRQDKEFFGLRDYYSLIKMVFAAAKASNRKPSPQDIAQAVLRNFSGKDDIQALDIFLANLPEAKCSEEVSPMQLIKQNIFGPSQKVPGGEQEDAESRYLLVLTKNYVALQILQQTFFEGDQQPEIIFGSGFPKDQEYTQLCRNINRVKICMETGKMVLLLNLQNLYESLYDALNQYYVHLGGQKYVDLGLGTHRVKCRVHPNFRLIVIEEKDVAYKHFPIPLINRLEKHYLDINTVLEKWQKSIVEELCAWVEKFINVKAHHFQKRHKYSPSDVFIGYHSDACASVVLQVIERQGPRALTEELHQKVSEEAKSILLNCATPDAVVRLSAYSLGGFAAEWLSQEYFHRQRHNSFADFLQAHLHTADLERHAIFTEITTFSRLLTSHDCEILESEVTGRAPKPTLLWLQQFDTEYSFLKEVRNCLTNTAKCKILIFQTDFEDGIRSAQLIASAKYSVINEINKIRENEDRIFVYFITKLSRVGRGTAYVGFHGGLWQSVHIDDLRRSTLMVSDVTRLQHVTISQLFAPGDLPELGLEHRAEDGHEEAMETEASTSGEVAEVAEEAMETESSEKVGKETSELGGSDVSILDTTRLLRSCVQSAVGMLRDQNESCTRNMRRVVLLLGLLNEDDACHASFLRVSKMRLSVFLKKQEESQFHPLEWLAREACNQDALQEAGTFRHTLWKRVQGAVTPLLASMISFIDRDGNLELLTRPDTPPWARDLWMFIFSDTMLLNIPLVMNNERHKGEMAYIVVQNHMNLSENASNNVPFSWKIKDYLEELWVQAQYITDAEGLPKKFVDIFQQTPLGRFLAQLHGEPQQELLQCYLKDFILLTMRVSTEEELKFLQMALWSCTRKLKAASEAPEEEVSLPWVHLAYQRFRSRLQNFSRILTIYPQVLHSLMEARWNHELAGCEMTLDAFAAMACTEMLTRNTLKPSPQAWLQLVKNLSMPLELICSDEHMQGSGSLAQAVIREVRAQWSRIFSTALFVEHVLLGTESRVPELQGLVTEHVFLLDKCLRENSDVKTHGPFEAVMRTLCECKETASKTLSRFGIQPCSICLGDAKDPVCLPCDHVHCLRCLRAWFASEQMICPYCLTALPDEFSPAVSQAHREAIEKHARFRQMCNSFFVDLVSTICFKDNAPPEKEVIESLLSLLFVQKGRLRDAAQRHCEHTKSLSPFNDVVDKTPVIRSVILKLLLKYSFHDVKDYIQEYLTLLKKKAFITEDKTELYMLFINCLEDSILEKTSAYSRNDELNHLEEEGRFLKAYSPASRGREPANEASVEYLQEVARIRLCLDRAADFLSEPEGGPEMAKEKQCYLQQVKQFCIRVENDWHRVYLVRKLSSQRGMEFVQGLSKPGRPHQWVFPKDVVKQQGLRQDHPGQMDRYLVYGDEYKALRDAVAKAVLECKPLGIKTALKACKTPQSQQSAYFLLTLFREVAILYRSHNASLHPTPEQCEAVSKFIGECKILSPPDISRFATSLVDNSVPLLRAGPSDSNLDGTVTEMAIHAAAVLLCGQNELLEPLKNLAFSPATMAHAFLPTMPEDLLAQARRWKGLERVHWYTCPNGHPCSVGECGRPMEQSICIDCHAPIGGIDHKPRDGFHLVKDKADRTQTGHVLGNPQRRDVVTCDRGLPPVVFLLIRLLTHLALLLGASQSSQALINIIKPPVRDPKGFLQQHILKDLEQLAKMLGHSADETIGVVHLVLRRLLQEQHQLSSRRLLNFDTELSTKEMRNNWEKEIAAVISPELEHLDKTLPTMNNLISQDKRISSNPVAKIIYGDPVTFLPHLPRKSVVHCSKIWSCRKRITVEYLQHIVEQKNGKERVPILWHFLQKEAELRLVKFLPEILALQRDLVKQFQNVQQVEYSSIRGFLSKHSSDGLRQLLHNRITVFLSTWNKLRRSLETNGEINLPKDYCSTDLDLDTEFEILLPRRRGLGLCATALVSYLIRLHNEIVYAVEKLSKENNSYSVDAAEVTELHVISYEVERDLTPLILSNCQYQVEEGRETVQEFDLEKIQRQIVSRFLQGKPRLSLKGIPTLVYRHDWNYEHLFMDIKNKMAQDSLPSSVISAISGQLQSYSDACEVLSVVEVTLGFLSTAGGDPNMQLNVYTQDILQMGDQTIHVLKALNRCQLKHTIALWQFLSAHKSEQLLRLHKEPFGEISSRYKADLSPENAKLLSTFLNQTGLDAFLLELHEMIILKLKNPQTQTEERFRPQWSLRDTLVSYMQTKESEILPEMASQFPEEILLASCVSVWKTAAVLKWNREMR

SEQ ID NO: 2 - DNA, 235000 bp, Homo sapiens
acccaggaggtggaggttgcagtgagccaagatcgcgccactgtactccagcctgggttacagagtgagactctgtctcaaaaaaaagaaaaaaaagaaaaagaaaagaaaaagaccacacattaatccaggactgtggggctcctcccaggaggaggtggccctgacccctgacctgtatgcttcctgggaaatgtgcttctggggaggacagagggaggaggaaggagagggaggaagaggaaggcaggggagagaagggaggcattacaggaggctttgagcccaggggaagaggggttttccctctggtctggtgggtccggcacggaggcggggggagagcatgatgggagcacagtgggaaggggacccaggtttacctccgggtcaggagcctggctgtggtctggcatggaggcagggggagggcatgatgggagcacagtgggaaggggacccaggtttacctccgggtcaggagcctggctgtggtctggcatggaggcagggggagggcatgatgggagcacagtgggaaggggacccaggtttacctccgggtcaggagcctggctgtctcctgtgtctaaggaacaggcccgggagtctgcagggcccctctctggggcgtgcttttccccctcagtctcctgggcacagcctggggacctgctcattggcaggacagaggctgcctgggctttcgggggagcacacagatgccgttccctccctccctcttttctcccgtatctcccacgttcagggtgaacccagaaaaccctggggaatggaagtgctttgggaatgtgggcttctgccaaggcctgtggtccctccatcgtagccactccttgagcagcctgtgccagcagggaggagaggagggggtgcagcatctcgccgggaacctgagtccacagccgaggatgactgaccctccctcttgtgtgacaacaggattctgcaggacagagagatgaaactggaaataattcagtccaaacagtcttccaagggacccttgctgctacgaaaaggtggctccgagaagtttttacaaagaacatgctcacatcttcaggtgcctcattcacatacgtcaaggaaattgaggtaggcatttggccgaaggcttctgggtagggatgtgactgatcgtgattcctcatggcctcaaatcatggggtacaggctgctggccaggggcagttgttggccttcacctgagcctcagttcttcatctacaaaatggagttgctcctgtctacctggcagggtggctctgaggcagacagatgccgggtgcaaagtgctgctgcactgcctcgtgtcccctggccgggcacagtccctgtggtgatggaggtgtggaggtgatggtgatggttggtggtggtggtggtagaggtgatggtggtggaggtgatggtgatggttggtggtggtggtggtagaggtgatggtggtgaaggtgatggtgatggtggacgtaatggaggtgatggtggtgatggtggtcatggaggtggtggaggtgatggtggtggtggaggtgatggtgattgtggtggaggtgatggtggtgatggtgatggttgtggaggtgatggtggaggtaatggaggtgatggtggtggtgaaggggatggtggtgatggaggtggtggtggtggaggtgacaatggtgatgctggtggtggaggtggtggaggtactggaggtgatggtggtggtgaaggtgatggtggaggtaatggaggtgatggtggtggtggaggtgatggtggaggtactggaggtgatggtggtggtgaaggtgatggtggaggtactggaggtgatggtggtggtggtggtgatggtggaggtggtggaggtaatggaggtgatggtggtggtggaggtgatggtggaggtactggaggtgatggtggtggtgaaggtgatggtggaggtaatggaggtgatggtggtggtggtggtgatggtggaggtggtggaggtaatggaggtgatggtggtggtggaggtgatggtggaggtactggaggtgatggtggtggtgaaggtgatggtggaggtactggaggtgatggtggtggtgtaggtgatggtggaggtgatggtggtggtgaaggtgatggtggaggtaatggaggtgatggtggtggtgaaggtgatggtggaggtactggaggtgatggtggtaaaggtgatggtggaggtactggaggtgatggtggtggacgtgatggtggaggtaatggaggtgatggtggtggtggtggtggtggaggtactggaggtgatggcggtcgtggaggtgatggtggaggtaatggaggtggtggtggtggtggtggtggaggtactggaggtgatggtggaggtaatggaggtgatggtggtgatggtgatggtggaggtactggaggtgatggtggtggtggtggaggtaatggaggtgatggtggtggtgaaggtgatggtggaggtaatggaggtgatggtggtggtggtggtggtggtggaggtaatggaggtgatggtggtggtggtggtggtggtggaggtactggaggtgatggcggtcgtggaggtgatggtggaggtaatggaggtggtggtggtggtggtggtggaggtactggaggtgatggtggaggtaatggaggtgatggtggtgatggtgatggtggaggtactggaggtgatggtggtggtggtggaggtaatggaggtgatggtggtggtgaaggtgatggtggaggtaatggaggtgatggtggtggtggtggtggtggtggaggtaatggaggtgatggtggtggtggaggtgatggtggaggtactggaggtgatggtggtggtgaaggtgttggtggaggtactggaggtgatggtggtcgtgtaggtgatggtggaggtgatggtggtggtgaaggtgatggtggaggtactggaggtgatggtggtggtgaaggtgatggtggaggtaatggaggtgatggtggtggtgaaggtgatggtggaggtaatggaggtgatggtggtaaaggtgatggtggaggtactggaggtgatggtggtaaaggtgatggtggaggtaatggaggtgatggtggtggacgtgatggtggaggtaatggaggtgatggtggtggtggtggtggaggtactggaggtgatggtggtggacgtgatggtggaggtaatggaggtgatggtggtggtggtggtggaggtaatggaggtgatggtggtggtggtgatggtggaggtactagaggtgatggcggtcgtggaggtgatggtggaggtaatggaggtgatggtggtggtggtggtggtactggaggtgatggtggaggtaatggaggtgatggtggtggtggtggtggtactggaggtgatggtggaggtaatggaggtggtggtggtggtggtggtggaggtactggaggtgatggtggaggtactggaggtgatggtggtcgtggaggtgatggtggaggtaatggaggtgatggtggtggtggtggtggtactggaggtgatggtggtccgtggaggtgatggtggtggtggtaatggaggtgatggtggtggtactggaggtgatggtggtggtgaaggtgatggtagaggtaatggaagtgatggtggaggtactggaggtgatggtggtggtggtggaggtgacagtggtgatgctggtggtggtggtggtggtggtggtggtggtggaggtggtggtggtggtggtggtggtgatggtgatggtggtggtggtggtggtggaggtgatggtggaggtaatggaggtgatggtggtggtgaaggtgatggtggtgatggaggtggtggtggtggaggtgacagtggtgatgctggtggtggaggtggtggaggtactggaggtcatggtggtggtggaggtgatagtggtgaaggtgatggaggtggtggaggttatggagataatggtggtggtggaggtgatagatatttgaacatgcctgacctaagaaaagttcattttcatttttggctgggcactatggctgatgcctgtaaccccaactctttaggaagcctaggtggaagggtggcttgaacccaggaggtcagggctgcagtgagctatgactgtgccactgcactccaacccaggtgacggagcgagaccctgtctcttaaaatattttttttacagtgcattttcatgtgtttcaacctcctagtgtccctgccaaaaatattttaatctgaatcaaatcatggggaaattatgagacaaatcaggtcaaaagacagtttacaaaacagttggcctgaacttttcaaaactgtcaacatgttaaaagaaaaaagaaaaaaatgaccagggaactcttctagaaaagagatgtgataagtaaatgcagaaaagagacgtgataagtaagtacagcgtgtgtcccaggaggatctaggatccagtcaggacaggagctggagcacgagaggaacgggggtggagaccacttcctgggggcagagtgtcctcctctccacgttgcacgtcctgagtgggaaacctgcactggagttgcacagggaagggctagggtcttaggagaccctgcccaggtatgtagtggcaaggtgccatgatgcctgcaacttactttccaacggtttgggagagaaaatgctcatttgtgcagagtccgaagagggagcacagaggagagaggaagagtgaaagcagctgtgygaagcgtgaatcgcacaggactggagttgaatgaatgtgggtggtggtgtgctattcttgcgacctttttgaaagttcaaaaattttcaaagtataacatgagagataagtcctttcagaataaaaagccggttgtgcagtaggaggaccagtagtggcccctggttgggtctctgattctagaaagccaacctgggctgctgtgaccggccactagcatggctgactgttgatgaaggttggggagagccctggtgttcgcggagtcccgcgctctctgtattgccgtaatgctctgtctttctcctttcatgggagtgtgccgtggcatttagtcgtgagagtgggtgtgacctgtgtgcgggttttgcaggtctggaggcggctggtggaaatccaattccccgcggagcatggctggaaggagtcgttgctgggagacatggaatggaggctcacaaaggtaccaaaagtttgggggcagtcttttcagggcgtgaaggcaagctggagaaccccagaccattagcgacagccaagagatctcagcagtgcctctctgtgggcagggatggggtgaatcacagctccgtgtttctgtttctgatccagcccttatagtctgtccctagaagagcgcttcggagtcttactgagaggacagagaagaacaagggcccaggatctcaccatcatgggggtgcgggatttttcctgaccccttcgttggacttgcgacagggatgctgtgtttacttggcccgccgtgctcaaccccttgtgggagggagcacgtgagtgagtgtggcatctggccagctgctttgggagctggcaggagcaagctccgtgcaggccctgcagcagtgcccagattgaggtgcctgcaacccccgaagctccagagggcatgttacagtgctctcttagctccgccgtctatggacagtagtgtgttatcagctcagtgggtcccttgccttgttgcctagggtggctgccctctgccagggccaatgtgacagcctcttttaggtacccacactcggtgggtcccaagctcttgtccagcatccaagaagaatggggtcacccggacacttgaaggatggtgaaggtggagaattttattaagcaatggaaatggctctccatggagaggggagctggagaggggatgggtcaggtggggaatcttcctctggtccaggtgtctggctggctcttccctgaagtcaggacgtctttccgaagttaaaccgtctctccttggaagtccagccatccctctgaagtcaagtcacctgtttccagtgaagctacttctctctcactaccaagtctggggtctttataggcacaagatagggggcagggtgggccctaggtagttttggaaaaagcaacattctattggtaaaaagacattactccgaaagaaccagtggggagagagtaggcacacggggatagaggttctcactttgggccatgggtttcaggcttttcggctcgaaggtggggttttgccagggacccgcccctgtctgcgtaggatttctctgcctcctgcctccatcaatggggccccaggcacaggaggggccaggcctgcccataggcaggaagtctaagtggcctgttggtgacaggaacctcatgacacacaagtggagaacacacagacatgcccctcatgctgaattaaggcctgaggctcaaggaaaattagtttttatgtgatatgagagttttatatgtaactttttttttttttgagacggggtctcgctctatcgcccaggctggagtgcagtggcgggatctcggctcactgcaaactccgcctcccgggttcacgccattctcctgcctcagcctcccgagtagctgggactacaggcgcccgccaccgcgcccagctatatttttgtattttttgtagagacggggtttcaccgtgttagccaggatggtctcgatctcctgacctcgtgatccacccgcctcagcctcccaaagtgctgggatgacaggcgtgagccaccgcgcccggcctatatgtaactgttaaatggatctttaaagggaaaagtcagaaacaaggaaaaatgacctatgtctcataacttacctgtttttgcaagccaggtgctttttctgcagagtgggaaaagagactcttttaaattttagcctttggtctactggtggggggcagggaggacatgctttgcgtgggccaggagaagcttaaaattgggggaaacagtaaaacagcaggaaagtaaaaatgatgaaagaattttcatcccatagagcactggagcgctgcatccgaaagctctgaaaccacccccctttgatttttgcaggaggaacccctctcccagatcactgcctactgcaatagttgctgggacaccaaaggcttagaggacagtgtggccaagaccttcgagaaatgcatcattgaagccgtgagctcagcctgccaggtgaacaatctctcctcctgggaaacggattcgggctcacagctgtgttctgccatgacccagctaagggctatgaagcacccgctgggtctcagctcctccgctaactcagagattgggaagtgggcaccctcctccctcgccaagggcaatggcgctgaaatctagttctctccggattcctcagtgtgctgcacagtccctgctgctcgcaccatcctgcatgtgttccatatggaatcacggccgtgcgcgtgtggcacaagtcacacgggcttgcaggccgttcctcagatggccctgtcatcactgtggctgctggtttgattgattgttaacacttgctcagtaggtgtgcgggaagagactccaaaggttgacagaacatttatggaagcaaaatatgtgaaatggaaaattgtatcaatttatttagctcttttcggcaaaggggaagagattgtgcccccctgtctcccaggaacagtctcgcaggcaatgccacatgaggaagctccctgctggccacggctgccctgctcacatttcctaattggacacttaacccctgtacaagcacagccttgcggccacaggggaagtccagaaacattgaggtcattgaattccggggaccaaggggttctaattttttaagtgactgatacctttgataaggttttcctttccctttttcgttaactctttgttgagatattgttcgtatgccatacggtacatctgtcaaaagttccagttcagcaggttttggggtagtcacagatatgtacagtcatcaccacagttaatgacagagcattttcatcacttcaaagagaaacccggcccctttagccatcatcctcctcccctctagtcacccactcccctctgcaggcataaacaattgctgaacataaacaactgcttctgtggctttctctgttctgactgtcatatgaatggaatcatatcatatgtggccttttgggtatggcttatttcactgagcataatgtttttttgttgttggtggtggtggttgtttgttgtttttgagacagagtttcactctttttgcccaggctggagtgcaatggtgcgatcctggctcaccgcaaccgctgcctcccgggttcaagtgattctcctgcctcagcctcccaagtgctggaattacagctactttttgtttaaagagtcttttaattgtttaaagaacaatgtgccgccacactcggttccttttgtatttttagaagagacagggtttctccatgttggtcaggctggtctcgaactcccgacctcaggtgatccacccaccttggcctcccgaagtgccgagattacaggtgtgagccaccgcgcccggccgagcataatgttttgaaagaccgctcaggctggacacggttgctcacgcctgtaatcacggcactttgggagcccaggagttcaagacaagcctgggtaacagagtaagaccctgtctctataaaaactaaaaaataaacaaaaaaaattagccaggcatggccgggcacacctgtggtcccagctacttgggaggctgaggtgggaggctcccttgggcccagaaggtcaaggcagcagtgagccatgatcacaccactgcacttcaacctgggggacagagcaagaccctgtctcaaaaagcaataacaacaaaagtccatccatattgtagcttgtgtccgtttacgttaagtattccttacccaaaatgcttgagaccagaagtgttttggatttcagatgttttcaaattttggaatatttgcatttacataatgagatgtcttccagatgggacccagagtctaaccacaaaattcacttgtttcatatacatcttatacacatagcctgaaggtaattttatacaatatttttaacaattttgtgcatgagacaaagtttgtattaagtatttgtatgttgaatttttcacttgtggcatcattatactcaaaaagtttggattttgtagtattttggatttggggattaggaatgctcaacctatatttcatttttttccatggccaaatattccccgtttatccatgtgtccattgacggccatctatgttgcttcttcggctattataaatctgctgatacaaacattagtgtacatatttatgtgtgggtgtatgtcttcatttctcctgggtgtatacctcgatgtagaattgctgaatcatatggtaactccatgtttaattgtttaaagaactgtccggccgagtcccacagcagctgaaccattttcaatccctccagcagcataggaggcttccaggttctccacatcctcactaacatgtgttgtctaactttttgattctagccatcctggtgggtatgaagtggtatctccttgtggtttttgtttgttttttgagatggagtcttgccctgtcacccaggctggagtgcagtggcgtgatcttggctcactgcgacacctccacctcctgggttcaagcaattctcctgcctcagtctctcgagtagctgggactacaggcatgtgccaccatgcccagctaatttttgtatttttggtagagacggggttttgccgtgtgggtcatgatggtctcgatctcttgacctcatgatccacatgccttggcctcccaaagtgctgggattacaggcgtgagccactgtgcccggcctcattgtggtttttaactgcatttctctgataactaatgatatcaagcatcttttcatatgcttattggatttgtctgtatcttctttggagaaatgtttattgagatattttgcccctcatcccccccacccccccttttttttttttttttttgagaaaaggtctcgctttgtcacccaggctggaatacaatggcacgatcacagctcactacagcctcaacctcctgggctgaagtggtcctcccactttagcctcccgagtagcagggactacaggcatgtgccaccatgttcagctaattaaaaaaaaaatttctgtgggccgggcatggtggctcatgcctgtagaccatcctggctaacacggtgacaccctgtctctactaaaaatacaaacaattagctgggcatggtggcatgtgcctgtagtcccagctgcttgggaggctgaggccagagaatcacttgaagccatgaggtggaggttgcagtgagctgagattgtgccactgcactccggcctgggcgacagagcgagactctgtctcaaaaaaaaaaattttttttcgtagagacagggtcttgagatgttgcccaggctggtcttgaactcctggactcacatgaacctccctccttggccttccaaagtgttgggattgaaggcatgagccaccgcacctggcctcttttgctcatttttaaattgggttgtctttttattatagagttgggagtctttattctatatacaagtcacttgtcagatatatgattgcaagtattttctcccattctatgtgttgttttttcacattcttattggtttcttttgaagcacagaagtctttaattttgaagaagttagcatatctgttttttcttttgttgcttgtacttttggtgtcaaagaatcctttgccaaatcctaggtgatgaagatttactcctatgtttatttatctctaaatgttttatagttttagctcttacatgtaggcctttgatccattttgagttcctttttatatatggtgtatatggtgtgaggtaagggttctgtttcattctcttgcatgtggacatccagttgtcccagcatttgttgaaaggactctacattccctgttgagtgatgttggcactcttgtcaaaaatctgttgacgatacatgtatggatttatttctagattttcaattctatgtggtgatctacttgcctatccttgtgtcagtaccacactgtttggactaccattgctttctagtaagttttgaaattgggatgtgtgagtcctcctactttgttcttatttttcaagattgttttggctattttgggtcccttgcaatttcataagaattttagaatcagtatgtcagtttccactgacaaggaagtcagctgggattctggtagggctggtattcaatgtatagatcagtttggagagcattgccatcttaacaaggttaagttggatttctttcattcaattagatttttttttttcccctgttagacagagtctcgccctgttgcccaagctggagtgcccaagtagctgggactacaggtgtccaccaccatgcctggctaatttttgtatttttagtagtttcaccatattggtcagggtggtctcgaactcctgacctcagatgatcaacccgccttgacctcccagagtcctgggattacaggcatgagccaccattcctggccaatttcttttaacaatgttttgtatttttcatagtttaaattttgcacttcttttgttaaatttctaagtacttttttttgggggggggtgctattgtaattggaattgttttctgaattctgtttccagattgttccttgctggtgtctagacacacagtgctccatgcgtcttgtatcctgcaacattgctgagctcatttggtagctcaaattgtgtgtgcgtgtgtgtgtgtgtgtgcatgagagagagaattaaattttctatacataagatcatatcatctgtgaatagagtttcacttcttccaatctggatgccttttatttctttctcttgcataactgccctggctagaacctccagtacaatgttgaatgaaagtattgaaagtggacatctttgtcttgttctgatcttaagagaaggtttcagtcttttgccaccaggtgtgatgtcaacgtgagcttttcatagatgccctttatcagtttgtggaagttcctttttgttcctcatttgttgaatgcttttttttctttaatcatagaagagagttggattttgtcaaatgcttttcccgtatctactgaaatgatcatgcgatattttcctaatgttattaactgatatttgatgttaaatcaacctttcatccctgagataaattacacttgatcgtggtgtatagttcctttaacatggggctggataaggttttcttttatccttttaaatgtcagaaagtatctattataaattaaccttgtggtttttactaattactgaaatgttttacagaaactttgaacctaaaggaagaaaaatattgtttctcggaggagaattaaaaagagaattaaaatgtatatgttgtttgtaggcagctttttaaaagaattatggtttaggaacataaaatatatgctatattaagggtttattcatttttggtatttttcatgatatgaaagtccaaaaaaaaatgtagaaactggcagccagaaggaaggagggtgactggttatgttggtgtgtgtttgtgtgtgtgtcttcacatctctggtgctggacacttaaggaggcagtatacaaggaaatgaaaattttcgaagaaatgtaaagtgttttactattttgctctttttgtggccttataaaacaagttacagtagatttcagaaatgctatcgagtaggtaatttgcttttgttatttcaaaacaaactaatgaaaaacttctaaatgtccctcttttattaattttcttgtagtctcagaccagtatccttcaggggttctcttactctgatttgcggaaatttggcatcgtcttgtctgctgtgatcactaagtcctggcctaggaccgcggacaacttcaatgacattttaaagcatctgctcacgttggcagatgtcaagcacgtcttcagattgtgtggtatgtttgcgggagtgctgggaacatcagctcaggcaaggtggtgtcttcctgattgggaaaggtgggaggcagaagatttgacatgatgatactgaattgggatgggctgatgtttgacaaaggctgcagtttggacagagagaccctggaaggctcagagtgtcttgcgttgtaaataataactaatgctaacagccactattcattgaatgcttgctgtaccctagcgactgtgctgggtcgcaagggaagtgcccttgaaacaacccttctgaaagtcccctttaaggcccccagttttcagatcaggaaacaaatggagaaggcaatgcgacttacctaaggtctcaaagattataagtagtaaaaactaaaatccagacccaggtatgtcttatttggagatcaatatagaatgtccaagccaacattgatgacctcaacagaaagtgaagtactgtgctgtctgatttccccccagccccccgcgccccaggtgttttgtggtcaaggagggagagagagagacattccatcctaaaaacgttagagtccagttggcgtgtgagaccttagattcatatctttcagtgagaaggcgctggatgtggaacagccaaaatcaatgctgagcttcctacacatcagggaagctgtggggatcaaataactgaattaatatgggagtgcacaagtaaaagctactctcttgaaaagatcatttttgtgttatttgtatttatttatttatttatttatttatttatttattttggagatggagtctcgctctctcacccaggctggagtgcagtgctgtgatctcagctcactgcaacctctgcctcccgggttcaagcgattctcctgcctcagcctcccaagtagctgggattacagatgtgagccactgtgcccagcagtttatttaattttaaaacaaggtcttctctgttgcgcaagttagcatgtagtagagtgatgccatcatcgctcaccgtaagcgctgacctcccgggctcacgtgatcctcctgcctcagcctcccaagtggctggcactccaggcgtgcaccaccatacccagcctgtttataagttggatagaattttttagagcactttgatgtgtacagaaaaattgagcagaaagtacagagagttgtcatctatctctgccaccatcacacgggcagtaccctgttagtaacatgcagcattagtgtggtgcttttgttacagctgagaagccaatattgataacactgttattaactaaagtccatagttcgttcctggtgttgtccattctctgaggtttggataaaccatggcctgtgtccaccattccagtatcgtgcagaatagttgcgctgcccctacaaaatcccatgctccacccctcccatttcctttagtcttttccagaatgtcctatggttggaatcttataggatgtcgcctttagactggcttccttcacttagtaatacgtatttatgtttcctccatgtcttttcatggcttgagagctaacttcgttttaccactgaataatattctattgtatggatgtgctaccattcgcttacccgttcacctcatgaaagacatcttggttgcttccaagtttttgcaatgattagtaaagctactatgtttgtttcttatggtgcagataagcttcatcttctcctaatattgtatggctctaatattactgggagagaaatcctaagtggttaaacaaaacatgtataaaaattctgccctctaaatttatcataaaatcagttccaagattttgcaagataatgaagttgtacagagtataaagttatcacatggctgtttctctctttgcatatgctatgaaagtgtctccctaagaccatcctgatagacattgctaggtatgtctgcatcctctggtgcagtttcttctgttgtaggtattcctagggcaggggctttttaaagaagattctgtgtattctatttaaaaaatatactcaggccatgtgaggtggttcatgcgtataatcctaatgttttgggaggccaaggcgggaggatctcttgagcccaggagtttgagaccagcctgggcaacaaagtgagaccctgttctctacaaaaaattttgaaagtttgccaggcatggtgctgtgcacctgtgttcctagctactcgggaggctgaggcaggaaaatgaggttacagtgggctatggttgtgccactgcactccagcctgggtgacagagagagaccctgtcaaaaaaaaaaaaaaaaggaagtcagtccatccgtatactgaaggggacgtgggttttaacctgagaaatgtgtctcccagctgattttggccattgcgggaagtcatcttcttggacaaggaagttgttcaggggatgaatctgggcaagacctaaccctcgtgggggaaagcagaagggagtggaatgattggcgcatctggagacactggccagccattgcctgtgtttgaggaagtgggggcagaacaagaggttatctggaattcccacggtaacggcaaagaggcaggaggtggggagcccactgtgttaactgtgttcttcatctattcaggaaccgacgagaaaatactagcaaatgtcacagaggatgccaagaggctgatagctgttgccgactctgtgttgacgaaagttgttggtgacctcctaagtggcacgattttagttggacaactggagctgattataaagcacaagaatcagtttcttgacatctggcaactgagtaagcatcgagtcgatacgcacttcaggctcctgaggcattaagtagctggaagacacgtttgtgtttgcgtgtgcatttttgtaaaggagataatcatcttagccttgataatgagtatctcttcttgctcataagttgataggggtggtttataattctcaagaacatcacgggaaagtgataaaataatggccatctcgaaaaagtgggtatgcgcaaaaccatcctagtccttctcctggtggcggggaaggtgcggcgctttcaaagcattgccatggaaaatggcatttgttgctgtattgggttactttattggtgttcttattttccaggggaaaaaagtctttcaccccaggatgaacaatgtgctgtggaggaagcactggattggagaagggaggaactgttacttctaaagaaagagaaaagatgtgttgatagtctcctgaagatgtgtgggaacgtgaaacatctgatacaaggtggtattcctgagaagtgaacaaagttgagggctctcaaagggttagaggagaacacagtaagaatggatcacagtagcagatctttattttggagagaaggctttaaaattttttttttaatttgcatttgctggggggatcgctttcacctcttttagattcttctttttggtatttatgtccacatttccaaataacatgcttaagtgtctcctggatttttccggagtcagcatgagctgctgacgcccgctgcagaagatgagggcttagagccacacatttctggtctgtgccctgcacacatcacctttccacctcctgttgcttcctgttttctcataacggagttgcatcattgtttagggttaaatcatcaggacccacacggatcacaagtgaaccacgaaagaaaccgtcatcattttacaatttttgcacaaactctctgttttccacagaattacatttcctgtcagatgctgttttcactgcgttaatgatacgctgttttcattgtattaatttctaattccatcgcaatctctgccgagtgtctgaatttccttgaagatcattggtttgacctttgaaaaggaacctgccaaccctgggctgcagacgtagggtggagtctccctccacctcctaggagcctttgcctggcacctgggttagacgtggttcccacgcccccctcccccgctactcactcctgcttcctctgttggcagagcacaccctcactggcttcctgagaaaagggcgtgtaagaggttaaaccctcgaatccttgtgtgtctaaagatgtctgtcctcttactaggtcagtcgctcggctaggtctagaattacagaccggaagtcattttccttctgaagtttcaatgtgctgcgccatcattgcctccacagtgtggctatgtatggagaagtccgatgcagtgccagttacacatccttcatgtgtgtgacctgttttctctttcttgaagctcttgagctctttctgttactcatattctgaaataataacgtgtcttggcctggatctatgttccatcgaggctgggcatgttggctcccacctgtaatcccagcacttcaggaggccaaggcaggaggactgcttgagtctaggagtttgagaccagcctgggcaacatagcaagaccccatctctaaaaaaaatacaaaaaaattagcaagacacggtggtgcatgcctgtattcccaactacttgggaggctgaggcaggaggatcacttgagcctgggaagtcgaggctgcagtgagccgaaatcacgccacaacactccagcttgggtgacagagtgggaccctgtctcaaaaaataaaatattttcagtcccctctattgggtattaataggtaggtctttcaactctagacatcaaaatccttgagttctgggaaattctcttgaatgatttaattttttcttctcttgctggaattcctgttaattggatatgggacctcctggatgatcatctaactgtcatcatttttctctcggattttctatctctgttttttgctctgctcttgagagatttattcagaaccaaactttctgcttaggttttcatgtcagctttcgtatatatatttaatgtagaggaattctttttctcggaatgttcctttttaaattctgtcctgttcttgttcacgttgcagcatcttcccttctgggttattcaccgttttgttttggaggttttgcttttcctggcatcatgtgctgcttcctccaggttcctttcacttgttgatttgttttgattttcctcctatgtctggacattcttgtttgtctgtgtttattaagagaaagccctcacaagtgggttggccctctgcggggtgctgccgtggggtgctgtggctcgacctggaagttggcctgtctcctgaagtcttctgtctcctgcctggagggtgtgggcctggctgccaggtttctgggagccaagagggagaagcgggctggcgggccttggcatcccgtgcgtggtcctcccatggcccgtgttctcagtgtggttctccgccacctccctctgtctcctgtgtttcacaccccatcctccttccccctgagattggataattcatgtcttctgttagcttcctgagaaaggggatgtgtctagaaaggccctttgttctgccatgtatctgtggcctccaatccacacgcccccctgcgttatcctttctagaaaatgaaccatctcttggctgctggggtgaagaagggggtccacctgctgtgtcagggcattggggtgacagttggtgtgctggatggaccatgtcttttttttgggacagagtcttgccctgttgtccaggctggagtgcagtggctcagtctcggctcactgcaacctctgcttcctgggttcaagtgaatcttgtgtctcagcctccctagtagctgagattacaggtgtccaccaccacgtccagctagttttttgtatttttagtggagatgggatttcatcatgttggccaggctggtcttgaactcctgacctcaagtgatccatccgcctcagcctcccaaagtgctgggattacgggcgtgagccactgtgcccagcctggaccgtgtcttaaacagactttgaacccagtcttccttatttcatcaccacctcccagtctgcagtgacctgcacaatgtgtgtctgctttttgggaatcctgtgacataaaactactcagctctcagtgttgcccactgggaccctaggactccatgttaccaggcctcatgtgtcatttaccactggttcagctcgttttcaacttctaaaattttgttcctgttatcctgttcgccattcctcctcctgtccttgtcagtttatgacttttttttttttttttgatatggagtctcactctgtcacccaggctggagtgcagtgacacagtctcagctcactgcaacctccacctcccaggttcaagtgattctcctgcctcagcctcctgagtagctgggattacaggtgcgctccaccacgcctggctaatttttgtatttttagtagagacagggttttgtcatgttggccaggctgctcatgaactcctgacctcaggtgatccgcccatctcagcgtcccaacgtgctgggtgggattacaggcatgagccactgcgcccaactagtttatgactttttaaatgcaatttatgttctgtcattggagtggggtttggcaaagagagtaaataggtgcgtgttttcccactgccatctttacccagaagtttatgatgctttctttatcatgaatattggttaacaaggccaagcggggagggaaagacctgtgaactcttcctgagaaagaaatcctagcagctgtgtgctcttgagttcgctcagagaagtgaggagagaaagtcttaggaaagtgaaataaaatggaatcatgattagagctttgacttctgacactttcttttcgcttctaaagtggactttggagtgcttgcagtaagacactcacaagacctcagcagtaaaagattaaatgacaccgtgacagtgagactgtccacctcctcgaactcgcagagggcaacgcattaccacctgagctcccaggtccaagaaatggctgggaagatagacttgctcagagacagccacatcttccagctcttctggcgggaagccgcagagccgctgagtgagcctaaggaggaccaggaagccgcagagttgctgagtgagcccgaagaagaatcagaaaggcacatccttgagcttgaagaggtgtatgactatttgtatcagccttcttacagaaagttcattaagttgcaccaggatctaaagtcaggagaggtcacccttgcagagattgatgtcatcttcaaggactttgtgaataaatacacggacctggattcagaacttaagatcatgtgcaccgtggaccaccaggaccaaagagattggatcaaggaccgagtcgaacagatcaaggaataccatcacctgcaccaggctgtccacgcagccaaggtcatcttgcaggtcaaagagagcctgggactgaacggtgacttcagtgttctcaacactttactaaattttgtaagttatttgctggggactgtgggggtttggaggggcgtcctgcctcagcctcttcaggagccatgggctttgtggctttgaaaaggggggcggatgacttagagcttccgtcaggggctcctgtgtgtgtggtacttgaggcctgaatatcccaagggctttgtttttacctttgttataaatagtaccactgctccttagccagagggagtttccttgggtctgcgtgaagatcaagaactcaggaaagggcaacaatcctcttttcataacgggcatttgtaaagggtcataggctctctctaggttacagaagccaaaaagcaaaacactttgtcggctgcccaactgcatctcccagttccacccaaaagcggtaaatcagttgtttctgttagagttaagaggtgactcttttgcttttttttttttgagatggagtctcgctctgtcgtccaggctggagtgcagtggcgcgatcttggctcactgcaagcttcgcctcccaggttcacgccattctcctgtctcagcctcccaagtagctgggaccacaggtgcccgcaaccacgcccggctaatttttttggtatttttttttttttttagcagagacagggtttcaccgtgttagccaggatggtctcgatctcctgacctcgtgatccacccaccgtggcctcccaaagtgctgggattacaggcgtgagccaccgtgcccggctgagtcttctgcttcttatgtttttaggtagaccgagataatgattagaatttagctgggcacagtggctcatgcctgtaatcccagcactttgggaggctgaggtgggcagatcacctgaggtgaggagttcgagaccagcctgaccaacatggagaaaccccatctctactaaaaatacaaaaattagccgggtgtggtgtcaggtgcctgtaatctcagctactagagaggctgaggcaggaggatcgcttgaaccacggaggcggaggttgcagtgaaccgagatcgtgccactgcactccagcctgggcgacagagtgagactcttatctcaaaaaaaaaaaccaaaaaaaaaacctggaatcccatccatttacacttgaaccctagtgcacgtcctcgaggaagcaactgctctgggcctttattgacaaagcagaaaaagcaacagtagttacttggcagggttatgtgaggaattgattttaaaatccactttattaaaatgcttagcagaatatcaggttcccaggaatcacagttcatcttgatcagggagaaacagccatgggggtttgatcatccaattcaggggtaagagcttggtgtgcctgttgtcacagcagtggcaaatctaggcattgagggagggtggggctgctgggacggtcagtgcttgcagctcacagattctggggttctggttaatgagttccagtccacagtagagctttttcttgcagactgataacttcgacgactttcgccgtgaaacactggaccagatcaaccaggagctcatccaggccaaaaagctgctccaggacatcagcgaggcccggtgcaaggggctgcaggctctgtccctgagaaaggagttcatctgctgggtccgggaggctcttggaggtaaaatcagcctttggggttgcgtggaagtgtggagaaagttttcagatggcgcactgtgtggcgttcctaaactctttggcaatacctccccttggccaaggactacgtaaagggcagaaagacgttgcctgcatgctttagggagatgggcactgatcatagagttcacaccggtacagtatttcccatgtgctgctgtaaaagccactcaccttgccacaccccgccacttcggaagctgtaaatggctcaggttccttttccttaaaatcttgcccccttcagctttttcttccagtagatttttactggaggacaaggagagttcctgcccacagagaggttttttttttttgtttgtttgtttttttgagacagagtctcgctctgtcgcccaggctggagtgcagtggcgtgatcttggctcactgcaagctctgcctcccgggttcacgccattctcttgcctcagcctcccgagtagctgggactacaggcgaccgccaccacgcccggctaattttttgtgtttttagtagagacggggtttcactgtgttagccaggatggtctcgatctcctgacctcgtgatcagcccgtcttggcctcccaaagtgctgggattacaggcatgagccactgcacctggcctttttttttttttgagacggagtttcgctcttgttgcccaggctggagtgcagtggtgcaatctcggctcactgcaacctccacctcctggattcaagcgattttcctgtctcagtctcctgagtagctgggattacaggcgcccgccaccacacccagctacttttttgtatttttagtagagatggggtttcaccgtgttggccaggctggtcttgaactcctgacctcaggtgatccacccatctcagcctcccaaagtgcaggcgtgagcaagtacaggcgtgagccaccgcgcctggccccacagagagatttttctcacttctccattgatgctgtgcgtttcttggcagcctcttcagttctgtggcttagatgtcgttggcagaggagagaggaagaggcagaggaggcaggaggggcacaggtggcagtgcaggtgtgctcgggggtctgggggccaccacctgctggatctcaggctctggctctcttgggagagccgctgctttcagtggtggactctctggtccacactgatgcacatccttcagtgagtaatttatctgaggggaatgatctgagtgtttcatagttaattgtgaacatgttatttacagctgagccttggtttctttttgtttaaatggtaatgttgtgtggattgaaggaaagagcatacacagaagtgcagtacagtgcctgggacattgaaattgctcaggaacggcaagatatcgtttagtgctattaacagacagtacaggctgggcgtggtggctcacacctgtaatctcagcactttgggaggccaagtcgtgtggatcacctgaggtcaggagttggagaccagcctgaccaacatggtgaaacctggtctctactaaaaatacaaaaaattagccaggcgtggtggtgggcgccagtaatcccagctacacgggaggctgaggcaagagaatcacttaaacctggaaggtggaggttgcagtgagccgagatcatgtcacttcactccagcttgggtgacagagtgagactctgtctcaaaaaaaaaaaaaaaaactacaaaagattaggtgttccggactcttactgaaagcagaaagtggacagaaccttaaaaattcatgaaatatccatttcagcttcagtaaggattactgcccaagaccgagtcttgtgctatcgtggaatagtcccacgctgaactcccctcttctcttccccaggcatcaatgagctgaaggtgtttgtggacctggcctccatctcagcgggggagaatgacattgatgtggaccgggtggcctgcttccatgacgctgtgcagggctacgcatccctgctatttaagctggaccccagcgtggacttcagtgcattcatgaagcatctgaaaaagctgtggaaggctctggataaggaccagtacctgcccaggaaactggtgagtcttattctgtctctaatgcagattttgttgaatagagcattgcttagcaacgttagggcagtgactgtggaaatggtggcacacctgtgtggtaggtttgtacacagccgttttcaatgatatttaaaaagcttttcttcataaatggaaattcaccatcttgttacctagaaagcaggatacaaaattacatgacacatagtaatctctaaggtgcaaaacacacgtggaaaaaagacagaagggaaatatgctgaaacgttgccaggagttttctctgaatggttgtaattgtcattttattctgttactttttgttattttctataattgatagaattttttatcagaaaaaagtaaacatttaaaattagccaggcatggtggtgtgcgcctgtaatcccagctactctggaggctgagacagaattgcttgaacccgggaggcagagatagcagtgaactgagatcacaccactgtactccagcctgagtgacaaagtgagactctgtctcaaaaaaaagaaaagaaaagaaaaaagtaaacatttaagtttaagtaaactacctgtagcagggtctgaattgacgtacgcagcttaactagatacctgggccagcctccggacggctgctctgcatgtgggtggaggaagaagtgacatatggaagggcaggtaaagctatcttcatttttctgctagaaagtcagcagagtttccaaacaaaaccctctgttgtgatttttcctaaacttctcttattgtacttggggaccacagaaggacactgaggacttgggactccacagccattgcttccagcgtcacccccagagggttctctgcattggggacctggcagaatgatggcgtctggtagagaggcaggcgggcggctgctcattcccctccactcctccacagatgtaagtcaagtgcccccatgtgccagccggggcaccaggcactggaggaaacgtggaacagcgagtacaaagcagcactgagtgacagatcatgttgtcagggtggcacctggtccagctggggcgctgggtgggcgactgcgtcctgagccctggggaagcgtggggagaaggctctgcagcgaggcagaggcgggaggccgaccacaggagggagaagggcaagatcctcgctccaaccgtggcccgtgttctctccttttgtccccatagtgtgactccgccaggaacttggaatggctgaagactgtgaatgagagtcatgggtctgtggaacgctcatccctgaccctggccacggccatcaaccaaagaggcatctatgtgatccaggcgcccaaaggtggccaaaaggtgagcggtccccagccctcggcgcagctgcggcccttctgcaggctgctgccagtccaggtcttctggccccaagaaagtgacttctaacctatgctcttcacagatttccccagacacggttctgcacttgatccttcctgagagccctggcagccacgaggagtcacgagagtactctttagaggaggtgaaggagcttttgaacaagttgatgctgatgtctggcaagaaggatcgtaacaacacggaagtggagaggttttcagaggtgagggcgcatctttgcagtggcgctaagctggtggtgtcatctcgtccgtgagggctgtgtactcccaagaaaacggaaaggatttgacttgggatttgactgataagaagggctctgctcttaggcccatggagagtagagttggcacatttgtagggtgcatggaactctagtccgcggccccagagaggtcttaaggcaggcaggcccctgggttagcagtccctccttacgatccgtaggatgaacgctgaagctggcagcctgtccaccgcactgagaaggaggttggtcacccagcaaatagcccttagaaagacacttgaattgcctctctcactctgtaaatcagcaagaatccaaataactaagatttagatggacaaaacagaagtaatcatattgagaatggtgtaccggtctaagtaatagtctctcaattttattataggttctttctctaaagttgatttgtactttacaaatggtatcctttatctctgctgggtgcagtggctcacacctgtaaagccagcactttgggagcccaaggtggaaggctcattttagcccaggagttcatgaccagcctgggcaacatagcgagaccctgtctcaaaaagtataaggtttttttttttcccaattaaaaaaaaaagataatttaaaaaaattaaaacagtccaggcatcatggttcacactttgggaggccgaggcgggcctatcacttgaggtcaggtgtttgagaccagcctggcgatatggtgaaaccccatctctactaaaattacaaaaattagccgggcatggtggcacgtgcctgtaatcccagctgctccggagactgaggcaggagaattgcttgaacccaggaggcagaggttgcagtgagccgagatcacaccactgcactccagcctgggcaacaatgcaagactccatctcaaaaaaaaattaaaaaaaaaaaaaaattaaaacaaatgataccatttatctctacaatgactctttccctgatgagagtgccatcttctcaggtgggttgtgtagatgaggagggaggccttgtgagcgcagatcatgcagtgggcctgtggacagggccgtcttttggcggtaggcctgtgtgctgttgcagagtagcgtgttaccctctcgcatggctctgtgagccaacctcatggttctgcctctccaggtcttctgcagtgtgcagaggctcagccaggccttcatcgacctgcactctgctgggaatatgctgttcaggacgtggatcgccatggcctactgctcccccaagcagggtgtgtccctccaaatggactttggcttggacctggtgacggagcttaaagaaggtggagatgtcactgagctgctggcagccctctgcaggcagatggagcacttccttgacagctggaagagatttgtgacccagaagcgaatggagcacttttacctgaacttctacacggcagagcagctggtttacctgagcactgagctcaggaagcagcccccgagtgatgccgccctaacgatgctatccttcatcaaaagcaactgcaccctgagggatgtcttaagggcctctgtggggtgtgggagtgaggccgccaggtaccgcatgaggagagtcatggaagagctcccgctgatgctcttatcagagttcagcctggtggacaagctgaggatcatcatggagcagtccatgaggtgccttcctgccttcctgcccgactgcctcgacctagagacccttggccactgtctggctcacctggcagggatgggtgggtctcccgtggagcgttgtctcccgagaggtctgcaggtcggccagcccaacctcgtcgtctgtggccactccgaggtgttgccagccgccctggctgtctacatgcaaaccccaagccagcccctgcccacttacgatgaggtgctgctctgcaccccggcaaccacctttgaggaggtggcactgttgctgcgccgctgcctgaccctgggctccctggggcacaaggtctacagcctgctgttcgcagatcagctgagctacgaggtggcacgccaagcggaggagcttttccacaatctgtgcacgcagcagcaccgagaagactaccagctcgtcatggtctgtgatggggactgggagcactgctacctcccctctgccttcagccagcacaaggtcttcgtcaccccccaggcacccctcgaggccatccaagcctacctggcaggtcactaccgggtcccgaagcagaccctgtcggcggcagccgtgttcaatgaccggctgtgtgttgggatcgtggcctcggagcgagcaggtgttggtaaggagagcggcagggtgggcaggccccgtctcccagggactgcccggggcccttcccccctccagcagatgaagcctgtctgcaggtggagaaaccgaggctcagggagggtgtgattcatctgtgggtgtcaatcagggtctgtgccagaaccaggcaccctggtcacctggtctcccacaatgggtattccctgggagccccattcccttcctcctctcagcaaacatctctgcagtgtactttgtggtttttttttttttttttttttttttttttgagacagagtcttgctctgtcacccaggctggagtacagtggcgtgatcttagctcactgcaacctccgttttctgggttcaagcaattatcctgcctcagcctcctgagtagctgggattacaggtacccaccgccatccctggctaatttatttttttttgttttttgttttttgtttttttgagacagagtctcactctgttgcccagattggagtacagtggcgtgatctcggctcactgcaaccttcgcctcccggattcaagcgattttcctgcctcagcctcccaagtagctaggactacaggcatgcaccagtacacccagctaatttttttatttttagtagagatgtggcttcaccgtgttagccaggatggtctcgatctcctgacctcatgatctgccctcctcggcctcccaaagtactgggatcacaggcatgagccaccgtgcccggcctaatttttgtatttttagcagagacagggtttcaccatgttggccagtctggtctcaaactcctgacctcaggtgatccgcccacctcagcctcccaaagtgctgggattacaggtgtgagccaccacgcccggcctgaagtttacttcttctgcccttaaatgccactggcctctttggggttgatgttggtcgtgtgagcaaggggaggtagaattgaagagaaagccatgcctcttgacccacccttcgtttgccatcttgtttcccttaaaaagaaatagtagcttctgttctgttgactttatcctatcaacagtacctcgtcctgttgggataaacattactcttcccctccctgctgaagcctctccttttgctgtgacttttattagctagtcactcaacacataatgacatttcagtcaacagcagaccacgtatatgaccacggtcccataagattagaaaactgtactttaaaaattagccaggcatggtggtgcatgcctgtgatcccagctactaggaaggctgacttgggaggatcgctccagcccaagaggtcgaggctgtggtgagctgcaaatacgacactgcactccagcctaggcaacggggcaaggccttggtcttttaaaaaaagagaaagaaagaaagaacaaaagattataataccatattttttactgtaccttttctacatttttatttttatttttgagatggagtctcactctgttgcccaggctggagtgcaatggcacgatctcggctcactgcagcctctgcctcctgggttcaagcgattctcctgcctcagcctcctgagtagctgggactacaggcacacgccaccacacctgactagtttttagtagagatggggtttcaccatgttggccaggctggtctcaaactcctgaccttgagtgatcacccgcctcagcctcccaaagtgctgggattacaggcctgagccaccgtgcctggcctgaatatgtttagatattcagatatttgccccgtgtcacagctccccactgtatgtaggatgcccccatgctgtcacggcttgcagcccaggggcaatgggctattccacctagctcgggtgtgtggtcggctctaccatctgggtttctgcaggcacactctgatgttcacagtgacagaatcatttcacaatgcatttttcagaacgtgtccctagtgctaagtggcgcgggactctgctttgcctgctgtcctgcggaggcaggaggtgaccaggagagtgagtaggtaacggctgggaaatgtgaaggttcaagagcgtattcttgaatctgatgttatgttgcatcagttttgaattttatacaaagagaagtaacttctagtggatctttatagattctcttactctttgtcctagccctgggacttaatttttctctctctctctctctctatttttatatatatacacacacatgctgtatatacatacactatatatattcgatacatattctatatagatttctatatatattctatatatttttacatatataatatatattctatatacttccatatatactatatatattctatatatacatatatagtatgtatatatatattatatatatattgtatgtatatatatattatatatatatattttttgagacagagtttcgctctgtcaccctaacgatgacaactgcaccctaagggatgtcttaagggatgcccaggctggagtgtagtggtgcaatctcagctcactacaacctccgcctcccgggttcaagcgattctcctgcctcagcctcctgagtagctgggattacaggtgcctgccaccacgcctggctaatttttttgtatttttagtagggacgggatttcaccacgttgaccaggctggctttgaactcctgacctcaagtgatccccccgcctcggcctcccaaagtgctaggattacaggtgtgagccaccactcccagccctaatttctgtattaatgttataggaaagtctctgtacgtgaagaggttgcacgacaaaatgaagatgcagttaaacgtgaaaaatgtgcctctgaaaacaattcgactgatcgaccctcaggtggatgagagccgagtcctgggcgccctgctgcccttcctggatgcgcagtatcagaaggtccccgtgctctttcacctggacgtgacctcctcagtaagtgccctccagcgtcagccgatggccacatcagtaaagacgtggtccccatgtctctgcgtgactcctccccgtgtatagaattccttgtttccacacgcacagtcctgtgaagcttaacagtgggaatgtgctctgagaagtgtgttgtcaggcagtttcctccttgtgtgaacgtcatggtgtgcgcttacactaacctagacgtagagcccactgcacacacaggctttatggtacacactgtcgctcctaggccgcaaacctgtgctgcatatggctgttctgaatcctggagccaattgtaaaacggtgtatttatgtgtctaaacataaaaatggtacagggaaatatagtattaggattttatggggctgcccttggagacatgggccgttgttggctgaaatgttgatgttgatcatcaggatcatcgtgacaaagagcaaaataaggaggggttacttagagttgggagaactcgccatcgtgtcgtgtgtttacacctcgtgcgattctgttcttaggtgcagactggaatttgggtgtttcttttcaagctcctcattttacaatacttaatggatataaatgggaaaatgtggcttcggaacccctgccatttgtatatcgttgaaatcctggaaaggaggacgtcagtgccgtcgaggagctcttcagcgctggtctgtactgtggggcgttttcgcctggcgtggggggctcttgggttctttctggtcttactgaagtggaatggcgcgtttaggagactccacatctttgccttacttagtgcagcagctgaatgcagtgttagtaaagaaaagcccatctttaaactcactttttaatcatgtctgggataaaagtactcttctgttaattagcagtgtcttcaatatttctgttcaattcaggcttcatgctgtatgcccagtctgaatcccagctgtgctctgaatctaggaaattcatctgttgaaagacctcacctttcctaatgccagctaagatgagaaggagatgtttgtcaaaaatttcttagcaaaagagagttttatgttcactcacatacataatacatgtatagcttagtggagaaacttagtttcaggaagttgggttagaaagtgatttcctggctcagaacgcagctcccacagcaatgccttacttaatgtgcaaaagatatgctgtggaaaaaaagactatacagaaagctacatattttcagtgcgtttttcataaaggtccatccaatatagataacgtggagggttaagacacaaaagcatttcttagaaagtcttcttgtaaccatttcattaatgtctctcctttccagcgtacacgtgtaccccagttcagttttcttgacatcttcccaaaagtcacctgcaggcctcccaaagaggtgatagacatggagctgagtgccctgaggagtgacacagagcctgggatggatctgtgggagttctgcagcgaaactttccaaagaccttaccagtatttaagacgattcaatcaaaaccaagacctagacacgtttcagtatcaagaaggctctgtcgaaggcaccccggaggaatgcctccagcatttcctgtttcactgcggggtaataaacccatcctggtcagagcttcggaactttgctcggttcctgaattatcagctcagagattgtgaggcctctctcttctgcaatccgagttttattggcgacacactgaggggcttcaagaagttcgtggtgaccttcatgatctttatggcaagagattttgccacaccatcactccacacctctgaccaaagcccggggaagcacatggtcaccatggatggggttagggaagaagatctagcgcccttctccctccggaagaggtgggagtcggagcctcacccatacgttttcttcaatgacgaccacacaaccatgacattcatcggcttccatctgcagcccaacatcaacggcagtgtcgatgccatcagtcacttgactgggaaggtcatcaagagagacgtcatgaccagggacctgtaccagggcctgctgctccagagggtgcccttcaatgtcgactttgataaactgcccagacacaagaaacttgagaggctctgcctgaccttagggatcccccaggccaccgaccccgacaaaacgtatgagctcacaaccgacaatatgcttaaaatccttgccatcgagatgcggttccggtgtgggatcccggttatcatcatgggagaaactggctgtgggaaaaccaggcttattaaattccttagcgacctgcggcgtggtggtaccaatgctgacaccataaagctggtcaaggtgcacggaggaacaactgcagacatgatctactccagagtcagggaggctgaaaatgtggccttcgccaataaggaccaacatcagttggacaccatcttgttttttgatgaagccaacacaacggaagctataagctgtatcaaagaagtcctgtgtgatcatatggtggatggccagcctctggctgaggactctggcctgcatattatagctgcctgcaatccataccggaagcactctgaggagatgatctgccgtttggagtcagctggtttgggctacagggttagtatggaggagacggccgacaggctgggctccattcctctgaggcagctggtataccgggtccatgctctgcccccgagcctgattcctctggtgtgggactttggacaactgagtgacgttgctgaaaagctctacatccagcagattgtccagagactggttgagtccatcagcctagatgaaaacgggactcgcgtgatcacagaagtcctctgcgcctctcagggtttcatgaggaaaacagaagatgagtgcagctttgtcagcctcagggacgtggagcgctgtgtgaaagttttcaggtggttccacgagcacagcgcgatgctcttagcgcagctgaatgcctttctctccaagtccagcgtcagcaaaaatcacaccgagagagatcccgtcctctggtcgttgatgctggccatcggggtgtgttaccatgcctctttagaaaagaaagactcatatcggaaagccatcgccaggttctttccgaaaccgtatgacgacagcaggctgcttctggatgaaataacacgggcacaggatctttttctggacggcgtacctctgaggaaaaccatcgccaagaacttggccttgaaggagaacgtcttcatgatggtcgtctgcatcgagctgaagattcccctcttcctggtggggaagcccggcagctccaagtctctcgccaagaccatcgtggcagacgccatgcagggcccggctgcctactcagatctcttccgcagcctgaagcaggtccacctggtgtccttccagtgcagcccgcactccaccccacagggcatcatcagcaccttccggcagtgcgcccgctttcagcaggggaaggacctgcagcagtacgtctctgtggtggtgttagatgaggtggggctggcggaagactcacccaaaatgcccctgaagactctgcacccgctgctggaagacggatgcattgaagacgatcccgccccccacaaaaaggtcggcttcgtgggcatctccaactgggcccttgaccctgccaagatgaaccggggcatttttgtgtcacgtggcagccccaacgagacagagctcatagagagcgccaagggcatctgctcctcagacatcctcgtccaggaccgagtccaagggtactttgcgtcctttgccaaagcctacgaaacggtgtgtaagcgccaggacaaggaattcttcgggcttcgtgactactacagcctcatcaaaatggtctttgctgcagcaaaggcttcaaatagaaagccttccccgcaagacattgcacaggctgtccttaggaacttcagtggcaaggatgacatccaagctttggacatctttctggccaatttgcccgaggccaagtgctcagaggaagtcagccccatgcagctgatcaaacagaacatctttgggccttctcagaaggtgccgggtggagagcaggaagatgctgagtcccgctacttactcgtgctgaccaaaaactacgtggcactgcagatcctgcagcagacattcttcgagggggaccagcagccggagattatttttggttctggtttccccaaggaccaagagtacacccagctctgcagaaacatcaatcgtgtgaagatctgcatggaaacaggcaagatggtgttgcttctcaacctgcagaacctctacgagagcctctacgacgcactcaaccagtactacgtccacctcggcggccagaagtacgtggacctcggtctggggacccaccgcgtcaaatgtcgggttcaccccaacttccgcctgattgtcattgaagagaaagacgtcgtgtacaaacactttcccatccccctcattaaccggctggagaagcactatctggatatcaacacggtgctggagaaatggcagaagagcatcgtggaggagctctgtgcgtgggtggagaagttcatcaatgtcaaagcacatcatttccagaagaggcacaaatacagcccctctgacgtcttcatcggctaccactcggacgcctgcgcgtctgtggtgctgcaggtcatagagaggcagggtccccgggccttgacggaggaacttcaccagaaggtgtctgaggaggccaaatcgatcctgctgaactgcgctacgcccgatgccgtggtccggctgagcgcctactcgctgggcgggttcgcagcggagtggctgtcgcaggagtactttcacagacagaggcacaactcctttgcagatttccttcaggcacacctgcacacggcagacctggagcgccacgccatcttcacagaggtgattgtctttctgcacttgtaccctatcccctgtcacccaagagtcctaaatccctcgtgtcaggattcaagattcttccccaggcagcggttagggatcctgcagggagatgctgagacgctgagctctcctccgcggtaagtgtggggagtaggtccagttaaccaggtgctgagtgcctggctctgtgcagtgcaccatgctggtaacagcaccatacacagtgttcacaccaacaaataaagtaccacgtcccaggactgggccaagggcactagggacgcacagatgaatctgtagcccttgtcctttgttagcattagggagcatggagagaccagcacatgaacagatgccatcaatatcatatgataaatgctgtgcttagaggcagcggggagcggtgcagaccaggatgcgagctcagatgagatgttccctaggagaagcccctcgccgatggggaagaatggccgggaagggattgtaggctgagggccttctgtgggcagagccgctgacagctcttagggttcaaggctgtagtgctgagatggggcgatgaggtgcagtcagagagaactactggaggccctcagtgactctgcagagccacatctcagccctatggagcgggcactggaggcacgcttgtctccctggctgggtgaagggagtctgatgaaaggctgttgtggaaaagacgggccggtagggaggccgctggaaccgtacaaaggagaggagcagatggaaccgagagatactcaggaggcaatttggcagggccaataccaccacccagtggggggatggggggtggaggggtccggggcgaccccctggattcctccttagatgagtggacattgcttcatcatgacagataccgcaggaacggacagcaggttgtgggaggctagtgatgatgtccgccggggacaggtttctatgcatcgtgcagctggacgtaggaatctgagctcagtggtgaggcagggattcctgggctagagcagcaggcctcgaggaacgtttcgacttgcactgtgatgggggagtgacctttgggacaggagaattgctgacatctgggggaaggggacagcaccaaccacgaactccatctgaaagcatgagtcgtactataacaggaaatggcatttgtgctgagccctaagggatcatgagaatctcaagtacggaccaagcaggcagagacacgggcctctggggaggagaggaaggcaccctcggggcagggtggggtcgtctctccaatgcctttggccctgtagggcctctcggccccagcgctgccgtgttttgggaaactcctttgggtttggattctgtgccgttgtgtggcaactgcaccgcgctgaacatcgcaggtttctaggatctgtctaaacctggcagcagacttgcaacctttccttgaagtctggcaagtaatttgcatttcttaactctgtagatcaccactttctccaggctgctaacaagtcacgactgtgaaattttagaatcagaggtcacaggcagggctccgaaacccacactcctgtggctgcagcagtttgacaccgagtactcattcctcaaagaagtccggtgaggttccctgccttccctgctgccctctccctccccagccgcagccgtgtggcttctgcgtggcgatggtacccgcgccggttaatgagcgccacagtcacgtgactgtgaaatagatccttgttaaatctctcccagcatccctctcatctccatcatcttctgcaacgcggtgtctggcctaacatcctcctccgagagcaccatcctcggacctcgttaggaggcattccttgggtttcctcccctggactatctgttcttactgaagaaaatcctacctgagtagctgtttctttgtagcctttatgtttcccatcactttggggaattttcttttccatttttttaagacagagaactcacttgaataacttcccttttctttcagaaactgtttaacgaatacagccaaatgtaaaatcctcatttttcagacagattttgaagatggaatccgtagcgcccagctcattgcctcagctaagtatgtttttagtatttttctcagaaactatgtaaaaaacccaaaacgtaggaagtcctagagaattttttccttgaagacagtatagtatcataatcagtatagaaataaaaataacaattcttcccccgcctcattcccccaagtgggattagggaaactgaagagagacaaaaagaaaaggccaataacattacaaaaatagtggaatgggcctgggcaacagagcaagacctcatttctaaaaaaataaaataattagccttccacagtgcacctctagtcccagctgctcagaaggctgaggtgggaggatcgcttgagcctgggaggtcaaggctgcagtgaactgcgatcccaccactgcagtccagcctaggggccagagcaagagcctgtcctccccacaaaaaaggtgaaatgcgagaggtgtaaataatggaacattaacaagctataaaaggatgcctttgaagcctgtgtttgtagacatgttttggtcattattaattttgaattatacaatttgaatttatttcaagttcgagggaaacaacagtaggtaaatgctttttttcctatttattggatatttttatagtgtttgtaggggaagtctgatgctcttagaattaactttgtgcctttattacttaaagtaaaagtgatttctggacctttttgaagcattggatgccatatttcagttcgtggcttaaatctcaacaagaaactggctttggccattggtcatctgaagggaatggcattgcgcacagaactctttgttgaaatggaaaggtccactgggtacaggggctgtttcctgtaatcctagctacttgggaagcagaggcaggaggatcgtttgaggccaggagtttgagaccaggctgggcaacacagcaagacccccatcgcttaagagaaaagaaggaatggaaagttcagctgtcctcacccttagctgcaccggggcaggcccattgaggagcacaagatagggtttctggtcaacgaaggaatgccacttcaaagcagaggacacaggaatggggaagaaaacgagccgctaagagaagatttctcatccccttgtgagagaagaaatcacgatcgcagggctctgaagtttatgtaaaactcatcggaacgggtggccgtgagaccgaaccaacagctcggagaacacccttctgcagctgcacagctttgctttgtttatttgcttgtttttgtgtgtgtgtttgtttttaaaataggtattcttttttttttctttaaataggtattctgttataaatgaaatcaacaaaatacgagaaaatgaggaccgtatcttcgtctatttcatcacaaaactgtcccgggtgggaagaggaacagcctatgtgggcttccacggaggtgagatcagaacatcagtcagggtatttatttatgtatttatttatttatttgtatctatttattttttgagatggagtcttgctctgtcaccaggctggagtgcagtggcgcgatctcagctcactacaacctcaatctcctgggttcaagcgattctcctgcctcagcctcccaagtagctggcattacaggcgtgcgccaccatgcccagctaatttttgtatgtttagtagtgacggggtttcaccacgttggcctagatggtctcaatccgttgacctcatgatccgctcgccccagcctcccaaagtgctgggattacaggtgtgagccaccgcacccagcccagtcagtgtatttatttaagtcatgttggaactgtttctcatttggttctgtgttgtacagtctgaaattagaatattattgttaacctcaagaggattctttccagatgcaattagatttcttaccactgattaccatgaaaacatgaatcctgggatgggaggctgcgtgcacctctcctggactgactctaggcaagctcctcacctcttagcatgtaggtcacacggcgtggagactcccgactcccgacagacactgtcaggaacgcaccgatacgcctgtttggcggggagggagagtggcgggggtcggactatcttcagatctctttttttccaaggaatggtcaccagctatttatgacatagaaaagttggtattccagaaacaggaaagtaagtttacttgaataatttcgtggagaatggaaggcagaccttgccagtgtcccccacccctcactaactgtgatacagcaaatacaacaatgcatgggtgaaagaaccacaggccttatccatgcgtccttcccacgctggccattgcaggtcttaccgttgctgcttatggtgaagctgtcgatgggcatagaatcgggtcgtgtatagtatcgggttgggtggtttctgcgcaggcccattccagggtttcggcttcagggtgccagtcattcattgcgcagcaagataatgacaagccagggtgtgcaatgtccctgcttagggctgagcagcagccgggaaagacacaaagacagttgtgggtggcttcactcttcacagggctgtggcagtctgtccacatcgatgacctccggagatccaccctcatggtttctgatgtgaccaggctgcagcatgtcaccatcagccagctgttcgcgcccggagacttgcctgagcgtgagtcacgaggcggagcccctgggggagcaggtggtggaagggcagatggcaggtgtggagcgtggcgtgcacatggcactaggagcagggccaccgtgtttcgtccctcggcaggagctcggggacacatctgcagaactgactggtggctcatcatagagcaccagggccgagcaggtgcgcttaccacaggctgaggtagagctctgtgagcaggccagccatggaagtgagcacctagaacacgccagagcccaggctggaaggaaggggctgccttgggggctgatcttcatgaccgtgatctctcatctggggacctagcaccacagcaggggccggggaagcctgcactcggaggctgagcacacagactcccagatggcaccgctgccagtccctgtgccaccttctgagtggtaacgcaatcacgtttgcttcgactgcagtgggcttggaacaccgggcggaagacggccatgaggaggcgatggagacggaggccagcacatcaggggaggtggcagaggtggcagaggaggccatggaaacagaaagttctgagaaggtgggaaaggaaacctctgaactcggaggcagtgatgtaagttctggttcttgggacctccccttgtgctgctggtgatgcttctgagctgcatctttaaacgcttttgcattgggagctagaggagtcgccgccgctgtgcaggggtgaggatggcgccccactttcctcacacagtgacggtcttgttgctggttactgggggtcaagggcatctgcaccggcagtttggggggtgcagggcggaggtcggcgtctcttctttgtccgtgaggaccgccgccctgtgctgtttgctgcattgagaccctcatcgcatacgggcggtttggcttttgcccactgtgtcagtggcagaaacggatgacccaaccgtctccaccaacaggtgtcgatcctggacaccaccaggctgctgagaagctgtgtgcagagcgccgtgggcatgctcagagaccagaacgagagctgcacgcgcaatatgcggagggtggtgctcctcctgggcctcttgaatgaggatgacgcgtgccacggtatgagcctccccacccctcttgcccctgcccccacgtgggctcttcctgaggagtgggcatcatttcactgtgtgttgggggacaccctctcaggtttccatggctcagcagaagcagtgacacagtgggaatctaagagcatctctcagattttgctctagaattggcctggccaacggacttcccttcctgggggaggtgggacagaagcactccggagccaacagctcagccacggccatgctgaacgtctgtttctgcctttgtacagcctctttcttgcgggtatccaagatgcgcctcagtgtctttttaaagaagcaagaagagagccagtttcaccctctggagtggttggcaagggaagcctgcaaccaggacgctctccaggaggcgggcacattcaggtactgtgactaaaggaagcaaggagtggctccaatctggtggcagcatggggacctgcctgcagggatcctctctccatccgggccatggggactgagctgtttctttccaaacctctcagccattcaaagctgtaaagtttttccccaagaagcacacagtaggtctggacagtggccaaatggcaggtgctggactcttcctctaatttcgtgtttacagtttttcatactaaaaacttaaaaaaagtaaaacatctgttcttagagtagaacatttaccgacattcatgttgataaccctagaaagggctgctgttttcagtcatgaagttaaaatgacgtgcagagcaggcaggggacaggcgggattttcccagtcctgctgtttgaagaatggtaacagcaaaaccaggacccagcagtatattcactgccatggttatttttctgtcacccatgagactaaccttgattatcttggatgtggccgtgtattgaggaaggaggggtgctctgggccccagacagagggtctctttaagacagactctcagacctcctgcctgaaagcctaagaaaccagtgacttggttatttgggcccagttttcccatctctaaaagacagcggactcctccatgagtccccttccaggtccaagcttctgtgccagtgataggttcgctttgggcctacagccaggtgtgatctgcaggagggaacgtgaggaagaagcggggtgaacggtaatgggggcttacaggggaagaagcggggtgaacgggaatgggagcttacaggggaagaagcggggtgaatgggaatgggggctcacggaggaagaagcggggtggatgggaatcggggctcacggaggaagaggggtgaccgggaatgggggctcacagaggaagaagcggggtgaccgggaatgggggcttacagggggaagaagcggggtggacgggaatgggggcttatggaggaagaagcggggtgagtgggaatgggggcttacaggggaagaagcggggtgagtgggaatgggggcttacaggggaagaagcggggtggacgggaatgggggcttacagggggaagaagcggggtgaccgggaatgggggcttacagaggaagaagcggggtggacgggaatgggggctcacggaggaagaagcggggtgaatgggaatgggggctcatggaggaaaaagcggggtgaccgggaatgggggcttacaggagaagaagcggggtgaacgggaatggggacttacagacaccttcctcgtgaccagagattttctgattctactttttctctagttatccttcgtttctctccttgtcacgccttctgaagggattttaggagatcagaaatattttcatttccctccgttcctccttgtggactctcttgatgcttttttttttttgagacggagtctcactctgtcgcccaggctggagtgcagtgatgcgatctcagctcactgaaacttccacctcctgggttcaagcgattctcctgcctcagactcctgagtagctgagactataggcacccaccaccacacccagctacttttggtatttttagtagaggcgagatttcaccatgttggccaggctggtctcaaactcctgacctcaggtgatccactcgcctcagcctcccgaagtgctgggattccaggcgtgagccagtgtgcccggcctgttgatgcttttgaatccctgactccactccccgggaccagccctgagtcactgaacctcctcagtctgctcttccccactctgctgttcacgcagcactaggcctggctccgtcgtgttctgtggtcaccgtgaggcctgcgcagccgctgagtggtggccagacccgcaccctcagctcacctgccccagggccagcagcaccctgactacctcgattcaccaccactgtgttctggggactggagtcctttgccctctcgggattaaacagcccaaatcctgagagtcctttgtgtcccgaactctgcctgcctgtccagatacagcagaaccaggctcagcggctccagatagggcggaaccaggctcggcggctcccgcctctcccagcgcttgtcccagcgcctgggttttccttctgccttttgtgttcgcgacacttttcctagtttgctgttttctccctctgtgcactgggatgctttattttcccaatagacattttaaaattcttgccaggttcctctgtactatatatttaaaaaatgtaggtatttctttttattctcttcaaaagagaggcttcgcttttcccccaacattaattaccttgtggggttttttttttttcgagacagagtctcactctcgtcccccatgctggagtgcaatggcgcgattttggctcactgcaacttctgcctcccgggttcaagcgattctcctgctcagcctcctgagtagctgggattacaggtgcctgccaccacgactgtaatttttgtatttttagtagagacgggatttcaccatgttggccaggctggtctcgaactcctgacctcaggtgatccgcccgcctcagcctcccaacgtgctgggattagacgtgagccaccgtgcccagccaccttttaattcttatcctttacacaagaattttgaaatagagctattattgttacatgtaaatatatacaacaaaatatatataacaacaaaatgtcttgtagttaaccaattatctttctgtctgcctatctgtgcattcatccatccctccgcctgcctgtccatccacccatctgtccgtccgcccatctatccatccacctgtctatcagacactcagatctagtgtacaaaaacatcaacaggaacaaatacttagttccagataaacatgtgccttcttgtttagtttgatctgggacaaattcattttggtctcttcatctgtattatagtctatctacaacagatttttaaaatatttattttttccaaatgaatcgtcataattttatttatttagagaacctcgatgacttgttctgaaaaatagttacatacaaaaattatgcagccttgcaaaaaataaagcagataagtgcggtggctcacacctgtaatcccaatactttgggaggccaaggcgggaggattacttgtgcccaggagtttgagaccagccagctagggcaacatagtgagaccccatctcaacaaaaaataaaataaaatttaaaaattagctgggcatggtggcactgggtgtgttgttcctgtctacttgcacggctgaggtgggaggatcgtttgagcccaggtattccaggttatgacgatctatgattgtgtcactacaccgcagttggcgtgacagagtgagaccctcacatacatgttttaaaaataaaaaagcaaacactacttcacccttccagtaacctaatgacagtttattagatgtagaattaagttccattccagttccgtctctaatcttagacatctttgtgagtctgttttctaggacttaaaaatgatggaaatgtcaagttattgctgtcgctgtagtgtggtaaatagtagactagttgttttgttaatgctcaagaaggaacaaaatgtcaaaaggtggcagaccgccactctggttcctctgacccgtggtggcccatctctcttctgtgctgcaggcacaccctctggaagcgggtccaaggtgctgtcacccctctgctggcgagcatgatatcattcatcgacagagacggcaacctagagttactgaccaggccagatactccgccctgggcaagagatctttggatgtttattttcagtgacacgatgcttctgaacattcctcttgtgatgaataatgaaaggtgagtggaaggctttctttccctggggagagaaactatcagaacacagcaggaccctaatatgctctcccaagtgctgggtgaaacgcagccctcaacttcagagcaacgttgacatgggtgctttctgcagtaacaataggaagtttggccgggcatggtggctcacgcctgtaatcccagcactttgggaggccgaggcaggtggatcatgaggtcaagagatcaagaccgtcctggccaacgtggtgaaaccccgtctctactaaaaatacaaaaattagctgggcgtggtggcgcgtgcctgtattcccagctatttgggaggctgaggcaggagaattgcttgaacctgggaggcggaggttgcagtgagccgagatggcgccactgcactccagcctggtgacagagcaagactctgtctcaaaaaaaaagaataggaagtttacgaagctctgagaaatttctgttttatctccaccctcctcatttccctgctttcagagcactggttctagccagacccagcatggcgtgacacggagacagtgcgctgcgaacagccccacaatctgtgctccttacccctgggatctgtgctccctccccgtccctcattccctggtggagcggcacagcccccacccccttccgcaggcctgatgatgtgcggctcaggctcagctcccaatagcttccacttcagctttcaggggcttcaattctggatgtagatttataaagaatatagaaataaatgtgcccaggctcggtggctcatgcctataaccccagcaatttagtaggctgaggcaggaaagtttcttgaacccaggggttgaagaccagcttgggcaacatagtgagaccccatcactacaaaaaatttaaaaattaggcataacgtggtgcgcacctgtagtcccagctactctagaagctgaggcgggaggattgcttgagcccaggagtttgaggtgagataggatcacaccactcactgcactctagcctgggtggtgacagagtgagactctatctcaaaaaaaaaaaaaaaaaaaaaaaaaaaagagtgagagagggagggagacagaaggaagaaagagaaagaaagagcgagagaaagagagaaagaaagtaaagaaagaaagagtgagagaaagaaagaaatggaatttgaattgccagtggaaacacttatttaaaagacaaggcgctgctgatgttcatgaagcaaagactctaacaagttgtgctctgtgttacaagttatcaaagccagacaggttcaggaaccagaagacttggttctgcccgagattttgggcaacacacttgacccctgtcagccttggtttgtttttgttttgcctgtgaaacaaagattgcataagacagttccctagttctgtcataacagaaaccctgtactgtgaaacagatcagcgccgtctgcctggcctgttggcccctttgtttttgaggtctgagaagagctggcatcagtggcagatcttatcttgtgttttgagtgacgtctcacaaaccctcttcttatcatccctcaccttattgcagacataaaggtgagatggcctacatcgtggtgcagaaccacatgaacctttccgagaacgcttccaacaacgtccctttcagctggaaaatcaaggactatctggaggagctgtgggtccaggctcagtacatcacagacgcagaaggtgaggctacctcaagacaggtcagattcagcacaggtgaatcacaaagggattgcctgacagtgaaaagcagatgggtggctagaattgcaaggtgaattttggagtttttagttttcacactttgtttgtgcagtaagcgtccaatttacgtcaccgcgtcattttaaagtttctgctgaaggagtgagaggccccggcgggaccagaaacctttcgatcctatggcagcaggagcctgtgagatggtttttagtgccgtgatggccagggatatggcgaaatggggtgtgttctggcaagaggaggactgtcgcctctatcagctggtcattcccattctgggccttctcagactggggcactggactcggaagatgcccccaggagaggcgttctcagaaggcacgagcgcctggagcatcccctactcccaggtgccccatcaggaggcacctcccagctgtgtctcccgagtcacagcccagacacagtgctgagctctgctgccagatgccagaagatttgccctagtccagaaaaaccaacgacaacgcaacacttttacaaaagccatctttcatacatgcaatagtcttttccacgtttacttatcgtaccatttggaagactcagggtggccttcttttctaggttaccattctgccactttttctcaaagcagtctctctcctcatgagcatattcctttctcaggacaataccccaactgaccctgcttttctatgacagcatgcgaattaggggctgcaccccgcccactagaagccagatctttctggcttgcatgctctgtgacccccagcccctgaagagcagcccacttgttcctaatttacttggacccgggggttcctgaagcatgctgtgacctgtagccatgcaacatacaatgtagccatgcaacatacaactcagtcttatctagatgtttctctttggggctagcttggctgtttctgacctaaatttgacactgtttccgccttcctttattttattccactggaaaaatatcctaggccaggcgcagtggttcacaacaccttgggaggccaaggccggcagatcacctgaggtcaggagttcgagaccagcctgaccaacatggtgaaaccccgtctctactaaaaatacaaaaaattagctgggtgtggtggtgcatgcctgtagtcccagctacttggtaggctgaggcaggagaatcacttgaacctgggaggcggaggttgcagtgagcagagatcacgctacccaccgcactccagcctgggcaacagagtaagactccatctccaaaaaaaaggaaaaagaaaaatatcctaaatgtctagacagtgactattccttcaattccctacaaaatcaggcagttggtacccctttgtctacgagctcccattcaacaaggcgtccccattccccttcactccggagccccctggaggcaggggagcgccatgacttagacgcactccaggccgctccttggtttcctctctgcaggactgcccaagaagttcgtggacatctttcagcagactcctctgggcaggtttcttgcccagctccatggagagccgcagcaggaacttcttcagtgttacttgaaggatttcattctcttgaccatgcgtgtgtcaacggaggaggaattaaaggtagatgtttagatactggctaagggtcaggtgtagagcttgcatgatggggactggatgcagacacggagacgtttcctcagctgcctggagctggtgctgtgaagtctgggaacagaacatggtcagcgaagggtgccggtgggtgaaggggtcgcccagtctcctctcaatattcagtgctacaccctcagtccaccgcccaggggctcctaaaacacggagctttctgtgaacatgagaagacctccaatttccttgacatagcttctttttaaatgttgagtttctcttgaaaaaaattaaatacgtaactaaccctacagggtaagaataaacaagtgaaattcaatagaaagtcatacttttgactacctaaaaattttaaacttcagtacataaaaaaggagagtgacaaatttaagaacatccttacagctaatatgactagaggttaatattcttaggaatcaataagaaaataccctaagttttaaaaaatgggtaaacgttataaaacaacaattttataatctcattaagcatatcaacatccaggaaacatttgaaaaattgcttagccttactattaatagcaaatcaacacaacagtttttagatatcatgtttacgtattcattttgcaaagctgatttttaaaaacaacagccgccggccagtgctgcaaagaaatagccaggccacgcggctgacagtgtagcgtgcacagtgggcgccctgcagggctgcgtggcagaacagttgagcaccttatccgatctgaactgctgactctgtcctgaggaaactactggagatgtggacaaatatttggttcacagggatgtccttttcagctttgcttcgaaaaataagaaatagtttaattgtccggaaatacaggatgggcaaattaaagtatggccgtatgatgggttattgagcagccattaaaaccatgtttttcaagtatactcagtcatagggaaagttcatgatgtcatgttcagtggcaaaaagcccagacgacacggtgaccttaacgtgggaggagaggcagatagaaaactggacagaagcaaacgggacagaatagcacacatggtttctaatttcctcttttatggttttccaatagttcggatttcccacggggaaaacataccatttttgtaatttaaaaatatattctaaaagcttttttgaatcccagtttctgcagatggctctgtggtcctgcactaggaaactgaaagcggcgtcagaagcgcccgaggaagaggtttccttaccgtgggtgcaccttgcctaccagcgtttcagaagccgtctgcagaacttttccagaatcctgaccatctaccctcaggttctccacagcctgatggaagcccgttggaaccatgagctggctggatgtgagatggtatggccctcctccgcctgccctgagcaagccttgtggtgcttccaactccactctgggaatggagagcttctggggctgtgttccaagtgggagatttcttcacaaggcacataccttagctgaattcagagatgttaaggacacatctcgctgacgcttctcacatcctagacaatgaaagtttaggtcgcaagcctgggacacaagtatacatgcagctaacagccggggcctctggtgggccggtgggaggggcaccgctcagccacgccctgctgtccgtctccccagaccctggacgcatttgccgcaatggcctgcacggagatgctgacaagaaacaccctgaagcccagtccccaggcgtggctacagttggtgaagaatctttccatgccgctggagctcatctgctccgatgagcacatgcaaggcagcgggagcctggcccaggctgtcatcagggaagtcaggtgagacccaggagccctcacccactgcttcatctggcgcgcgctcaccaggagcctgccaagtgccaggcatgtccatgagaagacgggcaggtgctcctgccatgggaggggctccgtcctcacccgttcactccgcatttgccgaacccttagtgagtgccaggcgtctcattagggtgctggagacacgagaggcggctgtgcattcagggagcgcactgtgtactggggccgtagaaaggcagacgggctattgtgatgccgtgtgagaaatgctgtggtagaaaggacaccccgggcgggcaggtgcacagagcaggacaggccctcagggccagagtccacttccctaaaggtgaggtgggacttgacaggcgcaggatcatgcagtgagcgggttgtggtggtaccccatgcagaggcatcacagtgggacaccgagtcccagctaagctgagcgtctgagagcgagagctgggggcgggccaggaagtatgtcacatagggcttgcttgtaatcccagccgctgggcctggaccccgggtcccgcatctcttctcccgtctccctcctcgtttcacccttgggttccttggtgggagccgagtgagtgagtgagtggcgccctcttttgacagagcccagtggagtcggattttctccaccgcactcttcgtggagcacgtgctcctaggaaccgagagccgcgtccccgagttacaggggctggtgaccgagcacgtcttcttactagacaaggtgagtacttgggctggcctcagcacatgcacggatccacccttttccgaaaggaagaacagcactgacagtgatctgcggctgggagacgctcttttggctctgaccgttttgtctagacatgcaaaggaaggtgatttcatcactaggccattacattttccatgtttaattgtttaattatttaccctctgctgattgctaagtcagagcatatcatttagaagtgattccaggaactgtgtgcagaaaggggctgacgcagggaacatgcttgtgaaaccttcggcagggattacctggcctttggagtgaccccagaacagaccactagcctggccatctgtggaagctgttaccattttcatagtcatggagacaaggatgtcagagaatgccaccggggtttggggtcttcaaaacagtgctgtcaacagaaccaaggaacaggaagccaccatgcctgtcactcaacacccttcataaagctactaatgtggggtggtctgcagaaagaaacctgtgaccctacagtgatgcccctctgtggccacgcagtggtgtccagcaacagtggccactcagccaaacacagcctcagcctctgcctcacccgcacccgtgaccttcacgtgagcacatccagagccctcaccgccagggggtctggaagaggcagcgctggctgtccgacctttggagcacaggaaggtttactaaaaggaggccagcatggatgctggatgtgttgactctgacctatcaaaacatcagcggcttagacacagtatcagctgatttcccacccaggatgggtatgagcagcccaaggctaaggtaccagctccccggaggctggcacggagtcaggtgcccacgctccgtcttgtcccttcattgtccctaatgttcagcttccaccccgtggcctggcgtcacagcctgtgggacagaaagcaggggaggtagaggacaggccgttttccttaagagcccaatcccggacacgcacacgccacaccagctcgtgtctcgtggtcctaaacttaggcactcggccttatctggttgcaagaaactgaggagtttttcgtggcatggtcatgttccaggtggaacttgagttcttctgctgaaggaaaaggcggagagaggatagtaggacacagccggtgccccccttccacccttttccccacacccacacagcatgcagcctctccaagtctcatcatgtctcggcgctctgggagatgcacagggctttcagccaggtccagcgtgggtcataagggccaggtgcccataaacccaaaggtaaaggagctggcccctgcctgctcacatgcccacaggatttgggaggttaggaacgtggggagggttttttttggcgataaaatggcctcagaaacttactgggtttctggtttatttccttccagtcagatctctgtgcaggtaaccacagccaaagaccttacctcgttagttttcctgctggaagcctgacttgtacccctacccagcccccgctgaagagcagcccttgggaccatttgaaacaagaggcttgggggaaagccctgccttccgccgtgaggctggctccctcggccgggcaggaagttgtcgacggaactgctgcaggatggcacgggtgggactgcgggcatggctggcttttagttgttttggatatgtaccttgaaggggaattcctggatcatttgataactctgtttaactttatgcagaggcactgtattgttttccacgggggctgcagcatctttcttacatccccaccagccaggcaccagggtctgaatttctccacatcatgggcaacacctgttactatctgatttttttcataatggccaccgtgataattcattgtggtttttatttgcatttctctaatgactagttatgatgagcatccttcatgtgcttgttgggcatttgctgtcttctctggagaaatgtccattcaagtcctttgcccatttttgaattgggttgttgattaacaaagttattaggatattaaaaatacacacatcacttccaagtaagattaatcccagcaattcaaggatggctcaaaataaaaaaaaaaaaatctgtaaatgtcatcagttatataaattaaaagataaactctatatgatcatttcaaaatgtagagagaaagcatttgataaaattatttggaaagaaatacccttgtcaaacttggaacaggaaggaactctccttgagctcacaatgcccaaggtagacataatattaagatgtctataccaacatcatgcctgcccatcaaattccattagtacagccattaaactatcttgtacagtgatgatgtgtattgcctaaaaaccagtgtttccacttctaactatatcctagagaaacttctcatttgctctcagacatatgcaagaatgttgaatacaggactgtcaggaattgcaaaaaacctaaatgcccattaaccagcagacaggtaactggtgtctttatcagtgggtacaggagtgatctggacctaaggtatcatgaagagaatgagttgcagaagcctttatacaacgtggtatcattgatatcaagttttaaaacattcaaaataataactccatgttcagatatgcttacagctatcctaaaagcagacatttagaaatatagttatctctggggagggacggagggatgtgaagttgtaactatatgcataatgttttgtttctctaaaaaatgatctatcgcagcagcttgggaggctgaaagaaaattttaaaaaatttaaaaattttaaaaaatgatctaaagcaaatattccataaatgctgagatttcttaaagcttggtggggggtatacagatggtcattattattacttaaagtttgaaatatcttagtgagagcggaggagaccttaaaagaaaaagaagagggaaaatcgtaacttgagaatgtagtcccctgacaagcagcataatcacaaaacgagcattcagagttcaagcgttaaacacaggatcacaaaggcctcgcaccccacaccccacacacacggcgcagccttggccctgaatgtggtgctccctctgtcgcccggcctggccgccctcagccctcccccctgctaatgactcctgtccctgcctttcttcagtgtcttcgagagaactctgacgtgaagacgcacgggccttttgaggccgtgatgcgcactctctgtgaatgcaaggagacagccagcaagaccctcagcaggtgagaccttaggtttggatctgtgaaggcgagaattctgaacctctcgtggttttcacttcttctggccagtttctctgcttcagaactgattgcccttcttggattctaggtttgggattcagccgtgctccatctgcctgggagatgcaaaggaccccgtctgtctgccctgcgaccacgtgcactgcctgcgctgcctcagggcctggtttgcctcagagcagatgatatgcccctactgtttaactgccttgccagacgaattctctccagctgtttcccaagcgcacaggtacaacaccctttctctcaagaaagcatcctttttttcattttccttttttggcaaacacaatattcagaaatataaactctattaaccttcatttggtagaaaacctgacctcagagaactatcataagagacgccacttacgtactttcataaatatctggtatctcaagattattctctccccaagacatacacatacagatttcagacaacaaattcagaggaactaagccagaaagtcttaacaaaggtcctcaccagaggcccaaggaatgtagacgccagtgtttttttttttttttttgagacagtgtttcactctgttgcccaggctggagtacagtggcacgatctcagctcactgcaaccttcacctcctgggttcaggcaattctcctgcctcagcctcccaagtagctgggattacaggcatgcaccaccatgcccggctaatttttgtatttttagtagagacagggtttcaccatgttggccaggctggtctcgaactcctgacctcaggtgatccacccgccttggcctcccaaagtgcagggattacaggcatgagtgaccacacctggccgacgccactggttttaatgcatcagtatcattcacagtcactgacctgaggatcaacccacttcttgtctagctttttggagctggttaaaagcagctctggctctctggactcagcaggcctcagcccggctctgtgagtggttggttttctttccttccccttccccccttctcaggcctcctaaaagtggggcaccaggcaagcccagtggatgggttctttgacatactttgggatattgaagggtattgttgccgggtccaaatgatcaaagtgtttccagaaagggatgcatgtaaatatcatctgctgaggttttgaagactagctctaacgggtgagaggagtgacttgaatctttcaatttaaaagtaaaatgccagccaggcatggtggctcacgcctgtaatcccagcactctgggaggccgaggtgtgtggatcacctgaggtcagcagttcgaaaccagcctggccaacacggtgaaaccccatctctactaaaaatacaaaattagccaggcgtgatggtgcgtgcctgtaatcccagctacttgggaggctgaggcaggaggatcacttgaacctgggaggtggaggttgcagtgagcggagatcgcgccactgtactccagcctgggcaacaagagtgaaactccgtctcaaaaaaaaaaaaaaaagtgaaatgctgtgcaaatctcaagtcattctgtaaggaggcatttgggaaacaccatccacctgtcttctgtttctcgtgttctaagggaagccattgaaaagcatgcccgcttccggcagatgtgcaacagtttcttcgtagacctggtgtccaccatttgcttcaaggacaacgctccgcctgagaaggaagtgattgagagcctgctctctctcctcttcgtccaaaaggggcgcttaagagatgctgcccagagtaggttgctttcttcctgtaaacctagcccctcatttcttcatctcgcttctgcatgtctcatgcagtgagctgcctttttctttctggtttaaggacactgtgaacacacaaaatctctctctccattcaatgatgttgtggataagactcctgtcatccgctcagtgatactgaaactgcttttgaagtacaggtaagaacaacatggagacttgcttctggaaagccctggctttttctcttcatcgcagcgttttgttaccaagtcttcttgtcgtgactagtagctagtacatgatcttggggagctttttcggtgagacacagcctggttacttgtactggttgtgtattctgacattttcacatataggcacatgagtttagtattcccacaagcacacgcatctagcaagaagccaacagtgccgtgaagggaccacagtggcgtgagggacatggaactcaagaggccagaatctgtggtgctgagcctggttccacagcagcgcaactttctgatttttcagatggtagcttagaatcctgctcaccctgaatgtcctagatgtctcctaagtttctattcccctgaacatgggctctatttccttggaaaggatgttgacatccaagaatagtatcacctgtggtacttgtatggatctaatattgcagttctcaaagtgttggtatggacccttgggtgtgaggtaggtctctgaggttaaaactattttaaaaattagattactaagataaatttgcccttttttaaaaaacatgttgatacttatgatgatgttacaaaaagcggtgatgagttaaactgctggcccctcacgtgaagacacagtagctggaggtattgttttcttcaccgccatgcacacgtggttttcttcatgccagtttcattttagttttactaaatcttgactcttgaggatttgacttttcaacgttttgtggaaatgggaagcacacacaaaacactcccactacacaccggagtagacagttgcctcacagaaaatcactcatgacaagccctgggttatcagccggctgctgtgttcatggaccgccacccgtgctgggaggaaggatgcacatgatacaggtgtccagggctgggcaggcctctcctggaaatcagcaaagtgagccagcctcagccaaacaactgacaatgttggttggtgatgataacattcaagcttttaagctgaaattaaaattttgaaaagcttatattctccactatgagcttgtgagcttttgaatacttaaagacattttggggcaatttgtggtgatattaatgaatatggatttttttttttaaaacgtagaatacaaatgtgtcaacctttacaagacctgcacagcccagtgaaccaatattttccaaatgaccaatgcctgatgtcatgaaatcatgtgggtcaaaaaagaaaaaaatctatttaaagtatgggcagacctttttgattttaacataattgactgaaaaatgcactgatacagtttcaggctccacattgcaactaacctctagacatgaccatttatcactgtgggcacagcattaaggaataatagccacaatcagctgcaaaaactattaaaatgttcctcgctattccgagtatatgaggacagattttcttcatgttctcccatatgctgcagcgatggggagcagggcaggcaggccgctgacttccaggagccaggccccagggccagcacaggaatggaagaccaaaccactcttctcacggattcatgttttggaaaatattttaaatgttatttaacatgtaatagctttattgttattttaaatgaatgtatatttttgaaaccttttgtttttaatgtctcataattcctaatatcataaatattgataatataacccacataaaagctcttcggggttctcaatttttaagactataaaggggtcctgaaaccaaagtttgaggacagctggtctaaaatgtgagttttatattcctccagtattatgctaaaaggttttccataaaatcttagaaaccacgtatatgtatgtatatgtttatacacacacacacacaggacagacgaccgatagatagatgctcttttttttagtaaggtatgtctccagatctgagagaaccaggaataatatttctctttctgcagctttcatgatgtaaaagattatattcaggaatatttgaccctgttaaaaaagaaagcattcataactgaagataaaactgaactgtacatgctcttcatcaactgcctggaggtaagtgaactctctcttccctgaatttcttttggaaactatctgaaccacatggtttaaaatttacagaatataattttagtcagtataattaacgaatgctctgttccgtgcagaaaagaagaaaggtttcaaagacgcggcttctgccttcagcaacgtatattttggcatttacttgcatatacatacaaaacagaacagcctaggagaatatactgccaaagttaacagtggttatcattgagtggggacattatggatactttttatcttcatatttttatatccaaaatttttataatgtaaataaattagataattttttttcctcataggttcacttttcccctccttcttgtccttaaaataaaaaggaatgttaggtttccataagtcaagaaatttacactgaaacctacagtaacattcttctatccttccttctccacagtcacattggcgactgtagaagcttggggcatccatgtttaaaaaaataatatccttttctttcttgttccttgttcctcaggattcaatacttgagaagaccagtgcttactccagaaatgatgaactgaaccacctagaagaggaaggtcgtttccttaaggcatattctccagcaagccggggccgagagcctgccaacgaggcctcggttgaatacctgcaagaggtggcccggatccgcctctgcctcgacagagctgcagatttcctctcggagcctgagggaggcccaggcaagtcttctctgccttgctttcctttgggtttaaagactccgttatttagaaattcaggaagtatgggaaactagttcttcgaatagactactactgggcttttctacatcccctctcccctggatgtgtttctgtgaatgcctgtgggtaggtccgatgccctctggttggccttgtgtcctacaataaatgccctaagtcaccagccactcacccgctttctcgttggcctctcagagatggccaaggagaagcagtgctacctgcagcaagtcaagcagttctgtatccgggtggagaacgactggcaccgggtgtacctggtgcggaagctcagcagccagcgggggatggagttcgtgcagggcctctccaagcccggccgcccgcaccagtgggtgtttcccaaggacgttgtcaagcagcaggtgagaagcggggccgggcagcacacaaacatgcacccccacagccccatacaccccaaacccacacacccccctaccctcacaccctaccccccccacaccccaccccctcacaccttccccaccacataccctcatacccccacacctcaccctcacacccccacacctcaccctcacacccagggatggtgccactgcttccgcgttccgcctgcccttagcatccccagagccaggaaggaaacctgccgcttgtctgtctggtcctttgtctctgtgcttcagctgatttgccccagcttgtgatcatgtttacactgcctgctgtagggccaaggtgagggcactcacctcagagcttctttaattaggcatcacttctaattgaatttcactcaaggaaagtagaacattcttgtggcctttgctttcagcgaattaaacgtagttcagcttgcaaggtgcgacctttttacctctggcaggcagagacaggcaagagcaagaactaggaatagaagaattgcagaagaggcttcaaggccaggctgggcacagtggctctcacctgtaattccagcactttgggaggccgaggcaggcggatcacttgtggtcaggagtttgtcaagaccagcctggccaacatggtgaaaccttgtctctacttcaaagaaaaaaaaaattagctggacatgatggtgggtgcttgtaatcccagctagtcgggaggctgaggcaggagaatcacttgaacccgggaggcagaggttgcagtgagccgagattgcaccactgcactccagcgcctgggcgatagagcgagactccgtctcaaaaaaaaaaaaaaaaaaaagaggcttcaaggcctctcagtggtcctttccagatacccagaaattaacaaattcttattgtccaggggatacaaaaatggtactaagaaccccggcactgtttgactaccacctggggtttagagttgacatgacttgagccacccggcctcaggtgtggtgcggtgcgccgcaaacggaggaagcaagcacctccccaggtagcgctgccgtcggcaggcattccctgcacttgcacctcttcgtctactgaggaggaagctccctgcgaagagggcaccagactgtcctgcctcaggccaggtcagggaatcccagcttggctgcctttaaacctcgaatgatcaaccacctggttcctgtacccgttcagacggttctttgcaagacattcctcccattgttcacccccaactaacctctgtattccaggggctgcggcaggaccacccaggccagatggataggtacctggtgtacggcgatgaatacaaggctctccgtgatgctgtggccaaagctgtcctcgagtgcaagccactgggcattaagactgctctgaaggtaggatgggcccgtggcttctctctgataccaggtggcatcccttggagcctgcatgttccctggtttatgtcaaatgatgcagggtgatggaccactgatgcagcccatcacgtgacactgtattggaagtcacgtgcccacagcctccagggcgcacctggctagtgtgcgtgaaccagtggctggaacaggacagctatgacagttgagctgggttgacagatttgctgtgtcttgctgatctgccaagtccatcagcagcgccttttgatttatgcccattacttcctttgagaccctgctggaaaacagtctaagaaagatgctgacacaactattaatagcaaaattcatacctagatgtattcataattcagatatgcatgtgtgtttaataaaaatacatatgttggtgtagtgtgggaagttaaaaaatataaagatgtttttagaatctaacctttaaattggtctggtcttgcttcctttttgagtccctgtgccaggaaaagaggaaatggaaaatcctctttctgctcagaagtttctaagaccagtgaaggagtcagaagaacagatggtgattccgttgcttctgtcattaagctggctgctaaatatgcgttgagtttaatctgagagtatttcaggaactttgtagcccaaaagttgctcgccagaagactgcccatgtggggcatgaccccaggtgtccaggcctggaaaaggctccttcccttttggtgggttgagggctgaggcatgtgaaaagtttcttttcacatgcaaacttcgtgggcctctggcccttaggtagatctggcaggcagcggtgcgggggcgtctatcctataatcctagcactttgggaggccgaggcatgcagatcacaaggtcaggagtttgagaccagcctggcaaacatggtgaaaccccgtgtctactaaaagaaaaaaaaaaagaattagccggacatggttgcgtgtgcctgtagtcccagctactcgggaggctgagttagaatcgcttgaacccgagaggcagaggttgcagtgagccgagatcatgccactgcactccagcctgggtgacagagcaagactccatctcaaaaaaaaaagatgtgttgagcttttaaattcttacttgatacccttgattttgcaggcctgcaagaccccccaaagccagcagtcagcctacttcctgttaacactgtttagagaggtggctattttgtacagatcccacaatgcaagcctccaccccacgccagaggtgagtaaccgcctgcagggctgtgttcaaagtctcagtatttggagggattttattgaatagaatgaaagttatcaacacaaatcataccataatttttttatctaggatagaggttctcaaccttgttatgtggaggaacacataaccaagctgtttaacccaaagcttgtagaattataatgaaaactaaagccaagaacataagataatgtttaacagaatagaatatcaaaattaatgcttacgtatgtggatgctgtgtttgtatatgcacaattctcttctctgaaaaatttccccctcaaatggtggtgatttctccatatttaccttgatatttgatgtgtatttggtgtcagtgtatgtctaagagcaattcacacaatattctttgatacatcttaatgttaagtttttttcctgtcagcaatgtgaagctgtgagcaaattcattggcgaatgcaagatcctttcacctcctgatatcagccgttttgcaacatcgctcgtggacaattctgtgccattgttgagggcggggcctagtgacagcaaccttgatggaacggtgacagaaatggccattcatgctgcagccgtccttctgtgtggacagaatgaactcttggagcccctaaagaatctggccttctccccagccaccatggcggtaagagtaggccacaattccatcggccttcactggaacaccccccagagcttgtcactgtagagaccgagctgcagctgtgggaactcttgagcatctttatctcctcagttctctgcctttgtgtcacctgattcttgagcacacaagatagtgacagacttgccttgccctttgttatttcacaggaagctgttctgttttcttcacagacagcttgagaagtccagcagaaggaaagcagagttccctttcttcctctaggcctcctgctgagcagcagcacccaggtgacaagctcacttatctagagctgtctctgtcttcttgtttttcagcatgcttttcttccaaccatgcctgaagacttgctggctcaagctcggaggtggaagggtctggagcgagtccactggtacagtaagtgttggggtctagatgaccccacactcctttgagcttcaaagggtgtccagatgctatgaagcattgggttcgacttggggtccacgtggtttgtgcctcagggtccaaaaccacctgcattctaccggtggcgtctgcagaagacgaatggcttgaaggagctggcactccgccggctagatgatccaaaccatttcatttctttgtcgtgtggaaaaggccctctgtgatgcacttctgttctctggaatatgccattttgggagaagggagggactgggaaccacatcagagacgcattcaaaggcccaccacaaaacaaagtttttgacacaaagctcttctgaaatatatgaaatatagaaccgggagactgactcatataacccatgacatgtaaattttgctgttgcctgagtaaatattttaaaactagtttttaaagttgttataaaatatgctcatgacaaaaaaaaaaaaatcaaggaaaatagaaaggtctagaatcctgtccaccttgcctccaaaagtacccctggtatgggcctatattctaattgctctttacttgataaaattaataaaattagactcagacatttttcactgacaacatacatttattaagcttcaggcaggtgtcatgtaacttaacaaattagcgtgggatgctctggacagtcattctcatattgtggtcagggccagagagtagagagttagctttcccttttcaatgtgggtcattgggtgaaacctcattagccaatgtgtgtcctgttctcttcacagcttgtcccaacggccatccttgctccgtgggagaggtgagtcttggtatttaagatagggtttgaggggtggcgtgcactcctgggttggaggagggggatcgtgggtcaggagagtgaggctctcggccttccaggagagcacaggctcaccgaggactgcccagggtgctctgagcagggcaatgccaatggcgctaagggtttctagcccagggcttctcagactcagcactgtggacgtggctctgcggcgtgggctgtcctgtgcactgcaggctgtctggcagtatgcctgacctcgagtccctggatgccaggagcacccactcctcccagtgtgacagctaaaaccagacattgacaaaggtcccctaggaaagaaaattgctactggttgggaactgctgctagccattctttctagccactgcagcatggggtcagtgagccttgtcttgatagaatggcaaggtgttgcctggaccacaggctgcattattagccctcagaggaggtgtggtccagatgccaaaggaaaaagccacgtgtaggctgtgcgccatgggggaagaactgttgattattcctctctggattcccaagcatctagcctagagccctccatacacagtatctgtacttgctgagcaagaggctggtgggttgagctgacacgtggtagttagttattagtattacttacttggttgagatatgtctggctctcttacccaggctggagtgcagagtgacatgatcatagctcactataaacattgaactcctgggctcaagcgatcctcccgcctcggcctcccaaagtgttgggattatgagccactgtgcccagcctgatacacgtcatttaataatgtatataagagatacggagggagaaaaactgttacatacacttcatttttctgagcttcaatcacttccttgtccatgaaatgaggacgctgaaatagagtctatccaagaatgctcccagctctgaaattctgtaactggtttggtgtgttatcagccgtgactctcagtatattgtggcacaatatctgattttgaaaaataagcctttggaagtagattatgtgattagatttttttaaataaaaaagtccaagccaggtgtggtaggtcacacctgtaatcccagcacttcaggaggctgaggcaggtggattgctagatcccaggagtttgagactggcccgggcaacatgttgaaaccccgtctctacaaaaaaacacaaagcatcagcagggcatggtggtgcatgcgtgcatgcgtgcatgcgtgtggtcccagctacttgggaggctaaggtaggaggattgcttgagcctggggaggtcaaggctgtagtgagccgtgatggtgccactgcactccagcctggaggacagagcgaggctctgtctcataaaaattgatatatgaaaaagtccaaatgaatataaggaagtcagtttggaagctcttctagtattcttggtattagatcatgaagtgccacaggctagtgtgatagatggagggatggaaaaagtgctgaatacaagaaaggaaggttgaattttatatatttgtctataaagtgtcactctgggaataaagcttaaattgtctcacttatttaaaaaacagttccagtttgcccacgtgcaacattagtgagtacctaccctgttcccatgggttctccacccacccgaaacaagcacacactgggacaatctgagggtgctcacgtctgccggtgataaggaggtgttcatttgcatgatggcatttgtgcataaaatggtactgctccagaagtggttctagtgccctgtcttcaccctagtgtggcaggccgatggaacagagcatctgcattgactgccatgcgccgattggaggcattgaccacaaacctcgggacggctttcatctggtcaagtatgtgggtcaggattctatttcctaagtactcaaactttggggtgttgggctgtttttattccagctgataaagtgatactcaagatagtactaattactccagtacatgtgggcctgtgtcatattgaatagcaatgccaaagtcccagcagtcacacagcagggaggcatctgatctgttgttggatccctgggccctgaaatgcaaagcaggctaccctttaacagctgttaatatgaatacaggtgtcttccttttgggccttgggtgcaccttctgtagaaatatgcatgacataaacagatgatctaattcacgaggacatctatgtctgcaggaccaaaacctgggcatccgtctgtctcagcagatgccatctcacttgccagcaatcttagctttctccattttttattttttgagaaaaatgaaggagacagacatagtcttatgaaataccagacttcagaatccaggtacaaatccgatcccacaatcccatgctgagatgaatgtggttctaaatgctcctttcagagagtgagtgggacctgcgtgggggcctcacattctgactgttccctgcagtcacagcttaaaatactttcatgttatctccagaaactgtcacagtgtcctccatcctctaatagttttgccactgagagcatcggccacactgcatctcctctctgccggactttagatcccggtttttcctttggggagatgagaacaatatggacacccatgggatgccgactgtcctcccaaactctagctgttcttagcccaaccgctcctctcctgacaaagacttcagtggaaaagacgggtccaggcctgcgtcttaacaccctgcccggggatcagcatcaataccacacagcgcagccacctgacgctgagaatgcggtcgggtctggtccaccggaaaggcgagtctatcatgagagagactcttaggaacatggtccaggctttgtggaacagcaagtactcttcacataactctcttctcagccgtaagcctcactccaagtgctgagaaagtgcagcgtgccaaagcggccagcctcagcctctgtcttgtgttctctcgttctttccagagacaaggcagacagaacgcagaccggccacgtgctgggcaacccgcagcggagagacgtggtgacatgtgaccgagggctgcccccagtggtcttcctccttatccggctactcactcacttggctctgcttctgggagcgtcccagagttcccaggtataacccagtgctgccaaacaatgggctcagttaagaacctgctttcgcttcttcccagggagtccttttgtcttgagtcctagctgccatctatgcgttttggagataattagtctacaaagtaatatacaagttatacatcttcatgtttccccctgtggcgtatgatggtatggggggaactactccccagattatacagaatccacttcaaattaacactctgtgtctctggtccacatctgggagtagagaggctgacatcttcattattttacctgtgtcttcattgtagctagggaagaaagagcacactgcataaccaagatggccaacagtacaatctggatattctttatctcactgtaacaacagaaaagccagcagccgttccgcttgctagccttcctttcacctggatcactccgccgttttcatcttagaaaccgccttccgactctatgctggtgctccactcccaatggcctctaccaggctcaccatcttctctacaaaccagatccccgctgaacactctgttccgttgcccccacaggctctgataaacatcattaagcctccagtgagggatccaaaaggctttctgcagcagcacatcctgaaggacctggagcagttggccaagatgctgggacacagtgccgacgagaccatcggcgtggtccacctcgtcctgcgcaggcttctccaagagcagcaccagctctctagcagaagtgaggaaaggggcaagggctgggcggggatcacacagcacaacggcagcgcaagcaggctcgctgtcttgtgggccaccccacacacagccagtctgagctctgtctgtgctgttgctggaaagaatgagagaacacacagagagaaaaccgtgtctagggaaggcgatgcctggggctggaaaaaggaagcactctgcttgcccagaggctggaaggagctgctctgttccctccattcacccctgcctggctaagacctgcggtattcgggagcgtctgcacgcaagtgttcagtgtgcatatatggggagtcttcagttccgaagtcagccccgtaggtcattcccctaccatgctgggagcacgtgtcgtccacacagagcgtgcgtgtcaaaggatcaatcaccatcacagttgtgtgctgatttattaaatgtttatatgtccacagttatgttaaaaaaaaaaagctataaaatattgtattcactgagctaactttctattttaaaaatttaaatccatacagaaagaaaaggtctgtaagacaacttagtaaaatggttatatggttatctgtgagtggtggaattaagagtgattttcttttgctaatctgcaaaaatcacaatgaccattgtgaggacaatgttaatttaaaataataacaataatagcaagaagaagtcctgccccatgacaagaaaatctgctcccacctcaggtacttctttgcctgaaactggcattcctatttattttccattttgaacaatctgattttattttaagcctcttattctggtgggatttcagcaaagtgagcacacccagagtcacactgatgaaatgacagaatcctggaagcaggtggacagttcccgcagcactccacatctgtgtggtctgcctcagggtttgggtggcagcagctgagacggctggcggtagcctgggccactcttactattgagctcaagttctgttctttcagtaatttttttcaaaatgggtctaagattacaattatgtaggatagaggaatttttagagattgatgttatcttcaaaaggtcattaagttggtggtaacaacaggaactacttcaaaatacttagattaatttagaaagctattacctcaaaacgagtgttatttctattaaacacttatttagggtttattatacgcagactgctgtgttctttgtgccttgggtaacctacacatttggagtttttttgtagggcttttaaattttgacacagaattgtcaactaaagaaatgaggaacaactgggaaaaggaaatcgcagctgtgatttctcctgaactggaggtaagcagtaagtgctgacagctgggttgctcctcggtccagaaaggaagggtcccggcgtctgctgaatgctccttgcctgttgcccctcgtagcggtgccagcaagagcctttgactgagccccagattccaatgctccccacctcgagtcactccatcctcctcctgtccccaagtgctcttccctcctacagctggtcatgaagtcagtccgaggaattcctgggaatccagcaagtgcaggcagtgtttttctctccattactttttacagcagaatgtaatgtacgaggtagagaaataaactatcacatatatacccattaaaaatgaactgacaatcatagattaattattcatctagcagccagaatgttagtaagagaaatataggtttctgaatctcattctgtttggggagacatacatctataaacaaacaacactgaaattagaagatcaaagggaatacctgattacatgctcagagcaggggacaaacgccctaatatgcagacacccactggtggagttactgggtgttacaatctgtttgtagcaatacctcacttccagaattttttttttcattttctccatccagcatctagataaaacccttcccaccatgaataatctcatcagccaagataagcgtatcagctctaaccctgtggccaaaataatatatggtgacccagtgaccttcctgccccacctgccccggaaaagtgtggtccattgctctaagatttggagctgcaggaaaagaattacagttgagtacctccagcacattgtggaacagaaaaatggcaaagaaagagtgcccatcctctggcatttcctgcagaaggtatgtctggcttactgtggctccctccctctttcggtgcagagttccccagcaggcctgaaggccctgggcttttacgtagtataatcattcttaacagactattccagtgctttggttttgaataggatgtagcagaagactcagggctagagatcttcatacatatcaaactgtatgattgccagaagcgtaaagtcacagacgaaatacacacccacgttgggattagcagtgacctgtggggaagctggaggccaggagaggacctcccctcatcactcagccagacagaaaaggatgcagctctttcccagccgctctgttcctctctgtccacaatgctggagcatgggcagatcaaacaaaactcctccctgtgcctaagcatcgaggcgtacgtggaagacgggagtgagtgggtgtgtgccggcggtgctggggtactggggagcagtcaagtgtactggccacatctgttggaattggaaatatactttcctaactttcacccatttgcactttcattttctacaacacattgtctaaacatttctctccatggactattatttgctgtctagcaaggatgaagacgagttggaaacttctgttggtcagttgaggagtatgagttcacaatgacaggaaggacttcctgtcaatactgtggcctctctctcagatgcagtttcactacgtcgaagatgcttagatcctaggtcttcggaaggacacccaggaataaggccagccctcgctgcctaagctctgggtctcttctttcagcagcttctctccgtacccagccagttacatgggcccaagggacagatttccttcatcagcctttgatggacgcggctgatgcgtcagctccatagaggctgggctccctcttcgccgccctccatttctagcacactgcactgtctgtagacttgtgggaagaacttttcccgttttcaagctccacgctgcatcacaggaaatgacactgaccagattaagctacaaatacaagtctcgcagccagtctcaaagtctgtaaccccaataacattttttaggtaaataaaaattgttactgggtggtcttcccttctccaggaagcagagctgaggctggtaaagttcctgcctgagattttggccttgcaaagggatctagtgaagcagttccagaacgtccagcaagttgaatacagctccatcaraggcttcctcagcaagcacagctcaggtgtggctctgctctgacaggaccaggactgtcccgcatttggcggttcgaaaggatcactgcataggggaacagggtggggcggaggggaggaggcgctgatgggtgctctatagcctaagcccttaccatgcggtgaagggtgcttgaaccccaaaaacatattagaagctttcgccacagctgaaaagcacctccctctctagctcctcaaactcggctcactccttcaaacagcacctcagacatgcttgtgactgcacaaagcaggaaagatgggctctcggcagagcatgtaactagggtggtttggccctttcagggggttgctatcatacggttcttaccaagtattctgttcaacagatgggttgaggcagctgcttcacaacaggatcacagtctttctgtccacatggaacaaactgaggagatcgcttgagacgaacggttagtatcctgtcccctgtaccactaagcgttccaggagagcctcgtctgaaagctcttctcgtcagcctgactcggcccatccctgtcaacacccagcactgtgtttgtttgtttttgagatggagtcttgctctgtcgccaggctggagtgcagtggcaccatctcggctcactgcaacctctgcctcccggattcaagcaattctcctgcctcagcccctagtagagacagggtttcaccatgttgcccaggctggtctcaaactcccaagctcaggcaatccacccgcctcggcctcccaaagtgctaggattacaagcgggagccactgcgcccagccagcactgttaacgcttaacttgtgtctcttttgatggtggtgtctggataagtaaaataatgtgtcaggaaaataaaccctcagtactcttcagtccctaaatatggcatcaaaagggagctgaaagcatttgaaactctatcagcatatgagatggggcagaaccgagacccggctcccggctgtgtgtggagctgatggcttctgcatccctcctccccacgcctagattctgtgaggagttggaactcctctctgcttaagcataaccctgtcgtttaaaggtgagatcaacctacccaaagactactgcagcactgacttggatctggacactgagtttgagatcctcttgccacgccgacggggcctgggcctctgtgctaccgctctcgtcagctacttgattcgcctacacaatgaaattgtctacgccgtggaaaaactctccaaggaaaacaacaggtttgtgcacgagccaccaggaagtggtgcctgctcagcccagagtccctaagcccagtggggcagacacaagcaagcttcagccccttccctaacagacactcactccttcagccgcccccaccagtgacccgccccatacttgggtagggttttccactgctccaactgtgagaaatgggagcctgctccctgcaacatagagccctaggcccgcatgcctggcctggacgctgagcgctgtctttctgcccctcagctattccgtggatgccgccgaggtcactgaactgcatgtcatcagttatgaagtggagcgggacctgactccactgattctctccaactgccagtaccaggtggaggagggcagagagaccgtgcaggagttcgatctggagaagattcagcggcagatcgtcagccgcttcctccagggcaagccccggctgagcctcaaggtagggctgactcctgccactgctgctcatttggtgtgtctgttgtgaacaagcagcccttggtgtgtttctcgagagagggaagcagcacctcaccctgcagtctggtctgtatctcctccctcacacctgcagctcccgaggccaccacgccacctgtatctccgggccgcagggctctcctgagccctctcctgcagggccatctgcacctcagtcccccaaggaactgttgaggtctttttttgaaacagggtctcactctgtcaccgaggctgcagggcagtggtgccaagcctccacctctgggctcaagcgatcctctcacatcagcttcccaaatagctgagatcacaagtgtgcaccaccatgcccagctaatttgtgtattttttggtagagatggggcttcaccacgttgcccggctggtctcgaactcttgggatcaagggatccacccgcctaggcctcccaaagtgctgggattataggcgtgagccactgcacccagctgattgttgaggtcttgattctaccagttaatggcaccaccacccccacagcccctcttccttcagcctccatgggtaacctgccagcagatgccattagctgtgccttctgtgtgtcctcacctgactgccaaagcccaccctgtagttaaaagcatcctgtacgttggactgctagccactcctaactggtgcccacctccaggttcttcagtccacggcctaaaacccacatctggctctcttcatcctgtgccggatggacctgcctggcctctcatggcatatgggataaagccgacactctgttgtctggtatgggaggccctgtagctcggcccagcccaaccttccagccctgcctccagccacttagcccatttccctgaactgcatcccgtgacctcatgcactgaagccacacagcaggcactttcccatctctgttctttgactggacgcacttgttccttccagctgcagtcatatctactgtacttatgtacggtaccacccgcaactgggccaggaactgtgagagcccatggattttttttttttttttttttttttgagacggagtctcgctctgtcacccaggctggattgcagtggcgcaatctcagcttgctgcaagctccacctcccgggttcactcatgccattctcctgccgcagcctcccgagtagctgggactacaggcgcccgccaccatgcccggctaatttttttgtatttttagtagagacggggtttcaccatgttagccaggatggtctcgatctcttgacttcgtgatctgcccgtctctgcctcccaaaggctgtttttaattcattctcacagccatttcctctggcagagtgactgcctgtgcatggtctgtgcctttagtggctgaatgaataaatgaatgggatatgcacaaatacctgttgccctagcgcctcctgtggcgaagcaggccacgatgatgtgacaatatcttgtcatgtaattggctgtgaggactccgctggattcccggttgtgttactatttccatagatggtgggttaatgacggagaggggcgctcttagccaagggatacacagggcagcgcggcactacgctgcagttttccggctgcagattctgtttgtcatctgctggaaatgtccacgatggatttaattttaaaaaacttttttctttcccaatttagggaatacccactctggtgtacagacacgactggaactatgaacatctctttatggacatcaagaacaaaatggcacaggtgatcccgataaacctgatcctgtgcacggggctttctgccttctcagtgtgtttccctccgtgttaggcctactccagccttgccccgggtgtttgctgtgagcccacagtttctgcggcctcccttacagagagcaagcaagacagcaaatcagcaagttttcttgtagagtaaaagcagattgacatcttgtgggtttgattcttaggaatgatgttgatgcatggccatgcctgctgaagcgggaagtccatggaaccgatttgttcctgctctccgcatgcgggtactgataggtagacctgtgtgtctccatggcctccaggaactgcctgcacaccgtgcgcccaagtgtcagctgttagagagttggccccccgcatgtggcttgggcagtgaaggggctcggctctcttaccaggtggtgagatgccagaaacccagcccacagacatccctctcctgcttttcatttcccaggactccctccccagctcggtcattagtgccatcagtggacagctgcagtcctacagcgatgcctgtgaagtgctgtctgtcgtagaagtcactctggggtttctgagcacagctggtggggatccaaacatgcagctgaatgtgtatactcaagacatcctgcaaatgggtgatcagacgattcacgtgttaaaggtgggtctcacaccgaaaaggaaatcagcacagcccctcaccctggtctcctgcttcccgcgagggataggagcattcagggagtgccactctaggcgctcctgaaaaggatggggagacaagaacaactgctcacccagtcagtccagcataaacaggacacaccagggacagtagagacagacacgcgctagaaatgccagcagtctagatagagcccaaatgtgactgagtacatgaggcccctgcaaacagcgtgatgtgagcgggctgtgaggtgccgcaggagggaactggtagataggacagagatggccttcacaaggagggagatcacattagtacagggcagaacgaagagaagaatgcctgtgtggagagcactcaggctccctggtgcacgacatgagtgggggtgtgagacctcagcccccactcaggaattctattccttctgcaggccttaaacagatgccagttaaaacacaccattgccctctggcagttcctgtctgctcataagtctgaacagctgctgcggctgcacaaagtaagtctggtctcttcctctctgctggacagagggactgcgctcccttctccctgcaggctttaatgcttcatttgctcttccgtcgttttaggagccatttggggaaatcagttcaaggtacaaagcggatctgagcccggaaaatgctaagctcctcagcacattcctaaatcagactggcctagacgccttcctgctagagctgcacgaaatgataatcttgaaactaaagaacccccaaacccaaaccgaggagcgcttccgccctcagtggaggtatggatttgcacacctatggggcggggcagacacaatgttgtgctgtctctcctgtgatcttctatagacacaaaaatagttcttttcttaccacagaatcccatttcctgctttttgtcattaccagggcacctgaggactggctttttggttttaatctctactgggttttctttaagcaattacacaagtgtttttcagacaagagtcttgccctgttacccaggctggagtgcagtggagtaatctcaactcactgcaaccttggctcactgcaacctctgcctcccaggttcaagcgagtctcatgcctcagcttcccaaatagctgggactacaggcgtgtaccaccatgcctggctaatttttgtatttttagtagagacagagtttcactgtgttggccaggctggtctcgaactcctggcctgaaatgatctgcccgctttggcctctcaaagtgctagaatgaaaaggcgtgagccactacacccagcctaaaagtcatatattctaattttataacaagtagactatgtcaaagagaggtacattttaaatttgatagaaaaagctatcctaggccgggtgtggaggcttaccctgtaatcccagcactttgggaggccgaggtgggtggatcacctgaggtcaggagttcaagaccagcctggccaactggtgaaactctgtttctactaaaaatacaaaaattagccaggcatggtggtacacacctgcagtcccacctactcaggaggctgaggcaggagaattgctttaacctgggaggtggagattgcagtgagctgagatcgcaccactgcactccagcctgggcaacagagggagacttcatctcaaaaacaaacaaaaaaaagaatagaaaaagctataccaatgtataaagagtgtatgagaatactcaacattggatattatccatcttttacatagttgccaatctaaaggccaaaatggtatctcattttcagttgtatttccccatttaccgtgaggctgaacatgttttttatatttatcacccatttgtatatcatcctctgaattgcctttatgttttattttttgagaccgtgtctcactcccacccaagctggagtgcagtggcgcaatcttggcttagtgcaaccgctgccttctgggttcaatcgattctcctgccttagcctccctagtagctgggattacaggtgccaaccatcacgcccagctaattttttctattttagtacagagacggggtttctccacgtggccaggctggtctcgagctcctgacctcaagtgattcacaagctttggccaccacacccagcctaaattacctcttcgtatctttttcttttgcgctttgtctctgtaatggttgtgagagctctttgcatagaacaggggttcatcttctctgccgtgtgccacagtgctctatttcagcctttaattttatgaagtcaaatctgacagaattttcctataaagggtctctatacaaaaacacttactaggatcttccttatcccaggattataaactagccttttttttttttttttttttttttttttgagatggagtcttgctgtgtcatccaggctagagtgcaatggtgtcatcttagctcactgcaacctctgcctcctgggttcaagcgattctcctgcctcagcctcccgagtagctgggactacaggcatgcacctgcccggctaatttttttgtacttttagtagagacgggtttcaccatgttgaccaggctggtctcgatctcctgacctcaagtgatctgcccgtctcggcctcccaaagtgctaggattacaggcgtgagccactgcgcccggcctgtcctctctcttcttgtgctttcacatacttgttttattcaccagtttatgtttaggtgtaaagtaaggatttaacttgatgttttcctgactagatcatcaaactccagtcttcctcaccaatatgtttcctatttgctatcttcctccattatgctatgtggcatataccatatacatttatgttctttcaactttttcaacaagtctttactagcctgtgcttttcatgtagataaaaaagatccctttcctagagaatttccagtctttgttaatgtgaaacatgtaatagaatacaaggttttttgaatggtgaggccttgtttatgcaaccctgtaaataaacatcaccttgctagagggcctaaagtcatgtgaaggggccagcacagggcttgctccgtgggcttctgtcgtgtttgtgtactgcatgtgggaagcagcagtcaggcaccagagccttgaacccaggatgctagtcccagttcaggagctcaggtcattgaccttaaactgctcctcaggatttctgggttttttttttttctcgctctgtcgcccaggctggagtgcagtggtgcaatctcggctcactgcaagctccacctcctgggttcaagtgattttcctgcctcagcctcccgagtagctgggattacaggcacccaccgccatgcctggctaatttttgtatttttactagagacagggtttctccatgttggctaggctggtctcgaactcctgacttcaagtgatccacctgcctcagcctcccaaagtgctaggattataggcgtgagccactgcgcctggcctaggattttaagtcttaacgagcattttggttctgctgatgacaacgggaaaaagtaacatgaggggactgattggtccccttatgttatatgggagcctttttttttgagacaggatctcactgtggcccaggctggagtgcagttgtgcaatctcagctcgctgtagcctccacctcctggactcaagtggtcctcccacctcagcctcctgagtagcttcggactataggcatacaccaccacgtgcccagctaatttttgtatttttgctagagacagggtttcgccatgttgcccaggctggtcttgaactcctgacctccactgacccacccacctcagcctcccacagtgctgggcttacacacgtgagccacacagtgctgggcttacacacgtgagccaccatgctgaggagactgttttaaatgctctcttctttggtttttcagcctgagagacactctcgtaagttacatgcaaactaaagaaagtgaaattcttcctgaaatggcatctcagttcccagaagagatactgctcgccagctgtgtctcagtgtggaaaacagctgctgtgctgaaatggaatcgagaaatgagatagaattatttcctcagctatctttggatgactttggagagaagactcctctctcctcgtctgcggcgtggacttgatcatggactggtgcctttgcattcagaaggagagctgtcagcgtagcaccgaattcaagaccaaggcgtgctacctgagctgacagctttttgaaagccgagctgtttctgaaccatgtacatacatgttctgaaactttctcatcattttatgagtactgttcattgagagatgacaatgaagattagatgaaattggaaataaaccaacattgtttacattccaggagacttgtagctcagccacacacgcagtaatgacctgtgcccgttcgcctctggcactgcccacccctctttttttttttcttctaattctgtactcacaaaagagaatctcattttcttctttcttccattcccttaaattctgagtactgtacatatatttctgggttcccacgatgatgtgaaaaactaccagactgttttttgtcttctcacaaagacaagaaaaatcagggcattttgtgagtgccttaagatcaaactaacaagatctgaccctctcccctcacagtgagccactgccccacttcagagggtaagagccaaaagcctcattgtgaaaggcactggacttggaccagggacaccatcagggccttggttttctcacgcataaaatggagagtggattaatcgccaaagattcttctgatctgacattttgaaattgtgagagaaactagatgactgtaaacttggtcacaggcctggttctggcagttctttgcggacttttttctagcattatgccaaataaacatgcagtctcagtgtgctctcgcatgtatgaatatctagtcctttctgtggttctcagccaagacataaaaactaggactcagagcacatacaaaaccagttatgtttcggaaagagggaaaagagtccccgagcccggatcttgtgctgcttttctcactgacgtgttgccttttttctttacaaaatctgctttgatacttaggacctctctggactaatttctcttcctagacagctcagcacagctattgatatgttagaggcagtatccttaatattcattctaaatgagttaacgacttaacttgaaattgggcctaaggagtgagaactacaaaaatacaaaatgcttgtccaggactcagccatgcacaccttgagcagcgccggcaggaggcacggaaggaactgtgctccgttctcctcactgtcatggtgccaccagtgtctgatgaagggcagagtgacccagactgcaggcagtaactgacttcacacagtccctggcatttagtcatctgtgattgttttatcactctggactgtgcagagccacctgccaccgagatctgcattccgactgcctatgaacgggtgtgggggccgggggctggcttgctgaagtcttcaacttgcactcggagctcctttgatacctcagagctggctgtcaggtggcagctcacacccagactcactggccacacctcagcagggggggagtcgagtgtcagtctctttctgtgaaggctttttttttcctttggcctgggaatttttcccatttttatgaaggggttttaaattgtttcattttgtgtgctgtgcttcaaagccttaactgtcaaatcttgcattatcttgtttgtacagaaatatactggcctagcagaggcaaaaaaaaaaaaaatgaattttattttacttgtcacacctgtcttaataaactggagttttgctgctaaagaactcttctctctgggggcagagcttctatttatggcacatagacatcagctaggcttttgggaatcgtttgtgttctttgtggaaatgtcctttagaagcacccatgaagtagtgtgttcagactgtgcacacagaaaacaggctctgccttcacatgtgagacggtggacttttcctctggacaaaatgacagcatcctggcgactccacagtggagctgagcgccactccctgtagccgatctgggactgaaacgcttacacctctgcctcagaaggagtcccccatgccctgcctgaaatgacttcactggacacagcggggctgcagctaacggggtacaggtaggagctaactaacttcacccctgagtccacttgcggggtaagagataaacagtaacccttccaggagcccactgacgttggagtgctaaaaatgccccttcagggggaaaactgcattttctcttccaaaaaggaaaggttcttccaggcgagaaacctgtggtctagaaccacagcaagaagaggaggcatgctggcctgcaccggaagactcactttgtctgccctgcgccagcctcacctcaccctgcagttcccgtttccgccatggatgcctcatcaccaaccctgaccttccccctcccaaccctttattcatcctcactcccactcatacccgcctccctggacagttccctgctgcagagttctttctgctttcagccctaccttggtggtgatttacctgaaaatcttcacaactgatcattatctccttctctttgagacctgactgaaaaaattaggtgtgcacacctgtaatcccagctacctgggaggctgaggtatgaggattgcttgagcctgggagtttgaggctgcagtgagctatgattgcaccactgcactccaacctgggtgacagagtgagaccctgtctcaaaaaaaaataaaataaggaccttattgtgtgtgcaaattatttgcctttcatgaatagttatgagactttgtgcatgtgttaaccgagacggccctgagctatttcagttactcctgtatgttcaggcaattccaatttagaaacaaacttccagtgataaatattgggaagaaaatgtcagctcagggccaagtaggtaaagccacccaagtctagccatgcacacaggcactagtgtgctgaaggaaaacacctctgatttgatacataaagtgtgaaggttgcataactggggaggtggctggagaaaacctgagcctcgagctgagtaacaatccaagccatgattagcatttatattgcattctgcattattctcatcctatcacgttcattaagttcaaccattaccattacgaatcccgtttccagctggaaaaacaagcgccaggaaagtgcatttcccccccaccccccccccccaaccagagcaactttggtcagaagtcacacctaatcaccctctctgcaggatatgctggggatagcccttttgaggaggtgggggtgtatgcgaaagcaacctggggccaaagcagggggaagcaggagagactggaggggccctgggtccagcaagtgctccaccaggctgcccctgctcaggtgctccacagcccataaaaaacgccttcagccctgctgtgcttcccctcacacctaaacttactataagggtcctaccttccccagtattccttcatgatcactgctacctgtgccctgaagcgcaacagaacctcaacccagaggacagccacttcctcagaacccacgatctttgccccatcgccacaggtccatgagtgactcctaattaccaaatccaagggctccttcaccctgaccactctggaacccaacaaagctgacacacagcccagcccagacaggcctgaatgtgggtcctggctctcccatttgccagttctacagccatgggcaagtggctcgacctctctaagcttcagtttcctcgtctgtgaaatatctgtcagtgctcttaagggaggagaccaccccacatattgtcttacgcccaatttctgcctccaaagagagaagtaaaaactaaaaggcagaaatgaaatccacaagcagacagcctgcaccacaccctgggcctggtagttaaagatcgacgcctgacctaatcagtgatgctatctatagattacagacattatatagaaaagcaccgtgaaaatccctgtcctgttctgttccgttctaattacgggtgcatgcagcccccagtcacgtaccccctgcttgctcaatcaatcacgaccctctcacgcagaccccttaagagttgtgagcccttaaaagggacaggaattgctcactcggggagctcagttgttggagacatgagtcttgccgaagcgcccggctgaataaagcccttctttaactcgtgtctgaggggttttctctgcagcttgtcctgctacatttcttggttccctgaccaggaatctaggtgattaatggatggttgaggcagcccctcagacggcttaggcctgccctgtggagcatccctgtggaggactccagccagcttgagcgacatggatcctgagagcgctctcaggcaggcaattgccccagtggaatgcctggccagagcagtgtgtagcaggcccccgaggaggatcaacgcagtggctgaacaccgggaaggaactgccacttggagtccagacatctgaaacttggtaagactggtctttggaacttgcccactccatttgaggggaagcgtggcctgatcacccacggcgtgcctgtactggcactctggtttttgtttttgacttgacttggattgcttgatactttggttttggttttgacctgacttggatatcctgatactctgattttggttttgattttggtttggtgtaaactgtaaaagtgtgtgtgtgccctttttacctgttctttgttttgtggtatgcgtgtagggtgagcgtaatgttttgtcttgaagaagcatgggtcaagcacaaagtaagaccaccccactaggaactatgctgaaaaatttcaggaaaggatttaagggagactatggagtactatgacaccaggaaaacttaaaactttgtgtaagatagactggccagcattagaggtaggttggccatcagagtgaagcctggacaggtcccttgtttcaaaggtatggcacaaggtaacctgtaagccagggtacccacaccagttcccgtacacagacacttggttacagctggttttagactccccacccgcccgccccaacagtggttgagagaacagcagcataagtggctggcagaggcaaggaaagaccagcagagagagaaagaggaagagacagacaaagagggagtcagagagagagagagaaagagacagaggcaaaaggaaagtcaaagagaaagagacagaaaatcaagaggaaaaaaagaaaaaaagagatatatatacaagtagttagaaacaacaacaacactgtaccctattcctttaaaagccaagataaatttaaaacctataattgataattgaaagtattctctgtaaccctgtaacactccaataccactttgttgtcagtgtaaacaagggcatatcccgaaagcactgaggccttcccatcaaaaatccttaacccagtaacccgtggatggctcaaatgcattcaatctgtagctgcaactgctttgctaacagaaaaaagtaaaaaaataacttttacaggaaacctcattgtgagcacacctcaccagttcagaagtagcctaaggaaaaaaaaaaaaggatgatttaactttaactactgaaaattcccttaacccagcaggtttcctaacaggggatctaaatcttaattaccgtacaaaggccctaccatacctaggaggaactcccttcaggaaaggatgatctatggttcctcccaggtaattaaaggaaaaaaaaaaagccatctatacgaattctaagttaattttgacaaaacgaggtcttactaatagcaaaggataattaaaatcccaagcttacaaggttttcaacaaaagtaaagtttgctaaaagttaacagtgtaacatgtattatagtaatttctgatcttgtggcctcagacagtctagtccacagacataaaagaagttcgctttggaaaagaatggttatcatcttcaggaaaaaaaaaaagaaggggcagaatttacataaaaagagtgttatatggtaaattcttgtcctgaaataaattaactggttgtttaaagaaagaaatgtttgtaataagtcagaaagttgaggcatgtcgaagaattgtctgcgaaagtcgtgaaagagaaaaatgttataaaaaattatgcaagaaatgttgtataatttaaaagtaactaggcctcctaaatgtaaaactattgaaagaaaaaaaaaacagtttatgtgcaagtgtcaggcctctgagcccaagctaagccatcatatccccagggacctgcacgtatacatccagatggcctgaagcaactgaagatccacaaaggaagtgaaaatagccttaactgatgacattccaccattgtgatttgtttctgccccatcctaactgatcaatgtactttgtaatctctcccacccttaagaaggttcttcgtaattctttgtaatcccacccttgagaatgtactttgtgagatccaccccctgcctgcaaaacattgctcctaactccaccgcctatcccaaacctacaagaactaatgataatcccaccacactttgctgactctctttttggactcagcccgcctgcacccaggtgaaataaacagccttgttgctcacacaaagcctgtttggtggtctcttcacacggatgcgtgtgacgtgcgtgacatttggtaccaaagatccgggacgggagtactccttacagagaccggtcccctgtcctcatcctcactccatgaggagatccacgtacaacctcgggttctcagaccaaccagcccaaggaacatctcatgaatttcaaatcgggtaagtggtcttttcactctcttctccagcctctctcgctacccttcagtctccctgtccttccaattccagttctttttcctctctagtagagacaaaggagacatattttatccgtggacccaaaactccggcgccagtcacggactcggcaagacagtcttcccctggtgtttaatgactgcagggacgcctgcctgattattcacccacactccactggtgtctaatcactgtggggatgcctgccttggtcattcacccacattcccttggtggcaagtcaattgcggggacaacctgctttggctgctcacccacattgcagcccacggctgctccctaccccccttctccgtgtctctacccttctctttaaactggcctccttcactgtgggcaacctttcaccctccattcctccttcttctcccttagcctgtgttctcaagaacttaaaacctcttcaactctcacttgacctaaaatctaagcatcttattttcttctgcaacaccgcttggccccagtacgaactcgacaatggttctaaatggccagaaaatggcacttttgatttctccatcctgcaagacctagataatttttgtcgaaaaatgggcaaatagtctgaggtgccttacgtccaggcatttttcagacttcgttccctccctagtctctgttcccactgtgactcatcccaaatcctcctcctttccctcccgcctgtcccttcagtcccaacctcgagcgttgctgagtcttgtgaatcttccttttctgctgacccatctgacccctcacctcctccccaggctgctcctcctcagctcgctccccgccaggctgatcaggctccagctcttcttcagcctctgctccccaccctataacccttctattacctccctcccacacccggtccggtttacagtttctttctgcgaccagctctcccccatctgcccaacaatttcctcatagagaggtggctggagctgaaggcatatgtcagggtacatgtgcctttttctctatcagacctttcccaaatcagccagcgtttaggctctttctcatcagaccccactaaatacatacaggaattccgatatctaactctgtcctacagtttaacctggagtgactgaaatgtcatcctaacttctaccctctccccagataaatgggaaagagttttttctctagcccaatctcacactgacaaccaccggcttcacgagccagacctccaggagggcatcagagcagttccccgagaagatccccagtggaactaccaggcagattccccaggtatagctacgcgagattacatggtttcctgcctagttgaagggcttaaaaaggcagcttacaaagctgttaattatgacaaacaagaaactacccgaggtgaagatgaaaacccagcccagttcatggcccacttggcagtaacccttagacgctttaccgccctagacccagaggggccaggaggccgccttattcttaatatgcattttatcacccagtcagctcctgatgttagaaaaaagattcaaaaactggaatccagccctcataccccacaacaggaattaatcaacctcaccttcaaggtgtacaataataggaggtagccagacagcaacgcatttctgagttacagctactcgcctctgctgtaagacagcccacaaccacgtctccagcatacaagaacttcagaacatccaagccacagctcccaggggctccttcaaaacatcctcgtgggccttgcttcaaatgccaaaagcctggccactgggccaaggaatgcccgcagcctgggattcctcctaagctgtgccctgtctgtgcgcgcccccactggaggtcggactgtccgactcacatcgccgctgctcctaaagcacctggagcccaaacccaacgttccttggccaactccttcccagatctcctcggcttagtggctgaagactgacgctgcccatcgcctcgggagcccctggaccatcacagacactttgggtaactcttacagtggaggataagtctgtccctttcttaatcaatacagaggctacccactccacattaacctttttttcaagggcctgtttcccttgcctccgtaactgttgtgggtattggcggccaggcttctaaacctcttaaattccccaactctggtgccaacttggacaacattcttttatgcacccctttttagttatccccacctgcccagatcccttactagattgagacattttaactaaattatctgcttccctgactattcctagcctacaaccacacctcattgccacccttttctccagttcaaagccgccttcacaccctccccttggatctccccaccttaatccacaagtataggacacctctactcccaccttggcaaccaatcatgcaccccttaccatcccattaaaacctaatcactcttaccccactcaatgccaatatcccatcccacagcacactttaaaaggattaaagcctgttatcactcgcctgttatagcatggccttttaaaacccataaacactccttataattcccccattttacctgttccaaaaactggacaagccttacaggttaatttcagaatctgcaccttatcaaccaaattgtcttgcctatccaccctgtggcgccaaagccatatactcttctatcctcaatacctccctccagaacccctctacatatattattatgtagatatgtacatatctacatatgtacataatctacataacccattctgttctggatctcaaacatgctttctttactattcctttgcacccttcatcccagcctctcttcattttcacttggactgacccggacacccatcaggctcagcaaattacctgggctgtactgccacaaggcttcacggacagccccccccccccccgttacttcagtcaagcccaaatttcttcctcatcctttacctatctcgacttaattcttcatgaaaacacagtgctctccctgctgatcgtgtctagctaatctcccaaaccccgaccccttctacaaagcaacagctcctttccttcctaggcatggttagatactccgcctttggatacctagctttaccatcctgactaaaccatcatataaactcacaaaagcaaacccagctgaccccacagatcctaaatcctttcgccactcctctttccgttccctgaaaacagccctagaagctgctcccacactagctctccctaactcatcccaacactttttcattacatacagccaaagtgcagggttgtgcagtcagaattcttacacaagagccaggaacggcccctgcagcccttctgtccaaacaacttgaccttactgttttaggctagcccccacattattcctgataccacacctgaccccccatgactgtatctctctaatccacctagcatttactcaatttccccatatttccttctttcctgttcctcaccctgatcacacttggtttattgatggcagttccaccaggcctaatcgccactcaccagcaaaggcaggctatgctatagtaacttccacacctatcattgaggctaccactctgcccccctccactacctctcagcaagccaaactcattgccttaactcaagccctcactcttgcagaaggagtacgtgtcaatatttatactgactctatgccttccatagagtcagcatgcaccaccatgctgttatatgggcagagagaggtttcctcactatgtaagggtcctccatcattaatgcctctttaataaaaattcttctcaaggtcgctttacttccaaaggaatctggagtcatttactgcaagagccatcaaaaggcatcagatcccatcattcagggcaacacttatgctgataagttagctaaagaagcagcttagttttccaacttctgtccctcacggccagtttttctccttctcatcggtcactcctacctactctcccactgaaacttccacctatcaatctcttcccacacaatgtaaatggttcttagacaaaggaaaatatctccttccagcctcacaggcccattctattctatcatcatttcacaacctcttccatgtaggttacaagccgctagcctgtctcttagaacctctcatttcctttccatcgtggaaatctatcctcaaggaaatcacttctcagtgttccatctgctattctactactcctcaaggattgttcaggccccctccctccctacacatcaagcgcggggatttgcccctgcccaggaccggcaaattgactttactcacaggccccgagtcaggaaactaaaatacctcttggtctggatagatgctttcactggatgcgtagaggcctttcccacagggtctgggaaggccaccgcagtcatttcttctcttctgtcagacataattcctcggtttggccttcccacctctatacagtctgataacggaccggcctttattagtcaaatcacccaagcagtttctcaggctcttggtattcagtggaaccttcatatcccttaccatctcaatcttcaggaaaggtagaagggactaatggtcttttacagacacacctcaccaagctcagcctccagcttaaaaaggactggacagtacttctacctcttgcccttctcagaattagagcctgtcctcaagaagctacacggtacagtccatttaaacttttatgtggacgcactttcttgctcagccccaacctcatcccagacaccagccctctaggcaactatcttccagtctttcagcaggctagacaggaaattcgccaggctgctaatcttctcttgcctactccagattcccagccacatgaagacaccctagctggatggtcagttcttgttaataatctgacccctcaaactctacaacctcgatggaccggaccctacttaaacatctatagtacccccaaccaccatctgcctgcagaaccctccccactgggttcaccgttccagaataaagctgtgtccgtcagacagccagtccgatatctcctcttcctcctggaagtcgcaagtactctcccctacttcccttaaactcactcgcatttctgaagaacaataacccttatgagcctaatacatcccttcattctattaggtctatccatccttaccctactcttcgcaacagggctttacgcagtcacccccactacttggactgtgccccaaaaacctgtcatccctactatcttctgtctagtcatactcctattcaccattctcaactactcataaatgacctgctcttgtttacactgccagtttacattgtttctccaagacatcacagctgatatctcctggtgatatccccaaaccgccactcttgactcctgcttggagtggatagatgatctttggtggcagggcaccctccaacacttccaccctgatgaagttctattctttacttttatattcactcttattctcattcccattcttatgccactctctacctctccccagttacctccagcatactatcaatctcacccactctctcctcactgcctccaatccttttctagcaaagaattgttggctacgcgtttccctttcctcctgcttttacacagccatccctgctctacaggctgactgggctacctctcccgtctccctgcaactcagaacctcctttaataaccctcatctttacccgcctgaggaacttctttattttctagacagatttggtgagaactccccagacatttcacaccaacgagctgccacacttctccgcatctacttacggcatctttctccttatgtcaattccactcctcccatatttggaccccaaccacacaaacaactatccctgttgccgtttctttatgcatctcccgacaacagcctgctggaatccctttaggcaaccttccaccgtccaaatgttcctttactctttgtctccagaacccagccacacacattaccaaacagatgggagcattccaacttcgcattactgacaagccctctatcattattgacaaactaaaaaacattggcagtcactattgtttaggaagacacctaccctgcatctcaccccatccttggctaccaccctcccctgctcgtctgaatctcctcctagcccctcctcttgcttgcttataccctgccccatgaatagcagcgaaaggttacttgtagacactatgcgctttctcatacaccatgaaaaccaaacccctccctctacgcagttgcaccatcaatccccattacaacctctaacggctgctgcccttgctggatcgctaggattctggatgcaggattcctctttcagtacaccctctcaccttttcactttacatttccagttctgcctgacacaaggtctcttctttttatgtggctcttccacctacatgtgcctacctgccaaatggatgggcacatgtactctagtcttccttacctccaaaatccagtttgcagatgggaacgaacaactgcctgtccccctcatgacatcaacacaacaaaaaagagtcatcccactaatccctttacttattggtctaggactttctgcctccagtattgcacttggaactggaatagcaggcatctcaacctctgtcacaacagtccgcagcctctctaatgacttctctgctagcactacagatagatcacaaactttatctgttctccaagcccaggttaactctttagctgcagttgtcctccagaacggccaaggccgcgatttactcactgctgaaaaaggaggactctgtatatttttaaatgaagagtgttgtttttacctaaatcaatctggcctgctatatgacaacataaaaaaactcaaggatagagcccaaaagctcgccaaccaagcaaacaataacattgaacccccttggacactctctaattggacgtcctgggtactcccaattcttagtcctttaatacctatttttctccttcttttattcggaccttgtgtctttcgtttagtttctcaattcatataaaaccgcatccaggccatcatcagtaattctatatgacaaatgctccttctaacaaccccaaaatctttcttcagttgaatctctcccactgtaggttcccacaccgcccctgatcccgctcgaagcagccttgagaaacatcgcccattatctctccataccacccccaaaatttgtcactgctccaacacttcaccactattttgttttgcttttcttattaatataagaagacaggaatgtcagccctctgagcccaagctaagccatcatatcccgtgacctgcatatacatccagatggcctgaagcaactgaagatccacaaaggaagtgaaaatagccttaactgatgacattccaccattgtgatttgtttctgccccatcctaactgatcaatgtactttgtaatctctcccacccttaagaaggttctttgtaattctccccacccttgagagtgtactttgtgagatccaccccctgccggcaaaacattgctcctaacccaaccgcctaccccaaacctgtaagaactaatgataatccaccaccctttgctgactcttttcagaatcagcccgcctgcacccaggtgaaataaacagccttgttgctcacacaaagcctgtttggtgggcttttcacacggaggcgctaacacaaggtgtataagaaaagtaaatatacctttggtaaaaggattataagaaggcataagaatgtaaatttttacctagattaaaaggttaaaaaaattgttttgaaggtttaagcaagttttaaaacattaattataaaaaaattctgtgtgtaaacgtattagctaaagttaaaggggtatcatccagtttttctgtgaactggacattaaagtaaaaacacaatgggtttttcttaaagcactaacttgctctttaacaaagattataaaaggttaaaaagagtctataaaaatcttaccttacggtctgacattaaaaattgaataaatatgtatacaaagttttattaaaactaagtttaacattaatagcacactaatataaaggtgaaatttagcttatctggtataaaaatcatacagaaagcattattaaatataaaacagtgtttggctttctttggtccaaaaactaataaaaataggtggtaaaggaaatttctcagtaaaaaggcaccaaagcctataaagtccactcttgatgtccccacatttgaaacaaaaaatcaatttcttagaaattatatacttggtttgtcttccactttcctttccctcaaaactaaaagccttttagcacaggtgccagccctagaatttccagtaaaccagcaccagcctgaagatcaccttcttatcaaagggaggaaagaaaacaaatttttcaaaggaaaagtttttcttatcaaaggaaagaaagaaaacaaagggaggaaagaaaacaagccagcctaggaaggacccgaccttgtgctgctaaccaccgagactgctgtccctacagtggaaaagggatggactcatcacacccgagtcaaagctccaccccctccggagtcgtgggccccagtccaaggggaaaaccctaccaaactaaagctaagaaatatttaactcttttatctattctattactctttcttctttcctcgctctattgctgaccatctggttattaacacaaccaaatcaatttcacctcaaactattgcatttaatgcttgtcttgttataccctgtggggacttgccaagtcgaagacagctctctacttcagaaaagtacttctgtccctcctgactctcctcagactgggcattagtaaattaggaccatttaatccgggggaatttcaataaagactccagtgtcaaccaggagtcttgctccccaatgtagagcttttatgccgtagttggtccaacattctgtggaccactaaagagcaagaatggactgccccaaccagtttttgtaatttcccaaaatcatacattcattttactagaagttcatattagttaaagacttaaaacaaactttggcaattaagacagcataccaagatgcaaatgcctggttgaaatggatcaaatattccatccgcacgttaaacaaaagcaactgttacgcttgtgcacatggcaggccagaggtcctgattgtcccctttccactaaggtggtcctccagtcgaccaggtgtggctgcatggtagctcttctccaggattctacagcctggagtaataagttgtgccaagctctctctgctgtatccgaaagtctggcgccttgcgggtcagcccccgagggccatcgagccgccgtctcccaatactaagttcacttcgtgtctctcatgacagggaggaaacttagcattccttggaaacctgaagggatgcagtgagcttaaaaattttcaagagcctatcaatcagccagcccttgttcatccctgagtggatgtgagcggatgtgtggtggtattgtggtggacctttactgggcactctgccgaataactggagtggcgcttgtactttagtccgattggctatccctttcaccctggcattccatcaaccagagggaggaaaaataagacatcgtaaagcaagagaagccccttatgggtctttcgactctcatttccatttagacgcaattggagtcccacggggaatactagatcaatttaaaacttgaaatcaaatagcggcaggatttgagtcaatattttggtagatgacagttaataaaaatgtaaattggataaactacatctattacaacaaacagcaacaagcttttcatgagttaaaagaaaaacgtaggccccagccctggggctatcagacctgacaaaaccctttacaccctatgtgtcagaaagagaaaaaatgcagttggagttttaacccagactgtgggaccctggctaaggccagtggcctatctctcaaaacaactagacggggtttccaaagcctggcccccgtgtctaagggccctggcagcaacggccctgttagcacaagaagcagataaactaacccttggacaaaacctgaatataaaggccccccacgctgtggtaactttaatgactaccaaaggtcatcactggttaacaaatgctagattaaccaagtaccaaagcttgctacgtgaaaatccccacataaccactgcagtttgcaacacgctaaaccccgccaccttgctcctgatatgagagagcccagttgaacataactgtgtagaggtgttggactcagtttattctagcaggcgcaacctccgagaccatccttgaacatcagtaggctgtgagtggtacgtggacggaagcagcttcgccagcccctgcaaagtgactctgaggaagacgacaagccctgctccagtcacaactggaagctgactggtccacacacggccgaagcatgagaaaactcatcgtgggactcattttccttaaaatttggacttgtacagtaaggacttcaactgaccttcctcagactgaggactgttcccagtgtatacatcaagtcactgaggtaggacaaaaagttgccacagtcctattattttatgattattgtaagtgtaccaggactctaaaaaaaaaaaaagtttgtataatgctattctatgcaaggtatgtagcccaagaaatgaccaacctgatgtgtgttatgacctgagtctcccatgaccacagcttttgaaataagattaaggactgaggactggtagggactcataaacgatacgagtaaagtgttagccaaaatgaaagaaaaaggggtgcccaaacaagtcactttaaagtttgatgcctgtgctgtcattaatagtaataagttaaaaatagaatgtggttcccttaattagaaaagaggccatatgacaaaaaataagtacatttgtcataaattaaaactgtgtggaaataaatgtaaatactggtcttgtgtcatttaggctacttaaataaaaaataaaaaggatcctgtccaccttcaaaaaggaaaaagtggcccttcctgtaccagtggtcagtgtaaccccttagaactagtaataaccaacccccttgatcctcgctggaaaaaaggggagcatgtaaccctaggaatcaatggggctgaactggatccttgagtaaatatcatggtttgaggagaaatttataaacgctctcctgagacagtatttcaaaccttctatgatcaactgaatgtgcctgcaccagaaattccaggaaaaacaagacatttgtttttgcaattagccgagcatgtggcccagtctctcaatgtcacttcatgttatgtgtgtggacgaactgtaatgggagatcaatggccacgggaagcccgagaattagtacctacagacccagttcctgatgaattcccggctcaaaagaatcaccctgataacttctgggtcctaaaagcctcaatcattagacaatactgtatatagcaagagtggggaaggacttcacctttcctgtgggaagactcagctgcttaggcaaaaactgtataatactactacaaaaacagccacctagtggagttcaaaccacactaagaaaaatccatttagtaaattcccaaagttgcaaactgtgtgaacccacccggagtcccacagggactggacagcccccactggattatactggatatgtaggcatagagtttacaccaaattacccgaccagtgggcaggtagtcatgttattggcactattaaaccatctttcttcctactgcccataaagacaggcgaactcctgggcttccctgtctgtgcttcccgcaaaaagagaagcatagctataaaaaattcaaaagataatacatggccccctgaaagaatcatacaatattatgggcctggtacttaggcacaagatggctcctagggatacccgacccccatttacatgctcaaccgaatcatacggttacaagctgtcttagaaataatcactaataagaccggcagagccttgactattctggcctggcaagaaactcagatgagaaatgctatctatcaaaatagattggctctcgactacttgctagcagctgaaggagaggtctgtaaaaaatttaaccttactaattgctgtctacacatagatgatcgagggcaattagttgaagacacagttagaaatatgacaaaactggcacatgtgcccgtgcaagtgtggcatggatttggtcctggggccatgtttggaaaatggttcccagcactaaaaagatttaaaactcttatattaggagttacaatagcaatagaaacctgcgttctgctcccttgtttgctacccatacttcttcaaatgataaaaagcttcatcgctaccttagttcaccaaaatgcttcagcacaactgtactatatgaatcactatcgatctgtcttgcaagaaaacataggtagtaaaaataaaagtgacaactcccactactgagtgagattctcagagagggagaataagggagaccacccctcatattgttttacgcccaatttctgcctccaaagagagaagtaaaaactaaaaggcagaaatgaaatccacaagcagacagcctgcaccacaccctgggcctggtagttaaagatcgacgcctgacctaatcagtgatgctatctatagattacagacgttatatagaaaagcaccgtgaaaatccctgtcctgttctgttccgttctaattacgggtgcatgcagcccccagtcacgtaccccctgcttgctcaatcaatcacgaccctctcacgcagacccctgaagagttgtgagcccttaaaagggacaggaattgctcactcggggagctcggttgttggagacgtgagtcttgccgaagctcccagctgaataaagcccttctttaactcggtgtctgaggggttttgtctgcggcttgtcctgctacactctggtgggaaagacaccgcagcaggggagcagggtgcgcagggtgcagtgggtgacgctcattcgctgaaaactgaccctgctttcaaagccttgtcagttcccaaagctgtatgaagcctttcctgagcctcaacctctagagtctaatttcatcctctgtattttcccctttgttagtccaaactgcgtcattttgtaagcccctttccattttgtagaccttggtcaaagtgaaacattccacgggggttcgagccatgagaaacagcctgcctcttaacatattctgctgggacaaagcgccaggaacaccacattctgccggaaccacctcatcctgggaacacgttaacatcttcccggacagcaagccatactgcctagatccctcctgcccaggcctatgagtaccccagcctgtaatcgctggtgggctctggcattaagctggcccccgacaggtgtttgcaatacacctgtgttgctgtagagccgcccgcgcgccggcaatctcactgtgcgtcattctttaaccttcaccttcccttcaaaacctaacacccttgacgtttatattcaggctattatatatacatacacacatacacccttctgaatcctcccaggagaatataaaaccatgggttaaactgctacacttcagcttcagtggctgcctttcagtgcttcaaatacaccaacctctttcccaccccgggacctgcacgagaaagctgctccactcacccgccgtgctctttcactcacccttcaggtgtcttcaatatcgcctctcaggcctttcccaaccaccggtttaaagtcggctccccacaaataaatacatattctccctcacagtacttcgcacaacacctggcacagcgagagcactgaaccaacactcattcaatgaggaacacgaggtaagaaccaagaccccccaaggtcgccgtactttgtctggtaggagaagctttctgcccagcaaggtccgacccagatgaaaccccgtccaagtctgcagcacccccgtgagtccagcccgctaaactgtgaagaatttaaatacacgacacctgccctcaaaagcttgagggcacccctgagctgccatcgaggtgggggcgtcaacgacaggcgctcagtaagtgctcctgaggcaactgccgggctgtcctggacctggtgaatcctttcggggttcagcaaaggcggacgctgaccgggccaaggagtgggacctgcagtgctggaccacccttttccaagcgcagaagtgccgtgcccgccggctctcaacagggcaagaggcccctctcgccgtgcagagcgcagcgcttgcggcggggacagcagaggcgggacgggcgtcacggagttcccgggtcctcgcgcggtcacttacctggcctcagctatccgctgaaaaaggggcggacggcctcccgggatccgagccgatatcgcgcgcggcgccgcggggcaacctgaagccgtgtgcgtgcgtgtgcgttcacgcaggggctcacccagcggccacgggcttctgaagacccgggtgggaagtgcggcccgggaaagcgcaagcgtctccatggcaacgagggcggacggcgtcagagcgagtcgcttccggaagtgacgtgcggaaggggtgcccgggacgaagccatggccctggaggcggcgggagggccgccggaggaaacgctgtcactgtggaaacggtaatgctgtcaggcgacgcgcaggaggcgggggccgaggccgggcggcccttcgcggaccctccgagctcatagtcttcaggctgtggaatcggagctgccaccgcccgagactccaaagcaaagcccagtttccggcgcggaagcggagggatctcaggaattgtagtccgcggggtgggcgcggttctcgcttccggccagtttgtctggcctgcgcttgcgcgggtctccgccgcctgggctcctagggactgtggcctcggcggtatgtcccttgctttcccttgaagcgggagaagacccggcagaggcgctctgtccgctgcagccgcgcgggtggaggaggcagagtctgaggtgtgaccccgaccaagtttgacgcttctgtcctcctagggagcaagctcggctgaaggcccacgtcgtagaccgggacaccgaggcgtggcagcgagaccccgccttctcgggtctgcagagggtcgggggcgttgacgtgtccttcgtgaaaggggacagtgtccgcgcttgtgcttccctggtggtgctcagcttccctgagctcgaggtaacctgggaggacgccgagctcgaggcgggcccctcggtgggctcgggcgtgcggtctccgggacagggagcagtgcaagcgtagaacccggcttctcgttttgtaggatgtcattaatagtctggggagttggccgggcgcggtggctcacgcctgcaatcccagcactttgggaggctgaggcgggggaatcacctgaggtcgggagttcgagaccagcctgaccaacatggagaaacccggtctctactaaaaatacaaaattagacgggtgtggtggcatatacctgtaatcccagctactcggaaggctgaggcgggagaatcgcttgaacccgggaggcggaggttgcggtgagccaacatcacgccattgcactccagcctcggcaacaagagcgaaactccatctcaaaaaaaaaaaaaaaaaaaaaaagtctggggagtcttggaagggaaaaaaaggcaaaaattaagggaattctgtgactattaagaatcagctcatgagctttatcttcaagtctgattgttatcttgggttcttcatctcagtgacctgtccaccacgctcccagttgcccaagctggagatctgcatcctcctgggcccctccgttcctctcgcgtccagttgcacactagctcgcttttattctgcctcttaaatgtcgctggaatctgtccccttgtctgtttcccctctgctccttccctggctgtgccaccatcactcctgtgggttcctgagcagcattttcatgggtgccctggctcaaatgttcctcctcagtggtttcttttccatactgccagcagaccaattttcctaaaaagcaagtattttcctcaaaagcaacaagtcccctccctccctccctccctcccttccttccttccttccttcctcccttccttcctctctctgtctctctttccttcctttccccttcccccactctgtgcccaggctgaagtgcagtggcataatcttggctcactgtcaagatctgatctcaacctcctggactcaagtgatcctcccaccttagcctgagtagctaggaccacaggtgtgagccactacacccggcttatttttttattttttgtagagacaggatctcactatgttgcccagggtggtctccagctcctgggctcaagggatcctcctaccttggcctccccaagtgctgagattacaggtgggaaccaccacacccagcccacaagtcctcttagaacattgaagtgcccttctcatgacctcatggtgaataaagttcactctggttggcatggcttcaaggcccgtcatgacctgtcccctgcgtaatttaatatgacgctggaggctcagccctcaccactattctcagttcttcccacctcctacagcccactgtgagtccctcacgtgtttgctacgctgtatttggcggcctgacctttgcagtaatgtttcttctggaagggtttccatctggctaactactactcatctctttgggctcagtttagacatgacgtcctccactaagctccctctcatcccacaagacgaggacttgtccctcagcaccatgtcttagtccgtttgggctgctgtaatagaataccttagactgggtggcctacccacagcaggaatttatttctcacagttctggaggctggaggctcaagatcagggtgccagcgcaatcaggttgtggtgacggcgctctttcaggttgcacagggccagcttcttaaccgtgttctcacatggtgatccactcacctccctagaggccccacctcgtaataccatcacctatgggttataatttcaacataggaatttggggaggacaccagcatttagaccgtagcaaccctctgcttcctcttgtcttgctgggatggaactggcctgtcttccctactggaatataaactccatgatggtagaaaccatgttgctttgcccaccatggatagcatgaggaagacgctggactcttccttcaagagatacgtgtgtgaatggacagtgggctcatgattcacataagagtcaacagctccggaagcctcttggcgtcactcgcattgctcctctgctgactcccgaaggtgacaaacgtcttcctgcttccatggctttgtgctttacttgggaggcccttttcagtccaaatcaagcaaaaatgtactgagtgcctactgtgtgctaagattgggatattcagcagcccctactctaagaggtcccctggccattgtgtaggctagcatagggagtggggacacagtcttagggtcctgactactcatgctcacacccaactctgccactccccactttgccctctaggcctctgtctccttttttctgacttgaggatgatagtggtattaatatccagctcacagaattgtgatgagcagtacatgaaatgcatggaaagcagttaacacagcattcccttccccctccaacttctcctggctgcaaattaatcacctcatctgcatcctgggcctcttttgaatgcacatgctgttagagcgcttctcacattgtattaaagttagtcatttatgattccatttttcccactgactgtaggttctcaagagtagggtgtaccatatttcttttacaaccccagcaccaagcaaagcacctgctacttcgtcggtattcattgttccggctatgaaagcttagactaggaaaggatcttggaacagtctggtccactggttcctgaatgcccgaccgtggacctgcaggagcagaatcacccagaagtgtgttgagaaacagattccgtaggcacatccacagagacagaaagtggatccttgggccttggagttggggtgggatggatgggaagtgactgctgatgaggatgaggtttatttaggggttaaggaaaatgttctaaagttagattgtagtgatattgcacaactctgtaaataccaaaaccatcgcattatatgttttaaacaagtgaactttatggtatgttaattgtatctcagtaaacctgttaggaaaaaaaacaggccgggcgcagtggttcatgcctgtaatcccagcactttgggaggctgaggcggctggatcacgaggtcaggcgatcgagaccatcctggctaacacggtgaaaccctgtgtctactaaaaatacaaaaaaaatcagccgggcatggtggcgggcacctgtagtcccagctactcgggaggctgaggcaggagaatggcgtgaacctgggaggcggagcttgcagtgagccgagatcgcgtcagtgcactccagcctgagcaacagagcaggactctgtcaaaaaaaaaaaaaaaaaaacagattccagagccctaccccacttactgggggaaaaactctcccagagtatagcataagaatctgctttttaaaataagctttcctgtgattctgaagcagagctgggctggagaaccactggcctgcttttcccaccgtgaagaacactgtccttagcatccctgacaaacgatggcggagcccgttctgtgccagatgctgagccatggctggtgtgtgctgctccgcaggcctctggcggagcccattccatgccagatgctgagcgatggctggtgtgtgctgctccacaggcctggtggagcctgttgtgtgctggacgctgagcaatggctagtgtgttctgctccgcagggagctgactcctacttgtctgtgactcccatcgatcagttctgccttcaggtagcaaaacagaacggatcttccctgccttctgcttgtcagcccttctgctgctggaaacagctgcctgctccttgaccacactgtcccaagaggctgcgatgacgtccagcttcttcactggtcagtggccctctggtcatgccctccgttcacacaatccatctttccgtctgcactcagaatcaaatgcaggaacttggatgtggtctacccaagtccctagacctaactgtcacccacgccatttcagtcggtgtagctggaaacactggtagcttttgaggggcaccaggtcactggcttatgttgagattgctggtcatccagacctgccccctacgctaatcatacagtgatttttcttttcccagatttttaatttgaaaatgtacatacatccccccaaattggaaaaggatacgttggacacctgtattccttctacttaggttcagctgttgctgctctggaatattctctctcgcgcacatgcgctctttgtgtgcgtgtgtaccgtacatcccagcgactgtgacagctcagaagacatcagcaggcctgtcctgagaatgacattctcctccacagccacagtgtcattatcatactgattacacatcatagtcatcccataatgtcacctgaatgataacattatgggagagattcagatgtaagcagccacgcccctcctgtgggaactcaggccacacttgaattccacatgtcactgtgcaaagtgacttttactctgtggggatgtcagtgatccagcctgctggcttctctgtgaacccaaattctgttatcagaatggaccctccccactttgggtcatgggcagatttgatcagtttccccattgagtcattgatgaagatgttgtgagaatgatccccaaccctctgtgctaatacagagcactccgtctgctttggtgtgtttaattattgatccagccacaagtgaggagtggaagttgtgatttgaaagctgtcaccaagagatttgggggattcagtaacaaaactgcaggaaggtagacaggtctgcagaatgtggacatagtgggaaacgtcttcacattaagggcagtgtgactaaggaaaacacaaggttaggagactcaggcctgccatgttgtgctctaaactgcagcctctgcctcctgggttcaagcagttctcctgcctcagcctcccgagtagctgggattacaggcatgcgccaccacgcccagataattttgtatttttagtaaacacggggtttctccatgttggtcaggctggtcttgaactcccaacctcaggtgatcgtccgcctcggcctcccaaagtgctgggattacaggcgtgagccaccacgcctggccaacactgtgtcttctttaagcctccaacaacccatggaggggtggggtggggcatcggcttcaatacacaaaggaggagacaccaggaaagcaaggggcaggcagaggtgatgcagccagcagccttagccttgcttctgatgaagaccaggtcctggggggctcccgcggacctgatcccagggaatcctcctatggaccaggtcccggaggctcctcctatggactagattccagggaatccttctacagtctaggcttcaggggctcatcctatgaactagatcccggggaatcttcctatggaccgggtcccggggaatcctcatacagaccagatcccggggaatcctcctatggaccaggtcctgggggctcctcatacagaccagataccagggaatcctcctatggaccaggtcccaggggctcctactatacacatcaggtctgggagttggggctcctccagtggaccgggtccggggtgggggttctcatatggaccgggtcccagggggctgctgctatggaccaggtcccggtgggggcggggtgggggtgctgttgctacagaccaggtcccgggggagctgctgctacggaccaggtcctgggggggtctgctcctatggaccaggtcccgggggtacctgcccaccaccaatttgcagggtacatcttctagttccttcttccaaacccatcatctcataggtgaggcagggaaggggagccatgagggtgacgtaagggagagggaaggatgaggggggcagggcatggacggactggggatggagcaggtggccgaaggctgtgcttcccactgagctgcgtgcctggtgtcaggtggtgtatgaggagagccgcatggtcagcctcacagccccctacgtgtcgggcttcctggccttccgagaggtgcccttcttgctggagctggtgcagcagctgcgggagaaggagccgggcctcatgccccaggcaggtgtctcatccctaggacgagagaaaggtccctccttccccctgggggagggaaggctgctgcaggtcgccaccaccacagtggcagggagagactcatgagcttcctggaagagaggaatgaggatttctaaaaagctggaccccagagacagtgcccccttcttgcctcagggatggccctgtcctgagggccgaggggcagctcagctgactgtgactctccctgtggctccataggtccttcttgtggatggaaacggggtactccaccaccgaggtaatcctgctcttggaggtccagggagggcactgtggggaagagcggggggagagggcacctctgtcgtcccccacagaaaatgctgggccctcggcctctgccgccagccccctccaatggcttctttctcggcgcaacggggacgcgcagtgcgctcagccccgcctcccccaccgccggtaaaggactgtctactttcgcttcaccccttacctctttcctgtccgccactgccttctctacagtgccctgtgctggctcatctccaggtgccactatccagttctgacctgatcaccaaaagatgtcccagggcccgggctcagagtggccttccctgccagccgcccaacctgcatggcccgtcaggccccttgtccacaactagacaggtgtacccaggcccccactgatgccaggaaccttggcctagggagcgtggctgagggagatgggctcttttttttttgagacggagtctcgctctgtcgcccaggctggagtgcagtggcgcgatctcggctcactgcaacctccgcctcccgggttcatgccactctcctgcctcagcctcctgagtagctgagactacaggtgcccatcaccacgcccggctaatttttttgtatttttaggagagacggggtttcacgtgttagccaggatggtcttgatctcctgacctcgtgatccgcctgcctcggcctcccaaagtgctgggatttcaggcgtgagccaccgcgcccggcctagggagatgggctcttttactgttacatacagagctgcgggcggcgcagcagcacccagagagggcccaggaggggtgacattgggccttcctggccagcaagtgtgactcggccctggctgctgccctcgcacatgggtgcagagccaaagggttgtcatgtgccgacccacatgctcgtgccagcgctggcaggccaccgtcgagggaagaggaagctgggaggtgcaggaaatgaccggagagctgcagtatcccagcacgccagcgaggtgctgaccctggcactgaccgtgtcagagagaaggaggcttcctccacggccttgcacccaggacatcgggcacagggggctttcgcccaggtctgcttgggacttagatctggtttccccagccctctgcctgtccacagacctctgctcacttagggaagcttttggtaagactggcttgtcctgaatccctgtgccaggccccagccccacctccccaaccccaccctcctttctctctggcaggctttggggtggcctgccaccttggcgtccttacagacctgccgtgtgttggggtggccaagaaacttctgcaggtggatgggctggagaacaacgccctgcacaaggagaaggtgaggaggggcctgctgcaggccatgcccggcagctcagtggcggggctgtggtggccctgggcatgaggacacttctggaccagcccttgcctgctgacgctgccctcctcatccctggcttgtggcctggagtaagaaagacctgctttcctctgggtgctgcattgcctgtctcagctgagggcgctctgagctcctctcctgcctcctgggtgtgcctgcctctctggtcccccttgcctctgagccaccgtggcacaccaagtagggctaagtcctccagttactggggacaggacgggcttccaggctgtcccgggcgcacttggtgctttccccaccccgccttgccccagcccctgtctcctcccctgcatgctcaggtaccacagcctccaggcccccctcccgccaagacctgcctggagctgccacccacactcttcctacctcaccttcccctgcatcttcctcctctgggaccccatgttggcactgggagtgagacactcacaatggagggagctgttgacacccccagctgtttcactggatggcagattctgagagcagagactgtgtctctcattcctcctagctctcaggtgcctgcatggagaagttgcttaataaaaatctgttgagttgatggggaccttagacccactcaatgagccagtccaccgccgccagaaataccagcctgttgagctgtgtcagatgtgcccaaagcaggagagaagccacaggaaggaagcgggctcagcacccatcactgggcagtggcggcacctgtgtggctgccagcctggcacagcggccctgggcagattgttgcccttgttctaagccccatcatcaggtttgaacagggtcttgtgggcagcaccctcagctcagggacaagaccacttcaccgcctgcacgctcagtgtcgtctcaggtggtgtggctttgcctgggctgtggctcacctccaagatcagtccccttccctttcctggcccagtgactgggttttaaaaataaagagacctgggccggttgcggtggctcactcctgtaatcccagcactttgggaggccgaagtgggtggatcacgcgattaggagatcgagaccatcctggctagcatggtgaaaccccatctctactaaaaatacaaaaaattagccgggcgtggtggcacatgcctgtagtcccagctactcaggaggctgaggtgggagaatcgcttgaacacgggaggcagaggttgcagtgagctgagatcgcgccactgtacttcagcctgggcaacagtgcgacactccgtctcaaaaaatagagacttgccttcagcccttcaccaagaagagaggggtttttttccccatttttccctccattttccagatccgactcctgcagactcgaggagactcattccctctgctgggagactctgggactgtcctgggaatggtgagtagctagggccctgagctcctccaaagccccggggtaggggatcctttgcaggcagacacaggtgcatgcagacacgcgtgcactcacacttgcccacatcaaccccccgcctgcccgtccatccatcctcctgcctctgactgagtccagggtgtgatgtttgtgtttctttcctccagccgtgctgagggagcccaccctcccccagggacaagcagcaggaaggcgccctgagggtgggcaggccctggcccctgagagcactgccccttcagcttcccccttgcccccaggacattttgtcagagctccagctgctgaggctgtcctgaccctggtcccggtggcccagcccacaggacagccctcgccttccttgtcacaggccctgaggagccacgaccgcagcaccaggcccctctacatctccgtgggccacaggatgagcctggaggccgctgtgcgcctgacttgctgctgctgcaggttccggatcccagagcccgtgcgccaggtgggtgtgctggggatgcggggaggcagcacaggctcctctgcttcctgccacctgctgttcagggctccccagcagctcagctggggtcagagtgcagtgacatgaggacgggggttcctgggccgaggacaggtcacagacatcttgctggagagcttgctgggccctgcagtcaggacagtagactggggggtgcagggctcccaagagttcatccccaggccctgcaccccatgcactcagcctcccagcctctctgagcctcctgcacccagatacaggccagcttggagaatcagtgatgttagctcaggtggtgaggtgtcctgccagttcccagccagcccctcaggtcccagagggagcagctgaacttgagccaggaggctcctgcaggaaagctgtgcaggccccagagacgccagtctggggacttgggtatgcccgccccaggacccagcacaaaactggtgttcggtcccccaggaagggtgagaactctgctgaagccatcagctcagctgagaaaggactcagcctgtgggccagggagagacagctggcacgagatgcccacagacagcctcgataggaccaggggaccacaagacacgggtggatgctgtcccagagaagcccacagagtgagggttcaagaaaagtcagtcttaggaagagacccgctggaggaaggggaaggacagccctgggcaggaaggagtttgatgtgctcaagaaatggggggcccggagggtggctcacgcctgtaaccccaacactttgggagacagaggtgggaggatcacttgagcccaggagtttgagggcgccctaggcaacatagaccccgtctctacaaaaaaaacgtaaaaacaaattagccaagtgtggtggtgcgtgcctgtggtctctgctacttaggaggctgaggcgggaggatcacctgagcccaggaggtcgaggctgtagtgtgccctgattgggccactgcactccagcctgggtgacagagcaagaccttatctcaaaaaaactttaaaaaacaaaaactcagcaggaaggtgtggccggagccagatggcagtgaagtacagagtgggtgggagcccgtgagcatcttggggtgcgccagggtttcagacgtcacccacagggacttggatggctgtgagttggcagcggggcgcccgcagattggcctttttaggtggccgctgtgcgtgctgtggaggctgtggacgcaggtctgcacaagccatgctgcttccagcagcccagcccctgagcctccctggtgtctgccccttccccatcttaccccaccgcttgccctgcagcctgccctggggcagccgcagcaccggtggaaaacccccaggccaccaccttcgggccggggccttccggctgctggcgcgcagtgtggcctccagggggcagcagcagcccacataccacaatggccctcgccctggggagctgtgctcagtggtggcgtctgagagcgggctccacagccttgcagggctttgctcagggcacaggtgtgtggactctcaagcctgtgagctgtgtccccagcccccaggaaagaggcctttcttcctccattccaactcagccgtgtggtcttccgagagagaacccaagaaactgtcccccaggccttcagccacaggaactttaggtcgtgctgctgctgcagacagcacagttcctggagctcctgtacctttttctgcctcagcgcgccccagagtaggacagtggcccatccttgcaagggaggggacacctggcctgtgagtaccttaagcatctagaaatagagcaaggatctgtgcctagcaccagcaaagagcctgccaaatcctgggcccaccctccagaggaaggaaagccgcaaccagttgggatatgcagccaccccgtaccaggagcccgcagcttcttactgttgggcgtgagccgaggatcgtgtcctgcagggctggctctgtctctcggtccattttcttcctgagctcactcacctctcaacaggttcacctcctgctctgttccagaaggtcctgggagagcacagacggggttagtgttggtctgctctctccctggctccgcgccgggccgtcttggtggccccatggtcacaggcgctgcggcgctcctggttgccaggtccaggctctcctgccaggccaggctgggccgctccaggggaaggtgagaggtgctggccccgcctaccgctgccccacttcccccatgaatggaagcaggagcaggctgggggattagccgttgctttccatgagtcgtgcagccctgaagctaatctaagctcatctgagctgggatcagggggacttcccagggccagaccccccatgcccccgggggttcccactcagacctgagccctgttcagtgtccctccctgctagggtctgcccccatcccagcctgtggcccaccgccctcttttatcttggtgattctggagacgggacaggggttactgctgatctagtcagggagaggacagaccagctgggctcagagcccccagagaagcaagtgttagcagggaagcctcggcttgcgcagagcctacagggaagcacagactggaaaccagtgaaggctgagcagctccaactgccactggtggggaggagacccaggaccccctcactcagcctgtccactcaccagctactcaggcccttcagggactgcccagctcggtttgagagctgccattgcggggctttgaccccaaagtcccaggcatgcctgtcccacactcgctgacagggccgcggctcctgagcctgaagaactggctgtgacctgtgtgtcctgagtgggcttctgtgggggatggaggcattgccagcagcagctcctggtgggaaactggtctagagaccagctcagcacccagcagcacgtctgtctcccccaggctgacatctgctcccgagagcacatccgcaagtcgctgggactccccgggccacccacaccgaggtgagcacccagggaggctggggcactaaaggggcatctcacagacacctgcatgggctgtttccggtggctcagcctctccttgttgcaatattgtggctcaggacagacagtgcagggccagggaaggcaggggacacaggagaggtcttcaaagagggaggagagtggagtggggtagactcctattctcgtgggaactctccagaaacaccactgcctccacgcaaagccctggaactacggccctgtctggtccaagccctacctcccaatagggctatccccagccttcctcagtccacttagcctgtgaggtaccaggacgtgggctctgcagaaaaggcgtggcctcggagtcagacatgatccagccctgcatagccagctgtggcgtttgcactcttcgagcctcagcttcctcatcgtcgggataggctctgtaattcttgtcccactggttcttgtaagttagagtgacagtgtgcctggcacagtcaccgctaacagtgcaaggcattgtcttaaaaccattgcagatggtttgatcctgctagcaaccttgatgtgagacaagtactgtgaccccactttacagatgaggaggctgggacactgagtgttgataaggagtaaagcagaattcagtctggctacttcctcttctctcagtgtgggcagtttggattccaggccctgcatctgagcctccaggctgagaggacacccccactccctaggcaaagatcgcaggaggccccaggcctgggattcgccctgccctcgcccacgattagttactgaagggctgcggttccttcgtgctcattttccctctaagccaggggctgttcccaggcctccctaggctgctttgagaactgagggagcaggtggcggggaccccccaagctctcccagagttggaggccggcccgcttgaatgcaagctgcgtgtctgagcagcgtgctctgccctgccctctgccctgggctgtagcaggtcctgagtgtccaggccaagcgcagttcccagaccccatctggggtgtgagggggtgtcaggtaggcgctagcatctgatgctgtccctttctcaatgtcatcaccaggagcccgaaggcgcagaggccagtggcatgccccaaaggagactccggagagtcctcaggtgagggccagcccccacaggaccacagcccaggccccaggacagccccaaggccaggctcccaggagcaggcgggcaaggactggcagtagggtggaactgggcaccatgaagacaagaaggccaccggccaccccgttctggcctcaggacactgaccacccctgggggtggtctagggacttaggggaacctcatctcagccgcaggtggagcacccagtccccaaagacaggctgaccgcaccaccccagggggacgccgcagcacagcccagcaccaggtggggcagaggtgaccacggcccctctttgctccgtcatcggctggtcagctgtggtcacggtgcctcagaggacagatctctatgggggcaagtgccagatcctgagagcgcatgagacgctttcccggagccgacgaaggggactcggagctgcagcctgcacgacccctgcagcctgtgctttgcccacccctttcaatagatggaacttgcttgctcttttttattcttctcaggattcattttttatttcacatatttgtttgtttgtttatttatttccattgatctctttaccctgaggtttacttgcaaaatcagggttcatttttaaagcaggtcctcagccccagccacactgaaccacctctgccctgacacgggaggggtcctagctggggtagctgggctttagcacctgctcactgcctgccatgacccctgaccccagtcctggccatcctccagcctgtcccaggaagagggagcagagagcctcatctcagcctggagggggccgaggacgggggcagcacacgggcagtctgggctgtggggcaggaggccagacagggtggcactggtgcctgaggggttgggtgccaggctctgtgcccggcgggggctaggatcgggggtggcaccgtccccacaggctctgaaagttgatcactgtcctctgtccttgcctgccttgccccagaggcacagcctcaaaactgtctccactgtgggaaagcagccctaggcccctgtctggccacttcatcttctcttggtgtgggctagaggcgccagctgaggagtgggggcaaaggggatggcccacctgcagcccagccccagcccctgggcaccttgcaagcaccccccagcccttgacatgccggctggcctccagatcccaggccccctgaggatgctggaatctgccacagagcatgaagaagtccctggaatcctttcttgccattcacctgacctctacaagaaggggccggtgatcatgatttgacagcttgggacaagaatccagggaggccgggtctctggcgcgcctccatgccccttcaagaaggtgctatcctggtgttcctgaggagcagggcgagcctgggcctgagggaacaggtacacaggcagcagcactcagggccggggagtgctggccccacccgcctgcgctcactcgaaagctgctttggagggctgggctgcaggggcagaggctctggaaaagaggcccaggctgtgtagctgcccttccttgatggagcagactctattctagaaatttctataagaagtagactcactccaggacaagtggggctggggggagcggatgccgaggggctctgcgggcaccttgtgcctaaagtcacggggggcacaggagagagcgggaactaggtggactgttgtggctgccatccatcggcaacgctggccggaaccagatgagcagtgtggggcgagagggagccccagtgcggcccctcaggccctccatgcttcagggtgtcagtggccccggactggcctcactcagccctgggcacttgggcctgtcggggatctagctttcagtgaggaccccaccggtggactgaggctcccctcaccgtggcagaggaatctctgcccttcccaccgaaaggggcaggagctagtggatggggacggctcacggccctccctttccacttgccttggaaagggagaggcagcagacaggaagggccttggaactcaggccagtgtggggagagtgtccccagagagtccttggggtgggagaggggtggagttgacagccggtgaggccggctttgaggttgtgtggaccctgtctgcctctagctcaagttgggaggggtgtcagctgtggagctgtgtgagcctcggggacagccacatgtggggtgcagaccggtctagatgtgcctgtctggtgggcgctgaaggtccccttgggagagtccagggcagcacctgataagccgagagctgcagccgtgtccacctggaacctgtttcctcaccgggaacatgaacacctgcttcagaagctttgggggttaaaggtggggatacacataggtctcttgcccagcggtggacactcggtacactcctaataaatggggcatggggtggtggagtccagcacctggctgggcccaggaatgaggcatcagtggtcggagggaagggcgtctgcccacctagcatctttggcaaggatctgcctcccagcacccctgtggagtcctgtctggatgggagggagagcagggccgtggacagcctccagaggtgttcccctgaaacctctgaatgtcaccagcacaagcaggctgggctgggctctcccggcagtaacaccaaccgagcacagccgttgtcctggggcatgccgtccaggttgggggacagggaggcagaggtcccgtgtcattgcccgtgagtccggggcttaggcaccgctctggggacccctcatgctggcccccaggcccactgaggcataggtgagaccattgcatggtccttggaagaacagggctcctcaggcagggagtggacagagccaatgctggcgatgggggaccttgcctcaagcagagtcgtgggagatggagtcatgggggcgtgtgggggtctaggcaggggagctgctgactgcaggtgrcccagcagtacccaggtttctgctcgctcccattccacctccccgagggcactcgtgggaaagggaaacatggcacccagctggtgcctcattccacctacctcctgagtcaaagggctccaccaccccagacacacccagagcctggtgtccaggatgccatgtgtgtgccccaagccagtaggacccttgggaggctcagcaccccctgccccaaacaaatgcctctcccctctccacactcactggcattcccacagaatgccctcctgacttctagagctttccctgtccagagctccgggagcacaggtggcagggaattagaggcattagaagcacagtgtggaagctgggcagagctggctgagatcccactgtctcttccaggctggccttgagcaaagctcaaaacgcatcccacaggggtcagtacctgcccatgtggtgggggatattccaccaggtgaagtgtgtaagggctcagcagggagccttggcacatggtgagtccttggtgagggaaggctgttggtttggagaggtgaggcccacaaggccgtcagtccgccatgctgtgccaaccccagtgatgagaggatgaagccaggtcagggaggtctcatctaccaggctcccacctgagctggcagctcctcacgccatcccaagccatcagaactcaggtcctcgaagggaggcctggctgcccccttcctctggagccagacaggaccacacctcctgcgccctggggcttcactccaacaggggagcttcccagggggccgggagactcagaagaaatgcccacaactgcctggcatgtggtaggctttgcacaaagagcagtagcttcaaccccaatgacccacctctgtgaggggggcagagatggctctgggctcctctggggctggaggtcaggctctgcttaaaaccgttggttcctcaaggaccatgggtgtcaggtgggcctccccagctgagggcctggccaggacctggtcagggggtccttcattttcccctccccttgcaagaaggagcaggccagagcctggagataagtttgcagacaatttttacgtgagtagtttatgtttgttttgagataaaagtcatcatttccactacaactcattgacttttggtatattcacaatcatgtgcaaccatcattactgtttagttccagaacggttttttaccccaaagggaaaccccatatccattagtagtcactccccgttctcccttccccagaccctggcacccagcaagggcttcctgtctctggacaggcctgttgtgggcaccatgccgacgtggaatcaccgtgtgtctctggacaggcctgctgtgggcgccatgcaggcgtggaatcaccgtgtagtcttttctgtctggcttccttctcttggcataatgttttcaagatgcacctgtgtcgtgccaggtgtcaggacctcattcttctttatggctgagtcatattccatcatatggatcagtataaacattttgttaatcctggtggatatttgggttttttccattcagggcttttgtgaataatgctgcaatgaagactgatgtacacgcttttgtgtaaacatgttttcagttgccttgggtatatatacctaggagtagaatttctgggtcatgtggtgaccctgtgtttaactttctgagaaactgccagacttttccatagcagctacatcagttcacatccccaccagcaatgcccaagggttccaggttctgcacaccctcaccaccacttgctattatctgccgcttagattctagccattccttggagtagaaactggtgtttcggctgggcacagtgagtggttcatgcctgtaatcccagcactttggaaggctgtgacaggaggatcacttgagcccaggagtttgagaccagcctgggcaacatagtgagacctcatctctacagaaaactaaaaaattaaccaggcatggtgatgcactcctataatcacagctactcgggaggttgaggtgggaggatcacttaagcccaggaggttgaggttgcagtgaaccgtgattgtgccactgcattccagcctgggtgacaatgagaccctgcctcaaaaaaaaagtggtatttcattgtgcatttgatttgcatttcctgatggctaatggtggtgagcgccttctcatgtctagtaaccatttgtatatcctcgtgagcaaagcatctattcaaatcctttgcccattttaaaaattgggttgtcatcttgttgagttgcagaagttctttatatgttctgaatcctagatattgatcacttctttagtttacaaatattttttcctgtgggttgtcattattttcttgttagtgtcctttgatgcatgaaagtttttaaatttgatgaagtccatctattttgttgcttatgcttttgatgttagatttaagaagccattgactaattcaaggccatgaaaatttacacctacatttccttctaagaaatgtatagttttagtgcttcttacctttaagtctttgatccattttgacttaaatttgtgtatggtgtaagggagcaggtcccatgtcatacctgtgagtagccagttgtcccagaccattttgctttgttgttgagacagggtctcgctctgttgccaagactggagtgcaggggtgtgatcatggctcactgcagcctcgacctcttgggctcaagcaatcctcctggctcagcctccagagtagctgggactacaagcatgtgccaccatgcctggctaatttttttttttgagacagagtctcgctgtgtcgcccaggctggtgtgcggtggcgtgatctcggctcactgcaagctccgcctcccgggttcacgccattctcctgcctcagcctcctgagtagctgggactacaggcgcccgccaccacgcccagctaatttttttttttttttcagtagagacggggtttcaccgtgttagccaggatggtctcgatctcctgacctcgtgatccacctgcctcagcctcccaaagtgctgggattacaggcgtgagccattgtgtccggcctagtttttcaagatttttttgtaaagacagggtctcaccatgttacccaggctggtctccaactcctgggctcaagctttcctcccacctcggcctccccaagtgctgagattacaggcgcgagccactgcacctggcccatttccacttcctcctttccactctgaacgccttttatttctttttcttgcctcattgctctggctggaacgtctagcactttgttgaacgtggtggtgagagcacacgtcctcgtctcattcctgatcgaacgcggtggtgagagcacacgtcctcgtctcgttcctgatcgaacgcggtggtgagagcacacatcctcgtctcgttcctgatcgaacgcggtggtgagagcacacgtcctcgtctcgttcctgatcgaacgtggtggtgagagcacacgtccttgtcttgttcctgatcttaagggaacgtttgcagtctttcaccactagatgtgatgtgagctgttagattttcaaggatgctcttcatccagctgaagacggtcccttctagtcctaatttgttaagtgtttttatccttaaagggtactggattttgtcaaatgcttttctggcctctattgaaaagatcctgtgttcttctgtgaatatgaggtgttacattgattgattttcatatgttgagccagtttccatttgtggaatacatctaactcggtcatggtgtataatccatttactctgctgctgaatgtgatttgcttgtattttggtgcggatttttacatctgtatttataagggatattggtctggtgtttttttatgatgtcttcctttggttttgatatcaggataatattagcctcatagattgggttaggaaatgttctctcctccgttttttttggaagagagagattggtgttaattcttgctagaacctttggtagaatttgccagtaagcctatctggtcttgggtctttgggaggattttgattcgattcaatctatgtatttgttatagatcgttcggattgtccatccttgagtcagttgctagcttgtgtgtttcttggagtttttccgtttcgtccatgttatctcatctgttggcataccgtcgttcacgggattttattgatgtgtgtgaggtctctgagaacgtccctgccttcactcctgcttttagtcactggcatcttcttttaacttgggccatctggccgtgcatggttttgggagagtcctccttgactctgcagatgctgagcagtgatctggctgggaaaggggaccaggagtgcagggcacctgctgaggactcacctaagcccaagagtcagagagtcggagctccaaccacatctgcctgcatgcctttcccaggctctccccaggcggacagccagcaccccctccccaaagaccgggcagttcctgaccagcaccccaccaagtcctcagtaaggcctgtctttgggggagcagggtttcaggaggacagtgggggtggtgtagaactcatggctggcggttccgggcctctcaggaagctttgccactgggcttggggttaagtgggtgtggggctccgaaccctaaatgggtgagagttgaaatgaaagcggcacctgtagtccgtttggggtgcaggtgtgtgccaggggtcttcagccccggctgatggccacatgaaccacatgaggagaggcagggcgtgtcagcagcaaacctaagtgtgtgctcatttctgtgggcccttactctgtgacactgcagctctccagagagacgctttgaaaacaaaacaggaaagaacacacggccccgcgtctgttgcctgagtcactgtattccttaaaaggtgaaggaccctggtccttggcttttcgtgcacatgagaaaatgttggccaaggttagcgattatgcttctgtaatctgtaaccagaagtgctcttatgcccaaaccttgatgtgattctgctgtaatgtaacttcggagccagctggatggatgtgactgtgcaggtcctgagccccggcccccgtaaccaagcagtgggccgagacactgagccgggcagtcaaacaggagctctctaaggctgctcccgggctgggacctgggtctaggattctcagtaagacctttgaataaaactaacttgaattcttcaaaagcttgattttttttcttcagtcaaaagcatcttgcagagatgccagcccaccgtggggatgtgcagtggcagccctgagctggcccagcccctttccccaaggctgctctggggaggggggcgggtttgggggagtagagtgcaggggattgaagcaggggaaacagggtgtgagggtcaaggggggctagaattgggggagcaggttcacgggccaggggctgggcaggggtgggcctgggcacctggtaggtgggggagccccaacttcccatccctgcgtcacacgcccttcccctgcagggtgggggtctgcagggttggcatgtgcccccctgccctgtccccaccctactccccggcggcaggcagaggggcgtgcaggggggccctaagtgcagtgacgtctcgactctgactccgacccacagaactttaaaaatagggccgtggtaaagtccgtctctacagaccacagcctgcccagagctaagaatattcttgaaccacttaataattcaagtcagatccaaatagattttcctgctcacttgcgggcctctgtgtccctttcccaggcccccaccctgcctggaggctgggagggaaagactccaagaagcaggtgggcctggcctttggagcctctgagaccccagctgtcctgcgtcccctctcctgtggcactcaggatcagaagacacaccagggctggagtggtcctttaggatctggccaaggtcaggccccagggaggggcccctctggtggtcctagctgtcccaagccccacccctgctctggccacaggcattggactatttgaatctgggtctgagctgggccttggccccttgcacagcatctgacctgagccacgcacctctcatgggggccccgtcctccagggccaagggcagcatgagctccaggagagccagcctcgcccctcacaccctggtagctggtacacgcccaccccagggcctgtgtcctgcctcctctgcggaagagtccctgaagggcctggcctggggctccttgtcctggtggaagtggctgtgcacaggcaaccagggccctgggttccagaccaaggcctctgtacttgtgtgctgtgtgtgcttgggtaagccacctcgcctctctgagcctcttatcagtgggaccgatgcttttagaataagctgtggtgtttggggcaggaaggggtatctgacgggtgccctgaggcctgagtcaccctctcctccaggggcagtgcccaagctgcagacacccctcacccatccctgtctccccccaggctctggtctttcaaacaggaactcagggtgaaggggtgccacctgggaatgtttgggggagcccacacttgctggcggggaagtgctcaccagcctggccccccaaccccggcagcagcccagccttagcagtccctgctgaacttccctggccaggccccgagtggagttttaccaaattattaattaagggcaaagccatgctgcagcgctgacatccagatgaatagcacgcagtgtaattaggcgaggtgtcatggagatgatggagtacagcctgctagccccaattcacctccccggcaagcaggagcaacttctctgtgtcctaccaccatgggcggctggtgtggggccagtaggggtgtggctaaaggggccagctgacagctattgcgggtgccacgggccctgtggacagggctgggctcctcccccagggacaggcaccccttcccctctgattgccatgggcagaacccaaaagggagtaaagtgcagaaaggggcaggcaaggtgggaggcaggctaaggcaggagggatgaggaccaggacaaatccacagatagacccggagcagcccagagaggggcatggagggagcacaggagggccgagaggggccggggaggaggtgcagaatccaggaagacaggacaggcctcggggccctaccctggggttgcctgggcctgacagagagctgggagcggggagcagcttgagaatttttggttttgttttttagttacttacattcatgtatttttggttgtttgagacggagtctcgctctatcgctgaggttggagtgcaatggagcaatcttggctcactgcaacgtctgtctcccgggttcgagcaattctcctgcctcagcctcctgagtagctgggattacaggcgcccgccaccacacccagccaatttttgtatttttagtagagacggggtttcaccatgttggccaggctggtctcgaactcctgacctcaatgatccacccgcctcagcctcccaaagtgctgggattacagacataagccaccgctccctgcctacatttcatgtatgcttttaatcaacatcataaattgacagttttaataatcgaatagtactacaaggcttctgacgaaaaaccacacctctttcctgattcccccttctcagaggcaaccttttctgtgattgtagctgattctctttggtatttacttccgaatagccatgctcatgttgctgctggtgctgcttttttttttctccttttcagattgagaaatttacccatgtccttcccacgatggcggctgaaagcttgttctctcacacgtgcccccccacactctcccaccagcttctccttttgattgaatcagaattcaatgtatgcactattatttctcagcacacactcttcccagctgagtaatgtattgtaattacatttcctttcttgtacaaccacttatttttcctggacttaataattgcctcatttaggggattgggttgatattctgtgtacccattacttatttatccccaaaccctgccatggaagtataaatctccttttggttcattaaaaacatccggcccatctgggcacagtggctcacgcctgtaatcccaacagtttgggaggccgaggcgggagaattgcttgagcccagtagttcgagaccaacttgacaacatagcgaaaccccatctccatcactatttagttttaaataaaagatatttttaaaattaaaaacatccaggccaggctgcgctgggcccgcctcactgtagggggctgcgctgggcccgcctcactgtcgggggctgcgctgggcctgcctcactgcacgggaagtgtgctgccgctgtccttggtgctgaagtcaaggctggggccggagcctctcctccagcctcactcctcttcagcttccgccctgcttcgcctccattcctgcccaagaccccctggggagcagaccccaggccctcctcaagactgccgaccccagccctcgtcccagcccaggccatctctcccaggccttgcctttctgtaccaggtggtcttcctccccagggggctcctccccgcagccctggcagacagcttgaatcctggagttgcagcaggtgggtgtctgagtggagctcccttgtgtataatcagaactctccaggttacatgtgacaaaaaccaacttaagcaaagctgggacctcgttagttcactccaggaggcagcgccgccctgcatggcctggccttggctccctccctctccgggaggctgctccctcatcagcccagcctcacttcctcacccacataacaatccttggcagagtgagccctgctgttccagcggccccagtggccccagcgaaggccccacgccgacacttgttggctggccaggcttgagtcacatgccttcccctgaacccctcactgtggcctggggtcggggggccaagtgcccagtgcagagctagggcctggagcagcctcagtgcaccttgtggagagggggacttctcagtagaacagcactcttgccaggagaaggaacacctggttgagaaaaccgaggtcaggtgaccttctcaagaagggtcagtggcttcaggatctctcataaagcttcatctccaaggcccaaggagagcaacagtgtcccatcttgtctccaggtaattctattccggtgccagcagagaacagcgtgattaagcctccattattaacttgccaatcttgtttctggggcacagacgctaacggcttaggggacccagttggcatataatgaggtctccatgacaaattacttggttatcaattaactatctggggaaatgtcaagcaggatccatcatgctggcaggaagagagagccaattaacccagggcagttcatccaacccaccgtcgctgcgtccagcagggccaggaatgagagaaagggcctgtggagcccagagcagggcagttcctccacccccccagggacggtgtgacaccggctgcggctacactggtggcagcttctgcagtcatgcctcgcgtgctcggcgcaccactcttgcctgtacctgtctctgtccattcattcattcattccacagtcacctgcatgtcagacctggaccaggcacggatgggagaggtgactgagggctgaggttagggagggacacacgtgcagactgcagacaggtgtccctgtcttgtgtggtctctgcagtcagcaagatgggtaacagcatcctcaggacagggaggaaagaagagttcattctaaaggtggtggggggacagagagagcatcatagctgggaggagagaagagttcattttaagggggtgggcgggggacagcatcacagagctgggagaagagttcattctaagggggtaacggggggacaaagcatcacagagctgggaggaggagagaagagtttgttctaaaagtggggggcacagaacatcatagagctgggaggagagaagaattcattttaaggcggtaaggaggggacagcatcacagagctgggagaagagttcattttaagagagggtgacagagtgtctcagagccaggaggaggagagaagagttcattctaaggtgggggacagagcatcacagagctgggagaagagttcattctaaggggcgggggacagagagacacagagagactctgtgacagcatcacagagctgggaggaggagagaagagttcattctaagtgggggttggggggtgggggcaggacagagtgcatcacggagctgggaggtggaggaggagaagccaggtccctgagacaaggggcagggtgggtggccaccaggcagagggtgcccaggcacagcaggggacggggctgctccaggcttgggcaagggaccattgactgtgcactgtccggacctgagcagaatctggctctgccgtcgctgtgctgggagaccttgaaacagtttagtaaaacctcgccgggcctctgttttcttatctataaaatggggataataacccccagttctgggagtgttgtgggaactaacactgtggcccccacttggtgctggctgaacctggttcctccttctcctccctacaagagaggccttggacccacctcactggtgaaggctcccagggtgcacatgccggagcagaggcgcagggaggagggaggcatcctggaggaggtggcttgggctgtcctggaggagggaggtggcttgggctgacggccggcacacggacgggcccgaggccgagtggcagggtcggggactttggtcacttatcaaagtcaaagcccaggactttttgggttttgctttgttttcagcaaaggcccttgggtcacctgcgatgtgtcccacccttgtgagaaccagtgacgaagccctggccctaccctgtcccctggcctttgcctgtgcccccaccccgcccctctggtagccccctccccttgcctacttgggggtgattggcagctgcctcttcccagccccccaggccatggcacggagcctcaagcctattcatgatgcggacatatggcacctccacggggggcttggaggaaactgccaattgcgagagtgcagcgtgtccccattccagccctgccggagcaaaacaaacgcaagtcgcgaggggagcagaggcggcccgggggcgtgcgcatgtgtgcgggtgtgcctgtgtgtctgcagctcacaccgcgggggtgccgggaggaggggggctgcccgatccttgggagtctgcggctttcagacgctcctgggggctcggctactggggtcctaaggaggcgagctggccccctcattccgccccgcaggccgtgcctctcctgtcttgaggccgcttttgcctccccacacctcttacccaggacgctgggctggaggctgtgaacagccctgggagcagatcccagagatgctctcattctttccggaatcaggacgttccccccagtgtcaagcccaagtccccagggctccaggcttagccaaactcttctgttttattggaggtggcaccacccccatatcagtgggggcactgcagccaccagtgcatggccccctaggcccctgccccatgctgggctcaccctcctgtgcccagctggggacccaggcatgacaaagccctgctcctgcggcaggggagggcgtgggggggtgggggggcgtggggagggagtggggagggctgcaggggagggcgtggggagggcgtggggagaggctggccaaaggccaggctctgggggtctcagcacactcctctcccgacccagggctggaggctgctgccatggaaaccaggcctgtccacccactggccatgtgcatccaggggccatggccaccacctcctagcacccagctctgccccccacccccattcgggggagtgagaagtcaggagagagaggaagggcgctaagcagaacctggagggtgggcctggggtaccagaggctgaggtgctcaggcaggctaagggtaaggggcctgtcccctgagccccagctgagggtctaaccctctgtgctgctgggaagctccagaccctctgccacaaaccttggcatgcacttcgggcagtggcagcccctgtggggtgtgtccgtggcacccatcagcctcctccttgtggctggggcctaggagcagaatctgcacgctttgggccttccccagggagtcagggaggcgagggtgcagcccgctagagagtggcccatccatcagttccctcccaggagcgctggctggggatgctggtggggaggggctgcaggggcggctctgactccagctctgccgacaccctgtccccagcactggcaggcacctgggtgtcctaatctgtctatagcctggccttggacgaggggcctctgccccagctgggtgggtgtttcagggcccaggcccccaccttggctggcagcgtgcacacccgccacaggcactgggccgaccaagttgcaggcaccactgtgcatccaagcatctgctgccgccgtctcggggcagatgggctgccagtgagctaagtgtgtgcgggggaaggggtgccataggaacccctccccgccgggagcgcgggggcgggagaggcaggcacccgccggtgggggagccgggagccatcagagggaggggggctgggatcacatgggctccagggttggggagaggcccatgcagcccgttcctgggccaggggctcaggggatctgggctcagcctcggaggatgcagagccttagaggtcctgatcagggctgggagacctgtcacccctggccaggtggccctgggcaagtcccttccttctgcaggcctcgggccccaggagtgtgtgtggatggtctagagggtgccttcccgctccggagccctgtgccttggcaggatgaaggcgtcctccgcacacagatgcctgccatctgcaggggcctcctgccaggggacaccagcacaccctgccagactgatccggggcacgtgggctacacctccgccccttgctccaaggctgtggctcccagaaaggctggggccatgctgtgtgttaagtgaggccacttcggagtttgccagctttccccagtccctgcctggccacagcaggagccccacggtgaggggacctgccaggctgggcaggatgctggccagggtagaagatcctgccctgcccctgctgccccccaagcccatcacactgtatccaagacccctcaggagtactgggatccgaggaaggagggagtgtgggagagttggcaaggagacagcccaaggccaccaacagccttggaagccgatttgttggggcatcaggagggccttgagcagagaagacgaccccacggacaaaccctgcagcatccctgctccaggagaggggtgcccacccggcctcagggggcttttctttcatgatggcagtaaacagagcaaagttgtctgtgctcagtccctgtcctgtgccagaggctgctctgagaactcagctcttctgtaattggcacaagaatgctaagagccacccttatgcccactagacagaaaaggagactgaggctgagcaagctggggcagctggccccggccaggggccaacgaatggctgagctggaatttgagcctgaagtctgcttgaccccttgggctttctgaccagagcaccccactggaagggcacgtgtcctccaggagaggctgcctggtgcctccaaaagtgcagggaaatggggcaggtggcccctctgtgccccctcccccaccaagcctaagggggcccccatcgccaccatggctctctcggattcaacagatgcctccgcctggctgtgactcagcctcttagtttgttggtggttgcctttttttttttctttttcttttccaaagtatgacatcgccactgagccttggcagagtggctcccaggacactggcaggtccctcccaggccttgctcaggagtgcccatgggtggccccagcttgcaggggccaaggtggctgatgaccagatgccatgacaggcaccccaaaagggcctcgcccttggactgtggccctgggcagactctgttaccttcaaagtaggcacagcccatcccttagttgcacctgactggggcacagtgccggggacacctgtccaaagtcaccctccccagctgcctcacagcccaaggacatctgtccaaagtcaccctccccagctgcctcacagcccaaggacatctgtccaaagtcaccctccccagctgcctcacaacccaaggacacggcgcgtacttgacgttgcagattgggccagaaaccctcctggggggaggggcccagagccctacttttcacgggtgggctaagaaacagaggatgtgtcttggagaagacttgcaggccaggagcactgcccattgccaaggccagtgggctgagttccacggagagaaaaaggctttccctccatgaaggaagctttcttggcatttgggactattggtagagcccacagttttttttgtttttttggttggtttgttttgttttatttttattattttttttttttttgagatggggtctcactctgtcacccagggtggagtgcagtggtgcaatttcgcctcgtggcagcctcgacctcctggactcaagcaatcctcctgcctcagcctcccaagtagctgtaactacaggtgtgtgccagcacacccagctagtgtgtgtgtgtatacacacacacacatatatagtagagacagcatcttgagatgttgctcaggctggtctcaaactcctggcctcaaacagtcctcccaccttggcctcccaaagtgttgggattacaggcatgagccaccgtgcctggccttagcctgtgttttgtgaggtggtgagttccctgtccccagaaggatttcagcagggatagcattcctcacgggactgagtccctgtggtgcaatcagagaaaagagtcactcacagctggagttggtcatttcaggttcaagtccaggcgtgtctgtgtgaactcaggagcagggtattaaacctctctcctcatctctgaaatgggatttgggtctccctcaaaggcagttctgaggtcaccctcaaaggcagttctgaggtctccctcaaaggcgagactgtgtgcagaatgtgtgcagacattttcatgccccagtctgactcatggccctgtgcatcttgacaggtctctgtctctgggagcctcatcagaagaaatggcttaaactccacagggcgggatttggggaggagaaagagctttgggctatggggagtcaggagacgaggggatggcggggcttactgtcctttcagtgacccgagaaagaggctggcttgggtgagaggaatggggcagcttctcttgcctctgggactcaggagaacccagggctctgaggcttggagggggtctggaagctgttggggggcaggtggggcgctcatggcatgtagaactgggacatgccaggcaacagtgaggaggccaagggtggtgggaaacaccatctggcagtggcaagcaggtctgggcgtaagaggacagacaatcccacgtccaggacgacagtcccaaggcagttggctcctcctgccccagcggtcccagccgacacccacgtccttccctcttctcctcctccctccccggctgcccgtcctgtgcccatttatcatgcccatggtagtgccccgaatggtggtcccaggtgtccacccacaacctcagaatgtgaccttttttggaattagtctttgcagatggaataaagttaaagatttcgagataaaatcatcctgcatgaagggttaacctgaaatccaatgacaggtgtcctcgaaagagaaaggcagagggagatttgagacccagacacccacaggggaaggtagaggtgggacagtgggagagatgcatccacaggccaaggagtgccgaggtccccagcacccacggaagctgggaaagcggcctgggacagggtccccgcagaggaccagaaggattttggaattctggtctccaaacctgttagagaataaatgtcggttgatttcagccatcaagttgatgctcatttgttaatggcagcccccagagagggagacagcctcccttcccgtctctccagcccagcagactgctgggtattggggttggaaaagtctttcaagggctcagggagtgctggggtggccaccaccttctgagcaccatcctgtgcctgggcctgcctcccttgcagcccgagcccaggcataggagccagctccactcgcacctgccagcctcagcctcagcctcagaggtgagcaatgccaggaagccgatgcctccagaatcccttgaccaacggggacggcagcggggccctggctttcagaacagcaggtgcgggctgctgggtccatgcccggtgggggaggctgcacggttgggcccttgttcttggctgtgtggccgccgaggctctccggaaggttctatgagcctcctaatatcttgtattgggttccattaagctggccagagtgggttctgccggttccaagtggtgtgtgggatggggaagtgctctcagctctgtccctgggacaggtggggggtgggcgcgtcacatgccagagatctggggtgaaggcctgaagacccttcccctcctgctggaggagagccgacctcccccacagggcctcgtgggagtacaggcggccagcaggcccgtgggagatcaggagggagatgtgttcatagccggcactcgaggcacctgggttcgggacacacctgggctcgggtcacagaagccccccattccccaggttgaagaggcctgggctttccctgggcctggctctcgctctcctcccccaacgctgctctggtccccggcggcctcctcagggcccaagtcctccctccccctgtcctccttctctccactacccccttcttagaaatgaagtcatctagaaaaataatcaaaaaatagaaaggaaaaaaacaccttagaaaatgggattttttttccccttcaaacatttggtgagacacctcccacacggatgagtggccacggggcctgttttggtgtgagagagacttgggcctgtacccttggagttgtgagaggcaggggggcctcccagtcccacagtgggggtctacactaggcctcccctctgcagcctggccaaggttggccccagaagcctgaggtccccgccgatgccctgagcaggaaggccgctgagtccccgaggccagggtgcaaggcagggtgggagggctggcgggggccaggccaggggctgcttagcacctaagtgaggccctgcctccggtctctcccctcttcccacccggcccacgccgctgcagggctctggtttgtttatttataattcatcctctgcctgcttccaagaaggctcccggcacttactggaaagctgcttcctgaagctccgttgtgctcctggcaggctcgggctcaaagcccgtcaacgcttccccatcactcgcaaaggcagacgactcagcctggcttccagggccctccagatctggcccccaaatgcagtctttccgccacttcctgatgtatctcctggccccagagggaccctcccttacaatgcagattgctcgagggaccccgggacccaggatacttcctcctaccttgggatcagaccctccctcatcccatggcgccttctctggtcccgacagtgggggacgttgacacctctcttctgcccattggctgtctgctgtcacagttacctggtacaggctgctccttctggccccagcccgacctccccggcccttggaggagcttaaccaagtcaaactattgttaaggttgatggtgacaatgccagggaaggtggaggctcccctctgcctccggagtggcattgaagacggaggtgcctgcagttggtccccacgctcgataggcaggaagctttggtcaggagtggggctgagaggagccaccctggtggcttagtctggacgcctgactgggggttccatgagtgtgtcctcgggggctgggtggcgaagtgtgactccacgggctggaagggcggggacacagtgtggtgagaaacagcatgcagggctgcaggcggcagctctgcagtcagggctctgggctcagggagctgtcccctgagcagggaagctcacaccagaattggtcaggcaacccagggatctgtgagggataagagatggtagttgtgtgaagcctctaggctttggggtgatttgttacacagccaaagctaactgatacagccaggaactgctgatggcacagccggaagtcccacccacccttgagagatcagcccctgccttgggcaggaagcccaggaggctgtgggaagatctctggcctgctggaggccccggttgctggtgctaagaggagggacaaggagggaccagggaaggggacaccctgaccactggagtgctgaggagcgagttggggggtggccagattggggttagcagctcgaggggcacacctggggggctgtttccaggtggatactggaggccagccagcaggctcccttgaagcaagccctcatgccaccttcccagcccatctgggacctcaccacatcttgcttctgggaggagggtcaccctcgatcctgggcctgctgcctcaggctgactcttccctgctgtggctgagtgagcaagctcggaccctggcctgagggcctgcttagtccccctctcagatttgcaccggtcagggccaaataactggcttagccaggggaccctggaaacctcacttcatttccttcaagccctaattcctccattgaaaaacagggccctgtgcggaggagtaaacgcgagaaagcccacggggcatccatgcatatccccctggctcagggcctcagctttcccttctgtaaaatgatgctatctcttccgcttctccctctgccagtctgtacctccaacttctacagagcagaccggatgcctctgcccgccggcagggcacgtcacaggtaaggggtgggggctgtgccccatcctccaccatctgtgggctccagatcccactggcctggtaccatcagtcatggccaactcccagcttctccctccctcagcttaagcagccactgcctggcagaaggggagccctggcatggcaggacccttggggactgccctcaggaagcctcagtggcaagggcagatgccagctgggctgtgtcagggatcctctgccacctggcagacacactacagagcgtggcctttccctgctgccgactctccttgtcactgagttccgtggggccacagatgtcctggccatgggggtcaatgcagaacagatgtccatccctggccacaatgctctgtgccagaaggacagtgccagaatggccccttgctgacacagccccagaacccctgactttggaactgggtggaagggaagagaggagccaggtgactccatctcctgaaggaactacctttgggagcccaggaccggcccagaagcatctggctgtggggtggcctacagcctcctccccacatggctggctggcagatggctgccctggcctgcccaagccacatgcccggcaaagagttcctctgccagtgccggggcccacgaggaagccggctgtccggacatttgcatgcaattccgggcagccccggatgcttgtctggcttccagcctgggaagtggcatcgagccctgggaagaggacaggcttcacggccacaaagagctcaactgaagatctgaatcctgcatttagtagtggtgtaaccggggtcaggggctttctgagcctgtttcctcctctgtaaaggaaggctaatgtgtaaatgtgagcttgacatctagagccacatccagcacagaggaggcgatcaatgatggctggctccttcccttcgtccatggctcacgccttacacctgctgctctaggcagaaatgcccccctgagaacccctgagaggacttccagagaccccaccctggaaactgatcccaccctcctccttctctgtgacttccacacagcctcttcctcatcattccttcctaccagactaaggggccttgagggtgataccaggtgttctccagacaattcaacacctagcagagtgctgcgcggggctgccagcataaatcaatgacatctttcagcccagagggttctgagagtgattgcatttgacagcacttcctgaggcgcccctggcctcctgaggaatgagctgttcagggcatctggcaagcagacaccagtccatgctggcacccttccttctggagagtcacccaagggagccaccgatcgctgctagaactggagcagagtcctcaaacaacagtctgcacacagacaagacccgggatatttctttgatccagctcctgccttacaggacttcatcttcacccattcctgaggaaggagagaggaaaaaaagacctctcatacaccccctcagtctagtgtttactgagcacacactgtgatctggataagagagaaactctgccgtcttggtgctttcagacaatacacatcaaattacattatcaacgatgctggatggcgctaagagccggggagggaaagcaagggagtacaggccacagaccgtcctggacttcgagtttgacttacattttggctttgccatggtacaaacgccatacacattcagtatactcctccgcttacagatggggttacatccggataaacctaccgtaaattgccttacaatgctttaacctatgatgggttgatggggaatctaaccccatatgacaggagcatctgtataggaagggggcacggttttcaacaggtggtcgcctcactaagtgggagtcacagggtttcaggggaggagcgttccaggccgcggtaacagcaggtgcaaaggccctgaggcaggacggggcagggaggtccagagtccaccgcgcagagacccgggaggccggcaggcacggtgaggaccttgacttaaacccaggaaaggggaaccacactggggcatgacctcctgtactttccgagatcgctctggctcctgtgttgaggagactcaggaggggaaggtggaagcggggagacccagctgagacgctactacagtcatctcggcgggaaaagacggctcggagcagggtggtggcagaggtgggagaagaggtcaggtctggggaaatttcgaaggcacagccaacaggatgtactgccgggctggatgcggggtgttaggggtcagggaggccgccaaggctctgcctgagaactggggagggccaggggcaccatttacggagctggggaacagcaggagctcgttttgttagcaggtgcgaggcgccgggcatttaaaggcggcctgacgggaccggccgaggcggaagaacggacagggccctgcgggaccgcgctcggatccgcctcctccctgacctcggcgtccccggctactgcctccagagccgcttcggggccgaccccggggactcttcccgcctgcggcctcgcgccagcccggccggctcgcctagtgcccggcgcccagaaacgcgtggagctgggagaggccacacaacccacgccggaagcctaaccccgtcggcccccgcggccggcaccggccggaagcccgcgaagcctaactctcgcgagacgcctcctgagtcccgcctctccgcggttccgccagtcgccgcgctcctgtctgcgttgctgtggcagccccgccgtgcgtccgcgttcccggagccagcgcggcggcgcgcctgtgctcggggcttggccgtagcttttcctcaggtcccgcgcggcgcgcccctgtgtcgcaggcgccctgccgctgcccgcagcttttggggggttcgggggcgggggaagcagggccgcagaaggcccaatcggagccttctgacacccgccccggcccgccgccctcccggagcttccgtccccgccgcctggctccttactgacacgcgcgccgcccccggtcgcccgttttccctgggctccggcctcctctcgcttccagccctggcctgaggtcctgagggctccctggagggggcggcttcccagcacccagcgtttaacagggccctttcggcctccctgtcccccaagctcagagttggggtctgcagcaggggaaagaaggagggagactgggcagctcaagttccccagaccgcacctatcagacggagggcggcccgctcccgtcagagcccgacctcggcggacaggggaggggatgggttgagaagcgggagaaggccactagctggaaactccctgacgacagggacaccgttctcgccccagccccagcgctcggcctccgcagagccggcccaacgaaggaagaatgaaaatatgaacgtgacatgatgtgggcttgagaggccgccttgttctttggattcacccagaaacctgactgaaaggaaagttactgcaactggggaaaagcacagttggctggagaatttggtttaaaaataaataagttttaaattacgtaactaacacgtgcttggtgtagaaaatttggaaaacatatgaaagtacggtataaaaatcctcgatcccactgccacatagaaccactattaacatgtcagaatcgtttcttcgggtgtttttttttcttaatgcatttatattgcatatacattttttctctaacaatatattgatgtgccccagtcccccacagaccctaatgtatgaatgggaatggaggcgtctgaaatgtgactcacaaagagttctctttcctccctaacaccccatccctgcctcaccccttcagcgcattcacagcagcactgggactccggactcagacaaatgtgcactgagctggctggtgctctcatagcctctggccccagagctccaccttcccctgcagagggggtcccgggagtgtcctcaagtgctccatgccccatgttcctttccatggcaccaaattcttctgacctccttctaggtgaggggtgagggggtgaggtagggggttggggaagagtgtccccatgaggcctgaggggcctgctagtgcaggggaggaagggaagttcaagcaagcaggctcaacggagggctccaccctccaggtacggccaggtcctgggagaaggaggggaagcagtctggtccaggagagaggcagcaagggcagatgctaagcctttgcctctgcactccctggggacctctggggacccccccccccacccagcactagtaatcaaaggaggacagacacctacacacgtgtcccatatgtctcaccctagcaggcagcgagaggatgcccagtgttactctggccccctggaaggctagcttgctctcctgggcacaagtcagaagcagtgtgaactggagtgggtggtgggggacacccctcccggtgagccccccctccgacccggctgtcagtgatccacaagtctggcccatcagccctccctgctgagcacacagtccactgacccttcttcagaattgtgggaatgacctcactgcccctccaagcaacgtgttgtgctgccgcccacatcagacatccggggaatggactgcacagcctccatgtgtgctggctccttggcgcctgcagagccgggttgattctgtctttcccttcaactcttgtcttaaaggatatcctgagcgtgccggctcctctgctcagagggtgttagggtggcggtggcagtgggcacctctctgctcctcggctgggtgctcagcctcgccaggctgcctggagctggtggagcaacagccttgtcccccgtccagctctctgcagctgccctctcctgtcagaagctcctggaaagcatttctggcacatagaggccatctgcatgttctcctcctgcccgccccaccccgagtagccacagaggatcttccccagctgccttcacctccgcccccagacagagcctcctggctactcccccagatagtgtctccaaaatcacttccatttctcctctgctcgcagccactcagtggctttcgttatcattcagaataaaaccaaagcgtccattaccatccaaccaaagaccccaggaacgcgctgcagtggaatcaagccacgtctgttgactggttgctgtgagaggatgttaggaaggacccgcaagtctcctcgtgttgggagagcatggtgggaggaggagttgggagaggctcaaggcagcagggctgactccacagctgggtatcttgataacttttatctagaaggcaggaagaacaaaatgcagctgaagctgaaattgatcaagcagcagcggtcattcaaattagccaggacggggtggtttggtgacccttgaggtctggccgatgctcatgttctgcctatgctctgacacgatcctgtgtatcttgccccatctcgggcacacagtggtcttgtctggtgttggtgttcagtgacattgcttagccatgaaattgtctccaggttagtgactagtgacgccagggtgggtggctcttctctttttcaagacctgggtgtcacctgacccccagctgcctctgactcacctgccactgcccttcttatccacactcttcctgacaagctggtttcctgtgtggccgaacagtttcctccttcgggcccttcgtggtcaccgggcccctgcctggagcctccgcctcaggaacccatgtggctgactccggccctgtggccttgccactctctctccatccccacgctgtccccttggtggttcttactgccaccttcctttccacctgctgaaaccctagggtcttctggtccctctgcccatctaagaccgaattcccacaacagggagagtctgggtgacacgctggtgcgtttattcctggggcagtgtggttcccacatggatcctcagatgttctttggtgttggtgaccccactgtggaccgttggttctgctccaagaccaaagctggctgctggtgggtgcagaatgcgcatgtggcttggaccagacagcctctaggcctcccctttctaccgccaggtgccgaagggccagcagcctggacaccccggagccctggagagctctgaccccagcctctgccccacctgaagtgggtctggccccttcttgcaacaggatcagcccccagctggagacagagaccccacagccagcccagacagccgcctagtactccccagtcccccagcctaaggacgctccactgtcctgggctgccaaaacaaatatcaccaactaggcacctcaaaactcatcctctggcagttctagaaggcagaagtccacagtcaagcggtcagcagagctggttccctctggaggcttggagggagagtccgtttcacgcaccccattttgcttctggtggctgctggcaatccttggtgtcccttggttcagagacatcgctgcaacttctgcctctgtcgtcccgcggcctccttcccaagtgtgcagctgtgtcctctcctcttcttaccagggccccagtcactggacttagggcccaacccaatccagcctgacctcatcttaacttgattacagttgcaaagaccctatttccaaataaagccctagtcacaggttctgggtgggcatggatttggggtgacatattccacccaacgcagctcccacccccatgtggtggaagcccagggtcagggaggatccccgttcccctgctggctcccagtgcgcttccaacttccaaggggggctcagtgggtggtctgagctggacacaacctagctccttcattcctgtgaagcatcgttctcacgggccgtctgggcagcaggcatttactgatgtttggagtaatggggacagcactttacaggtgacaacctggggcacctcttggcagctgggtgcctccctgtgacctcagctgtgacctcagcagtgacctcagctgtgtctctctgcccttcttgaccccatcacctcttttcttcccctaactcctggttccatgggctctcacctcctctctcccaggatgtctgagaaaaccacagtccctctcactttttgtccaagcagtccagcgctctctgcccttcaccttctcctccggcctggacccctgaaggcaagggagagggtagagatcacctctcagatccagaagatccaaaaacacccctggggtgtaggagtcatgctgattccctcggctgtgaagcagtgcgttcttatcgcaaagagcacttctgtttttttcttctttctacatctgtgctaacattctgtgattcttagagaacgaaaattctgaagagcaaagcccagagccaagaatgttcaagccagaagggatttctatgaggaacaaaacagacaaggcctctgcctgcagggtgcatatcccgagtggagagatggggctgtacagacccagagagcgtggctcgccctggaagggaaggtcaggtgcgacgggacagaatggccaggaggactagtttatactagggggtgagggtcagggatgagcaggatttagtgaagcaaatgatttcaaacatcaaaagcacaggaaataaaaggaatatagatcagacctcatcccaatgaaagccttctgtgcattaaggacactatcaataaagtgaaaagacaacccatagggtgggagaaaatacttgcaaattatctaccagatcagggattaatatccagactatataaagggcttctacaaattcactaactacaaacctgatccaaaaggccaaggatgtgaacagacatttctcatgaggagacacacaaatggccaatgagcacatgaaaaattctcaccatcactcaccaccaggaaaatgcacatcaagaccacaatgaaaggccgggcgcggtggctcacgcctgtaatcccagcaccctgggaggccgaggtgggtggatcacctgaggtcaggagtttgagaccagcctggccaacatggtgaaaccccgtctgtactaaaaatacaaaaattagccaggtgtcgtggcaggtgcctgtaatcgcagctaatcgggaggctgaggcaggagaatcgcttgaacccaggaggcggaggttgcagtgagctgggatcacaccactgcactccagcctgggtgacagagtgagactccatcccaaaaacaaacaaacaaacaaaaaaacacaccacaatgaagtaccagttcacacccattaggacgactactgtctaaaaaccagagaatcacaagtgttggtaaggatacggagaaatcggactcctgtgcactgctggtgggaatggcaaacagtgcagctgccgtggaaaacagtttggcagctcctcaaaaagctaaacatagaattttcatatgacccagcaattccacttcagagtatatactcaaaagcaaggacaccaacagacattggtacacatgttcttagtagcattattcacaacagcaaaagctgggcatgacccgtgtgtccctcaacagaggaatggacagacaccacgtggtttgtatacacagaggaatcttattctgctgtgggaaggaatgaaacactggcgctcactgcagcacggatggaccttggcaacaccatgccgagtgaaagaaaccaggcaccagggcagactgtaggattccactgctgtgaggcgcccagagcaggcaaacttgtaaggagagaaagtaaagtagaggttcccaggggctgggctttggagtggaacagggacttagttactgcttcatggggacagagtttctctttgcagtaatgaaaaagtttttgaaatagacggcggtgatggttgcacaacattgtcaatgtcctgaatgccactgaattgtacacttaaaaatgggtaacatggtaaatttggtgttatttctatttaccacaattttaaaaaattagtactgtactataaaagccatggaatcgcacgctttccataggtgaattgcatggtttgtgaattatgtctcattagaagaggagcatttaaaagaaagagggaggccaggcacggtggcccacacctataatcccagcactttgggaggctgaggcgggcggatcacctgaggtcaggactttgagaccagcctgaccaacatggtgaaaccccatctctaatcccagctactcaggaggctgaggcaagagaatcgcttgaacctgggaggcggaggttgcagtgaaccgagatcacgccactgcactccggcctgggtgacagtgagattctgtctcaaaaataaagaaagaaagaaagaaagaaagaggggcagcagcccccacatgcagcccccatgcaagagcccccaaggagaggcttgatgcatcagaggtgctgggtggtctggcaggctggagccaggccactggggagctgggagcagagggaagtgtgtggagggcagccctgccccgggttctgcagcagcccccgccttgtctgcctgcactgctctcggctggttctgtgctgagcgcagcctctccttggaagccaattttctgcacagtgaaatgttttttttgtgcccactttagcagatggccccccaccttggcctgaattgggaagcacagaaatgcagcaggggacagacggcttctttggcaatgtcttacaggcacacgagcacgcagactcatacccgccccccacaactcgccttcacggactctgggctgcagccggcctccggagacctctcctcttctgctgccgaccagctgctggacgcctggcccaggtgcttgcttgggatggatgcagatgactgcatgtctccaaggccgggaccggggcgcttcctctcctgcacccgcccctctcggccagtccctccactccccctaatgggagaatgctgaagggatcctggcagggactcctgacctccagtgtggagaccccgccccaggcacctgtctcaaccccatttacagggtagctgagaaacttctggggagacagcctgaccttggcccaccactggagtccagaccttaaacccactgtggtcagtaggcaggagtctggcctgaggtttgaggaagggcaacagagtctccagtggggaagtcgccattgaggaaaagtctagtgaaagcagatcctaggcccacgggaccaatggtgtgtcagggccgagttccagggccagcagggaggagggggctggtgcagggagggttggtagcctccacagagggatgccaccggtcctcatgggttggagccagcccttgggcaccagagagagcctgaaggggctacagagaccatctgatcctaccctgttgttctaattttttttttttaaagacggagtctagctctgttgcccaggctggagtgcaatggtgcaatctcggctcattgcaacctccacctctcaggttcaagtgattctcctgcctcagcctctggagtagctgggattacaggtgcttgccaccacacatggctaatttttgtatttttagtagagatgggatttcaccatgttggccagactggtctcaaactcctgacctcaagtaatctgcctgcctcagcttcccaaagtgctgggattacaggcgtgagccaccgcgcccggccctaacctattgttctgtgctggggtggggtggggaagatggaggcttgggaggccactgcccactcctcatgtgaccaggtcagtctcaggaaatgtaagtgtccttcttagaggaactgggagacaaagggattttcaggtttcgtttttgtttttgagacagggtctcactctgtcactccaacacacctcagcttgcagctgtgtccctccagcctctgactccatcatcacagggccgtctgtcttcctctgtgtctcttcttttttaaaatttttttgtttttaaaattttttttagatggagtttcactcttgttacccaggctggagtgcaatggtgcgatcttggctcaccgcaacctccacctcctgggttcaagcaattctcctgcctcagcctcccaagtagctgggattacaggtgtgtgccaccacacccagttaatttttgtatttttaggagagatggggtttcaccatgttggccaggctggtctcaaactcctgacctcaggtgatctaactgccttgacctcccaaagtgctgggattacaggtgtgagccaccacacctggcctctccttttcttataaagacaacacatctgcaaagaccctgtttccaaatacagtcaaattcacaggcaccatggttaggacttggagctgtatttctgggggaaacagttcaacccataacaaaccaatgtgggggtactttttttttgacacagggtcttgctatgtcacctaggctgaagtgcagtggtaggatcatggctcactgtagcctccacctccctggctcaaggtatcctctcacctcagcctccacagtaggtgggactacaggcacgggccaccactcccagctttttgaaactttctgtagagatgggggtctcactatgttgcccaggctggtctcggactcctggactcaagtgatcctccaacctcggcctcccaaagtgctgggattataggcatgtgcttccatgcccagccgatttccaggttttaagggtcaaactgttggaaacatttatttaaaaatccagtaggagggaaatgtgtgaccagtgagtgcagagggagtctgtggcatttccaggaagcaccccaggccaggccttgagcatctgggagaggaaggatgttcaggccacggggcagacgtctccacaagccgaggccacagggacttagctgtgcgtgagtgtgggtgtgtgagcagccagctcctcgagctgaaggagctcgtgtgccagcgagagggtgctgctggggatgagcccacccccggctacctcgtagggaatgggcacagggagaaagcaggactccctcaactgggatgcccctgctccaggagcccacagctgggctcaaggccgtacacatgcccggcttcctggagcaccaaagacaggaagagggcagctgggggacctggagccggatgccctttcccaggataaatgtagggcttccagaggccttgcatttgcagggggccactttaagccacatccccagaccttccagggcttggtccagcttcccctgctgctgcttatgggacctgggtgggggatttgacaagtcctggagcagctcagtgggtggggagggagagatccaaggacaggtggcctgcagtcctgcgtgggggcgggggacgcagccttcaggagctgagaaggggacctccagacatccggagcttcctggtatggatgacatgggaggcactggagtccgagggctgcagctggggtgccccatggcccaacctgaaggcaacatttcccagctccccttggttctttggcatcccaggagggactggtgcatggggaggggcctgtgggcagtgggggcggcgtccggccttcaggagaggaacctggaggagggtatcctggcatggcagaaggtagtggggtctgcccccaattgtacctgctcgcaccaggccctgtgcatatagctcctcatcccatcttcacggccctgagagagagtactgccagccccacagcacaggtcaggaccctgctgtttagccaggccaggcgagaacactgagctgggagtcaggaatcctggattctgatttctggatgtcaagctgtgcaactctgaatgagccacacagcccctctgrgcctcggtcccctcatctgaacagtcggggagacagactagacgaccctcaaggttctccaggcccagacatcctgcaggtcttggacgtagcatctctgtccatagcgacagagacagaagtacccgccatgcaggcaggctgcccctggcccagtgcaatttgaggaggctgctgcatgggggcagagctggtgaagactgagggacccccgtcttcctgtgttgggacagatgggcaggagctgtagctcaatgcaaggaaccaagcatgaaatgacagaaaaagaagagacgggagcgcaaaagaaaacttgccgatagtcagagccgtctgaaaataggacagactacctctctggggtaatgagctccacatcaccagaagtgtgcaagccgagaacagtttcaggcacctgagaggctgctgaagttaatgacttccaagacaccctctggctttgacttcttgcgcccgtgtcccgctgcccagactcccacctcactccaccgagctgcaaggcccgtggcttctttccaagcagctgttcttagctccacggccccctgaaggcattcatggatgagccaaaatcaggcgtgagaagtgcagaaagtactttgcatctaatgatagttaagaacccaagtgtttgaatgagctctcacatagagattagaaatgtatgcgtgacgttatcacagacgtaactctgcactttcttgagtgggaggtgtgaggcggtgtgtccattggagccactggaatcgggcataaccctgggcccgaccgtgagcgtgcaccctggggtgaacaccacccagcgagaatggctctgggaacacaggccaggggcctcccgcctggcagggcctctgtctcccgaccaaggctcttcctgcagggacacagggggttccctgcctgctttgctgaggccaaaccttctccctccacaccatcccaggtgacaggtggctcactcacacccactaaacccccgctcctgagtcctgggaacaccagagcaccgggcagagaatccccgggcgatgccaacaggaagccagagtgaccagtcccttccgccaacccagtgtgtgtgtgtgtgtgtgtgtgtgcatctgtgtgtgtgaggtggggggcaccaaccaaaccaccgggcaggtgattttggctccatgtcccctgacttcaggttgtaagtgtgctagaatcactcaccccagggcacttgggtcctggttactctgtcccttggaaggaacagctcgcaagctccaaacacatccagagtcaagccaggtccacaagggggacagggaagtaagctcagcccaaggcccagcgttcccatggctatttatccctttgtagggacactctcagacactctcagacaagtgcagggaaggggcctgggactctgaaagcgggaaggcaaccacacgtggctaaggccccgctagcagaggctgaggcagagaactgccagagttgcttgggcacgacaagcccgggccacctcgaacccggacccagctgtggaccgcgagtccacccaggaggggaaggggaatgcggagtgaccccacatcccaggccgagtggcctcactgagttggggaaacactgggccactctctgcctctgcgtcagctgcccctgggaaatggagacccctccacccaacatccagcatcaccagacggtgtggggttgtcaaagggcacagggcaatgcacaagcgaggcacacaccccctgcaggtgagggtcacctgcacctggctggggaagaggggtggctgcagggggaggtggcattggcaagcctcggaaaagaagagacatcctgaggagaccacagtctttctccactctttactgtgtaaaaataaactcaacaattcatgtcatttcagcaagaaaatgaaaaaaaaaaaaaaagcaagaataccagtagaaatagtgcatttccagaattgctgatgggaggtggcaggtcccatggttttgacccatttgatacccaaaaggctgcagatcatatgaaaaatgtacagctttggttagcaaataatgaaaaagtgagatacatcgattctctttcatattatacagtatatacattataacaatatcaaccatagggggtttgctttttttttttttttttttttggcaaatgagacaaaattgggactctaagtattgggctttttttttctcttcaatgacttgttccctttcaacaaattgcacttcagtccactgtgtacagaagtaatacatttcccatcgttagttgaataaatttacacactgtgtttaagagatgaaaccacgacttcgtcaagccacatttggttgccataagattccaaaggattctatcctcttaatgtgaaccacatgtaacccaaggttcttctatcaaggttagaactgatattcaaatcctcggctcattccacttagtaagtcagctccaagacacaggttcaccataaatggccagttttacctcctggtccccaacatgcttttgagaacaggtggcccctctttggagctcactggaatgactgaacctcatgccagcctctcctcaccccctcactgcccacaagacacccccttggtctcaggtcccagggctggtgacaaagtgatctttgcagtcttagaaaataaactgggaatctacaaaaggaaaagaaagcatacaaaatgtatctctttcttccaaaatacatgctttcagttctacgttttggtggagcctgaaagtattttcaagggagatataggttgagttaatttataggtttgtccttttctttcataaaaatactcttgtttggtaataaactttttcttctacagtaagagaaataatactgtgttatcaaaagcaagaaggcttacgactcaagtgggatctaagcccctgagggcccggagagcttctgtcaacagctctcccaccccggacacccagggagctgtcggacaccagctcggcaaaagcaaacactgcctcaacagtgctttttatcttcttgttttataaaatggcttacctgagagaggcatgtattattcagtgcaagagggaacatcaatcagattaaggagtaaatgggaactacgtggattagatttgcggctgtgtcatcgacctttctcggggccagcaaagtgccaagtgtcaacacagtgagggagtcgaggggcttggctcagaccctagcaaggggcaggagggagctaaaggtcagcttcggccctggcaccatgggtgggtcagaggaggggtgaggaaggtgagcgggccaggaggctgcaggggtggaggcgtaagcactggggtgtgctctgcccctcgctagtcagggcagaggatggctcatggtcctgaacggacagctcccacctggcccacgggggcagaaggtggtctgcggtccagtagatcggcaggggcggggctggccggaggtgctggcaggggagcaccccacacagcccgcaccgggcgttgcctacacaagggctccgagatggctggagtgagggtggggctagatctggtctagagaggcgactccaagctctcttgctggctcccagctgtgggaatcctttaggcttgttctcaacctacacgttaaaaatgcttcttggtgtgtttggggagggggagagggaaactgagctctctcttgacctcctccaacacccttgacttgcttacccagccattttcagtagctacacgggtggtcacagaacactgggcggcactcggcacacaacacagaaccggggcagtccatgcaggtgcgggaacacatgtcggacccagggagcaaggaacacgccaccccgaggaacatgcaaacggagaagatccctgcagatccaaggatgccacaacccgacgggcgctgaggaagcacgattatctaagaaaaagccactgttgaacaacaacgcgcagcacggcgcctgatctgcacccccctctgcgggagtgatgcattgcagaacggcggccgggtaccgtgcagacacccacgagaacactgacacagagacactgacagacacacacagatgcactaggttcttgacacaacgtggacctgtgtttaaggggacaggtggccctagaaggaacagaagacagaaacacctgctcctcctgcagaaatgtctgcagctgcagctgagccaggcagtgcagtagaatttctagaaagacttgtcagcggtttgctgatgagactcctggagacagagtcggggtgccctcagggcgtgcgaccaccagatggtgcaaagcaggaagctcctcctaccctgggcagggcctcccgagcctgctctctttctcacttagcgcacacacacacacaacacacacacacacacacaaatgcacatacacttctctcccctccttggaaggactagaaggaaatgtccatgttgcttttcacggggaacaaaaaaaggcaaaaaggttctggacactgctcaggtgtcagtgtcgccggccagtggcagcttgtgggctgctgtaggccgtgagccccatgagatggaggagtgggtttcaggcccctgccctggccaggcctggctccacacgtgaggtctctggagtacccctggaggggtctgaaccagctcacctggcgcctggtgtgctgggagggatatggctgtcaaagtcccccaaaaaggaccttcccaccgatgggctgggttgaagtcccagtggccccatgtggccaggtcttcatcgaaacacggcttctttccttaaaaggtcccctctggtctgcactgccggaggccaaaggagaggggtgctggggcccggcttcccgggctgtgggtggcacgctcctctctcagggccctctgggctgaagcagtgcagcacagctccagaggtgagaagggaggggcagaccccagccttgcaggatctgaggaggtgggaaggtcgagggcagggccgtcctggggcagagctagctgagctgggcacacaggccgacctcccaggccccatgctgctctcagatgcaaacagcagggatggatgctgtctggccctggggtaagggccagtgtcctcagccatcccctctcctccacgggagagcacagggtaccatcccaggacggcctgccttcacccttcagagacagttcacttctattacaaaataaaaattctctgtagagagcccccgcccccggcctttgacaaaccactaaaagacatttattacatcaatataaagatatgtcgctctgagaaaggtttgcaaacaaagacaaatgcgtgtactgaggttgcaccaaccccaaagcacgcacgcagctaacaaagttggcaaatatgaattttcatcatgagatttttccaaagccagaagaaagaaggaaatgaggtacgtcctcaaagaaaaggcctcccccagccacgcacgcacacacgtagacacacacatgtagacacgcacacacagatcctctcaccaacttctgccttgttcaagacgttgcagcacccacttcagctgtgaatggggcatgcctgagagagtccgggctcctacagagaagtggggcttcctcagggacagatgggagcaggggctgcttcgtgtgcatgaggacaaaaccaggctgtgtgcgtgtgcgtgtgcaggggcgacggcctcggttcattcctggggaaaagggagagaagagacgcacaaaacagatcatcgccgcacaagcagggggcgagggcgggcgggctcagcctggcctgccgtgctcagttgatctggcgacaggcctcgaaggtccacttggtggcccctccgtagatctcgatgtgggattcagcccaggcgatgacattgccggacagagccttggtgctgcaggtggccaggttgtacacctccccgggggtcagcttgcggtcccagatgttgaagtgggccagctcacccacaaatgcctgggtggcatcaaacccaccacccagagtgtccttcagagaaaaacagaggatgagagtggaggggtcgccaggtggtctgggggcccatccctaggccggggatctgagtgcctctgtgcctgcccgatcacggactctcacggacactcctaggtgccgtttcgtctccctgtccctgactctccagccctacatccatccctcctctgggggagcccaagaaaagggatggcaccgaccacctctgccctggagcttctcctggggggacctggactctgcccctcctgcagctggtccctcagccatggacaaccacagcagctgggcctggggcccctgcccagacatcactccatgccagctaaagctgcccaacagggacagacgtccccgattctccatcccttcgtcttcctcccaggtgcccaacaggagacaaaccacttcacgggaactagattatgcatcactaaaagcagaactggccttcccatgccaaagaaagtgttgctgcagctctgagagcgttgaaaccagagctcactcctgcaggaagttcaagtgcatgtccacccagggcacaggccttgcccagcctgctccccgggctgcttctcaggggaaccacttctccggctacctccctccctccttgctcctcttccccttcccagcctctggttctgaagctctctccccttccccaccacatccagcacagtacctgctcctggcccagcaccagcacgccctggggcttgatggggtgatagggcgccaagttctcgccactgccaccctgcgtgccatcctggtaggcctcccagaccccgtcccgggtggtccaggtgacacagatgtggtgccacttgccatcattgatgacaaaaggcaacttggccacctgggaaggagaatgacagacgccagctggtgagtacaggggtacagcccagaagccataatcttcactgcctcagttgttcctatctctacttgcaaagtaaatgtcaataactattactgaattctcacagcacctactgccccagacttcagcgtctgtcagccccgcctgggcgtggggcacagaatcacacaggaaatgtaaggctctaccgagggaacccccaagggccatgtgggtgtgcagtctcttcactcctactcaccctcctccctctttgctagtctacacaacaagccctagtctacaccagggagagccgtccctggtgtctgagttcactgctgcctctcaaggctccgtgcgacatctgtgcagcctccttgctggcctctcctctctcttccgatgtcccatctctagctgttacaccctctcactctacaacacggcaagcttttgccgaggatctgagagatgtcatggagcacgatgccttttaaagctcagaccagcctccaacctggaatttcactctcccactgccattccaggtcgggaggatgacggtccagcagacagtgaggaaagaaacttcacaccctttccgcttggggaaagggggcccagaatggcacagccctttgtggggaagagcaacaggaacaagggcgtggaatccacttcccagcccaggttttagccattgactaccactgctggcctggacttgagacagtttgaaagctggcagctagttccatagctaacaggggaggatggagaatgttcactagagggaaaggctgcctgtgctgtcctgttccaagggatcacacagacctgtcctcctcccgagagttagggggctgagtgggatagacttggactcaggaaaaatctctaaggacccctcaccccgctagagcatctctgggtccctgctatggaggggcaaagccagttccaaggccagctggtggaggagtccagatcacatcatcgggcagggccaggccccatggctcgttctccgccatctcccgtacccaccccaggaggcatccgggaaggtccagctctccctgctctcccagtacaggtgagggggctcctaccacttctcctggtcttcaccctccccctccctcagtcccacctggcaaacactttccttggggccccatcaacatctgaggccaccatagccctgtctccgcatgcaacatgagctggggccctggggcctcgccctgccgccctgtgagcccgggggctgctctaccttgtcattgatgaggatctccatggggttgttgccccactcaatgaggaccagctcgttggcctggccgggcacagcgtaggagaagggcgtgcccacacctggcgtggcgctggacttgagccacatgcagacagtgaaggcgtacatctctggcaggctcttcttcaccttggcatacatatagttggtccgcagtgggaatgtgagctggaacttgtctccagggcggttgtctttctgacctgcggaagacacagtcctgacatctgcacctgcgtgaagtgcttatcggtgtctgagttgagttcggtccccaccaagctctgtgatgcgtggccagggcaggctccggtctccattctggagacgcagagcgggtagcttcccaggtcaagtactttgccccaaggcacagggccagtctgtaaagagctgacccaagctcttcatgctccaggtctggcctgctgtcacgcccatgtgctggcatccttggggagatgggggtggggggcagtgcctggtgggctgctgcatcaacactgggctgcgtggtgggcatgagaagggaatggggacagccgccaagccaagtcaacccaaggagcaggggtggctgcagaggaggaacgaggctagctaccctgagagtaaccgacctgattccaggatggggcggggctcagagcctctcctcgctcaatcccaggctctgctcctgcctctccccacccgacagccaggccctggtgcccacacacttccctccaggccagatgtgcctctgacccaccaatgcctcttgggtgtgaggctgggcagctccccacctccaaggcccttccttggagcagggactctgcctggagctctgctgagagggcagggcagcaaggagctgggagtactctgggcgccagggctccccaaggtgactgctctaggatgccgccacactctcaggtgagtaagtgtcctcacagctcaggttccaggcctggtggcctccctttccacaaggcccaccttctccctccccaccggccacttctcccgtaagcgccaacccccatagcccatctatatactgtggcaccctgggtatggtgtgactgactccaggcaaaacattcctggccagcaatgggcacagtagcaggcccagagcccaggaacacacaccccttggtcaatccctgggtgagcagccgtggtcctgccaggaagcccagaaagaaaccagttccccaggccaaaaattatttctagaagacatcctgcccggtcagcaaccaccccctcctctaaaccaactggcagggagcagagcagggcaggccccctcagtgcagcaccccagcggctaccccaacccattgaggcctgcagtgggggatagggaggggcccagcccctggcctttctccgggtgtttttcattccccaatctccgctccttccctctccctccctcctgaggtccttccttctgatctgtttttcagttacataacaagccttaagatggcactgtttaaatctgtccggcacattttgcggtctgagcagggtcacgtggccttgctccgcaccgggcacccccccccctccccttttacaaccttgtttgatttctgcgacttcaagacagagcctccagcgctcatccaggcgccctgggtgatagactggctggcctgagttggtgactgagaaacctcagggatttgaaagatgccaggacttgggtcactcagaaaagaaaacctcagggtaggggttcaggacagaagtggggaggagctggagatcctgagaggacaccgggggcatggcagccgggaagaaacctgcttaacagcctcagcggcccggggaacgagagcgcccagccccagcctgcacccagctgtacgctgaggctggggaccagggagagacaggctttcttaagcagggagggtttgagccagcgacagccactgtgcaaagttactgaggattaaacaaatcagacttgaaaaatccagcgagtgtgtgagtgtgtctggatgggggagggggcactatgcaatcccctttcccagagacagccccagcccaaggcacccagccctgatagaaacaccgactagaagcccagaacctgggaaggggtgcggggaatgagaaagcggtgcaggggttgggggtagcggagcggcttgaggcttgcacagtgcagagctgggctgttagggatcgggaccgaggcagggtcgggcaagcaggtgcaggcaggcagcacggctccccctggccccagtacctttctcgagctcgctgatccgctggtgcagggaggtcagggcggtctcgatcttgaccctctcctcggtgtcgttcctggggccccccttgccctcctccagggtgttcacccgggacagcacctgcctctccagctcatcgatcttgctctgcagcagatccttgaggctgttggtctggctggaggaattgaggcggctgtactgctgcggggtgcaagggcgggggaaaccacaccgttaggcgaggcgcggggaccgtgctggaaggcccgcggcgcggtcccctctgggcgactgcgggggcggcctggcagggagctggggcgagtggccgccgcggggcctcgcaggcgcggggaggcaccggccgggcacccgcgcggctgaggcgagggcgggggatgcctggccgggtagggaacggggtgggggagggcagaggggcgagcgagccggagggggaaccggagccgagggcgcgcgcggacctcgaggttctccaggcgggttttgagcgattgcaaagtttgcccgagttggctgagcgtctcggcggccggtgtccgggacaggtcgcccatggtgttcttgcccgagccgggctgcttgcggccgccgcccgcccgggcctcgccggctccggggtccagcgtgctctggctctcgcagcggcccagcttggcggtcagctcgcggatggtctccttctggctcaggatggtctccttctgctgcagcaccgtctcgcggagctgcagcacgctgctccggagctcctcggcgccgccggcggccacggacgcggcgcacatgtcggcgtccacgggcaccgaggtgcagatgaagcgcgtcggcccgaaatcctgggccccggcgcccaggaggcagagggcgagcagcgcacaggtgcgcgcggcgcggccggccggcatggctgcgggcaccgggcgctccgggcccggctcggctcggctggggctcggctccggctgggacccggctcgggctgtggctccgcgagcggcccgcgctctgggcgccgcgctcttcggccgcgcggtccacaccgccgcgctatcaggcccccactgccgcctgcgctgcggagcccgcgccttttatgccgccgcgggaggggcctcgagccgccgacgtcagcgggggaggaatcccgctttaattgtccgcggcccgggcccgcggcgcggcgggggtctcacgagcgcgccaccggggctgcctccgctcgcgtcccccccgcccttctgggagctgccgcggccccctcctcccccgcccccgcctgggccggcgctgctgccccgaaggctccgagagctcagggggaaggaagtcgggctccggccccggactcgaggatggaccacctgcgtggtgtccccgaacctccgacggggctccaggcgggcgggaaaccaccgcgtccgggagatagaattcggttcggccctcctggctgcactgaacacccttgggccggggtcgtttggagccgggctgggtgcccaaggacccaggtcgtcaacacccgagtgcccggtggtcaacggctcggagtggggcctcgcggcgccactaagtccagagcgggggtcaccgggccgccccgcgtttcttagatgtccgtgtgctattgagaagccgccgggcagcctgggtacagggacttcccacgggtcggggcccccggcgcctcccctcccccagctcccgggctctgtggacccggccccgtccccgggggactgtcagtccccaccccgtgcgggcggggcgctggggacttctcggaggcagacctagctgatcgagcctttcctgtccgcggcgccggcccgatcgcgggcaggcggcgtcgccggggctggacgttcgcagcggcgcttcggaagggggccccgcgggagcagccgcccgcgtctccagcagcttccccttgccaggcgccgcgcgcgcccggtatccccgggtgtccacctgtgcgtggggggctgtttcccgtctgtccagccgcgcccacttctcaggcccaaaggccagcaggaagggtcccggaggtggctgggggcgtccacctgagaagctccgctctcgctcagacaccccacccggcacccgcgcactcgcgccttcatctggaccgcggggctcggctcccgagatcgcggtccgggttccggcgccgggctgggggctcaggaccagggctggcgcagccgccccgccccgccccgccccggggctatcagagcggtcaaagcctcggaaatccctccgtccacgctcccgtggtacagagaaggaaactgaggctccgagactgcggggccgggctacagccgtggccccccgacccctccccgccgcccccggcttctctggtctctgcgttctgcagcctttccctgcgcgcagccgggggctgggggtccccagccagcagggacccacgggcagtcccgggtgccgggatcgggcgtccgcccctcgggcgggagcagaggccggctcgatggagacctttttgagaaatggcggcctctacccgccccccagcccccgagcgcccgccgtctgcgttcagcatctcagcttttaagcctccctgagccggggctgcgttctcgttgtctccttcggccgccgcttcctggcgccagagggaccccgcggcccgcacgggcctgtcctcggctccgctcggagggtgtgcgggttcccgctccgctggccccgcttgcccaccacggcgcgcccaaacccacgcggcccgcggccccttgcgtgctctgccgaccccgggccgcaggaggggttagcctcgccggtccccacgcggggtctgcgccggcagcgccacgggctggaacgcgcctcgggagaaattacccgcgcggggtcgcggcgtcttctgccccaactcccgtagggagccgctaggcgggtgccctcctggccccagcgctttggcctcgccgccctcccgcacgcgtccagtggggaggccgcgcccgtcaggcctggagccggaccgtgcccagccgagagcgcgcgggcgcgggagaggagccctgccctgcccagggcaggctgtttagacggggcgggaacccttagcctggccccgcgtctctatccccagcagccgtggagccttcctagccgcccaaggccagggaccctgcctggcttttcctccagggcagcagatctcagcggcctggcacctcggcacagaggagcgtgtcccccttccgcagaggaagggagtggacggcgtcagaaagggaggggagcctggggcggtgtctggggctagcgggacctctgggcactgtggggccccaggagaggagaccctcgggtccagcctgcaactgttgtgcaggaggcgctgggcgggacacctgcaggcgctgcacacacacgcgagggccaccatgccgcgtttctccttcacaagaccctgggcaagcggagggtcgtatttaggattccctcggcctccccagtgttctgaaagtatctgagagggagggagcaggaaaacgaagggccgccctgcccaaactcatcggggtgtctggttactaggtcggaagaggggggatggtgttgtgaacagccagtgatcccggccctgcctgagtcccacaccccagatgcagctggggtgagggaggcgatcccgtaatattcccagccgcgtacatttgtggctgcctgtgacacgacctcactcattgggaaaccatgcacactagcacactgttattttatttttgagacagggtcttgctctgttgcctaggctggagtgcggtggcgcaatcacagctaactgcagcctcgacctcttgggctcaagcgatgcttcctgcaccaccacacttcagctaattttttttgtttttttcgtagagatggttgaactcctcttgccttggtcttgggactacaggtgcaagccacagcgcccggcctagcacaatagtacttattatactactcccaccataacggtgcaatagatgtgaataaaactcaaaagcatagacaaggctgggcgcagtggctcacgcctgtaatcccagcactttggaaggccaagctggatcacatgaggtcaggagtttgagaccagcctggccaacatggtgaaaccccgtctctaccaaaaatacaaaaattaaccaggtgtggtggcacacatctgtaatcccagctacttgagaggctgaggcaggagaatcacctgaacctgggaggcagaggttgcagtgagccgagatcgaaccactgcactccagcctggatgacagagcaatactgtgtctcaaaaaaaaaaaagatagacaatagttcaatgtaagaaaataattagaaagtgatgagtttgtagtatttattgcccatttaaaatataatttatttaattataagtttacttagtttaaaatgtaatagtggctgtgtttaaccatcgacctgtgaaattcctgcaaatgccaccatcagtgggtactggccggctcagctcactcctggtggaagggcacggagaggcctgcagaggttagttggtagtgggagggggagtgtagagaaggcaaagggggcaggtgtccaggagaggccctggttagtggccagtatggagagggtggccaggcataagaagactagacagaccgggtgcaggggctcacgccttgaatcccagcactttgggaggccgaggtgggtggatctcttgagcccaggagtttgacaccagcctggacaacatggcaaaatctcatctctacaaaaatacaaaaattagccaggcatggtggggtgcacctgcagtcccaactactctggggactgaagtgggaggattgtttgagctcgagaggttgacgctgcagtgagccgagaccacgtcactgcactccagcctgggcaacacagcaagatcctgtctcaaaaaaaaaaattttaaaaaaagaaggctagccgggtaggttgggaggtattaagaggacctcagtgcccatcaagagtctttactcagtggacggtgggagctcatgcaggggtgtgcctaagcctggtgggtgcctgtgccccttgctcatcctgccgtatctccccggcctctcatcttccaggccatgccttgagcttatctggaacctccacccttagcaggaagaagggtgagcaatgggtgggtgctgggagaatgctgggagggcctgagagcactgcggggtgctaggcagaggcacccttctctgccccacttgcctcagcccttcccgcatgatgttccatgctgcctctggaggggtgaggctgggaggggagggtatggtggggtgggagaggccctggtcccccacccgcagaagagaagcaggagccctcgatgatcacagccctgggatagacacagtgcctcagccaagggtcaggtacatgcccctctggaggacaggctgtacccctcggagactcattgcattggcagctggtctaggatgtgggacagtctgcggtaaggtgagtggccggggttttagtgtgtgagatgtgcaacgccattttcacctgcatctgagtttccagcagctcgcagggtaccggccatattgtttctctatctgggtgctgatctcatggggtgtttgtcttgtgaccattcactgggctgtccaggtgcaatctgtgcacctttctctgtggttctatgcctcacacagggtgtgcagggcacagcatccgcagggtgtcctgggcagaggagaggggcttctggccgcaccaggggagaaggcacagggacctctggcagcccagccctcaggacacagtcaggccacgtgaccttccccccaagtgaggcgcccctggacccacagggttgggtgtggggctgctctgacggcagccagtcccacggggcctctgagtcacagcctcagcccacctgtgccttggagtcactcctgacagccccacccaccagctctgtgtgataagggacaggtccctgctccccggctctgctgagggaggcgccccacccgagccttcccctctgtcctctacatgacacccctgcggtggtaacctaactgtgtcttaaaaacaagcacttgtcccctttccaagtcctgggtccacattcacgcatttgcctttcaaaggcacagctgggctctagtcctcgctcaccacagaggaaacacagggaagcccagagaagccaaggtcgcaccgtcgcccagcgaaggcagagctggggccaagccctggccttcgcactccatgtggccactttatctctgcccagcacaggagctgcaaggaaaagactccaggagaggagccctgaggggacagtgccccagcccacccatgtccagtgagtgggcagggcaggtggggctgcggaccccaggctcaagaccttctcaccagccatcttcagaggagaagccagccctgcctcaccgcaggactacgcgcttctgcccactatgcaagtgtggcgaagcctgcagcagctgcagccaggagctgatgggccggggcagtgccccattctcctgcgacagctgagctcccaccccagattccctgctcccaccaggggcttccccacagcaaggaccaccttcctcctctgagccagccaggcggccactggcccagtctggctggggacagggccctgggtggtctctgtccccaccctgcctgttccttctcttgggctgagcccagtgctggggtcaaggatggggaatgggtgctcttgaactgctgactcactgatcccaatcaacaggagtttgcaggggaaaggtgagcccttcccacctggggaaggcgtgtcttactctccaagtaggcatttcatggcaccaggcagatgcttgggcatttgccatagtgtaactgttcagaacagttgctctcagttgaacaaatatttccagagcatccactgaggggcagaccaggtgttggggccaggatgagtaaaacaccattctccgtctctgaggccaggcgatctggcaggctagacagggctgaacacaccttgggtaccatgcgtgagggccacaggacgggagaggtcctgagccagcaccaggcgcatgggttagggtccagccatcagctggaagggtctcctgtggaggtgataacaggggcttgaagagtgagccaagggggctggcactagcaatgccagagcaatgaggggcaggccgtggggagacggacgtagttcatcctgccaagagtgagctgcaggggccagggtagatgtgggggagttgtgggcaaggtgggccaggaccttggggagaacccacagtcctgtggaggcgctgtctgggagcccttggtgagggcggagctgagcaggagcagggcttgcagaagagacctggagggtgatgtcacaggagccagggaggaaggggactgggcttgagaggcttgagggcatttgggagattccttggcaggggcttggtgatggcttgggcgtgggaagtgaaggagagggaggaacctgggaagatccccggcttctgggcgagggggtggtggtgattctgcctgcagagaaggggtggggagcctgggagggcctggagggagggaggagtccacagttcacactaggacgcctgggttttccagtggctatctgacatccgggaggaggccagagcccagcacacggaggctctagagattccaagacgatgaggaggctggtcctgagctgacctgagcggaaggggcctgagggtgcagagggaggacggggctgacagcagagtcctgggagcatctttagaggccgtggaccacagccaagccagtagggagcccatccagggaggcagtgggtacaaaaacagaagaaagctgaaaagagcaccctgggactccgatattcaaggaccattgttttaaggtttctgatctccgcattcaacagtgacaagggcagcagacatcggtactctcaggcatttgtacagcaccttctgcacttcaaaatgccttccctgcacccccaacacagggcttgcaaatacaaagccaggtgaggaatggccctagggcccccgtcccagccctccccagtttgcattcatttggttcctgagctaggacgctccctccagctaccggcatctggctgctgaggtgggggccgggggcctgccagcctgctgtgtccttgcccatcaattttaccacctgcgagacgccaggagagggcacatgaaatataaaatgagttgaggcataaaagtgtttcggaataaaaatgatgtgtactggcaagagattacagttattacaaggaccgtgcgctgccgccatcactcatacctgggagaggaagagtgtggtcgcctgccgaaggaggcagcagccaccgtgcctcctcctctggggacagggccgcagactgcagggtgccaggctgcaggatgggctgaagccagccctccgggagcatccagtgctctgggggatgaatgacactcccatgtcacagttaggcacaatgcagggccgggcgccgtggctcatgcctgtaatcccagcactttgggaggccgaggcgggtggatcacctgaggtcaggagttcaagaccggcctggtcaacatggcaaaactctgtctctactaaacacacaaaaattagctgggcatggtggcaggcgcctgtaatcccagctacttgggaggccgaggcaggagactcacttgaacctgggaggcagaggttacagtgagctgagatcacgccactgcactccagccgggcatcacagtgagcctctgtctcaaaaacaaacaaacaacaaacaaaaactattaggcatggtgcaaatatgctcctttttctgttacattttccaaggtgtcttggacgtttaccagacggctttggcaggtggcattcttttttttttgagacgaagtctcactcttttcccacaggctggagtgcagtggcgtgatcttggctcactgcaacctccacctcctgggttcaagtgattctcctgcctcagcctcctgagtagctgggattacaggtgcccaccactgtaccaggcgaatttttgtatttttagtacagacggggtttcaccatgttagtcaggctggtttcaaactcctgacctcaggtgatccgcccaccttggcctcccaaagtgctgggattacaggcgtgagccaccgcgcccagccggcaggtggcattcttatgtttgattctgcccattctgcagaggagtcaagcaaggctcactgtatgttagtgccttggcctaagtagcaatattaattctagtactgcgagtctagccttctgaattccattcattcattcattcattcattcgttcattcattggatgaacattcttccaggcctactatgtgccaggaactctcccagatggtagagacacaaagatgtttccagctaattattattattatttttttaagacagggtctcactctgttgtccaggctggagtgcagtggcacaatctcggttcactgcagccttgacctcctgggctcaagcaatcctcccacctcagccccctgagtagctgggactacaggcgcatgccaccacacccagctagcttttgtattttttgtagagacagggttttgctatgttgcccaggctggtctcgaactcctgggctcatgtccctatttttttttttaagttgatggatagaattttcttggttttaagtttctctttcatgcacttttattaaaacattggcatctgttatccgtaactacattaagatccctctaagatagtatctggctgggcccagagtaccaacattgaatgacctgtggtttcctagggcaggatttcgtgggattttgcagagagacccctcaccccggctctgggccaccctgaataacaatgtcaaaaacgatctggtgggcaccctctggagcagggtaaggtgagttgggagagaacatggctctctgcagccagactcagtggctcacacctgtaatcccagcactttgggaggccgaggtaggcggataatgaggtcaggagttcgagaccagcccggccaatatggtgaaaccccatctctactaaaaatacaaaattagccgggcttggtggtgtgtgcctgtaatcccagctactcgggaggctgaggcaggagaatcacttgaaccagggaggcagaggttgcagtgagccaagatcatgccactgcactccagcctgggcaacaagaggaagactccgtcttaagaaaaaaaaatggctctgtctgcaagctcaggtgtctcaacgtctggttctaccactaccggctgtggggctctgggcaagatgggaacgtcacaccagatggcccggggaggccccaggggagtaccctgctcacagtgagcacacactccacggcagctgctgttactaggatgatgtgatgttgttgtgttgttggtaggactaggcccatccagggagggtctcaaagctgggtggggagagctggcgtgccacttggggagcaaggagctccgtctctcagctgaggctggacgtctacctctctgacactgcaggtgtgtgcgccccctccctcctggggatatcatgaggatgaagcagaagcacgcattgtgtgtgtgcatgtgcagggggcagggtgaagaaatcttacaattttaaactgctcaggtgtctgggaagtaaagaaaagaaggaaggaatgaagccctccttttgggatgtcttggggtcacagagacccttttctgggaagatttttcatttccccatcttttttcatttatttgagacagagtctcgctctgtagcccaggctggagtgcagtggcacgagctcagctcactgcaacctccacctcccaggttcaagcaattctcctgcctcagcctcccaagtagctgggataacaggtgtgagccactatgcctggctaatttttgtatttttagtagagacagggtttctccatgttggccaggctaatctcaaactcctgacctcaggtgatctgcctgcctcggcctcccaaagtgctgggattacaggcgtgagctgcagcgcccggactccgcacctattttctgctctgcgtagggacagcctaatgcattctgcagcagggagccatggcaaattggtaacaagccggcccctggccttcatcccacccctccccgttctcagacctgccctgtcagctgacagtcctttgagccagtctcagctcacagcttggccttggcagtgatcatcccaggctctgagaagggctggcccctgctagtctcgcaggaaggaccctgctggctgtgtgtggtgtgcgccgcgctcactgacccctccgggccccctccttccacctcccttgctgcccttccgacgggtgagcggggagagcaaggcatgagcccccagcgcgttctagtccttcctgcgtggctgtttcctgagtgctgcccactcagcatgcactacccccccaggccaggatgatgctgggcacaccagggaagcagggagagaacctgtccttgcctttgtgaagggggaatcgtcgtaagcagggaatgattgcaacgtatgagagactgtggcatggattgtacctgtcacctctcggcctcacctttctgagcctctgtcggccctcaggtctatgcagactgcgcctctccctccccctcaccaggcagccaagcagccccttgcttcttcgtctgccctggggcctcttggacctcacctgcaggacgtgctctgcacagtttcctgcacacctgcaagtgtgtgggggcctatgcctgtgagccacccttgagcaaaggtgtaaagaccgaaggacaaggccccccaggcccctgcaagtgggtggttcatgagcttccttggaaggtcccaaaggacggagctccagccaccccagcagtagccagctccatgacccccgttcccttccactcctgtgtcactccctatccctcaggggaatgcagctaaagcagcagtgtctggggagcacccagcagggccgtgactcaggctggggatcagggagggactccctgcagaggtgacatttaagccaagctctgaagcaggaatgggagttgtccgggcaaagaggcatcctgggtgaggaaagcagcctgtgcaaggcctcgaggtaggtgaagctgcttgggagtagcaacggcagggtgggaggagtaggcagtggagaggatggacacggtggaggggagggatgtggtggagagggatggacacagtggagaggaggtacatggtggagaggatggacgtggtggaggggagggacgcggtggagaggatggacgcagtggagaggagggacatggtggagaggatggacgtggtggaggggagggacgcggtggagagggatggacgtggtggaggggagggacacagtggaaaggatggacacagtggagaggagggacatggtggagaggatggacgtggtggaggggagggacacagtggaaaggatggacgcagtggagaggagggacatggtggagaggatggacgtggtggaggggagggacacggtggagaggatggacgcagtggagaggagggacatggtggagaggatggacgtggtggaggggagggacgcggtggagaggatggacgcagtggagaggaggtacatggtggagaggatggacgtggtggaggggagggacgcggtggagaggatggacgcagtggagaggaggtacatggtggagaggatggacgtggtggaggggagggacgcggtggagaggatggacgcagtggagaggagggacatggtggagaggatggacgtggtggaggggagggacgcggtggagaggatggacgcagtggagaggagggacatggtggagaggatggacgtggtggaggggagggacgcggtggagagggatggacgtggtggaggggagggacacagtggaaaggatggacacagtggagaggagggacatggtggagaggatggacgtggtggaggggagggacacagtggaaaggatggacgcagtggagaggagggacatggtggagaggatggacgtggtggaggggagggacacggtggagaggatggacgcagtggagaggagggacatggtggagaggatggacgtggtggaggggagggatgcggtggagaggatggacacagtggaggggagggaagtggtggagaggagggacgtggtggaggggagggatgtggtggagaggatggacatggtggaggggagggaagtggtggagaggatggacgtggtggaggggagggacacggtggagagggatggacacagtggagaggagggacatggtggagaggatggacgtggtggaggggagggacacggtggagaggatggacgcagtggagaggagggacatggtggagaggatggacgtggtggaggggagggacgcggtggagaggatggacacagtggaggggagggaagtggtggagaggagggacgtggtggaggggagggatgtggtggagaggatggacatggtggaggggagggaagtggtggagaggatggacgtggtggaggggagggacacggtggagagggatggacacagtggaggggagggacgcagtggagaggatggacatggtggaggggagggatacggtggaggggagggaggagaggggagggacgcggtggagaggatggacacagtggaggggagggacacagtggagaggatggacacggtggaggggagggaagtggtggagaggagggacgtggtggaggggagggatgtggcggagaggatggacgtggtggaggggagggacacggtggagagggatggacacagtggaggggagggacgcagtggagaggatggacatggtggaggggagggatacggtggaggggagggaggagaggggagggacgcggtggagaggatggacacagtggaggggagggacgcggtggagaggatggacacagtggaggggagggaagtggtggagaggagggacgtggtggaggggagggatgtggcggagaggatggacgtggtggaggggagggacacggtggagagggatggacacagtggaggggagggacgcagtggagaggatggacatggtggaggggagggatacggtggaggggagggaggagaggggagggacgcggtggagaggatggacacagtggaggggagggacacagtggagaggatggacacggtggaggggagggacgcggtggaggggagggacgcggtggaggggagggaggagaggggaaggatgcggtggagaggaatggatgtggtggaggggaggaacgcattggagaagatggacacagtggaggggagggacgcagtggagagagacggatgcattagagagggatggacgtggtggagaagatgagcgtggtggacagggatggataagatgctcagcaatggacgcagtggtcatcggcgccttcttggcaggccacagggaggagtttagactgcgctagggcaagggggagccacacaagggtttggagcaggaactattttggccacttgatggagaccggatgcagtgttggggccagagcaggcagggagacccattaggaggctgttccggtgacccggtaggacgacaagcaagaacaacagtgggggtggggagaatacccccaaattgtgccatagtcagcttcagcatctcattccgtgtgaattacgaggctggtcatggtgagtgtcggggcctccatggatgctcggggtgatttctcatggatcctgcaaccacgtctccccttcacaagcctcttgccaagccctgctggccctggtcctctgtgttcttgctccttaggtcagtctcagacattcctgacacttgtgcttcgtggccctttcccagccctggctggcatctcgcgatgtagaagggagagaggccagaaggagagcagctcagcccacgaggaggtgcccatggaggctggcaggggcccaggggcccccctgcactctccccagttttgagaccttagggcagtctctcaccctccagtcttggtttctccacctctgaaagaatcacttttccttcctgccaggaactgaagtcaaaggcacggcattattgagacacattggggagcagtgggcccagcactgggacccatgctgggtgtcccaagagcacagaatgttggcagggaggggcgggggccaggcagggagctccctgctgctctacggctgccccacctgcagcccttcccttcaccccacgccccagtttcctctgctcttgggtggccagtcacccaggggtggtatccagggaggttcttgcaggcccagcttcactggcctggaagggctaagctacctggcccaggggtgtgtccactttgtcctgggcaggctatgggcatcccacaggtggagtcccatggcagggctggagccagggggtaactcggcaggacagaggcccagccaccaggaagctcgcctgctgatggccaagctcaggacaccttgcaccactgtacctgaggacaggctgtgactgatgacagtggcttcatcctcagaggggaagtgtcctggagggatgtggctggaggcacccagccctgcgggagcccctttgaatgagcagatacctcgggaagcagggtgaagggggcagaaactgggaactctggatttcaagacttgaaagaaaagtgctgcattgtgcggtggcatctgcttgtggggacaagggctccctggtgagacctctccatcccctccctccctccccctctgtctcacactctttctctctctccctgtcctcctatctcggcctcgcactcgggtcgtgtttggtctgtgatgtcaccactcccctccctgtggctgccattcagagctggcagtaatgaaaagcacaggattcccactcactgggccccagcagcactacagagacatggagccctttgttgcaaataattaacttggtcccatgttgcataacatcctaaaaagctttcctgagcgagcacccgccagacactgtgatctcatcaatactgcatcgcatcggacactactgctgtgtgatttcattaaaaatttaatttgtgttttaaatctgcagtgttccctttaaaaggatggcgagggcgggggatgaagagagtgacaggcttgggtctgggggtgggcattccaaagcagaggaaaccccacgccccacaggaggtgggggcggggcttcccactggagcagcggcccctctccgattggctgggcttggtctgtggcctctcctggtgctggagggagagtgatggaccattgatggaaaatgcttttctgaagcttaatggttttccctttctctcggccacctcaggcagagggctgctcctgggaacccagacacaggaggatggccaggtggtggtgcaggtggtggtgcaggtggcggtgcaggtggcagggcaggtggcggtgcaggtggcggggcaggtggtggtgcaggtggcggtgcaggtggtggggcagggcaggagtgccaggtggcagggcaggtggcggtgcaggtggcggtgcatgtggtggggcaggtggtggggcagggcaggagtgccaggtggcagggcaggtggcggtgcaggtggcgggggcaggtggcggtgcaggtggcagggcaggtggtgcaggtggtggtgcaggtggcagggcaggtggcggtgcaggtggcggggcaggtggcggtgcaggtggcggtgcaggtggtggggcaggtggtggtgcaggtggcggtgcaggtggtggggcagggcaggagggccaggtggtgggcaggcggtgatgcaggtggtggtgcaggtggtggtgcaggtggcagtgcaggtggtggggcagggcaggagggccaggtggtggtgcaggtggcagtgcaggacgtgttttccggagaccccgtcctctgtgcccctccagacccagtgcctacagtggtcaagaccagagctgcagcctcactcctgccctcctggctgtgtgacctcaggcaagctgcttgtcctctctgagtacagatcactgcccggagtgtgagtgggggccttgcctgtccccagctccaggttgctgacaggagagtgtgagtgaagggccttgcctgtccccagctccaggtcagcacccgcagtgtcactctcacccccgtgtggctatggagcaggcagccattggtgactgggtagcagaccctccggaaaggctgcggttctgttttctttctttgtctagacttcactctcttccagagaggatttaagctggccttcatcatcggtgctgcctgcagacccgtccctgggatttcgctgcgaatgggatcctgttgtttctagacagggacctccattgtgttggggggggattggtttcactcctgtttccagtgagcggggagggaaagagagctcaggttctggttgggggaggagagagaggaaggtgaggggagcacggagaaggagactctggggcaacttcgtgatgctgcctgcctggctctgcctggggactctttggtcccgcccgccctggccaggccaattgtttcctgcggaaggcagggatttgtgcatctccaactatctcctgacagtatgccagagtgattaagctaagcctcccggggccagcagaagagaggggacaaattgctttgtgaaattgatttgctgctgttagcatttacagcttgccctgggtacgcccacagtgtgaggcggctgcgtgggcgtcctgctcccccttccctctcccgcagcacacgggctctcacttggcacctgcgcacacatcacctgtcgtgtgtcacatagccaggcctcaggacctggacttcagtaccactcacctcagggcacaggtccaaagtgtcccaaatgcccatggaggcagacggccacccacagctgcagcctgcagagagaattgccccagagactcttggataggtgggtttccccaggccagcgtgagagctggggccacgagatctgagaggacacctggaggcccagagaggaggcgtgagctagccctgggccagggtctccccatctcttttttttaaatttgttttgttttagactgagtcccaatctgtggcccaggctggagttcagtggcgtgatctcggctcactgcaacctcctgcctcagcctcccgagtagctcggattacaggcgcatgccaccatgcccggctaatttttgcatttttagtaaacactaaaaatgatttcatcatgttggccaggctggtctcgaactcctgacctcaagtgatccgcccgtctcagcctcccaaagtgctgggattacaggcgtgagccactgtgtccggcctctccccacctcttggggggccccttctgccacaccggctgccctcctccattccaggggaaggaagacaaggagaatattgccctggtcagcaacagttctcatagaagctgtatgaactcaggtgagcgtcatgaacccacacgcctgggtgctggggccattcagagaagaggcggtgctgggactggtggatccttccagacagagaaatgtggctgagcatcaccacccaggaaatagaaagcactgacaattcgcccaggtgggtcccactatgcatttcaagaaatgcgtgcttggtaacggaggcagcatggcctcggtgcaccccaggagatgggagactcaggctacgggcgctgctgggtatagtagcctgctttcaggctctccttgggcaccagctctgagcaggctctaggcatgactagatggtgggaagggggtccagggagccatgagaggagcagcctcagcgtctctcctgggctggagccaggtccatcgctcagctgctgtgtaacctcagacaggtgacttcacctctctgggctgtggtaagggttacattagacaaaccccacaaggctcttagaacagtgccggttggtaggcggggcacggtgactcacacctgaaatcccagcactctgggaggccaaggtaggcggatcacctgaggtcaggagttcaagaccagcctggccaacatggtgaaaccccttctctacaaaaatcagccgggagtggtggcacacacctgtaatctcagctactcaggaggctgaggcaggagaatcacttgaacctgggaggtggaggttgcagtgagctgagatcacgccattgcactccagcctgggcaacagagcacggactccgtctcaaaaaaaaaaagaacagtgctggtcagtggaagcatttggcaaatcctagttgtgactgttgctgttttcccctcccttgagatgctcagatgtttgtcatcatacaaggacgtgagggtggggcacaccagcagtgcttctgaggttagggggctcttcagtcccaggccagggggaaggaaggaaagtgggtctctcagcagcaccacagactggaccctagcacagcaggcacgtccctttaaaaatttaaattaaaaatatttaaatgggggtaaaatatacataacataaaatgtaccatgttaactgtttttgagtggagcgttcagtggcattaagcacattcacgtgattgtgggaccatcaccatgagtccccagaactcttttcatcctcccaaactgaactccgtctccattaaataccaactccccattcgccctcccccaggcctgggcacccccccattctacctctgttccagttaaaccagtgcccagcgagactcatgtaagtggaatgatacagtgtttggcccaggaagccttgtgtaagtggaatgatacagttttgacccaaggaggctggtgtagcttctacctttcctctataatgaataagactgctgtgaacacgggtgtagaaatagctcttcaagaccatgcttttacttcttgtagatgtgtacccaaaagtggaattgctggatcatatacctcccttttaaaacaaatgcagaaactgaggctatacagaagggctcagaggtttgccgagggtcccagccacctcagtggcggagcaggcagcatttccccaccccaactctgtgggtcgctggcactatgctctccctccccagcgtctgtgtggcctgttttattcccaggctttatagaaggctctctggaagacatgagctgtgatggatgcaccaaaggagggtcatcaccattccttctttttttttttttttttttttctggagacaaagtctcactctgtcacccaggctggagtgcagtggcgtgatctcagctcactgcaacctccgcctcctgggttcaagtgattctcctgccttggcctcctgagtagctgggattacaggcacccaccaccacgctcagctaattttttgtatttttagtagagatggggtttcaccatgttggtcaggctggtctcgaactcctgacctcaaatgatccacccatcttgtctttccaaagtgctaggactacaggcatgagccaccatgcccagctgagtgtcatcaccattctgaactagttttactcacactctgacaccaaatgtgtgagggttttttttttttttcctacatcgaccagttcttcaactttccagacagctgggtggcctacaactcacttcaattcttacactgactacctgtagttagcacagacctcacatttaagggtcagtcccacaagattgccccctccttcagacaccaatgacaagtcccaggttgtcaccagttcttctggttgactggctataaatcagggttcccataacccccatctcaggttcaacagtttactagaatgactcacagagctcaagaaagtgctttacttactattgctggtttattataagggctacaactcagaaacagccacaaagaagagatgcataggacaagatacgggggaagaggcaccaagcttccatgccctccccagcaccttgacctgttccccaacccagaagctctttgaaccccatgtttagggctttttacggaggctgcatcacacagtcgtgcttgattaaatcatccgcagttggtgatcaactcaatctccagcccctcgcccctgtccagagctcattgaatcgggagccttaagaaccccccacccccagttgctctttctcttccctctggcatggtggctagatccctaggtgatgaagtggaaggaggcctcctgctgatccacgatgaacctactgtgggaaagaaccagaaagaacagctgccgcttagagcaggtggtatctcaagcctgtgtgttactgcagcaaaacctatggccgcctgggtcctgaccacctgaggggtacagtgttctcaggggcgcatttctgccttcacgtccctgagcctggggaagtcctgcccaaactcggttctcctggagtctctttcatctttgaggcaaccactacctttccgtcaactccctcttctgcttcagttaaccacaggtaggttttgctccttgcaactgacaacctgggctgatggaggaagctgtttcagctgttatcaagcaacacctcgagcaggtgagtctgtagctggaggcctccaaggtgctggtgtctgaagcaaagataggtcactggaaaaagaggtggcgtgtgtgtgtgtgtgtttagaaacagagtgtagttgtgtgtctgtgcatgtagaaagaggacatggttgtgtgtgtgtgtttgtgtgtgcgtgtatagaaagaggatgtggggccaggcacagtggctcatgcctgtaatcctagtgtataattgggaggctgaggtggacagactgcttgagcccaggagttcaagaccagcctaggcaacatatcgagagtctgtctctacaacaaaaaataaaaaattagctgggcatggtgatgcatgcctatagtcccagttactcaggagactgaggcaggaggatcacttgagacaggaaggttgaagctgcagtgagctatgatcacgacactgcactgggtaacagtaaaactccgtctcaaaaaaaaaaagtgtttttttgtgtttgtgagcatgtgtgtgagtgtataggaagaggatgtggtcatgtgagtgtgcctgtgagtaatgtatatgtgagtatacctgtgtgtgtgtgttgtgtgtatatgtgagtgtgcctgtgtgtgtgtgttgtgtgtatatgtatgtgttgtatgtatatgtgagtgtgcctctgtgtgtgtgttgtgtatatgtgagtgtgtgtgttgtgtgtatatgtgtgtgttgtgtgtatatgtgagtgtgtgctggggcagcatcacaggaattcgcctgccataaggccgggctgcgtcacggctaaggccgcagttcccccaccgtgcctgggtttggatcctggctgctccggtcgcctgctgtgggtccttgcacacattaacctctgaacaccagtggccgtatctgaaatagcgcgagaacaaaactcaggattgctggggagctgtaggagtggaattatgcaaagcatggcatgtcgcccgtaacagacttccgggccaccgtggtggtgccgacacaggccggccggggcgtgcgtggggtctggctttgtcttcttgtctccctctgctcctccctcctcccctggccgagggagcccggcctgtcttcctggccacagttcgggaggccgccgtcctcaaagctgggcaccagctatggctcagtgtctccagcctccaggattcctgtggtcgggatccagcgtctctttatcgtttttaaagtgttttgctgctgggaagcctcaagcctctccgcagagggacataaataaaggcaggaacttggccattagtaggcttcatgtggcgtttccacctgggagcgccctggcgtgctgcaagctgccctcactggccaagagcagggcgggaggggagcctctggagttctccggttctgggttaggtgcctcttactctctgggctgcaaacagacgggggtgcctggcaatgtgccccgggcggagcacccctaacagttgcagttcaaatttggagccaggctgaccctggttcaagtcctgttgctgtgtgctggccgtgtccctggacgggtggcacaaggtcttggtctccagtgtgcagatgagagatggtgattatctactcctgggtgctgggctcagagccgctggcaccagccctgggggctgccagacatatagccctcggctctgtcacgcccggaactcaaacctcgcaggcagtcaggggagaggtctgtgtgtggagctcagaactcagtgagcaccccatgtgcctgtaaggcctcgccatctcctacccaacccagatgaggctcctgcttcccacccctggcagggagtctgatctcatcctagctcaaaggtggaaactgaacgtgggccaggtggcaaagttccccagtgggttcagcttccgccgggtggaggaggtgggaggcatggctggaggcctggcatcaggctggatgaggtggacccctcaggcttgaatccaagagggctcagagggtgagtggggccccaggccacgtctccaacagctgcccacctagccttgcgcctccgccttccttccttcctttaaaaatggctacaggcacattttctttgattacttttatactgaataacactatcctttttaaagcaaaaaatacatgctcatgataaacaaatggaaccggacagatctttatcaaggcccccgcaatgtgaccttgtgactccaagacagaataactttccatgtgtgttttcaagaggttgtggggagccgctgaacctgggagcagtgagggcacaggcgagagctgtggccaggagagtcacaggagcggcaaagacgaaagggagtgggagatgggaggccaatcagaaagcagtgcccgaagctgggtgaggagcagcaaaggcgggggtttccttgaggatacctgagagccatgctagggtggggaggtctggggggagttgtgggctggccttgagtttgagattgaggcccaggctgagagcgtggtgacccctgcactgtgggtctgctcgagctgtgtgcttcccaggagcaagagcaaagaggccagagggaggggggcagaacaggccagcaagggagcccccagaacaggacacgcgttccttggccctccccaggcagccctaagagggaagagaagctctgccccctcctgccccctcctgccctctcctgttccctcctgccccctcctgctctcctccatggagtcagggggccccaggtggccatggccagcccattcagggatctgagccccagagcctgcatgccagggtgagcagagccagcttgactgcagccatgctgaccccgcaaccttccccgaggtctgaggacaccacgctcccttcctgccggcaggttgtaattgggtttcacacggggccttctggcgctgacctacatgttccggtccgtgcctccttggtttctgagtgtgtgcccctgatggttctggaacctctgggcgtgggcctccttccctccccgcctcatcagctgctctgattccaccgggcaaggtcaggctggcaaggaaggaattggagctgcagtctgggcgccaggcccagagtgtgcttcccaggggctgcctgtggggaggctcccgatctgcccactcccgaaataagaaggggcgcccagagaggaggagactctggatgcgtggtcagcaggcaccggctgccatccccaggagagtcattgcctgtgaggacgttgaccctggtaggacccgcctctctgaagctgtctcgggtgtgagtgaggaggaacggggtctctggggactgagccctcatctccagtgcaaaatgcagaggcatgggagggaatcaaatgaaacagtgtgtgtggaacagaaggcgccaaatgggaatgtccccctgcctgtccatctccctccctcccccgcctccttatttgcagacggagggactggatatcctggcagagcgtcttccgtgatgcgtaacctcccaggagatgcatgttctggggttccccacttacagttgagaatacagaggctaagaagggtgaagggactgcctccgggtcccaaggctgttccaagtgatctaggactcagctttggtcctcctgccctcagcacccttggtcatcacctccactcggtgtgcaggaggctcctgctgtaggtgcccagatgttcggcagctttgtacttagaaatttcgagggggcggtcgggcgctgtgtctcacgcctgtaatcccagcactttgggaggccgaggcaggtggatcacaaggtcaggagatcgagaccatcctggttaacacagtgaaaccccgtctctactaaagatacaaaaaaatagccgggtgtggtggtgggcgcctgtagtcccagctactcgggaggctgaggcaggagaatggtgtgaacccgggaggcggagcttgcagctagccgagatcacgcactgcactccagcctgggtgacagagcaagactccctctcaaaaaaaaaacaaacacacagaaatttcgagggggcagagggtgtcctaaccttgtccctcagcaggtcttcaccctggcaccttcaccagacacccgccatggctctcggctcagcgggtagctgagtgtgttctctttgggttctgacaggttccagcttgttttttgagaccttgtcagcgtggggcctgccgggaagcaggtgcttgtctttaatgggatgccttatcctgggctccacaggctgcacggggcgttgttcaggtgactcctacacgccgaggcgtctctagaggaggagcctcaaaaggactagcagggggtgcagataactttgtttttaaaatgaagccaaagctttcaccacatctcagagacagagtgacccgaatgtggaggtttggctggaaccaggcaggtaccatccactacggctcccacctctcctgctcggaatcccccgctgccctcccccaggccctggtctctgatttcagactcgcctctgctctgtttcacgtctcctgcaagcagacaagcatgaagcctgccctccgaggctgcatgaataatagaactgaaatcatccctagttaaaaatagatactcttcttaataaatattttacatactgtctttaggatatacaggtcccctggctgaagacttaaagcaaaccctgttatcgaaaccaaaagatgtaggaaaggctctgcccgggctctgccagtgtgttcctgcaccacggctcttgcctcaaagccacccactctgcggtgacgtccgacgcagacagcgcacacacggattacgttggaactcatagccaggagcggcttgggaattcttgggtgtattagtcacttgtgaattttatggaggcaaatcaattataaagtgccccttggttagtgaggcttggagttcagaaaacccaaaagcaggttcttacgttactgctgcgaggcaaggccttcaccgtgaatggaagctggggtgtctgggggcagctgtgtggaggaagagaaaattctgggcagaagcagttggagtttaaagtgctggcccatttctgaaaactttgcaaactttgcaaaaactttggatcggacgtcactgcagagtgggtggctttgaggcaaacagagcaatcttgaaggtctccattaatgaaggcatggcgtggtctggatgcagcaaatgcatttgaatgatctgcaggaaggcctcgtagccttgctccccccacccatgtgcttccccaggaagacaggggcagctaggaaatgctggggcctgcccagtgtgcagaccgcttccaggtgaccccacgctgcatggcccaaggcaaaggctggagcgtccttatttaacaggagaggaaagtggatactggaagggcagagcgtgctggggctggaacagacaagccccaggctggagggcccatgtctcactccctctgccccacacctcagctggtctgcctatgtggtattcttgaactgagtgaagagacccttctctgtcagggccacctgccccacagaggggctcttgtcttccttgtgctcaacataccttgaggacattttgctttagcacccagcaccctggatggcatgtaggggtccaggtgtgtctctgctccagacagtgaatccttgagcacctggcttagagctgggcccctggtacatgttgttacatggatgggtgggtaggtggatgggtgggtgggtaggtggatgggtaggtgggtgatatgatttggctttgtgtccccacccaaatctcatcttgaattgtaatccccataatccccacgtgtgaagggagggacctggtaggaggtgattggattatggggggtggatgggtggatgtgtggatggagggatgggtggatagatggagggatggaggggtggatgaatagtgtgtatggataagtggatggagggatgggtggatgggtagatggagggatgggtggaggggtagaggggtggatggacggatggatgggagcatgtatggataagtggatggagggatgggtggaggggtagaggggtggatggacggatggatgggagcatgtatggataagtggatggagggatgggtagataggtggatggagggatgggtggatagggggatggagggatgggtggatgggtggatacatggatggagggatggtggataggtggatggatggatagagggaggggtgggtggatgatggagagagggatgagtggatggatggatgagtagatgaacagatgtgtggataaagggatgggtgaatgggtggatggagggatgggtagatgggtagatggaaggatggatggaggggtggaggggtgggtggatggatggatggattgttgtgtgtatagataagtggatggagggatgggtagatgggtggatgtgtggatggaggatggtggataggtagacggatggatagagggaggggtggagggatgatggagagaggaatgagtggatggatggatgagtggatgaatagatgggtggataaggggatgggtggatggagggatgggtggatgggtagatggaaggatggatggaggcatggaggtgtgggtgggtgggtggatgggtgtgtgtatggataagtggatggagggatgggtggataggtggatggagggatgagtggatgggtggatgtgtggatggagggatggtggataggtggatagagggaggagtggatggatgatggagagagggatgaatggatggatggatgaatggatgaatatatgggtggatgaagggatgggtgaatgggtggatgaatggagggatgggtggatggagggatgatggatggtaggggtagatgggtggatggagggatgggtgaatgtattgatggtagggtgaatgaagggatgggtggatagatggctgggtggaaggatgggtggattggtggatagataggtgaatggattgagggatgggcggatgatggatggtggatggatgggtgggtaggtggatccatggatggatgcacagagggaaaaaggcacattgtatcagactccaaagggagaattaattgcagcataaaatgggagtccaggacctctcttatttcagagttcatcgtgtagatgggaacagtgtttaacccttccctctcccactgccctctgtcagctgcttcccagttcctaaattattggctctgaaagtgggggctgggggagtggggaaaagagtggagatgctgagactttcccatgtgtccagcttccagaggcttccaggacagactgtggcaccatggttagtagccaggagcaggttagggcattatggtacctccccaaacaaagcagggtgctccagcttcaggcagtcaggaccccatctgctcagtgagtgtctggtcagcacccactcaggccaggctgtgtggcaaatacaaaagaagttaaaaaaaaataaagacattccattatttcaatagaaagaattttttaaaaatgtgtcactggaggtacacttgctttacattttttgctttggaagatgtagggaaaaaagtcatgaaatgttttcatttctctgtgattgagcagactcttaaaatattaaactattgcttaataattgattaaaactaatctctgtgggagccaaccacccatttggtaggatctctttaaacttttaggggcttttagctccccctggccgcctaacctttctcttgtctgattcattctgggctcagcagacgtttggtctcttgtggattgatgatgtcaggatgcttctgtcgggccgtgggatggctgatgaggctggattccgaacaaggaacatcctagggacacagtgtctggagaggcagtgagggaagaggaaagaaatgtctgtgaataattgtactgcagcttctaaaactcgggacgggagtcagagaaaacagccctgtggccttcagagccgcttaagttgtctgtgacttgagtttttccacacaatgacaatggtgcctgcctctccattttcccaggaacgctaagccatcgagcaaagtggtttagaatgtgggcgcaggcatccgagtgtctggcttcaaaacttccccagcaagttaccgaatgtgtaaccttggacgcgctttttattttctctatacctcagctccttatcaataaaatgaggatagtaatattaatagaaaccctcttctgtgcagatgaggtgaggcaatgcatgtaaagttcttagcactggccagggctgggcaaaggcagggctcactatgcatttaagagctcaaagcccgggtgagctggctcacgcctgtaatcccagcactttgggaggccgaggcgggcggatcacctgaggtcgggagttcaagaccagcctggccaacatgcagaagccctgtctctactaaaaatacaaaaaaattagccgggtggggtggcacacgcctgaaatcccagctactccggaggctgaggcaggagaatcgcttgaacccgggaagcagaggttgcggtgagcccagatcacgccattgcactccagcctgggcaacaagagtgaaactctgtctcaaaaaaagaaagaaagaaaaaagcccaaaggagactctgaattttaaaacaactactttgaggccagtcacggtggttcacgcctgtaatcccagcacttcgggaggccaaggcaggaggatcacttgggccccagggttcgagaccagcctagacaacatggtgagaccccacctctacaaaaaatgtagaaattaactgggtgtggtggaacatgcctgtagccccagctactcaggaggctgaggtgggaggattgcttgagccagggagttcgaagctgcaatgagcgatgatcatatcactgcactccagcctggtgacagagggagaccctgtcttgaaaaaataaataaaacaaaaacaactactttgaaaaagggatcattaggtgaacaactgaagcctctgaaactaaacagtctcagccaggtgcggtggctcacacctgtaatcccagcactttgggagtccgaggcgggtggatcacctgaggtcaggagtttgagactagcctggccaacatggtgaaaccccgtctctactaaaaatataaaaaattggccgggtgtggtagctcacgcctgtaatcccaccacttcgggtggatcacctgaagtcaggagtttgagaccagcctggccaacatggcaaaaccctatctctactaaaaatacaaaaattagccaggtgtggttgtgcatccctgtaatcccagctacttgagaggctgaggaaggagaatcgcttgaacctgggaggcagaggttacagtgagccaagatggcaccactgtactccagcctgggcgacaggagcaaaactccatctcaaaaaagaaaagaaaagaaacgaagcagtctcaaagaaatgtttgtacacttgtgtttatagcagcactattcacaacagccaaaatgtggaaacaacccagatgtccatgggcgcatgaatgggtaaagcaatgtggtatgtacgtacaacggggtattatttagccctgaaaaaggaagaaaattcagaggtcgggtgcagtggctcacaactgtacctataaccccagaactttgggaggccgaggcaggaggattgcttgagcccaggagttcaagaccagcctgggcaacatggtgagatcctgtctctatacaaaaaaacaaaaaaaatcagccagatgtggtggcacatgccagtggttccagctacttgagaggctgaggtgggaggattggttgagcccaggagttgaggctgcagtgagctgtgatcataccactgcactccagcctgggcaagagagcaggaaaacgcctcccccagcatacacaaaggaaggaaattctgactcgtgctacaacatggatgaatctcaagacatgctaagtgaaataagccagtcgctggccgggggtggtagctcatacctgtaattccaacactctgggaggctgaggcaggcagatcacttgaggtcaggatttcaagaccagcctgggcaacatggtgaaaccctgtctccaccaaaaaatacaaaaacaaacaaaacaaacaaaacaattagccagatttggtagcacatgcctgtagtcccagctactcaggaggctgatgcatgagaatcacttgaaccctggaggcggaggcaacagtgaaccgagattgtgccactgcactccagcctgggcaacacagaccccgtctgaaataaataaataaataataaggcagtcacaaaggggcaaatactgtataattccacgtctatgaggtatttattttattattgttattatttttgagatggagtctcgctctgttgcccaggctagagtgcagtggtgcaatcttggctgactgcaacctctgcctcctgggttcaagcgattctcctgcctcagcctcctgagtagctgggattacagtagcctgccaccacacccagctaatttttgtatttttagtaaagacagggtttcaccacattggccaggctggtcttgaactcttgatctcaggtgagccacccgccttggcgtcccaaagtgctgggattacaggcatgagccaccatgcctggccctatgatgtatttagagcagccagaaaaatagagatagaaagtggaatggaggttgccagggctggggaggggaatgtggagttgtcatttaatgggtgcagagtttcagttttgtaagatgaaaagagttctggaaatgaatggtggtgatggttgcataacaacgtaaattaacttaatgccactgaactgtgcactcaaaaatggtaatgatgataactttatgtgtagcttaccacaattcaaaataataaaaggatgggtgaggtagctcacgcctgtgatcccagcactttgggaggccaaggtgggtggatcacttgaggtcaggagtttgagaccagcctggccaacatggtgaaaccccgtctctactaaaaatacaaaaattagccaggcgtggtggcgggtgcctgaaatcccagctacttgagaggctgaggcaggagactcacttgaacctgagaggcagagattgcagtgagctgagatcgtgccactgcattccagcctcggcaacagagcgagactccatcatcctaggccttgcacagatgaggtggggcctttgattaagagctcagcggggaccaccatgccccctccccacctgccccgagacatcctcagaccagcctcaaaggcccacagctggattggccagcctcaactttgcatgctgtcggtgggcgtggaaatggatccagcctccttgaaggtgccactcaggaggatgtccaggaaatcttaaacgtgagtgaaggcaagagctcctgagtggtgggttgggatctgaacacagccccacctggttctagcacagccctggtcctcattcttcttggtccagccaggcaccgtgggctacatcatcctaagaaaggggaataaaaccagagagggtagagtcagggcctggatccaccctgattaatctcccatccagagaatacgaaaggcaatgccaggaagatgttctaaggtcaaatgaaccatctatgaactcacgcttccttctacagagcctgtggcttacaaagcactttggctgggcgtatctcttccatttccctggagctgccctgacctggggaacaccaacctcgtctccctctctacagacttctccacttaaaatggccaagacaggggctgccttttctgggggttctggggtattcctgggggagatcaaggctgggggcaggttgatggtggaggtgcagggtgccctgaactgggagtcacactgtaggctccgcagcagccggaagagggctcaaaatacaataaggataaataaaactcaggcttcctttctctctctgtctctttttttgagacggggtcttgctctgttgcccaggctggagtgcagtggcacaatcttggcttgctgcggccttgacctcccattctcaagcgatcctctcaccccagcctcccgagtagctggaaccacagcacatgccaccacaccttgctaatttttttctattttttgtagagatgggggtctcaccatgttgcccaggctggtcttgaactcctggcctcaagctatcgtcctgccctggcatcccaaagtgctgtggcgtgagccacactgcacttggcagccttcgtttttttctttcctctttttatcagaccaatacaggcaggtgatttttttaaaagtcagatttttaaaaaacctgacagtacaaagaggtttataaagaaaaggattccctcctgctccgcctttctcatccccggagataacctcttttgacggtttccgttttaaatcatgatggtggtcacctcctcaggtccaaataatatggctacagtgctgttgcttgatttatggaccttagaaatggcccaccgatttcccgctgcgatcgatgaggactcagctcgtcggcactgccactcccagccccagttcagtttccgagtctacctctagttcgtctgctgcttacttgctaacttggaattccatctgagacccagcctgaggccgtcacctgtgggcagcgtctgctctcctccttggggatggagggtaccacccttcctggaccctcccatggtttcctcagctcctgtctgcttttctctgccaaagttggaagttctctcatcctgctctgcagggcagctgcctccatgcctggtgggcagagcgggtctttcactccacagcgtgcgctgaagtgttcgcagcgggtcctgcacggccaggcatgtcctccccacgttcccagctgcgcactggacacacccaagtcacactggacccttcctgatgcgccaccgccacggagggtccaaaccacttccggctttagatggctttctgcttggcccacgccttcctcatgctcaccccactttgtggggagtagcagctgtggccctaatgtgccccgtgccaccctgactttaggtcgtggactctgtccaggcttctcctcaaagttcacatcagcatcttcatatctggaccaaaactttctggcgcagacttttgttgtttctgggtttctaatggccttttttttttttggcgctgcctcgttggttgtgccctggagtcctgtcgcctcgtgtacttcagccttcgggttgaggctgaggttcctgttggactgggggtgacttttgtctgcccacgtgggtggcaagtgaggagcagcatggggttggctctggccagagccgagatcttcagggcagtggccaagccctgggtgcaggggccgcccatgccaccaacctgacacctccacttccagaaggggaagctggtccgagttttatggccgttggctgtcgaaactggacccacagagccaagaccctgtgctgcgcccgctgcaccctgagctgtccagaggcccagcccctccctacactgtgtcctccatggcccagaaagcacttatgcacatgctcacacaacacacacaccacactcaatccacacacatcacacactacactcaatccatacacaccacacagcacacaccaatccacatacatacaccacgctcaatccacacacactatacagcacacgccacactcaaccacacctcccccacacacaccacacacaacactcatacctcacatcacacaacacgctcaatccacacaccacacacactcacaccatgctcaatccacacacgccacacacaacactcataccacacatcacacaccacactcaatccacacacaccacacatcacacactatactcaatccacacacactacacagtacacaccatgctcaatccacacaccatatgcacatcacactcaatccaccttcaatccacacacaccacataccacactcaaatccacatacttacaccacactcaatccacacacaccacacacactcataccaccctcagccacacacaccacactcaaatccacacacatcaaacacatacacacaacacacacacaacacacaacattcctaccacacaccacacatacataccacgcacacacacactcacaccaccctcaagccacacacacaccacacatcacacacacactacacacaacactcatgccacacacatcatacacaccactcaattcactcccacacattcacacaccacttacacacacaccccacactcaatccacacacaccacacacactgacacacatactacacacacaaacacacacacatacacggcatgtgcccctctccctgggctggtgggagcggaggcccctcgagaggaaggcaggccctgtgcccaggagcatcccgtctgcagaaggagtctgccctccagcgggaggagtgaggtaccgaagccagggccccgtgagaccccggcaggtgggcggtgagcgagttcgcaggtgaggagggcgtgggcactgcagggcagcggggacagcctgtggagccgccaggaaccaaggcagcaggcctggatgccgggtgcaggggcttgggcttcatggagaccaaaagggctctctggagggtcttgagaaggaaagggctgaggccagactccagccttcttagagctcgtcctggctctgggtggagaatgggcctggcaggtgagggagctgcctaaaggcagtgtgcgctgaggccgctggccagggacctggaccacctgccatggaacagtgcgtactgaggccgctggccagggacctggaccacccgccgtggacagttacactgcttccctcacccatcctctggtgacacatcctccggtgacacatcctctggctgctgttagagatggaatttcactcggtcaggcctggggcaaaccactcagaggacacagggggccaagggagcccaggcaacttcctcgattcgcacctctgtacgggggatccaggtcactctattttctcaccatttgaggggccgggtgcagccatgccgccgttgccctgcatagagacaggtggctgggctcagtggctcacgcctgtaatcccagcactctgggaggctgaggcaggtgtattgcctgaggtcaggagtttgagaccagcctggccaacatggtgaaaccctgtctctactaaaaatacacaaattagccgggcatggtggtgggtgcctgtaatcccaggtgctcaggaggctgaggcaggagaattgcttgaacctgggaggcagaggttgtagtgagccgagatcacaccactgcactccagcctgggcgacagagcgagactctgtctcaaaaaaaaaaatagagacagggtttcaccacgttggccagactggtcttgagctcaggtgatttgcccacctcagcctcccaaagtgctgagattacaggtgtgagccaccgcgcctgggccctttctagtcttcttaaaattcagttttctgtcaagagcagcaacccaactgtaaattactgtggtcagggactaagtcaccttggtgtcatcctggtgtcaccctgatgtcaccctcgtgccccagcacctagcatggcgccaaattggtttttgcagcaagaataagtgagtatggcacttacaatggaaaaaaacccaatagactttatttaattaaaaacattaactttctctggcagtcaaggttggagccacccgggagagccgggcgtggcaaacacacaggcccgtctggaaagctctgccagcggggctcctgacccaggtccagcaaaagcagctcaaggtcagcctcagggcctgacatctcccaggaagaatccaaaacacccccgctcctcagcagcactgtcctcacttgcacaccgtcctcgcctgcgcaccgtcctcgcctacgcaccgtcctcgcctgcgcactgtcctgcgccctcccaacctgcacttagcaaggtcatgccctgaggtttcagaacaaatccaactttcaggacaatttgactagaaaatacaggccaaaaatccaccctgggatctttgctgcagtatagattttcttgtttttgccctgaacgagtcagttaacgattcagattaacaagagatctgagcctgggagtatctgggagtcatagagagaacagctacgggccagggccattagccaaggggctgctgatgctgggaggcctggggcaagtggacgggctcacaggccagctttgagggggatggggtggatgactctgagcccagaccagacatctccccacctcaggcgctgtctcacgtgccgccttgtggggttaactttgaatgtcaaagcccagaattttcactagggtcgccttccatcctgtcccttcatttgagaagcggaaagcgaggcacctgtgtgggactggcccaggctgtgcagtggctccccaggtgtcctggtggcacgtggggcaggaaagtcgcatcagtcagggttcccagagaggccgaaccaccaggatgtaacacacacagggatggattgtcaggaatgggttcatgcacttgttggggctgtaaacccggagtctgcagggcaggctgcagcctggacacacccgcgggagttgatgttgctgttcctagactggaggcaggttccctcctctcccaggaaccgcagtctttcctcttgaaggcctttcttgttttgtttggctttttcacagtgttggaatgtacatttcctttttttctttctttcttttttttttttttttttgagacggagtctcactctgtcaccaggctggagtgcagtagcatgatctcggctcaccgcaacctctgcctcctgggttcaagcgattcttctgcctcagcctcccaagtagctgggactacaggtacaggccaccacacccagctaatttttgtatttttagtagacatggggtttcactatgttggccaggatggtctcaatctcttgacctcatgatccgcctgtctcggcctcccaaagtgctgggattacaggtgtgagccactatgcccggccctggaacgtacattttcttagcagaacttattaagaacataattacaaaagaatttcaacaagtcaatgatagcatacaaaagaaagaagggaaaaaatatatattttattgagacagagtctctgtcacctagacaggagtgcaatggcatgatcttggcccactgcaacctctacctcctgggttcaagggattctcccacctcagcctcccgagtagctgggattacaggcacccaccatcatgcccggctactttttgtgtttttgtagagactggatttcaccatgttggccaggccggtcttgaactcctgacctcaggtgatccgcccacctcggcctcccaaagtgctgggattacaggtgtgagccaccacgcccagccaaaaatatcttttaaagtactcattattttcactttgttgggaaggtaggtatttatatttaatagttgagggaatatcgaatgtttctttttaatagatgctaagcaggatagttagtaaccgacatgtttaatattcaaagttaaaatacttgagaacaagtattaggtatataggaaaatggaatggaagtttctccacgtttgtctttccatagccaaaagaggcagaaacttttcttctgagtctctcagaagaaattccaagaagactagaagccgctggcatgaagagtaaacgtctaactatcaaaaccatggcacgaaagccgggggcccctgtagaaactgcaaaatttggaggccgtggaatttgtgataatattgccaggtgagcacatctcaaaaatgatattggcaattatatttcagtataggttgttataactaacagaaatgaagaaaacccgcgagccgggaccgcgggggaaggtgtccccggcgtttgccgcctgccgagcggttcggttctcggctccctggcagccggccttcaggtcgggcgtcttcgttggtgacttctgctttggccctcccggtgtggccgctggttgggctgcactgctgtgacccatcatcatctcctgaagctgcatcctgtcctgacttgcctccacgcttgcacactgagaaatgcagcacgttgattcacttgcttaaggaatgtcacacaaatcacagcattctgaaatttcttggtcattgtaaccatcttgattgggagatgagaaaatgcctgaagaaggagtacatagaaaagagttctggccaggcacggcagctcacgcctgtaatcccagcactttgggaggccgaggcaggcagatcacgaggtcaggagttcaagaccagtctggccaagatggcaaaaccccatctctactaaaaatacaaaaaaattagccaggcatggtggcacacgcctgtaatcccagctactctggaggctgaggcagagaattgcttaaacctggaggggtgaaggttgcagtgagctgagatcatgccactgcactccagcctgggtgacagagcaagactccgtctcaaaaaaaaaaaaaaaaaaaaaaaagagttcaagaccagcctggccaacatggcgaaaccctgtctctactaaaaattagctgggcgtagtggtgcgcacctggagtcattccccagagctggaggtgggtcctgaatcaccacaggcctcatggatgtcccgctctcccctgtgtcctgagtctctgacggtcaggccccagcacccagccaggcctcctctgaggaatggccacaatggcaggtttggaaagcggcccagacatcacagatttctgtaataattcgagatcatttggggtccagctcccaggcactgtctcacttcattcccgaagtgtgaaactgtgatattaggaatttgaatacagccagtccctcctgactgctggcgcctgtcctttcaggagggtgacgcaggccactccgcatcagggtggataactctgggacctgttttttaacccttgatatttaccgactttggtcagttcaccaatccccagtctcaaatttgcgaaagtaaatgctatgcccttttcatagagctgtcatccataaaaactgccactgtgttccctcactctgtcccccaatgagccctgcagcaggtggcctgctgccacgctgggaggtgctctgaggacaagcccatgcgtgctggtgacagctgcagccccttccctctgtggtcctgcctggctgctggtccagagccccttccccagcccaggatggctctccatccctttgggtgcctgagagagcaaacacctggtctcatatcaccggcagttgtggactggctggacggtgctgagagcgggaaggaaagaggggcagagggcatctgagcctggattcctgctctttctgccagctctgacctggacccacacctcctcccagcccccatccaaagcagagaggaggcacaatcagaaagggaaaagtaaaagaggaaagaggggtgcaaggaccaaagaggcacgcttcaggtaccacaaagtgatgaggctgcaccccaaaccccatcaccataatcatctgctatggactgagcatgttcatctgtccaccactgcatctggctctctgtataaattatctcacatacagaagcaggtcacagcccaccttgtgcctcccctctcttctctgctgcccccttgcaatcatgcaaactcatcgtccatcttaaaaatacggctggaatggggagcataccccttcaaatatcagtctcttaattgaaagatgtgcaaagacacagttcgtactcaatgcttcattctgttaatttctgcattgttgtctatttgtaaagttctgtgttcataacaaactcttttttaatgttatgatttggttttttgtttgtttgttttgttttgagacagagtctcgctcggtcacccaggctgatgtgccgtggcacgatctcggctcactgcaatctttgcctcccaggttcaagccattctcctgcctcagcctcccaggtagccgagattacaggtgcctgccaccacgcctggctaatttttgtatttgtagtagagacggggtttcaccatgttatccaggctggtctcgaactcctgacctcaggtgatgtatctgccttgacctcccaaagtgctgggattataggcatgagccactgcacctggacttgttttactgaaatcactgcagcaggctttgagtgcctattagtgactcactttcactgagcaagaggtagattttaggtctgcaacacagcacatgcccttgacaagctcacaaacttgtaggagggataggcctagacctgtagaccatgtgtaggagggaccaggcatagctcccagggagggggctgcccataaggtggtcctcttgtccaaagtgaaatggctcatatgacgcagcctccactgttctgaattctgcctctaaagaacccagaaaatgctgtcccgcaagaggattccttaggtaatgacttgagtccatgagttctcctagaagcatttctcaagttagactgaagaaaacattttcttttgagccaaacttcctttcattggtataatcctatataggtagccaaagcccttttacttgtatcatctacttttattcttacaacaaccttagggagtgggtaggaaaaaaacctactctcaccccatctcacaaataagggaaattacaagggtattgagtggtgaattttgggaaagaaaatctgttccttgtccagaaattcatttattcatttatctttgcatccatccatgcatccatccatctacccacccacccatccacccatccatccacccatccacccttccacacgttcacccatccatccatccatccacccatacatccatctacccacccattcatccattcaaccattcatctatccacccatccacccacccatccatccactcatctatccatccatccatccatccatccatccatccatccatccatccatccacctacccacccatccatccacccacccattcaaccatttatccacccattaaaccatccatctactcatccatccatccacccatccatctatccattcacccactcatccaccatccacattcatccaaccatccatcctccatatatgtgttgggtgggtccctattaaccagtcccagggctatatgctggaggacgaaggaggccctagctttcagataactcccagtctggaatgtttggatcctggagattatggcagtggctcattttctccttaacaactcaatattattaccttacccaaatacagtctgaggacaggcaaagcaggaagcagattcctttcgttggatttggggtgtgggtgcagctgcaagcagtaaggcacagagaagaatactggtgtcccataagcccctgagggtcaccacactaagtgctgggtcgtaagttgtatcaaatgtactgcctgagcctttagtcaatcctgtgagaggatttctggtctctccctcaacactaagaggtagcaaatcttctcttgatggattaatggatctaagagtcacgagggctcctgatttatagcctgcattcacgtctgccctctgcatagcctggcagaatcttagaggaaagttcttgggcagcagggtcaagtaaaggttgttattttaaagagaggggagaacattgtgtcaacacaggaaggatgtcatgaagaatgggacttggcagttccctgcggctgggaggagggtccacatagtacgacagctctctgccttcccaatccagtcactcatcccttttagagctcagactctggcaaacagactcataataacccaactcacaagagactgaatgatggcaatgtgcttcttcctagtaggaggtaggttcccagatctgcagccagggcctctggcctgcagtaggtcatccccactggagccgagccaggaaagaagagcctttcatgtatgttaccacatgtaagcctcttacagccaactccttaaggagctatgactgaggttcactgaaggggcaggaagtatcccaacgtggcccagcttgccagagaagagctggactttaactcaggcccagggctatttcccggcctgccttccttcttctctacctggcccagtggcccaccatagcctagttgttagaccctagaggtcaataaacctagtctcttccctggagactgaactgtagtccagaattgagattgtgcgtatgtgacatgtgattcctggatacataagacaacacacagagcagagggattgatccttacattttcgcagtcagggagatgctgagctgtgtctctgacggtgaatatagaattgtaaccaagggagctgatgttctcaggagatgtttgcagccgggtggagaagctcagtgcatgcttttggggctacttctggttaaataatagagaataagtgagcccacaatgtagagataatgtgagcgagtgttttgttgcgtaaggatgtgagagtctgagggtgtgtggcgtggatgcagaatgtgagattattgcagagagtgaggctgaggaggctgcagcgcaggcttgggggataattggaggatgcagttcccagggagagaaagaaggagtccagtcatggttagaaacaccaaatgattgtactcattatttctggctcagtagcaaattactcccaaacttagtgacttgaaacaactttggttattatggcacagatttggcaggtcaggaatctggctgggagagctggtccctctgctccgtgtccccccaaggccgtggtcatctcaaggcccctctgggaaggaccctctttcaagctcattcacacggctgctggtgggtttcagttccttgcaggccgctggaccgaagcctccctgggttccttgccatggaggctgccccgtagagcatcacacaacacggcagcttgtgttgtcagagcacgcaatcaggagggccaaaagagaggggaccagtaaggcggagagtgacagcaggatggaagtcatagccatccgtaacttagccatggaaatggcatcccatcagctttgctgcagtctcttcctagaagcaagccactaggtccagcccacgctcaaggtatgagcagcaggaatgggaccactggggcctgtcagaagctgcctggcaaaacgactaaacaggaatcaagcatttgctgtgtggtggacaccagctaagccttttatgtgcactctgtgctatgacccttcacaaagccctaccaggaagaggtcctgttattattctcatttcatagatgaggacattgaggctcagagaggttgggtaacctgctcaaggtcacacagccagagaacagcagagctggaacatgaacccaggcctgctgactacaaagccaggacctctccaaagcagaggtctccctgtcctcccttgcgagcttggacaggaagtggggcgaggtggggccgggaaagccgccttcccagccagcctgcctgccctctggctccaccgtctctgatctttttccactgtctttgctcgtttgacttttcccctgttgttcctactttcttcatccaaacaaatcctgtctgctcacatttcaatcttccagctgggtgccagccagagtcctcccttcccttgaatatgcaagattggcatgccccatgcagatttgccacaagcagagtgggctgctgcctccgcatggagcagggggcaccaggggactctcttgctgtccccagtatctcccgaatggcctgcccccacgcctgggtggtcaccattagcaatcgcttcatttgggttatagcagcgaggatgctggcggttcctgctgaaaccaggggaggcagaagcagcaggcagggaggcctttccctacgcacacctgagactcccagattgctttggtagccaacagccagctgggagctgggcctcaaggagttgggatataggacaggtcactttaaaaaaaaaaaaaagtgtggtaagggccgggtgcggtggctcatgcctgtaatcccagcactttgggaggccaaggttggtggatcaagaggtcaggagttcgagaccagcctggccaacatggtgaaaccccgtctctactaaaaatacaaaaattagctgggtgtggtggcgggcgcctgtaatcccagctactcgggaggctgaggcaagagaattgcttgaaaccaaaaggcggaggttgtagtgagccaagatcgcaccactgcactccagcctgggcaaaaaagcgaaactccatctaaaaaaaaaaaaattgtagaaacatacaaataacataaaatgtacccttgtactcacttttgtgcacagttccggggcatttggctcattcacatgattgtgcagccttcaccgccatccttctccagagcattttcatcttctcaaacagaaactgtccccatgaaacataaactctccaccccacccaccgcccccaggccctggcagtttccattccactttctgtctctgcaagtctgactgtgttaggggcttcatgcgagtggaagttgacaggatttttttttctttgagacggagtcttgctctatcgtccaggcttagagtgtagtggcttgatctcggctcactgcaacctctgcctcccaggttcaagcgattctcctgcctcagccttcagagttgctaggattacaggcgcccgccaccacacccggctaatttttgggtgtttagtagagacagggtttcaccatattggccaggctggtctcaaactcctgatctcaggtgatccacctgccttgcctcccaaagtgctgggattacaggcgtgagccaccgcacccagctcagcatttgttcttttttatttggcttctttcgcttagcataatgtcctcaaggctcctctgtgttgtagcctgtgtcagtgtttcactgcttttcgaggctaaataaggttccattgtatggagacaccaagttttgcgtatccgttcacctagcaatgaacacttgggttgcttctgccttttggttgtgtataaagatgccaggaacatgggtgtgcaaatatttgttcaggacaagtcacttggtgatgaaccaagaaggggacaggtgtggagggtggggctgctggagctgccagccttggagggaaaagcagagtggggggtggtgggctggaggaggtccggagcctgggggaggtgcaggggagggagtagggagtggggatgaggtgggcagcagcagggatatctgtaggagacgctgccttcccatgataccacctgccttgggcagaactatgttttctccgtaactggggtctccccagcattccaaagaggtagggatatttggggaacctgagagctgtcaagaaacagctgtcaggcagccgaacctagacttgctggcccatattcaggattgcttccaaggtcgggatcagccaagcctggcgggaaagaggcaaatgctggtcactgtctggcaatgcatgatgtggcaaagggactgggctgctgtgatacagagcactgctcatatgcatgggtcagctaatgcctggtaacaaacaggacctcaggggctgcactagcactcagggcttagcaagtttctgtattggccagttattggcacaataatgatgaatgccaaaccagtcccaagctcgggggctagctcagcatcactgatttcctgggcctgtggctggcatggctctgcctcacaccacagattgggctgggtctcccaccctcctgggctcgctcggcttactgaggcagcttcttgtcctggtgacggcagaggcaacagaaggcaagcccacccacgcacgcacgtctgcaagcccctggctttgctctgcctgtcttgctggccaaagctagcctcaaggccacactggacgtccagggtcagggaagcagattctgcctatgtgggaggagctgcaagtcacatgacagagggcagggacacagggaggggtggtgagaattggggcaaagctgccacctttctggaatgttccaaaagaagatggacgactactgctcctcggccctggaagggagagttcttttttccccactataaagttttctgattccaattccaaaatgctatgactcttaatcaatgcctgatgaaccatacttgagcaaacataatttattctctgtcaagatcatcctgaggggatcgggtgtctttgtttccctcctaaatcccctgcgaattcatcttttttattccaggcttactaggaaataaaagattttgtctgagggctgatgggtattagcagagctatatggagcttgagttgaaacttggaatgtttgtggatcaaaaccgagctgtgctggcagcgtctgggaggagggtgcactcgctgaaactctgcccgagttgtggctgtcagctgcctcgactgccagaccagactcgctgttttggggccgggtgggggggacggcggggggagtgttgttacggattgtgctggaaggttttcttgctcttggcttttttggtgaagggcctggattactgcagaaaatccagcttatgtgtccaagggcaaagcaggcctgtttatctttaacttttacatgcttcacacatctcctgaggtttgactctaagcaggatttgtgtcttccctgatgcaagaaaacaggaggcaggcgctgagcttgcaggaggccacacagccagtgcacagcttgccatatgcttaacgtggctctgagaagagagggttgtttttttaatattaaaaatggctatgggaaaatctgccctaatgctattaagcagtgctgatatgagatgtgtgtggctgtaactttctgattggatttgggatctaggctgcagccagccagacgctggagacacattcctgagccacctcagcccttgcctctttgccgagctgcggctattctttggggcacgcaggtagatgcagagagactgaagtcttcagaggtgggtgtgcacagaagagtgattcccagatcagggaagactttggcccccagatgacatttggcaaagtctagaggccttttcaattgtcacaattgggaggagggaggtacagtagcatctggtagatagaggccagggacgctgctcgacgcctacagcagatggactcacagccaagaatcatctgcccctaacattattcagccttaacttacaaaggaaggaaagtctggtgcatgtgtgctgtggtttgagtgtgtcctctccaaaattctgtgttgaaacttcatgtccctgtgtcctaattgccttaagaggtgattaagtcaagagaacgcctccctccataaagggattagtgcccttataaaagggctttgaggctggatacagtggctcacgcctgcaatccagtactatgggaggccgaggtgagtggatcacttgagcccaagagttcaagaccagcctgggcaacatagtgagaccctgcctctaaaaaaaaaaaaactgggagtggtgtcacatgactttagtcccagctatttgggaggctgaggcgggaggatcacttgagtacaggaggtcaaggccatggtgagctgtgattgtaccactgcactccagcctgggaaacagaacgagaccctaccataaaaatactactactaataaaagagctttggtgaagggagtttgtccctcttgcctttctgccctccaccacgtgaggacacagcgtcccccccaccaggaggaggcagtgctccaagtgccatcttggaagccaggctggaccctctccagacagcgaacctgctgagccccggatttcggactcccagcccccctggcaaaggcccgtgttctcttccaacatggtctagtggctacatctttgggagggggcgaatgctgaggcctcacatggtggacccactccctctggccctttgatagggaccctaattccatccgtgaagtctccaccctcgtgacctcatcacctcttacggtgtcacctcttaatcccattacaatggtgagtaaatgtcaacatatgaatttttgaggggacaccatcatgcaaaccatggcagtggtgccggctatgtaagtagctatgactgtgcagaggtcaccatggccatctcacatgcatgtcactcaaccatcaaaactcacccaacagcagttgcccaaagccccccaccctgccccacttgtacccacaggcactccagtcccagctccaaattacatttgtgcagccttatttctccaagtggcttcccacaagcttgagatgcaggtggtcgtcagggaaggtcctcatctctccccattggccaacttggttttatatcacctgccctgaagtccctctgccagaggagagccagtgactgtgccaggtgcttccttggacacacccaagctgccgtcttaaagaatcaccccgtcaagctctgccacgttttgtcatccggccccacatttccttcagctgtatctcccaccgttcccaaatccaacaacggcgcctgtgcgtggccaggagcgcagagaggcaggggtgggagttcccccagagcctggctccagctcacagctcagacaccccgtggccctcctgccctgggcccggatgagtcgggaatacgggactgctctccaaggtccccccttccccgtctgcagccagtcagcctctgagtccttatcatctgtttcccaaatagttccagatccctccctctctgcccttcccccctcatgcccaggtttgggccactattacaatttacctggattacctctgcagattcccaggctgccgttggcaacagtggaaggtgcagatgggggcgaggaagatggtggcgcactgcctctgtccttctctgctctgagttggagccaaccccgaggtctccttgcccctggcacgcccaatctcattgtcatgcaccagagaagtggccgggaagctccccggggcctggtcactcggcttagtcggctctgtcacttccccccccgcacctgctcagcccattgactctctgactgcacctcagcccctgccccccgccaggcagtcacctgcacttctgcatcctccaggggcctctctttcccacctcagggctcagtgcaaacggtgccttctttctcgaatgtggataatggaggaaaaatgttgcttccccacgaagccttcccaaccgcctaatttggatgcagtgtcccatcctgggcccttagccctgttgcagtgcacggtggttgacggcccatcatccttctcccctcaggacagaggccatgtcttgcttgtggacccttgatcctgtgtgagggacacgtgcctcccaaatgcctgggctcagccttctctggaggcttcttcactttgtggggatcctctaggtgtgtcggccacatgattccccttcactgattcatccagcaaattcattcccctccactgattcatccctgagcattgactctgatgggcaccagggccgtagcaggcccaggcagacctcgctctcactggcctctcctgcggaggagaagcagacgataaacaatggctgggctatgatgggcggtggcggcgcagtggagagagatcagacagggggagggcacagagagaggctgagggggctcttttgggagcagccatagaaaacttctctgagtgacgacgttagagacctcaaggaagtgagggaacaagcaagtgttgctggcgttgggaacggcaagcgccgggcttggggtggcccagggagagcacgggtcagcacgcctggagttcagcagcgtcagggaggtaacagggtcggatcctgtagggcttaggggtgatgttaaacactctggaatcatcctgcatgagatggggtcattggacgttttcgaacccttggggttcctcctctgatttaagctttgagagacccactttgcaaagttcccgctatacactctatcattacatcctcacaaattgcctgggcacagtggctcatacctgtaatcccagcactttgggaggccgaggtggatggatcacttgaggtcaggagttcgagatcagcctgaccaacatggtaaaaccccgtctctactaaaaatacaaaaaaaaaaaaaaaataggtgtagtggcaggcacttgtaatcgcagctactcgggaggctgaggcaggagaatagcttgaacccaggaggcagaggttgtagtgagccaagatcatgccactgcgacagagtgagactccatctcaaaaaacaacaacaacaaaaataaatcaacatatgggtaatcttttccccattctacagttgagaaaactgaggctcagagagaaagggtagtgacttacctgagggtgcagagcttgagtgtgttgaactttgctggaatctggctcagattcattctaaaggccccactccagctgccctgcagtaggaatggcttgaccactctgctccacaggcccaaagacaggatggggtcagggccaaaatgagtggtggtaaggtccaactcggttcttcgagaagtaaatcagctcctgagacgtcgatcttccagctgcaccctgtcactattgagacctagccaagcacttgggggcaattatatgggtggctgtggctgtattggaaggcctgagagaggagggagtcacaggtgaccctcttgagcaggagactcgggcccaagattcagggactgccaccttggtgacagtgtcaatgttggatgattttaggtaaatgatgccttcacagaggcctttaacccagcggaaaccagcttggcaggtacatgcttttttccttccttttcggtttcattctaccaacctccctgattgggttataggtggggttctccgaagctccctggaaggcccctcaggcttactcgggagtgtggaggtggaggggattatttgctcactccccaggagcagaggagggagactgtcccttgattgggggatgctctgagagatgccgtctgctgtgaggcgggaagaggaggggctccccggtcctcccggctgctcttcccacctcttttgggagcatgggatggaagatgcataaaaattcaaaccatcctgaaaataaatgaaaggaaaatatatatatccaagaaggagataaccatagcaatcactttcactcaaaggtaataaaaataaggatcgtaaacataaactcagaaattgcaagctgcactccctgccgacgtttgctgccatgagagagcacattccgagggaaggcaaggactttccagactttccaggacactctggacatgtctctctgattgaaacctccagaaggaaaaatatgggaggaaaatagtgagttatgggggggaaataaggacccaaactataacaaaaatggagaacacaaattatgtttacaaacgtttccctggctgacagagactctgcctggaacagatcacatatcataaggtggccaccttcccaggggtgggggctcctgggtgagtacagagtgttctgtgagtgacggccccccctccctgcccgcccccctccgccagcctcatggagctgcactggagatggcttattctgagcgtcagctgccgccttcctcctctgcagccatcagaccgaagctgccgctgtgacaagcaaaccagacaagatggaccgcggcagctgggctgtctgcagagggccatccaggcctccttgctagaaacatgcattttagatggacaaacggaacattttgtcctctttgccttctgatatttgtcttctgccagaagagagctaggacccaggcatcaaactacagccagtgaaatgttcctaactccctgcttgggttgcatcctgcgacggctgctcattgcccgacaaacgcttcgtcctgcctgggcatcatccttcagctgtaaccggcgatgcgattcccaagcactgtttaaactttccagtcaaaaatgagacaccactgctcacttatcagatttgcaaaaatcaagataataaagctaaggaaggctgggtgtggtggttcaggcctggaatcccagcaccgtgggaggcagaggagggacgattgcttgagctcaggagttgaaagttgcagtgagccgcgatcatgccactgtcctgcagtgtgggttacagagtgagaccttgactcaataaaataaaatataaaataaaataaaaaataaataaagctaaggaggaaaaccaaaacatggctagtcagtttggggaaggtgggacgacacggtgtcctcatgccggttcgtttgcaaactggaatgatcttttcggaaggaatgtaggagtgtctcaggaatctgtaaaaagttcacagccaggcatggtggctcacgcctgtaatcccagcactttggaagtccacagtggcagtatcatttgagcccagcctgggcaacatggggaaaccctgtccctacaaaaaatacaaaaaatatttgctggatgtggtggtgcatgcctgtggtcccagatgctcaggaggctgaggtgggaggatcctttgagcctgggaggcagaggctgcagtgagccatgattgtgccactgcactccagcctggatgacagagtgagactctgtttcaaaaaagaataagttccccaccttgatttgctatgagctgaattgtgtcccccagaaatccagttccaagtcctaaccctggtgcctgtgaatttgactttatttggaaatagggtctttgcagatgtaattaaagttaagatggtatgtctgctgtctttattttatttatttattttttctgagacagagtctcgctctgttgcctaggttggagggcagtggggcgatctcagctcactgcaacctccatctcctgggttcaagtgattctcctgcctcagcctcccaagtagctgggattacaggtgcacatctggctaatttttgtatttttttttttccagtagagacagggtttcaccctgttggccaggttggtctcaaactcctgaactcgagtgatccgcctgcttcggcctcccaaagtgctgggattacaggtgtgagccactgcacccagccgtctgctgtctttatgagaggagagacacatagggagatggaaggccctgttaggatggtgccagaggtaggagggatgcagccgcaagctgaggcatgtaggaggttgctaaggacaccttgaagctgagagaggggcacgaacagattcttcctgggagcctggaggcagcagaaccatgctgacatcttgatgttggaattctggcctccagaaccgtggagcaatacacttgttgctttaagctacccagtttgtggtactttgttacagcagccctagcaaactaagacactctatttttatgttttttgagacggagtcttgctcttttgcccaggctggagtgcagtggcacgatctcagctcactgcaacctctgcctcccgggttcaagtgattcttctccctcagcctcccagtagctgggactacaggctcccaccactacgtacggctaatttttgtatttttagtagagacagggtttcaccatgttggccaggctggtcttgaactccttacctcaggcgatccactcgcctgggccttccaaagtgctaggattataggtgtgagccaccgtgccgggttacaaactaatacactctttttgggcttttactttcacttctaggaagaaatattgtagctcattattttccacatttatttttatttccacatagtcagactatatttcccagtgtcctctgcagttagctatggccatgtgatcgagttctggccaatagaatgtggccagaagtgacataaacgccagacccggccaatggaaaccttgtgctcccgttcgggggtctgtgcccccaaggttaccccctaacacctactgaactactgtatgtgtgaaaaatggccttctattgatgctggggagttactgttccagtacttagcacagcttggctaatacaaggaaaaatcagagataaagaaaagcaaagattcacaggccaggatgtttccatattccacatcatttactgggggcagcagccggggcacaagacaaactcacttttcttttttttttcctttttttttgagacagagttttgctctgttgcccaggccagagtgcaatggcgcgatctcggctcactgcaacctccgcctcccaggttcaaatgattctcctgcctcagcctccctagtagctgggattacaggcatgtgccaccatgactggctaattttgtatttttagtaaagacggggtttctccactccatgttggtctggctggtctcgaactactgacctcaggtgatctgcccacctcggcctcccaaagtgctgggattacaggcatgagccaccacccctggccactcacttttcttttaatattcagggcagcatgaccccaagtctttgtcctttatatttctgggcagccattccttttgggatgtcataggggtccctttaggccctccccagatcagcctcctgccccactgctccctccatctggggtttatcccattgctgtggacagagagagtcgtcagtatgccttggggccagtgttcactctgggctgtctgagctgttcaaactctccctgccaatgtgtgttccctgctcctgtttctgcttctagcagtagctccttggggtcctgctcattggagtcatttgacaatactgggccaggcatggtggctcaagcctgtaataccagccctttgggaggcagaggcaggcagatcgcttgaggtcaggagtttgagaccagcctggccaacatggggaaaacctatctctactaaaaatacaaaaattagccaaaaatcgcttgaacccaggaggcagaggctgccgtgagccaagatcgtgccactgcacttcagcctgggcaacagagtaaaactccgtctcaaacaaaacaatactatgtgcccacactgcataccaggcgctggctctctgcagggggtaaaagggagaatgagacagatatgatccctgtcttcgtggagcttctatctggcggggcaaagggacattaaaacaatgtttaattagaatagtgattagtgcaatgaatacaatgtatagggtcccatgggagtggatacaaggtagggtgactatgttttatcatccaaatcagaacacctttgagaatgtaagaggacactattaataattatgccagaggaatgagtgaaaaccagggctgcctcagggaaggcagaacctttggccacctaggaggagaccaacccagtccccgacaagctgtctctgagcagctccctcccagctttggacccagccctgaccaatagctcgggggagcccacacagcttggatgagttctcccaggcttgactgaacctcactcttcagaaaacaccttaccacctccttcagggcccaggtgggctggaaacaagttccctgtatgtgattaacacaatggttgagaatacaggacgaagctgtgggtctcacagtttgctacgcatggactcacctgggattccaggctgactctgggctgccatcccctaacattgtgatgaaatgggtgcaactggggcatctggaattttattgattgattgattgattgagacacagtctcactctgtcacccaggctggagtgcagtggcatgatctcggctcactgcaatctccgcctctggggttcaagcaattcccgtgtctcagtctctctagcagctgggattacaggcccacaccaccacacctggctaagttttgtatttttagtagagacggggtttcaccatgttggccaggctggtctcgaattcctgacctcaagtgatctgcccgcctctgcctcccaaagtgctgggattacaggcgtgagccaccgcacccggtcattatttatttatttatttatttatttatttttgagacagtctcactctgtcacccaggctggagtgcagtggcacgatctccactcactgcaacctctgcctcccaggttgaagcgcttctcctgcctctgcctcctgaatagctgggattacaggcatgcgccactacgcccagctgatttttatagtagtagagatggggtttcaccatcttggccaggctggtctcaaactcctgacctcaagagatccgcccgcctcggcctcccaaaatgctgggattacaggcatgagccactgcacccaaccgatgatcttttattttcaaaatgtttcagaataattttttttaaatgtcaaataatttttaaaaacgtggcaaaaagaaaaggaaaaaaggaaattcacaaaatcagaacactgattcctctgcttaacttttattttcttcatttttgggaacattttccaatttcctccaacaagcatctattactttttacttttaaaatttaaaataaaaatggtcaaagtatgacgtgatgctgtttaaaatgtcaaatggtatttctgggatcataataccagtgggtccctggcctccccatttctggtcctgtgacccttggtccacatgtctgggcagagtgagatcctgtctttaaaatacatctcagcatttctacacgtgtctacggtctttttctttgcgttttaaaaaccaggttacaggccgagcatggtggctcacgcctgtaatcccagcactttgggagtccgaggcaggtgtattgtttgagctcaggagttcgagaccagcctgggcaacatggtgaaaccctgtcactacaaaattagtcgggtgtgggctaggcgcggtggctcatgcctgtaagcccagcactttgggaggccgaggtgggcagatcacttgaggtcaggagtttgagaccagcctggccaacgtggagaaaccctgtctctactaaaaatacaaaaattagccgagtgtggtggcacacacctgtaatcctagctactcgggagggtgacacaggagaactgcatgaacctgggaggcggaggttgcagtaagctgagattgtgccactgcactccagcctgggcaacaagagcgaaattccatctcaaaacaaaaacaaaaacaaacaagcgaacaaaaatgtgattgtgctacagtattatgaccactacagcctcactaggtgataggaatttttcagctccattgtaatctgatgggaccaccgtgctatgtctagtccacctttgaccacaactgttctgccgctcatgagtgttacttacaacatccttatgaaatagacactgttagtttcccaatgttacagatgaaatggggctggggtaagtccagctcctctcccagagcctcagggcaaggaattgacagagtgaaaactggaactgaggcctgtgtgactcccagcacaagcaggcgctggagggcccccggcagccacttgcaggctaatgggtgctcttgccccatgctgggcactgtgccacacattgcgagggtctgcgcttagcatcaggcagctgtgcttggaatctttgcccagacttggtcagtggagagatcttcagaaagttatttcatccttgtgcgtctcaacttccttgctgatacaatggatctagaacacaagctcttcccaggggttattaggagggttacaagtgttaattctcaggaaaaaggcttggctccccatcaatactcactaagtgttaggattatcctattttacttacaggcatatttctctaatctcccctatgaaaagcagaagcaatggttggcctacctttgtcccctcaggatctactgttcagctgagagcttgagaatcactgatgttatgtaaatagtacataaaataacaagtggggagtttaaaaagagacagttggtcacattctttgtattttcatagattattaaaatgaagggttacaaaaattcattttgcatattattattattattgttattattattattattattgagacggagttttcgctcttgttgcccaggctggagtgcaatggcatgatctcagctcactgcaacctccgccacctgggttcaagcgattctcctgtctcagcctcccgagtagcggggattacaggcatatgccaccacgcctggctactttttgtatttttagtagagacggggtttcaacatattggtcaggctggtctggaactcctgacctcaggtgatctgctcaccttggcctcccaaagttctgggattacaggcgtgagccaccgtgcccatccagtttgcatattatatattggataataaattttctcaacatagtctcaatcatataaactttcctcccccaccccatttttcttttttttctttttttttttttgagacagagtcttactctttcgcccaggctggagtgcaagctccgcctcccaggttcacggcattctcctgcttcagcctcctgagtagctgggactacaggcgcccgccaccatgcccagctaattttttgtatttttagtagagacggggtttcaccgtgttagccaggatggtctcgatctcctgacctcgtgatccacccgccttggcctcccaaagtgctgggattacaggcgtgagccactgcgcccggccccatttttctttttcttttcttttttttttttttttggagatgcaagttcttttattaaattctcaaattacccaatttgtgtgggccatctgtttcctacctggaccttgacacattcagaagtcaacaggacactgtccgaatgggttttggttagtaacttccttatagcgtctctcactgtggctcacccatctgtgttgttctcctcatccattcaggaaatgagatttttttttttttttttttttttttttttgagacagagtcgcactccatcgcccaggctggagtgcagtggctcgatctcggctcactgcaagctccgcctccccggttcatgccatttccctgcctcagcctcccaagtagctgggactacaggcacccgccaccacgcccggctaatttttttttaattaattaatttatttttttattgatcattcttgggtgtttcttgcagagggggatttggcagggtcataggacaatagtggagggaaggtcagcagataaacaagtgaacaaaggtctctggttttcctaggcagaggaccctgcggccttccgcagtgtttgtgtccctgggtacttgagattagggagtggtgatgactcttaaggagcatgctgccttcaagcatctgtttaacaaagcacatcttgcaccgcccttaatccatttaaccctgagtggacacagcacatgtttcatagagcacagggttgggggtaaggtcacagatcaacaggatcccaaggcagaagaatttttcttagtacagaacaaaatgaaaagtctcccatgtctacttctttctacacagacacggcaaccatctgatttctcaatcttttccccacctttcccccttttctattccacaaagccgccattgtcatcatggcccgttctcaatgagctgttgggtacacctcccagacggggtggtggccgggcagaggggctcctcacttcccagtaggggcggccgggcagaggcgcccctctcctcccggacggggcggttggctgggcgggggctgaccccccacctccctcccggacggggtggctgccgggcagagacgctcctcacttcccagacggggtggctgcccgacggaggggctcctcacttctcagacggggtggctgcagggcggagggactcctcacctcccagacggggtcgcggccgggcagaggctgcagtctcggcactttggggggccaaggcaggcggctgggaggtggaggttgtagcgagccgagatcacgccactgcactgcagcctgggcaccattgagcactgagtgaacgagactccgtctgcaatcccggcacctcgggaggccgaggctggcagatcactcgcggttaggagctggagaccagcctggccaacacagcgaaaccccgtctccaccaaaaaaatacgaaaaccagtcaggcgtggcagcgcgcgcctgcaatcgcaggcactcggcaggctgaggcaggagaatcaggcagggaggttgcagtgagccgagatggcagcagtacagtccagcttcggctcggcatgagagggagaccgtggaaagagagggagagggagaccgtggggagagggagcgggagcgggggagggggagggggagggttctttttcttttttattattttttgagatagggtctctgttgcttaggctggagtgcagtagcgtaatctcagctcattgcagcctccacctcctgtatataagcaattctcatgccccagcctcccatgcagctgggattacaggcatgcaccaccacacccggctaatttttttgtgttttttacatagagacggggtttcgtcatgttggccaggctggtctcgaactccccacctcaggtgatccgcctgcctcggcctcacaaagtgctgggattacaggcatgagccactgcacccggccccttttttctttttaaatctgccatgggtacagaccacatctttgtgattctacctttaggatctgggggaggaaggaagggactcccaataatgggatgtttgacctccaacggacactgtaccttcatcccttctccacaaagaatccgtagggccgtagacagcagcgtctgatgccgtaaagccttgcagatggctccccaagtccatccggactcatcggtcagggcgcccaggctggaaggccacccagcggggactgggcctgggcacctgctctcccagctgctttgcccttgtcttgctccacatggcaggcgtctcagagaggctcttcactgcccttcgcaaaacagcccagggtgggaaggagaggctggggtgcaggtcccagctccgtgttgggctctcaggcagtcccctccctgatgcatctccttctcggctctgtctttgccgtgttttccttggctggctttttaaggctagggctatgcaaaaatcctcacttctgaaggaagaaatgtggacagtgggtgaagtttattcagtaagaggcacttttcaccacagtccctatgctacccagggacccttttaaaaatgggaataactttgtttctgctaattgatctacatgcttattatataaaaaaatgaataaaatagaaaggtgtgctgtagaaggtgaaaatcacatgcataaataccaccatatttttagacgggtggagagggggagacagccttgctgggctcctggcagcctcacattcccagctgaaggcaatgctgtggccgtgttacctccttttccttcctcttctcacttcactgtcaacaaagcatctgacctgcaggggggagtggctcgggttttactaaaaacggagtcatgatgctcagacaaaagcgacgttcttcatgtctattttggtttcctttaagttaagcatagatttttctggaaaggaccggagctggctgttctccctgcatggaaaacaacagctcagcgaagcacctggttttctcaagttccctgacctccagccttgctcctgattcttgatgacaaaacacattcagtgcccttgattgatttagcagagaagtggcaatgctgccgcatggaacgcagcccattctgcctgtgaatggaatgcctcggctgtgatggtcttctgttttgggaaaagattaggtcagttgcagtgggtcaatgctgagctcggagactgaatatccagccaccttagttcttcctggtaaaggctatttatctgagccatttatgcaacccccaccccctgcatttctccctgcattcatttctgccttctgtcatatttgccccccacactcaggtttttatatttgttctcttgctcactttgctgtatctttaattccatttgtcgaacagtctaagttttagggtaagaaggagtcacctggtctacctttacatggtaggacaaggtgccccactctgttcaagcaggttattctgcttttttttttttttttttttttttttgagacagagttttgctcgattgcccaggctggagtgcagtggtgcaatctcagctcactgcagcctcaaactcctgggctcaagccatcctcccacctcagcctccctagtggttggtactacagacgcatgtcaccatgactggctaatttaaacaattttttttgttgttgaggccaggtgtggtggctcatgactgtaatcccagcactttgggaggctgaggcaggtggatcacctgagatcaagagttcaagaccagcctggcaacatggtaaaactctgtctctactaaaaatacaaaaattagctaggcgtggtggtgggcgcctataatcccagctactctggaggctgaggctggagaatcacttgaacctgggaggcagagtttgcagtgagctgagatcacgccactgcactccagtctgggcaacagagcaagacaccatctcaaaaaaaaagaattattattattttttttttgtcgagacagggtttcccggtgttgcctaggctagtctcaaactcttgggctcaaacaaacctcccacctcagcttcccaaagtgtgggattacaggtgtgagctactgcgcatggtctgttccactctttttggatcctagctgggcctggaagccccttcagccccacatctgcaggctgacgttcaccaccagcaggaggctttcgtggcatgtggtcttacaaacacctctgtgttcaagtcttgaataagtgactgtaagagactcatggagcaggttgttgccaacaccctcccccactttctttcttaatttctgcattttttaaaaattaagatgggatcttggtctactgactaggctggagtgccgtggtgcaatcatagctcactgaagcctcaaattcctgggctcaaattcctgcttcaacctcacaagtagctgggaccacaggtataaatattaaatttttttttttggtagagacagggtctcactattttgcccaggctggtcatgaccgttttcatagcttgaatcaaaccaggtttgtgtgttttctgagattcctaacattttgtttttaagattaaagtattttagctggacacggtggctcatgcctgtaatcccagcactttgggaggccaaggcgggtggatcgcctgaggtcaggagttcaagaccagcctggccaacatggtgaaaccccgtctatactaaaattacaaaaattagccaggcgtggtggcgggcacctgtaatcccagctactcgggaggctgaggcaggaaaatcgcctgaacccgggaggcggaggttgcagcgagccaagattgcgccactgcactccagcctgggcgacaagagcaagactctgtctcaaaaaaaaaaaaaaaaaaaaaggatcttatttaaagtctctgtggacaatagatgtccttgcttgtgaagaactggagacattggaaattggaactgcagccccgagtggccagcccaccacatgccacagtcatggaatcacccaggtgagacaagtcagcccttgaatttccaggctgccattactcacagatgctcactgcctttcaaaaacctagagccacttccaaatgcttggctgggacttgaagagaatcatccgtgacatactagaacgaggtttcttttaaatgtcaagatgccataaaatgattcaggcccatccttgaaccttcagggtgagagctgagcagtcacaagcccctttctggtctttacatgaatcacagaatttcaaactggaaggatccttagaaattcgccttctccaagttcctgatgtcacagacgtggagaccaagggtggaggagggcagaggctttgctgaaatctgcaggaattggtggcaggttggggaccatagaatgtgtgtgtgtcttcatcctcagtccaggacccgagtggacccctggctcggggtcacatgtgttaaacagtagagggtgtgttttgttctgctgttttgaggagaaggtgagtctctttctgctttgtcagatggaagtctggacaattacttcatagcattcattcatttgccacacatattgattaagggcccaatatgggacccgaactgaatcactgcaggagactttagagcgaatattgtctaagacctccaagtctgcctggctgtgaactatcctcagagcgacctgatttaagaattcagattctggctctcttccttggcgctgcctatggagggggcagccatctcctccttggcatcatggccgccctcagaccccctgtgaagcccaagatcatcaaaaagagaaccaagaagttcatccggcaccagtcagaccgatgtgtcacgattaagcgtaactggcagaaacccagaggtattgacaacagggttggtagaaggttcaagggccagatcttgatgcccaacattggttatgggagcaacaaaaaaacaaagcacacgctgcccactggcttctggaagttcagtctacaacgtcaaggagctggaagtgctgctgatgtacaacacttcttactgcgctgagatcgctcacagtgtttcctccaggaaccgcgaagccaccacagaaagagccgcccagctggccatcagagtcactgacccccatgccagtctgcgcagtgaagaaaacgagcagacagctcatgtgcatgttttgtgtttaaataaaactgtaaaaactgcaaaaaaaaaaaaatgcagattctgaatcccggtctccctgctccccgatctggggctctgggttggaagctgggcatcctgaattttagtttcctgggcgattctgaccacctggtgggtttggacttcaacgtgctcactgacaccttcgtttagaatttcagatacgaaaaatggatttcccaaggtcacacacgttataggtgaggaaactgagtctcacacagattcagtgggctttcccaggtcacagagctagttagggatgaatgggtttagcgttgctgcttttctgtaactgtgacttcctgcaagagctgctggaatgcagctgtctacagccaccggaggtggggagcaaggtgacaaggcagaggacaggagtgattttgttgccttcagatgagaacggggtcttcctaagaccctgcatccctgaagcctctgctggtaactgtgaggatagtctctgccaacagagagggtgccctgagtttcaataggagaattggctccatggggtgtaataaccaaacgaatgtgcggaatgacaatgatttaatcctgtagcctttattaaaggcttgctttgtgctgggtgagaaaggtctgggattgtattcttcattctcattttgaaaggaacacaaatgccatcaagattttgatatggatggccacatgtgtctaagccccggaaaaccatcctgagagatcaagaccatcttggccaggatggtcttgaactcctgacctcatgatccacccacctcagcctcccaaagtgctgggattacaggcgtgcaccaccacgcctggcctcgttttattcttatttatttatttatttatttatttatttttgagatggaatctcactctgtcacccaggctggaatgcagtggtgtaatctcggctcactgcaacctctgcctcccaggttccagctcttctcctgcctcagcctcccgagcagctgagactacagacgcacgccaccacgcccagctaatttttgtatttttattagagacggcgtttcaccatgttggccaggctggtctcgaactcctgaactcgtgatccaccctcctcggcttcccaaagggctaggattataggcgtgagccacggtgccctgcctacatttggttttatagtccaccttacacagttctctatacattgtaaatgagccaaatttcctgaattgaaaaagagagtcacacaagaggcttgcctccacgcacagacatgtagttttagatgtctgcacgtgaagctatatcaaaataagaataaactactgctaccaagaagtgggaaaaaaatttaaaaaggataaactgctcagaaaacgaaacagattaaacaaaaaattgtattagatgaaattttttttcatcgaatcttgctccagactcttcttctgttgcttcagacccttcttcatctgaagagtcttgctctgtctcccaggctggagtgcagtgrcacgatcttggcccactgcaacctccgcctcccgggttcaagcaattctcctgcctcagcctcctgagtagctgggattacaggcgcgcaccaccacgcccagctaatttgtgtatttttagtagggacggggtttcaccatgttggccaggctggtctcgatctcttgacctcgtgatctgcccgcttcggcctcccaaagtgctgggattacaggcgtgagccacggcgcccggcctttcacttggttctctatgaaaaaattaaagagaaaatctgattgacactgtggaggacactacactccaaaaaaaaaagtactatattttctaatattatcagtgctttttaatattttctaatattatcagtgaagcttttgccgctttttcaaaatctggaagaaacaaggcggttgattcctttaatttcaggagcatgtaacttcatttataatgacaatgtttttgaaaaaataaattaaaccgacagtttcatttgtaggattgactttaataacacgcctctactgaacaaatggcgattaactggggaaaacggaattatggtaaccgaggtaacggggtcggggactctttctccttcgctgcatgctgggtggcggggagagaccgctctgaggaggtttggtctgtcggccacgccgtaggccgcggtgcattctgggtcctggcaatatggcgtcctccttgatgggctgatgagatgagtttcactgtagctccaaaccagagggcaaagctcccatgacccaataagcccacattgtccctttcctccgtggttccgtgtcgcccgtttctcaggactcgttctcaggcaggagagagcctcggggctgaaggccaggaccagccaggccgcgcggacctgaggttgaggaaccgggtgcaggcgagcacgatgggccggtcgtggctctggttgcagcagctcagacgagtgcgggacccgcagggctgagagtggctggaggagacccagggccctttgaacccgatcccttggccggagacctcagcccagtcggcccagtgggcgaaccggcaccaagagcggcctgcctgtcttcggaactgctgaggcggtggaggccgagagcagggtcatcgtgaggcctgaagtctcttacgcttttggcagctcccctcgcagcccctctggaaacgtacagcctcaggagcagccagtggcttgggacctggggtggtgtgtgtctgcggagcttcttgggctgccccatttcctagcggcccccacctccccacttcccgctcagagttagagataaggatctcagacttttgcctgagtaagggtctccgcactctttatccatttggttttcgatttcccgtttttgtttcttatttcaccaattctggtacacgctagtttttaaggctggaggttctcgagcgcttgctgccaaggactcccccaccccctcccccactgatggagtccgaaatgctgcaatcgcctcttctgggcctgggggaggaagatgaggctgatcttacagactggaacctacctttggcttttatgaaaaagaggcactgtgagaaaattgaaggctccaaatccttagctcagagctggaggatgaaggatcgggtaagtggattctgaaggcaccccttgaacttggtagtttccccaacaaagaaagtttacagcccgaaaagtgtccttgggaaagcgccgacatttccccctagccacacgttgtaagtcaactagaaagcaaaagccatttcagactgcagtttttggttgaaaatggaaaatgctgttttggttagtcattattcactgggtagtgttaattcaattccaactaagttctaattcagataataaaagttaagcaaatttgaaaatagagcattgcgagtatttacatgctctttggcatactacttagttctgtaaacacttagaggaaggacaacgtgcgatataatctttatacccacatgattagtctactttttaaaatatccaagctgggtgaagtgcagaagtctgatatactatgctaggaaatgagattactggaaaatgttcaactagtaacaaattagtgcaaagttattataaagttacactgaacttcattctagtcttctaaacaatttctcttgaagctagattttcctccttttatgtagtgtcctgttgctgtaacttggaaacaggatttctttaatcgtgagttagggtatgaggcgtcgagacttgggtgcaggttttagggtgtccgcatggtgtttggagtgtggagagtgactcacaagcagctggtaggaaagaaaacagccgaattttgttggatttgtccttcataaaacacccatctatttcctggcctcaaaagcaaaagggtagtactgcttctcctctatgcacacttataatttgtctttttgtaagttattttctccctccaggctgcttttatttgcacatttcagtggtataaaagaggaagttggtgttacaatgtttgattttctctaggcttatgtatatgtttatggaatattattgttttttagtatatgatgttgaacatatataagattacacttctgggctgggcgcggtggctcacgcctgtaatctcagcactttgggaggccaaggcaggcctcccaaaggcaggcggatcacctgaggtcaggagtttgagaccagcccggccaacatggtgaaaccctgtctgtactaaaaatacaaaaattagctgggcttggtggcaggtgcccgtaatcccagctactcgagaagctgaggctggagaatcgcttgaacctgggaggcagaggttgcagtgagccgaaatcgcaccattgcactccagcctagggg

(19) United States
(12) Patent Application Publication
(10) Pub. No.: US 2020/0190590 A1
(43) Pub. Date: Jun. 18, 2020

(54) PREDICTION METHOD FOR RISK OF ISCHEMIC STROKE ONSET

(71) Applicants: NATIONAL CEREBRAL AND CARDIOVASCULAR CENTER, Osaka (JP); SHIMADZU CORPORATION, Kyoto (JP)

(72) Inventors: Masafumi IHARA, Osaka (JP); Shuhei OKAZAKI, Osaka (JP); Daisuke KAWAKAMI, Kyoto (JP)

(73) Assignees: NATIONAL CEREBRAL AND CARDIOVASCULAR CENTER, Osaka (JP); SHIMADZU CORPORATION, Kyoto (JP)

(21) Appl. No.: 16/708,543

(22) Filed: Dec. 10, 2019

(30) Foreign Application Priority Data
Dec. 13, 2018 (JP) 2018-233549

(51) Int. Cl. C12Q 1/6883

(52) U.S. Cl. CPC C12Q 1/6883; G01N 2800/2871; C12Q 2600/156

(57) ABSTRACT

Provided is a method for predicting probability of ischemic stroke onset. A method for predicting a risk of ischemic stroke onset, the method comprising: a detection step of detecting presence or absence of a RNF213 p.R4810K gene variant in a sample derived from a test subject who does not develop ischemic stroke; and a determination step of determining whether a probability of ischemic stroke onset of the test subject is high or not, based on the presence or absence of the RNF213 p.R4810K gene variant in the detection step, and gender information of the test subject. A genetic marker for predicting a risk of ischemic stroke onset using gender information, comprising a RNF213 p.R4810K gene variant. A biomarker for predicting a risk of ischemic stroke onset using gender information, comprising a polypeptide encoded by a RNF213 p.R4810K gene variant.

### TECHNICAL FIELD

The present invention pertains to the prediction for risk and age of ischemic stroke onset, and those research field. In particular, the present invention relates to a prediction method for risk of ischemic stroke onset, and a prediction method for risk of ischemic stroke onset using gender information.

### BACKGROUND ART

Non-patent Document 1 discloses that an RNF213 gene variant (c.14576G>A, p.R4859K, rs112735431), which is a susceptibility gene for moyamoya disease, is significantly associated with not only moyamoya disease, but also unilateral moyamoya disease and intracranial major artery stenosis (intracranial stenosis) of atherosclerosis. In contrast, Non-patent Document 1 discloses that the RNF213 gene variant is not significantly associated with cervical carotid artery stenosis, cerebral aneurysm, and intracerebral hemorrhage.

Furthermore, the description of (c.14576G>A, p.R4859K, rs112735431) for the RNF213 gene variant in Non-patent Document 1 is based on the naming by Tohoku University Group. In the present specification, based on the naming of Kyoto University Group, the same RNF213 gene variant describes as p.R4810K variant (c.14429G>A, rs112735431), or simply the p.R4810K variant. Both are due to differences in the counting of amino acids, and are the same variant.

### PRIOR ART DOCUMENT

### Non-Patent Document

Non-Patent Document 1: “RNF213 and Moyamoya syndrome”, Satoru MIYAWAKI, Niche Neuro-Angiology Conference 2016, http://nnac.umin.jp/nnac/NNAC_2016_files/% E5% AE % AE % E8%84%87% E5%85%88% E7%94%9F.pdf

### SUMMARY OF THE INVENTION

### Problems to be Solved by the Invention

An object of the present invention is to provide a method for predicting probability of ischemic stroke onset.

### Means for Solving the Problems

Brain infarction is classified into three types: atherothrombotic brain infarction, lacunar infarction, and embolism.

As a result of the investigation by the present inventors, it was found that a RNF213 p.R4810K variant significantly associates with atherothrombotic brain infarction. As such, provided is a method (or, a supplemental method) for predicting a risk or age of ischemic stroke onset of a test subject who has not developed ischemic stroke, by detecting presence or absence of the RNF213 p.R4810K variant in a sample derived from the test subject.

The present invention includes the following aspects.

(1) A method for predicting a risk of ischemic stroke onset of a test subject who does not develop ischemic stroke, the method comprising:
a detection step of detecting presence or absence of a RNF213 p.R4810K gene variant in a sample derived from a test subject who does not develop ischemic stroke; and
a determination step of determining whether a probability of ischemic stroke onset of the test subject is high or not, based on the presence or absence of the RNF213 p.R4810K gene variant in the detection step, and gender information of the test subject.
   (2) The method according to (1), wherein when the presence of the RNF213 p.R4810K gene variant is detected in the detection step,
in the determination step, the risk of ischemic stroke onset of the test subject is determined to be high, compared with a person without the RNF213 p.R4810K gene variant.
   (3) The method according to (1) or (2), wherein when the presence of the RNF213 p.R4810K gene variant is detected in the detection step,
in the determination step, the risk of ischemic stroke onset of the test subject is determined to be higher when the test subject is a woman than when the test subject is a man.
   (4) A genetic marker for predicting a risk of ischemic stroke onset using gender information, comprising a RNF213 p.R4810K gene variant.
   (5) A biomarker for predicting a risk of ischemic stroke onset using gender information, comprising a polypeptide encoded by a RNF213 p.R4810K gene variant.

### Effects of the Invention

According to the knowledge of the present invention, it becomes possible to predict a risk of ischemic stroke onset of a test subject who does not develop ischemic stroke, and to take prevention and measures on the test subject, from an early stage.

### BRIEF DESCRIPTION OF DRAWINGS

FIG. 1 shows a flow diagram of the patient selection procedure in Example 1.

FIG. 2 is a box-and-whisker plot concerning the mean age of stroke onset with and without the RNF213 p.R4810K variant in Example 1.

FIG. 3 is a bar graph showing the sex-specific differences in the carrier frequency of the RNF213 p.R4810K variant among stroke subtypes in Example 1. The left bar indicates men, and the right bar indicates women.

FIG. 4 shows a flow diagram of the patient selection procedure in Example 2.

FIG. 5 is pie charts showing the prevalence of the RNF213 p.R4810K variant. The left chart is the pie chart concerning 70 patients in total in Example 2, and the right chart is the pie chart concerning general population as previously reported.

FIG. 6 is pie charts showing the difference of the prevalence of the RNF213 p.R4810K variant between gender in Example 2. The left chart is the pie chart concerning male (N=44), and the right chart is the pie chart concerning female (N=26).

FIG. 7 is pie charts showing the difference of the prevalence of the RNF213 p.R4810K variant between patients with and without M1/A1 stenosis in Example 2. The left chart is the pie chart concerning the patients with the stenosis (N=48), and the right chart is the pie chart concerning the patients without the stenosis (N=22).

FIG. 8 is image data of the representative case with the RNF213 p.R4810K variant in Example 2 (Case 6 in Table 6). (A) is an MRI diffusion weighted image (DWI), (B) is an MRA image, and (C) is an MRA image at 5 years after the stroke onset.

### MODES FOR CARRYING OUT THE INVENTION

RNF213 (Ring finger protein 213) (GenBank accession number NM 001256071.1) has recently been identified as a disease susceptibility gene for moyamoya disease, and exists on human chromosome region 17q25.3.

A RNF213 p.R4810K gene variant is a Single Nucleotide Polymorphism (SNP) of 73097 G>A on a nucleotide sequence represented by SEQ ID NO.: 2. The RNF213 p.R4810K is known as a moyamoya disease susceptibility polymorphism as shown in the above mentioned prior art document.

As a result of the investigation by the present inventors, it was found that the RNF213 p.R4810K variant increases a risk of ischemic stroke (or, brain infarction). In the present specification, ischemic stroke is synonymous with brain infarction.

Ischemic stroke is one of causes of early death in recent years, and a major cause of disability and early death especially in Asia. However, a genetic association of an ischemic stroke remains unknown. Moyamoya disease, a cerebrovascular disease which is mainly found in East Asia, is associated with a RING finger protein 213 (RNF213) susceptibility gene. Dysregulation of the RNF213 disturbs cerebral perfusion in the mouse brain. The present inventors, therefore, hypothesized that the RNF213 plays a more common role in ischemic stroke, and investigated associations between a p.R4810K variant of RNF213 gene which is the most common risk of moyamoya disease, and ischemic stroke and its subtypes.

A RNF213 p.R4810K gene variant is a Single Nucleotide Polymorphism (SNP) of 73097 G>A on a nucleotide sequence represented by SEQ ID NO.: 2, and is possible to be a gene marker for predicting or assisting in predicting a risk of brain infarction.

SEQ ID NO.: 2 is a partial nucleotide sequence of human chromosome 17 DNA containing the Mysterin gene and genes in the peripheral region thereof [FLJ3520, NPTX1, CARD14, and Raptor (KIAA1303)], and corresponds to the 43560001-43795000th nucleotide of Contig # NT010783.15 registered in NCBI.

In the nucleotide sequence represented by SEQ ID NO.: 2, in addition to the 73097th SNP, which is G or A (abbreviated as 73097 G>A in the present specification), the 4766th SNP, which is T or C (4766 T>C), the 120764th SNP, which is G or A (120764 G>A), the 152917th SNP, which is G or A (152917 G>A), and the 232102th SNP, which is G or A (232102 G>A) may exist.

In the present specification, a position of a SNP is described based on a position of a nucleotide in the nucleotide sequence represented by SEQ ID NO: 2. For example, “the 73097th SNP” means a SNP of the 73097th nucleotide in the nucleotide sequence represented by SEQ ID NO: 2. When described as “73097 G>A” and the like, a base of the main allele (in this case, G) is described before a symbol of “>”, and a base of the minor allele (in this case, A) is described after the symbol of “>”.

In the present specification, a nucleotide sequence is described as a DNA sequence, unless otherwise noted. However, when a polynucleotide is RNA, thymine (T) is appropriately replaced with uracil (U).

In the present invention, a polynucleotide may contain any additional sequence in addition to the continuous partial sequence of the nucleotide sequence represented by SEQ ID NO: 2 or its complementary sequence.

In the present invention, a polynucleotide is preferably isolated or purified.

In the detection step of the above-mentioned method of the present invention, a SNP of 73097 G>A is detected in a sample derived from a test subject.

A human race is not particularly limited, but is preferably East Asian (Mongoloid).

Here, a race is a group of Homo sapiens species that can be distinguished as a specific subgroup. A race has a unique and distinguishable combination of genes and is identified by features (both mental and physical) created by the combination of genes. Members of the same race share a common genetic ancestor and, as a result, share a similar gene combination. Consequently, the members of the same race share a distinct genetic feature.

For example, a genetic relationship is examined based on information of 23 kinds of genes of the main human population in the world, and the main human population in the world is classified into five types: African (Negroid), Caucasoid (Caucasian), Oceanian (Australoid), East Asian (Mongoloid) and Native American.

The East Asian means a person who has any of Japanese, Korean, Chinese, Taiwanese and Mongolian origin. The East Asian is preferably a Japanese, Korean, or Chinese.

A person skilled in the art can easily specify a race of an individual based on information about physical characteristics, a country of birth, and an origin of an ancestor of the individual, and the like.

As the living body-derived sample used in the above-mentionedmethod, any tissue, cell, body fluid or the like capable of collecting genomic DNA can be used. From the viewpoint of easy availability and low invasiveness, hair, nails, skin, mucosa, blood, plasma, serum, saliva, and the like are preferably used.

Methods for detecting SNPs are well known in the art. For example, an RFLP (Restriction Fragment Length Polymorphism) method, PCR-SSCP (Single Strand DNA Conformation Polymorphism analysis) method, ASO (Allele Specific Oligonucleotide) hybridization method, sequencing method, ARMS (Amplification Refracting Mutation System) method, and Denaturing Gradient Gel Electrophoresis method, RNAseA cleavage method, DOL (Dye-labeled Oligonucleotide Ligation) method, TaqMan PCR method, primer extension method, invader method, and the like can be used.

Further, a polypeptide encoded by a RNF213 p.R4810K gene is a polypeptide containing the amino acid sequence represented by SEQ ID NO: 1, wherein the arginine at the 4810th position in the amino acid sequence represented by SEQ ID NO: 1 is substituted with lysine, and is possible to be a biomarker for predicting a risk of brain infraction onset.

That is, as described above, a mutation of RNF213 p.R4810K SNP is accompanied by an amino acid substitution (arginine→lysine) of the 4810th amino acid of the human Mysterin.

Accordingly, it becomes possible to predict or assist in predicting a risk of brain infarction, by isolating a Mysterin polypeptide from a test subject and identifying the 4810th amino acid of the Mysterin polypeptide.

### EXAMPLES

Hereinafter, the present invention will be described specifically with reference to examples, but is not limited to these examples.

### Example 1: Association Between Atherothrombotic Brain Infarction and RNF213 p.R4810K Variant

Although stroke mortality has decreased in the past decades¹⁾, stroke remains the second leading cause of early death and a major cause of disability worldwide²⁾. In Southeast and East Asia, representing 31% of world's population, stroke is the leading cause of early death and the incidence and prevalence of stroke are steadily increasing^{1,2)}. Epidemiological studies suggest substantial geographic and racial differences in stroke subtypes^{3,4)}. Cardioembolism is a common etiological subtype of ischemic stroke in Western countries⁵⁾, whereas large-artery atherosclerosis due to intracranial artery stenosis is the predominant etiology in most Asian countries⁶⁾. Differences in environmental risk factors and genetic backgrounds are considered major reasons for the high prevalence of large-artery atherosclerosis in Asia. Recently, a large multi-ancestry genome-wide association meta-analysis identified 32 loci associated with stroke and stroke subtypes⁷⁾. The study tested about 8 million single nucleotide polymorphisms (SNPs); however, the analysis excluded region-specific rare alleles, with less than 0.01 (<0.01) of minor-allele frequency (MAF) and Asian-specific genetic determinants of ischemic stroke remain unknown.

A ring finger protein 213 gene (RNF213) on 17q25.3 has been identified as a susceptibility gene for moyamoya disease^{8,9)}. A RNF213 p.R4810K variant (c.14429G>A, rs112735431) has been detected in more than 80% of patients with moyamoya disease, while the allele carrier frequency in healthy subjects in East Asia is around 2%^{8,9)}. RNF213 encodes a 591 kDa protein, functioning both as an AAA+ ATPase and an E3 ligase¹²), and has been associated with the development of intracranial steno-occlusive lesions and compensatory adaptation to lowered cerebral blood flow^{13,14)}. Recently, two single center studies reported on a high (20-25%) frequency of RNF213 p.R4810K variant in East Asian non-moyamoya patients with intracranial internal carotid artery stenosis or proximal middle cerebral artery stenosis^{10,15)}. The present inventors thus hypothesized this genetic variant can be associated with overall ischemic stroke in Asia. Here, the present inventors analyzed patients with acute ischemic stroke who participated in a study of National Cerebral and Cardiovascular Center (NCVC), and examined the association of the RNF213 p.R4810K variant with ischemic stroke and its subtypes.

### (Method)

### (Study Design and Participants)

In a case-control study, the present inventors used a single hospital-based population (NCVC biobank) in National Cerebral and Cardiovascular Center (NCVC), which includes extensive clinical and radiological data for all patients. Written informed consent was obtained from all study participants and the study was approved by the ethics committee of National Cerebral and Cardiovascular Center (NCVC).

### (Study)

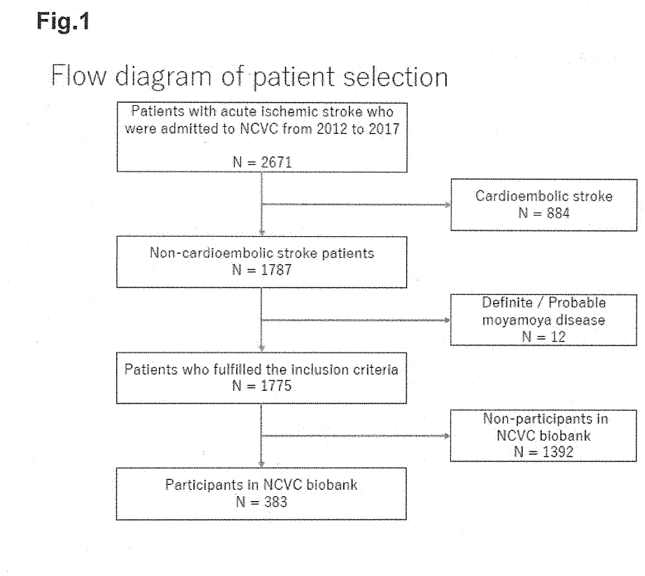

Participants in the present study were recruited at the National Cerebral and Cardiovascular Center (NCVC), a 600-bed tertiary referral center specialized for stroke and cardiovascular diseases located in Kansai District, Osaka. A total of 383 Japanese patients with non-cardioembolic stroke (large-artery atherosclerosis, small-vessel occlusion, and others) were included in the present study, who were admitted from June 2012 to May 2017 and had signed a comprehensive consent form at the NCVC. Patients diagnosed with cardioembolic stroke, or definite/probable moyamoya disease, according to the criteria of the Japanese Research Committee on Moyamoya Disease of the Japanese Ministry of Health, Labor, and Welfare¹⁷⁾, were excluded. A flow diagram of the patient selection procedure is detailed in FIG. 1.

Demographic data, atherosclerotic risk factors, radiological findings, and medical histories were derived from a prospectively-collected database of stroke patients. Stroke subtypes were classified according to the Trial of Org 10172 in Acute Stroke Treatment (TOAST) criteria¹⁸⁾. Cerebrovascular lesions were identified by magnetic resonance imaging (MRI) and MR angiography, or CT and CT angiography. Anterior circulation stenosis was defined as a diameter reduction of over 50% (>50%) in an intracranial internal carotid artery, middle cerebral artery, or anterior cerebral artery, according to the Warfarin-Aspirin Symptomatic Intracranial Disease (WASID) study criteria¹⁹⁾. Similarly, posterior circulation stenosis was defined according to the above criteria in an intracranial vertebral, basilar, or posterior cerebral, artery.

Control subjects were recruited in the Kansai District, Japan, between 2007 and 2015. Among 1,027 potential controls, 16 subjects who had a history of cerebral infarction or moyamoya disease were excluded; 1,011 control subjects were ultimately selected. Venous blood samples were collected on admission and stored at −80° C. until analysis. Genotyping of p.R4810K was performed using TaqMan SNP Assays (Applied Biosystems, Foster City, Calif.) and a 7300/7500 Real-Time PCR System (Applied Biosystems, Foster City, Calif.), as described previously⁸⁾.

SNP genotyping was performed at the RIKEN Center for Integrative Medical Sciences using either a combination analysis of Illumina HumanOmniExpress BeadChip and HumanExome BeadChip, or Illumina Human OmniExpressExome BeadChip analysis. Haplotype phasing and genotype imputation was performed using filtered samples with genotype data, which passed quality control criteria, including success rate and the Hardy-Weinberg equilibrium test. A genome-wide association study (GWAS) was performed using imputed allele dosages and fitted to logistic regression model with additive genetic model. The present inventors incorporated 10 principal components, age, and gender as covariates. Details were described previously⁷⁾.

### (Statistical Analysis)

Continuous variables were expressed as mean±SD and compared using Student's t-test. Categorical variables were expressed as numbers and percentages and compared using chi-square test and two-tailed Fisher's exact test, as appropriate. The present inventors tested the associations between the RNF213 variant and the risk of ischemic stroke under the assumption of a dominant model in the present study because of an insufficient number of homozygotes. The present inventors also examined the associations under a log-additive model for meta-analysis to compare with replication studies using imputation methods. A multiple logistic regression model was used to calculate the odds ratios (OR) and 95% confidence intervals (95% CI) for each stroke subtype after controlling simultaneously for potential confounders. Variables considered in the models were age (continuous), sex, hypertension, dyslipidemia, diabetes mellitus, and smoking. All analyses were performed using JMP Pro 12.2 software (SAS Institute Inc., Cary, N.C.).

Probability values were 2-tailed, and p<0.05 was considered significant. Meta-analysis was performed with the Review Manager (RevMan) 5.3 software (The Nordic Cochrane Centre, Copenhagen, Denmark) using the inverse variance fixed effects model, with p<5×10⁻⁸ considered genome-wide significant.

### (Results)

The present study specifically enrolled non-cardioembolic stroke patients. Among 1,775 non-cardioembolic stroke patients who fulfilled the inclusion criteria, 383 patients (21.6%) agreed to participate in NCVC biobank and were analyzed in the present study. Patient characteristics of both participants and non-participants in NCVC biobank are shown in Table 1. Baseline characteristics of the present study participants are shown in Table 1.

TABLE 1

| | | Non- | |
|---|---|---|---|
| | | cardioembolic | |
| | Control | stroke patients | |
| | (N = 1011) | (N = 383) | P value |
| Age, yr | 63.1 ± 15.2 | 68.5 ± 13.5 | <0.0001 |
| Female | 433 (42.8%) | 110 (28.7%) | <0.0001 |
| Hypertension | 266 (26.3%) | 314 (82.0%) | <0.0001 |
| Diabetes mellitus | 81 (8.0%) | 116 (30.3%) | <0.0001 |
| Dyslipidemia | 112 (11.1%) | 261 (68.2%) | <0.0001 |
| Smoking incidence | 307 (30.4%) | 231 (60.5%) | <0.0001 |
| Extracranial ICA stenosis | — | 46 (12.0%) | |
| Intracranial artery stenosis | | | |
| Anterior circulation stenosis | — | 111 (29.0%) | |
| Posterior circulation stenosis | — | 58 (15.1%) | |
| Stroke subtypes | | | |
| Large-artery atherosclerosis | — | 131 (34.2%) | |
| Small-vessel occlusion | — | 132 (34.5%) | |
| Others | — | 120 (31.3%) | |
| RNF213 p.R4810K genotype | | | 0.0044 |
| GG | 990 (97.2%) | 363 (94.8%) | |
| GA | 20 (2.0%) | 20 (5.2%) | |
| AA | 1 (0.1%) | 0 (0%) | |

Baseline characteristics and prevalence of RNF213 p.R4810K variant of participants in the primary study. ICA indicates internal carotid artery.

The RNF213 p.R4810K variant was found in 5.2% of non-cardioembolic stroke patients and in 2.1% of healthy controls (crude OR 2.60, 95% CI 1.39-4.85, p=0.0019). After adjusting for age, sex, and atherosclerotic risk factors, the association of RNF213 p.R4810K variant with non-cardioembolic stroke remained significant (adjusted OR 3.90, 95% CI 1.62-9.24, p=0.0026). Large-artery atherosclerosis was significantly associated with the variant (crude OR 5.19, 95% CI 2.53-10.64, p=2.6×10⁻⁶; adjusted OR 11.45, 95% CI 3.46-36.17, p=0.0001), in comparison to control subjects (Table 2).

TABLE 2

| | | All non- | | | |
|---|---|---|---|---|---|
| | | cardio- | Athero- | | |
| | Control | embolic | thrombotic | Lacunar | Others |
| RNF213variant | 21/1011 | 20/383 | 13/131 | 4/132 | 3/120 |
| (whole) | (2.1%) | (5.2%) | (9.9%) | (3.0%) | (2.5%) |
| Crude OR | 1.0 | 2.6 | 5.2 | 1.5 | 1.2 |
| (95% CI) | | (1.4-4.8) | (2.5-10.6) | (0.5-4.4) | (0.4-4.1) |
| Adjusted OR | 1.0 | 3.9 | 11.5 | 1.3 | 2.1 |
| (95% CI) | | (1.6-9.3) | (3.4-36.2) | (0.3-5.4) | (0.5-8.4) |
| P value | | 0.003 | <0.001 | 0.71 | 0.29 |

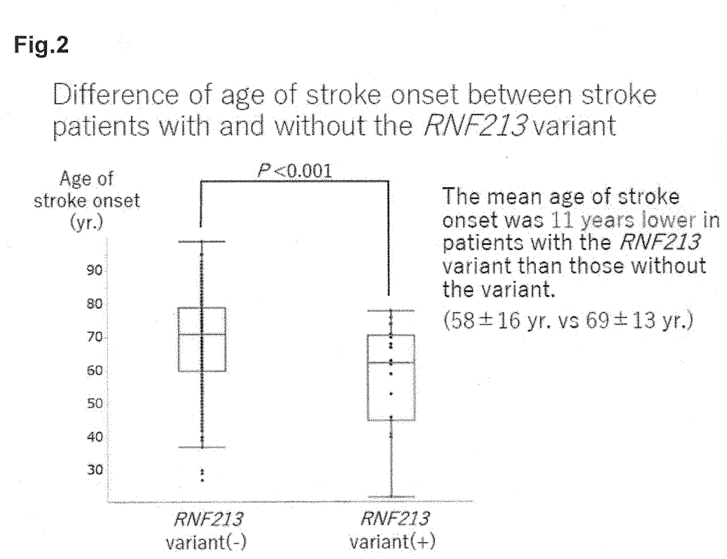

In comparison between stroke patients with and without the RNF213 p.R4810K variant, the mean age of stroke onset was 11 years lower in the RNF213 variant than non-carriers (58.1±15.5 years vs. 69.1±13.2 years, p=0.0003) (FIG. 2). FIG. 2 is a box-and-whisker plot concerning the mean age of stroke onset with and without the RNF213 p.R4810K variant.

The RNF213 variant carriers included more women (55.0% vs. 27.3%, p=0.011), and showed greater frequency of intracranial anterior circulation stenosis (60.0% vs 27.3%, p=0.004) and large-artery atherosclerosis (65.0% vs 32.5%, p=0.012) than non-carriers. The prevalence of extracranial internal carotid artery, and posterior circulation stenosis, and conventional atherosclerotic risk factors, such as hypertension, diabetes mellitus, dyslipidemia, and incidence of smoking, did not differ between variant carriers and non-carriers. The above results are summarized in Table 3.

TABLE 3

| | p.R4810K | p.R4810K | | |
|---|---|---|---|---|
| | variant (+) | variant (−) | OR | |
| | (N = 20) | (N = 363) | (95% CI) | P value |
| Age (yr.) | 58.1 ± 15.5 | 69.1 ± 13.2 | | <0.001 |
| Female | 11 (55%) | 99 (27%) | 3.3 (1.2-8.1) | 0.01 |
| Hypertension | 14 (70%) | 300 (83%) | 0.5 (0.2-1.3) | 0.23 |
| Diabetes mellitus | 5 (25%) | 111 (30%) | 0.8 (0.3-2.1) | 0.80 |
| Dyslipidemia | 12 (60%) | 249 (69%) | 0.7 (0.3-1.7) | 0.46 |
| Smoking incidence | 11 (55%) | 220 (61%) | 0.8 (0.3-2.0) | 0.64 |
| Extracranial ICA | 3 (15%) | 43 (12%) | 1.3 (0.4-4.7) | 0.72 |
| stenosis | | | | |
| Intracranial | | | | |
| artery stenosis | | | | |
| Anterior circulation | 12 (60%) | 99 (27%) | 4.0 (1.6-10.1) | 0.004 |
| stenosis | | | | |
| Posterior circulation | 3 (15%) | 55 (15%) | 1.0 (0.3-3.5) | 1.00 |
| stenosis | | | | |
| Stroke Subtypes | | | | 0.01 |
| Atherothrombotic | 13 (65%) | 118 (33%) | | |
| Lacunar | 4 (20%) | 128 (35%) | | |
| Others | 3 (15%) | 117 (32%) | | |

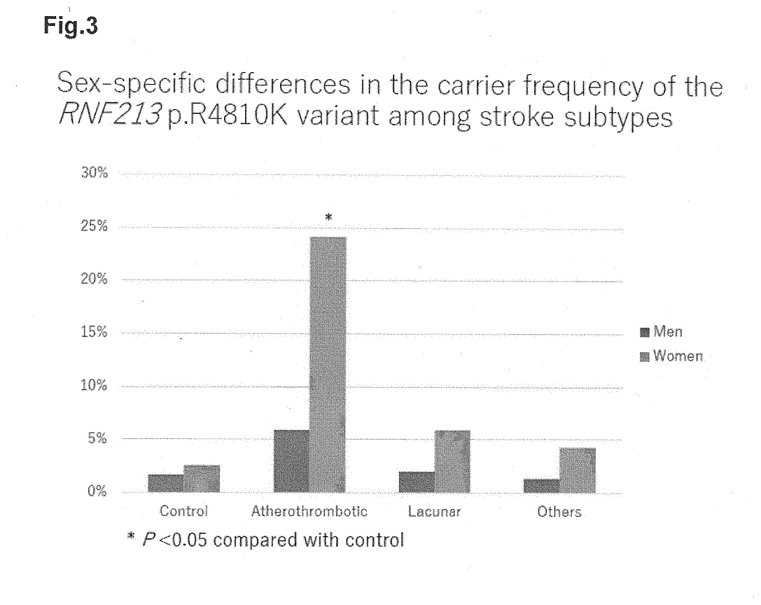

Details of the carrier frequency of the RNF213p.R4810K variant stratified by sex in each stroke subtype are shown in Table 4. FIG. 3 shows the sex-specific differences in the carrier frequency of the RNF213 p.R4810K variant among stroke subtypes in a bar graph. The left bar indicates men, and the right bar indicates women.

TABLE 4

| | | All non- | | | |
|---|---|---|---|---|---|
| | Control | cardioembolic | Atherothrombotic | Lacunar | Others |
| RNF213variant | 21/1011 | 20/383 | 13/131 | 4/132 | 3/120 |
| (whole) | (2.1%) | (5.2%) | (9.9%) | (3.0%) | (2.5%) |
| Crude OR (95% CI) | 1.0 | 2.6 (1.4-4.8) | 5.2 (2.5-10.6) | 1.5 (0.5-4.4) | 1.2 (0.4-4.1) |
| Adjusted OR (95% CI) | 1.0 | 3.9 (1.6-9.3) | 11.5 (3.4-36.2) | 1.3 (0.3-5.4) | 2.1 (0.5-8.4) |
| P value | | 0.003 | <0.001 | 0.71 | 0.29 |
| RNF213variant | 10/578 | 9/273 | 6/102 | 2/98 | 1/73 |
| (men) | (1.7%) | (3.3%) | (5.9%) | (2.0%) | (1.4%) |
| Adjusted OR for men | 1.0 | 1.3 (0.3-4.6) | 1.9 (0.3-10.2) | 0.56 (0.1-3.8) | 0.9 (0.1-9.3) |
| (95% CI) | | | | | |
| P value | | 0.74 | 0.47 | 0.55 | 0.93 |
| RNF213variant | 11/433 | 11/110 | 7/29 | 2/34 | 2/47 |
| (women) | (2.5%) | (10.0%) | (24.1%) | (5.9%) | (4.3%) |
| Adjusted OR for women | 1.0 | 9.8 (3.1-30.9) | 58.0 (10.3-327.3) | 5.7 (0.7-37.3) | 4.4 (0.8-24.9) |
| (95% CI) | | | | | |
| P value | | <0.001 | <0.001 | 0.11 | 0.09 |

As shown in Table 4, in both men and women, the RNF213p.R4810K variant was significantly associated with all non-cardioembolic stroke (all non-cardioembolic) and atherosclerosis (atherothrombotic), respectively. More preferably, the significant association was observed between women with the RNF213p.R4810K variant and all non-cardioembolic stroke (adjusted OR 9.8, 95% CI 3.1-30.9, p<0.001), and between the RNF213p.R4810K variant and atherosclerosis (adjusted OR 58.0, 95% CI 10.3-327.3, p<0.001), respectively.

As such, the present study reveals that the mean age of stroke onset was 11 years younger in the RNF213p.R4810K variant carriers than in non-carriers, and that the carrier frequency of the RNF213p.R4810K variant is significantly higher in acute non-cardioembolic stroke patients, especially in female patients with atherothrombotic brain infarction.

Consequently, the RNF213p.R4810K variant is a genetic risk factor for non-cardioembolic stroke, and can be more preferable genetic risk factor for atherothrombotic brain infarction by combining with gender information.

### Example 2: Association Between Juvenile-Onset Stroke with Intracranial Arterial Stenosis and RNF213 p.R4810K Variant

RNF213 is identified as a susceptibility gene for moyamoya disease, and 90% or more of Japanese patients with moyamoya disease have the RNF213 p.R4810K variant. The RNF213 is considered to play an important role in the regulation of vascular endothelial function and angiogenesis, but the mechanism of causing specific vascular disorders such as moyamoya disease has not been fully elucidated. Alternatively, recent studies have reported that 2-3% of healthy Asian adults including Japanese have the RNF213 p.R4810K variant, and 20-25% of patients with intracranial arterial stenosis, who do not meet the diagnostic criteria for moyamoya disease, have the gene variant. It has been suggested that the gene variant may also be a risk factor in cerebrovascular disorders other than moyamoya disease.

An object of the present study was to examine the prevalence of the RNF213 p.R4810K variant in patients with juvenile brain infarction younger than 60 years old, and to clarify the association between the gene variant and juvenile brain infarction. Here, in the term “juvenile brain infarction” in the present specification, “juvenile” generally means up to the age of 60 (before the 60th birthday).

### (Study Design and Subjects)

The present study is a single-center cross-sectional study subject to the patients enrolled in the biobank of the National Cerebral and Cardiovascular Center (NCVC). A total of 70 enrolled patients who developed non-cardioembolic stroke or transient ischemic attacks (TIA) at 20 or more and less than 60 years old with Intracranial Arterial Stenosis, were applied. Cases diagnosed with cardioembolic stroke or moyamoya disease were excluded. Written informed consent was obtained from all subjects and the study was approved by the ethics committee of National Cerebral and Cardiovascular Center (NCVC).

### (Selection Criteria)

Selection criteria for subjects were as follows.

[1] Patients who consented to the biobank project of the National Cerebral and Cardiovascular Center (NCVC).
[2] Patients who developed ischemic stroke or transient ischemic attacks at 20 or more and less than 60 years old.
[3] Patients with 50% or more (in diameter) of stenosis or occlusion in at least one of the following intracranial arteries.
Intracranial internal carotid artery (ICA),
middle cerebral artery M1-2 segment,
anterior cerebral artery A1-2 segment,
posterior circulation stenosis P1-2 segment, and
basilar artery.

### (Exclusion Criteria)

Patients with cardioembolic stroke, and
patients with moyamoya disease.

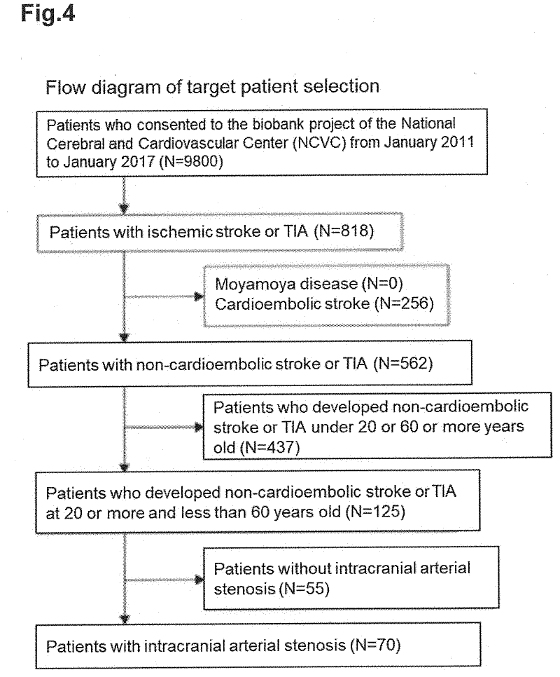

A flow diagram of the patient selection procedure is detailed in FIG. 4.

### (Method)

Brain infarction subtypes were classified according to the Trial of Org 10172 in Acute Stroke Treatment (TOAST) criteria.

The presence or absence of the RNF213 p.R4810K variant was compared with atherosclerotic risk factors, family history of moyamoya disease, and characteristics of intracranial stenosis.

Venous blood samples were used as DNA samples. After the DNA samples were anonymized, genotyping of p.R4810K was performed using TaqMan SNP Assays (Applied Biosystems, Foster City, Calif.) and a 7300/7500 Real-Time PCR System (Applied Biosystems, Foster City, Calif.) in Department of Health and Environmental Sciences, Kyoto University Graduate School of Medicine.

### (Statistical Analysis)

Continuous variables were expressed as mean±SD and compared using Student's t-test. Categorical variables were expressed as numbers and percentages and compared using chi-square test and two-tailed Fisher's exact test. The odds ratios (OR) and 95% confidence intervals (95% CI) for each stroke subtype were calculated. Variables considered in the models were age (continuous), sex, hypertension, dyslipidemia, type 2 diabetes mellitus, and smoking habit. All analyses were performed using JMP version 11.2.0 software (SAS Institute Inc., Cary, N.C.). Probability values were 2-tailed, and p<0.05 was considered significant.

### (Results)

The association between backgrounds of 70 patients in total and the RNF213 p.R4810K variant is shown in Table 5. In Table 5, categorical variables were shown as number of patients (%). The characteristics of 17 patients who are the RNF213 p.R4810K variant carriers are shown in Table 6.

TABLE 5
Assoation between patients backgrounds and RNF213 p.R4810k

| | Patients | Patients | | | |
|---|---|---|---|---|---|
| | with | without | | | |
| | p.R4810K | p.R4810K | | | 95% |
| | variant | variant | p | Odds | confidence |
| | (N = 17) | (N = 53) | value | ratio | index |
| Age of stroke onset | 44.4 ± 2.3 | 46.6 ± 1.3 | 0.21 | | |
| (mean ± SD) | | | | | |
| Female | 10 (59) | 16 (30) | 0.04 | 3.3 | 1.1-10.2 |
| Hypertension | 9 (53) | 33 (62) | 0.57 | 0.7 | 0.2-2.1 |
| Dyslipidemia | 7 (41) | 34 (64) | 0.16 | 0.4 | 0.1-1.2 |
| Type 2 Diabetes | 2 (12) | 13 (25) | 0.33 | 0.4 | 0.08-2.0 |
| mellitus | | | | | |
| Family history of | 3 (18) | 0 (0) | 0.01 | 25.8 | 1.3-529 |
| moyamoya disease | | | | | |
| Smoking habit | 8 (47) | 26 (49) | 1 | 0.9 | 0.3-2.7 |
| Anterior circulation | 17 (100) | 46 (87) | 0.18 | 5.7 | 0.3-104 |
| stenosis | | | | | |
| M1 or A1 stenosis | 17 (100) | 31 (58) | <0.01 | 25.0 | 1.4-438 |
| Multiple M1/A1 | 5 (29) | 4 (8) | 0.03 | 5.1 | 1.2-22 |
| stenoses | | | | | |
| Posterior circulation | 1 (6) | 10 (19) | 0.27 | 0.3 | 0.03-2.3 |
| stenosis | | | | | |

Categorical variables were shown as number of patients (%).

TABLE 6
Characteristics of 17 patients with RNF213 p.R4810K variant

| | | | Family | | | | | | |
|---|---|---|---|---|---|---|---|---|---|
| | | | history of | | | | | TOAST | |
| | | | moyamoya | | | Diabetes | Smoking | Category/ | Intracranial stenosis |
| Case | Age | Gender | disease | Hypertension | Dyslipidemia | mellitus | history | TIA | vessels |
| 1 | 22 | female | − | − | − | − | − | UE | Lt M1 |
| 2 | 33 | female | − | − | − | − | + | TIA | Lt M1 |
| 3 | 40 | female | − | + | − | − | − | LAA | Rt M1 |
| 4 | 41 | male | − | − | + | − | + | TIA | Bil A1, Lt P1, Lt P2 |
| 5 | 41 | male | − | + | + | − | + | TIA | Rt M2, Rt A1, Lt A1 |
| 6 | 41 | female | + | − | + | − | − | LAA | Bil ICA, Rt A1, Lt M1 |
| 7 | 42 | male | − | − | + | − | + | TIA | Rt M1 |
| 8 | 42 | female | − | − | − | − | + | LAA | Lt M1 |
| 9 | 45 | female | − | + | + | + | + | LAA | Rt M1, Lt M1 |
| 10 | 45 | female | + | + | − | − | − | UE | Lt ICA, Lt M1, Lt A1 |
| 11 | 46 | female | − | + | − | − | − | LAA | Rt M1 |
| 12 | 47 | male | − | + | + | − | − | LAA | Lt M1 |
| 13 | 51 | female | − | − | − | − | − | LAA | Rt ICA |
| 14 | 53 | male | − | − | − | + | + | LAA | Rt M1 |
| 15 | 54 | female | + | + | − | − | − | TIA | Lt ICA, Lt M1 |
| 16 | 55 | male | − | + | − | − | − | LAA | Rt M1, Lt ICA |
| 17 | 59 | male | − | + | + | − | + | LAA | Lt ICA, Lt M1, Lt M2 |

Bil; bilateral, Rt; right, Lt; left, UE; undetermined etiology, LAA; large artery atherosclerosis.

The descriptions in Table 6 indicate as follows.

Bil; bilateral
Rt; right
Lt; left
UE; undetermined etiology
LAA; large artery atherosclerosis

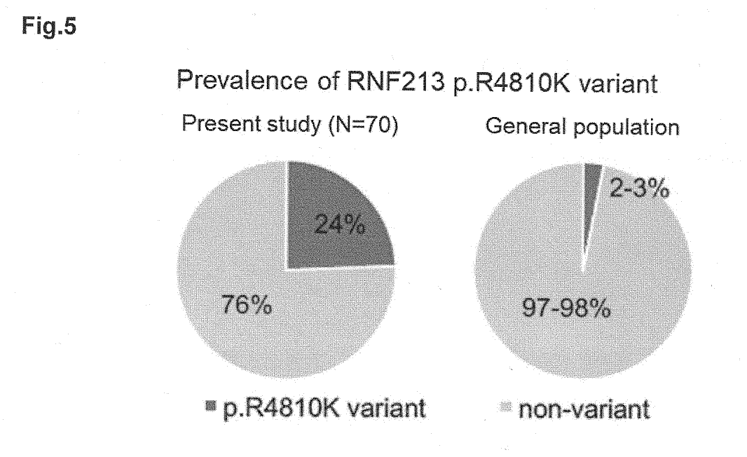

FIG. 5 is pie charts showing the prevalence of the RNF213 p.R4810K variant. The left chart is the pie chart concerning 70 patients in total in the present study, and the right chart is the pie chart concerning general population as previously reported.

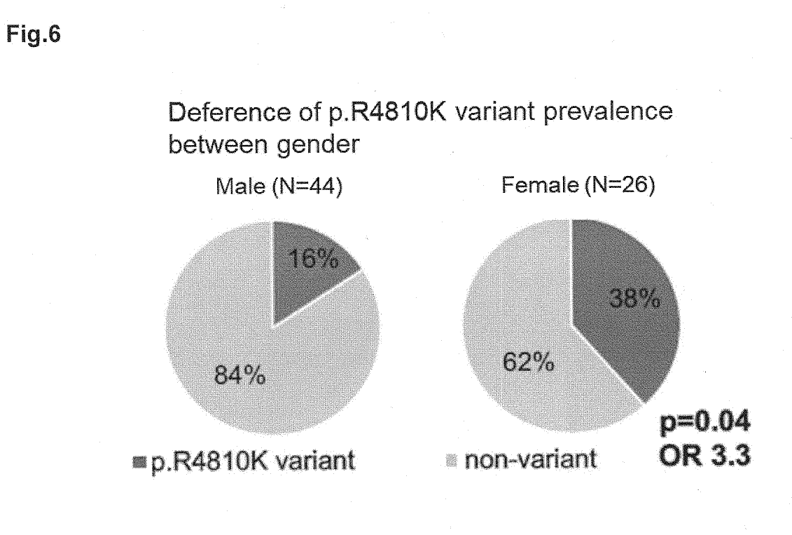

FIG. 6 is pie charts showing the difference of the prevalence of the RNF213 p.R4810K variant between gender in the present study. The left chart is the pie chart concerning male (N=44), and the right chart is the pie chart concerning female (N=26).

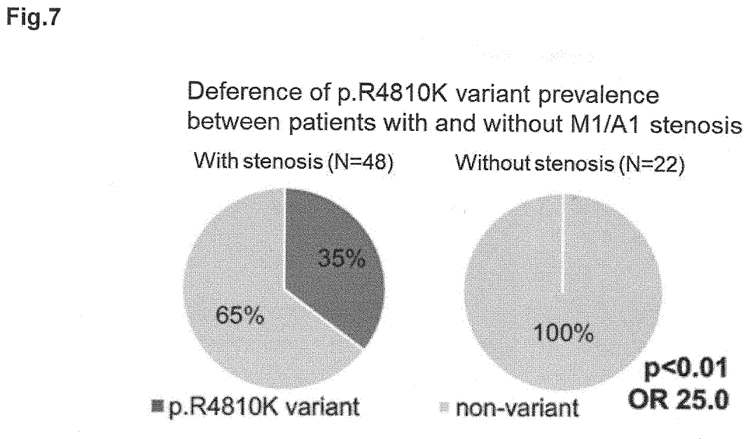

FIG. 7 is pie charts showing the difference of the prevalence of the RNF213 p. R4810K variant between patients with and without M1/A1 stenosis in the present study. The left chart is the pie chart concerning the patients with the stenosis (N=48), and the right chart is the pie chart concerning the patients without the stenosis (N=22).

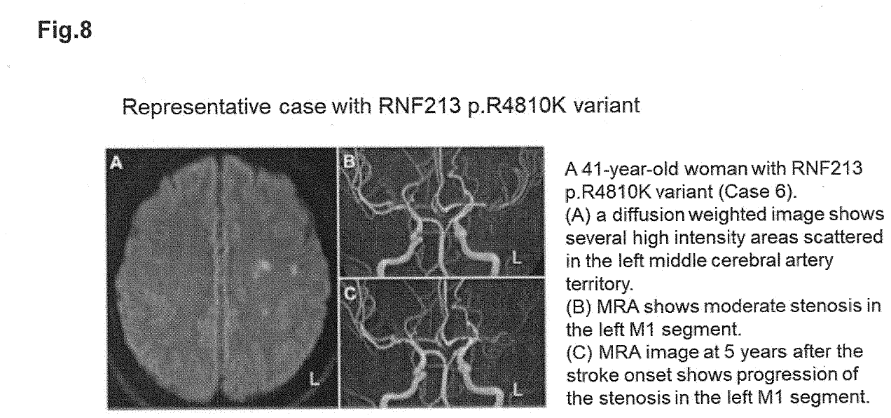

FIG. 8 is image data of the representative case with the RNF213 p.R4810K variant in the present study (Case 6 in Table 6). (A) is an MRI diffusion weighted image (DWI), and shows several high intensity areas scattered in the left middle cerebral artery territory. (B) is an MRA image, and shows moderate stenosis in the left M1 segment. (C) is an MRA image at 5 years after the stroke onset, and shows progression of the stenosis in the left M1 segment.

The RNF213p.R4810K variant was found in 17 patients (24%), and was found more often in women (women 38% vs. men 16%, OR 3.3, 95% CI 1.1-10.2, p=0.045). The variant was identified in 35% of the patients with stenosis in the proximal middle cerebral artery (M1) or the proximal anterior cerebral artery (A1) (OR 25.0, 95% CI 1.4-437.8, p<0.01). In patients with multiple M1 or A1 stenoses, 56% of the patients carried the variant (OR 5.1, 95% CI 1.2-21.9, p=0.033).

### (Discussion)

The present study indicated that the prevalence of the RNF213 p.R4810K variant was high in the patients with cerebral artery stenosis of anterior circulation, especially M1 or A1 stenoses. The high prevalence of the p.R4810K variant was observed in young patients having lower vascular risk compared with geriatric population, and it is possible that the p.R4810K variant associates with brain infraction onset. Further, in the present study that involves the patients with the stenoses in the intracranial anterior or posterior circulation as well as in the distal arteries, the above mentioned gene variant was observed in many of the patients with M1 or A1 stenoses. The above mentioned gene variant was found to be associated with the position of the intracranial stenosis.

### CONCLUSION

From the description above, the RNF213 p.R4810K variant is found to associate with the juvenile-onset non-cardioembolic stroke patients with cerebral artery stenosis of anterior circulation, especially M1 or A1 stenoses.

### REFERENCES

1) Feigin V L, Forouzanfar M H, Krishnamurthi R, Mensah G A, Connor M, Bennett D A, Moran A E, Sacco R L, Anderson L, Truelsen T, O'Donnell M, Venketasubramanian N, Barker-Collo S, Lawes C M M M, Wang W, Shinohara Y, Witt E, Ezzati M, Naghavi M, Murray C, Global Burden of Diseases, Injuries and RFS 2010 (GBD 2010) and the GSEG. Global and regional burden of stroke during 1990-2010: findings from the Global Burden of Disease Study 2010. Lancet. 2014; 383:245-254.
2) GBD 2015 Mortality and Causes of Death Collaborators. Global, regional, and national life expectancy, all-cause mortality, and cause-specific mortality for 249 causes of death, 1980-2015: a systematic analysis for the Global Burden of Disease Study 2015. Lancet. 2016; 388:1459-1544.
3) Bang O Y. Considerations when subtyping ischemic stroke in Asian patients. J Clin Neurol. 2016; 12:129-136.
4) Gulli G, Rutten-Jacobs L C A, Kalra L, Rudd A G, Wolfe C D A, Markus H S. Differences in the distribution of stroke subtypes in a UK black stroke population—final results from the South London Ethnicity and Stroke Study. BMC Med. 2016; 14:77.
5) Kolominsky-Rabas P L, Weber M, Gefeller O, Neundoerfer B, Heuschmann P U. Epidemiology of ischemic stroke subtypes according to TOAST criteria: incidence, recurrence, and long-term survival in ischemic stroke subtypes: a population-based study. Stroke. 2001; 32:2735-2740.
6) Mehndiratta M M, Khan M, Mehndiratta P, Wasay M. Stroke in Asia: geographical variations and temporal trends. J Neurol Neurosurg Psychiatry. 2014; 85:1308-1312.
7) Malik R, Chauhan G, Traylor M, Sargurupremraj M, Okada Y, Mishra A, Rutten-Jacobs L, Giese A-K, van der Laan S W, Gretarsdottir S, Anderson C D, Chong M, Adams H H H, Ago T, Almgren P, Amouyel P, Ay H, Bartz T M, Benavente O R, Bevan S, Boncoraglio G B, Brown R D, Butterworth A S, Carrera C, Carty C L, Chasman D I, Chen W-M, Cole J W, Correa A, Cotlarciuc I, Cruchaga C, Danesh J, de Bakker P I W, DeStefano A L, den Hoed M, Duan Q, Engelter S T, Falcone G J, Gottesman R F, Grewal R P, Gudnason V, Gustafsson S, Haessler J, Harris T B, Hassan A, Havulinna A S, Heckbert S R, Holliday E G, Howard G, Hsu F-C, Hyacinth H I, Ikram M A, Ingelsson E, Irvin M R, Jian X, Jimenez-Conde J, Johnson J A, Jukema J W, Kanai M, Keene K L, Kissela B M, Kleindorfer D O, Kooperberg C, Kubo M, Lange L A, Langefeld C D, Langenberg C, Launer L J, Lee J-M, Lemmens R, Leys D, Lewis C M, LinW-Y, Lindgren A G, Lorentzen E, Magnusson P K, Maguire J, Manichaikul A, McArdle P F, Meschia J F, Mitchell B D, Mosley T H, Nalls M A, Ninomiya T, O'Donnell M J, Psaty B M, Pulit S L, RannikmAe K, Reiner A P, Rexrode K M, Rice K, Rich S S, Ridker P M, Rost N S, Rothwell P M, Rotter J I, Rundek T, Sacco R L, et al. Multiancestry genome-wide association study of 520,000 subjects identifies 32 loci associated with stroke and stroke subtypes. Nat Genet. 2018; 50:524-537.
8) Liu W, Morito D, Takashima S, Mineharu Y, Kobayashi H, Hitomi T, Hashikata H, Matsuura N, Yamazaki S, Toyoda A, Kikuta K, Takagi Y, Ilarada K H, Fujiyama A, Herzig R, Krischek B, Zou L, Kim J E, Kitakaze M, Miyamoto S, Nagata K, Hashimoto N, Koizumi A. Identification of RNF213 as a susceptibility gene for moyamoya disease and its possible role in vascular development. PLoS One. 2011; 6:e22542.
9) Kamada F, Aoki Y, Narisawa A, Abe Y, Komatsuzaki S, Kikuchi A, Kanno J, Niihori T, Ono M, Ishii N, Owada Y, Fujimura M, Mashimo Y, Suzuki Y, Hata A, Tsuchiya S, Tominaga T, Matsubara Y, Kure S. A genome-wide association study identifies RNF213 as the first Moyamoya disease gene. J Hum Genet. 2011; 56:34-40. 10) Miyawaki S, Imai H, Takayanagi S, Mukasa A, Nakatomi H, Saito N. Identification of a genetic variant common to moyamoya disease and intracranial major artery stenosis/occlusion. Stroke. 2012; 43:3371-3374.
11) Miyatake S, Miyake N, Touho H, Nishimura-Tadaki A, Kondo Y, Okada I, Tsurusaki Y, Doi H, Sakai H, Saitsu H, Shimojima K, Yamamoto T, Higurashi M, Kawahara N, Kawauchi H, Nagasaka K, Okamoto N, Mori T, Koyano S, Kuroiwa Y, Taguri M, Morita S, Matsubara Y, Kure S, Matsumoto N. Homozygous c.14576G>A variant of RNF213 predicts early-onset and severe form of moyamoya disease. Neurology. 2012; 78:803-810.
12) Morito D, Nishikawa K, Hoseki J, Kitamura A, Kotani Y, Kiso K, Kinjo M, Fujiyoshi Y, Nagata K. Moyamoya disease-associated protein mysterin/RNF213 is a novel AAA+ ATPase, which dynamically changes its oligomeric state. Sci Rep. 2014; 4:4442.
13) Koizumi A, Kobayashi H, Hitomi T, Harada K H, Habu T, Youssefian S. A new horizon of moyamoya disease and associated health risks explored through RNF213. Environ Health Prev Med. 2016; 21:55-70.
14) Morimoto T, Enmi J, Hattori Y, Iguchi S, Saito S, Harada K H, Okuda H, Mineharu Y, Takagi Y, Youssefian S, Iida H, Miyamoto S, Ihara M, Kobayashi H, Koizumi A. Dysregulation of RNF213 promotes cerebral hypoperfusion. Sci Rep. 2018; 8:3607.
15) Bang O Y, Chung J-W, Cha J, Lee M J, Yeon J Y, Ki C-S, Jeon P, Kim J-S, Hong S C. A Polymorphism in RNF213 Is a Susceptibility Gene for Intracranial Atherosclerosis. PLoS One. 2016; 11:e0156607.
16) O'Donnell M, Xavier D, Diener C, Sacco R, Lisheng L, Zhang H, Pias P, Truelsen T, Chin S L, Rangarajan S, Devilliers L, Damasceno A, Mondo C, Lanas F, AvezumA, Diaz R, Varigos J, Hankey G, Teal P, Kapral M, Ryglewicz D, Czlonkowska A, Skowronska M, Lopez-Jaramillo P, Dans T, Langhorne P, Yusuf S, INTERSTROKE investigators. Rationale and design of INTERSTROKE: a global case-control study of risk factors for stroke. Neuroepidemiology. 2010; 35:36-44.
17) Fukui M. Guidelines for the diagnosis and treatment of spontaneous occlusion of the circle of Willis (“moyamoya” disease). Research Committee on Spontaneous Occlusion of the Circle of Willis (Moyamoya Disease) of the Ministry of Health and Welfare, Japan. Clin Neurol Neurosurg. 1997; 99 Suppl 2:S238-240.
18) Adams H P, Bendixen B H, Kappelle L J, Biller J, Love B B, Gordon D L, Marsh E E. Classification of subtype of acute ischemic stroke. Definitions for use in a multicenter clinical trial. TOAST. Trial of Org 10172 in Acute Stroke Treatment. Stroke. 1993; 24:35-41.
19) Chimowitz M I, Kokkinos J, Strong J, Brown M B, Levine S R, Silliman S, Pessin M S, Weichel E, Sila C A, Furlan A J. The U.S. patent application Ser. No. 16/708,543 Warfarin-Aspirin Symptomatic Intracranial Disease Study. Neurology. 1995; 45:1488-1493.
20) Kim B J, Kim J S. Ischemic stroke subtype classification: an Asian viewpoint. J stroke. 2014; 16:8-17.
21) ParkMG, Shin J H, Lee S W, Park H R, Park K P. RNF213 rs112735431 polymorphism in intracranial artery steno-occlusive disease and moyamoya disease in Koreans. J Neurol Sci. 2017; 375:331-334.
22) Miyawaki S, Imai H, Shimizu M, Yagi S, Ono H, Mukasa A, Nakatomi H, Shimizu T, Saito N. Genetic variant RNF213 c.14576G>A in various phenotypes of intracranial major artery stenosis/occlusion. Stroke. 2013; 44:2894-2897.
23) Kuroda S, Houkin K. Moyamoya disease: current concepts and future perspectives. Lancet Neurol. 2008; 7:1056-1066.
24) Kuriyama S, Kusaka Y, Fujimura M, Wakai K, Tamakoshi A, Hashimoto S, Tsuji I, Inaba Y, Yoshimoto T. Prevalence and clinicoepidemiological features of moyamoya disease in Japan: findings from a nationwide epidemiological survey. Stroke. 2008; 39:42-47.
25) Roy-O'Reilly M, McCullough L D. Sex differences in stroke: the contribution of coagulation. Exp Neurol. 2014; 259:16-27.
26) Williams J E, Chimowitz M I, Cotsonis G A, Lynn M J, Waddy S P, WASID Investigators. Gender differences in outcomes among patients with symptomatic intracranial arterial stenosis. Stroke. 2007; 38:2055-2062.
27) Liu W, Senevirathna S T M L D, Hitomi T, Kobayashi H, Roder C, Herzig R, Kraemer M, Voormolen M H J, CahovA P, Krischek B, Koizumi A. Genomewide association study identifies no major founder variant in Caucasian moyamoya disease. J Genet. 2013; 92:605-609.

1. A method for predicting a risk of ischemic stroke onset, the method comprising:
a detection step of detecting presence or absence of a RNF213 p.R4810K gene variant in a sample derived from a test subject who does not develop ischemic stroke; and
a determination step of determining whether a probability of ischemic stroke onset of the test subject is high or not, based on the presence or absence of the RNF213 p.R4810K gene variant in the detection step, and gender information of the test subject.

2. The method according to claim 1, wherein when the presence of the RNF213 p.R4810K gene variant is detected in the detection step,
in the determination step, the risk of ischemic stroke onset of the test subject is determined to be high, compared with a person without the RNF213 p.R4810K gene variant.

3. The method according to claim 1, wherein when the presence of the RNF213 p.R4810K gene variant is detected in the detection step,
in the determination step, the risk of ischemic stroke onset of the test subject is determined to be higher when the test subject is a woman than when the test subject is a man.

4. A genetic marker for predicting a risk of ischemic stroke onset using gender information, comprising a RNF213 p.R4810K gene variant.

5. A biomarker for predicting a risk of ischemic stroke onset using gender information, comprising a polypeptide encoded by a RNF213 p.R4810K gene variant.

6. The method according to claim 1, wherein the ischemic stroke is atherothrombotic brain infarction.

7. The genetic marker according to claim 4, wherein the ischemic stroke is atherothrombotic brain infarction.

8. The biomarker according to claim 5, wherein the ischemic stroke is atherothrombotic brain infarction.
